(19) United States
(12) Patent Application Publication
(10) Pub. No.: US 2025/0101524 A1
(43) Pub. Date: Mar. 27, 2025

(54) COMPOSITIONS AND METHODS FOR CELL-FREE DNA EPIGENETIC GASTROINTESTINAL CANCER DETECTION AND TREATMENT

(71) Applicants: City of Hope, Duarte, CA (US); Baylor Research Institute d/b/a Baylor Scott & White Research Institute, Dallas, TX (US); Baylor College of Medicine, Houston, TX (US); The Regents of the University of California, Oakland, CA (US)

(72) Inventors: Ajay Goel, La Cañada Flintridge, CA (US); Wei Li, Irvine, CA (US); Jianfeng Xu, Lake Forest, CA (US)

(21) Appl. No.: 18/684,140

(22) PCT Filed: Aug. 17, 2022
(86) PCT No.: PCT/US22/40555

§ 371 Date: Feb. 15, 2024

Related U.S. Application Data
(60) Provisional application No. 63/233,957, filed on Aug. 17, 2021.

(51) Int. Cl. C12Q 1/6886

(52) U.S. Cl. CPC C12Q 1/6886

(57) ABSTRACT

Provided herein are, inter alia, methods of detecting DNA methylation levels in patients at risk of developing a gastrointestinal cancer, methods of diagnosing a patient with a gastrointestinal cancer based on DNA methylation levels, methods of monitoring DNA methylation levels in patients at risk of developing a gastrointestinal cancer, and methods of treating patients having a gastrointestinal cancer.

### CROSS-REFERENCE TO RELATED APPLICATIONS

This application claims priority to U.S. Provisional Application No. 63/233,957, filed Aug. 17, 2021, which is hereby incorporated by reference in its entirety and for all purposes.

### STATEMENT AS TO RIGHTS TO INVENTIONS MADE UNDER FEDERALLY SPONSORED RESEARCH AND DEVELOPMENT

This invention was made with government support under Grant No. CA181572 awarded by the National Institutes of Health. The government has certain rights in the invention.

### BACKGROUND

Despite improved overall survival rates due to the recent advancements in cancer therapies, cancer remains as a second leading cause of mortality world-wide (1). So far, only for colorectal, breast, cervical, lung and prostate cancers, average-risk or asymptomatic population screening is recommended in the United States (2). Population screening in low prevalent cancers is challenging due to the lack of cost-effective diagnostic tools (3).

Circulating tumor DNA (ctDNA) released into the blood stream by tumor cells carry both genetic as well epigenetic signatures of the cell of origin (4). However, the diversity of mutations across cancers and the prevalence of these mutations across large genomic regions makes it challenging to develop mutation-based pan-cancer diagnostic tests (5). DNA methylation changes appear in the earliest phases of cancer development (6, 7). Yet, most studies so far investigated plasma cell-free DNA (cfDNA) methylation patterns in individual cancers for biomarker development (8-10), while few of the recent studies investigated multiple cancers (11, 12). Gastrointestinal (GI) cancers, including colorectal (CRC), esophageal squamous cell carcinoma (ESCC), esophageal adenocarcinoma (EAC), gastric (GC), liver (HCC) and pancreatic ductal adenocarcinoma (PDAC) constitute the second leading cause of cancer-related deaths worldwide; yet there is no blood-based assay for the early detection and population screening of GI cancers. Due to the low prevalence as well as lack of cost-effective screening tools except for CRC (13), most GI cancers are presented at late stage leading to high mortality rate.

### BRIEF SUMMARY

Provided herein are methods of diagnosing cancer in a patient comprising detecting a level of methylated CpG sites within a plurality of gene regions in a DNA sample from the patient, and diagnosing the patient with cancer when the DNA sample has an increased level of methylated CpG sites, relative to a standard control, within the plurality of gene regions.

Provided herein are methods of treating cancer in a patient in need thereof comprising detecting an increased level of methylated CpG sites, relative to a standard control, within a plurality of gene regions in a DNA sample obtained from the patient; and treating the patient for cancer. In embodiments, treating the patient for cancer comprises administering an effective amount of an anti-cancer agent to the patient. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein are methods of monitoring treatment in a patient having cancer or monitoring risk for developing cancer in a patient comprising detecting a level of methylated CpG sites within a plurality of gene regions in a DNA sample from the patient at a first time point; detecting a level of methylated CpG sites within a plurality of gene regions in a DNA sample from the patient at a second time point, wherein the second time point is later than the first time point; and comparing the level of methylated CpG sites at the second time point to the level of methylated CpG sites at the first time point, thereby monitoring treatment or monitoring risk.

Provide herein are methods of detecting a level of DNA methylation in a subject at risk of developing a cancer comprising determining the degree of methylation of CpG sites within a plurality of gene regions in a DNA sample from the subject.

In embodiments of the methods described herein: (i) the cancer is gastrointestinal cancer and the plurality of gene regions comprises at least 50 different gene regions in Table PGI; (ii) the cancer is colorectal cancer and the plurality of gene regions comprises at least 5 different gene regions in Table CRC; (iii) the cancer is hepatocellular carcinoma and the plurality of gene regions comprises at least 5 different gene regions in Table HCC; (iv) the cancer is esophageal squamous cell carcinoma and the plurality of gene regions comprises at least 5 different gene regions in Table ESCC; (v) the cancer is gastric cancer and the plurality of gene regions comprises at least 5 different gene regions in Table GC; (vi) the cancer is esophageal adenocarcinoma and the plurality of gene regions comprises at least 5 different gene regions in Table EAC; (vii) the cancer is pancreatic ductal adenocarcinoma and the plurality of gene regions comprises at least 5 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

Provided herein are methods for preparing a DNA fraction from a subject at risk of developing a gastrointestinal cancer, said method including extracting DNA from a substantially cell-free sample of biological fluid of the subject to obtain extracellular DNA; and determining a level of DNA methylation in a subject at risk according to including any of the methods disclosed herein including embodiments thereof.

These and other embodiments are described in detail herein.

### BRIEF DESCRIPTION OF THE DRAWINGS

FIG. 1 is a study design depicting the tissue discovery and plasma validation of EpiPanGI Dx. Genome-wide 450k tissue DNA methylation analysis across all gastrointestinal (GI) cancers led to the development of GI targeted bisulfite sequencing (gitBS), which is depicted in the circus plot. Subsequently, gitBS is evaluated in cell-free DNA across the GI cancers for the development of differentially methylated regions (DMR) panels which can robustly detect individual GI cancers, pan-gastrointestinal (panGI) and tissue of origin using machine learning models.

FIGS. 2A-2E present exemplary data showing individual GI cancers detection accuracy using informative plasma DMRs identified from gitBS panel. FIG. 2A is a boxplot showing the prediction accuracy of the machine learning model trained for each GI cancer. Samples were randomly partitioned into training set (70%) and test set (30%) for 10 times. DMR calling, feature selection and model training were performed on training sets. Boxplot shows Area Under Curve (AUC) scores of prediction models on test sets for each GI cancer.

FIG. 2B is a boxplot showing the use of informative plasma DMRs from FIG. 2A to predict TCGA (The Cancer Genome Atlas) GI cancer tissues. Boxplot shows AUC scores of 10 independent runs. FIG. 2C shows representative receiver operating characteristic (ROC) curve and AUC scores (10 runs) for the pancreatic ductal adenocarcinoma (PDAC) independent validation set. FIG. 2D is a boxplot showing AUC scores of prediction models on early stage (Stage I-III) plasma samples. Late stage (stage IV) plasma samples (CRC: colorectal cancer, HCC: hepatocellular carcinoma, GC: gastric cancer and PDAC: pancreatic ductal adenocarcinoma) were used for DMR calling, feature selection and model training. Normal plasma samples were randomly split into training sets (70%) and test sets (30%) for 10 times. FIG. 2E is a boxplot showing the use of informative plasma DMRs from FIG. 2D to predict TCGA early stage GI cancer tissues.

FIGS. 3A-3B present exemplary data showing pan-GI cancer detection accuracy using informative plasma DMRs identified from gitBS. In FIG. 3A, plasma samples of each GI cancer were randomly subsampled into training set (70%) and test set (30%) for 10 times. Training sets of all GI cancers were pooled for training pan-GI cancer prediction model. Representative ROC curve and AUC scores for the combined test sets were shown. FIG. 3B shows the use of informative plasma DMRs from FIG. 3A to predict TCGA pan-GI cancer tissues.

FIGS. 4A-4D present exemplary data showing multi GI cancer tissue of origin classification using informative plasma DMRs identified from gitBS. FIG. 4A is a bar graph showing a classification accuracy of the plasma samples from GI cancer patients. The number of y axis refers to the ratio of samples being correctly predicted. Lower bar: sample labels were the same as the top prediction. Upper bar: sample labels were among the top 2 predictions. FIG. 4B shows the use of informative plasma DMRs from FIG. 4A for the classification of TCGA GI cancer tissues. FIGS. 4C-4D show t-distributed stochastic neighbor embedding (t-SNE) plots for plasma samples (n=300) and TCGA GI cancer tissue samples (1774) generated using informative plasma DMRs.

FIGS. 5A-5C present exemplary AUC scores vs. feature number plots with variable number of informative DMRs across GI cancers. FIG. 5A presents AUC scores vs. feature number plots showing the cancer prediction models for colorectal cancer (CRC), hepatocellular carcinoma (HCC), esophageal squamous cell carcinoma (ESCC), gastric cancer (GC), esophageal adenocarcinoma (EAC), and pancreatic ductal adenocarcinoma (PDAC). FIG. 5B presents AUC scores vs. feature number showing the pan-gastrointestinal (panGI or PGI) cancer prediction model. FIG. 5C presents AUC scores vs. feature number plots showing multi GI cancer tissue of origin classification model (colorectal cancer (CRC), hepatocellular carcinoma (HCC), esophageal squamous cell carcinoma (ESCC), gastric cancer (GC), esophageal adenocarcinoma (EAC), and pancreatic ductal adenocarcinoma (PDAC)).

FIG. 6A-6B shows workflow for training machine learning models for cancer prediction, based on the analysis of genome-wide tissue methylation data across gastrointestinal (GI) cancers. FIG. 6A shows a flow chart of the study design describing tissue discovery, followed by plasma cell-free DNA validation process. FIG. 6B shows circos plots showing the covered regions across the chromosomes.

FIG. 7 presents a heatmap showing hierarchical clustering of colorectal cancer (CRC) and healthy plasma samples.

FIG. 8 presents a heatmap showing hierarchical clustering of hepatocellular carcinoma (HCC) and healthy plasma samples.

FIG. 9 presents a heatmap showing hierarchical clustering of esophageal squamous cell carcinoma (ESCC) and healthy plasma samples.

FIG. 10 presents a heatmap showing hierarchical clustering of gastric cancer (GC) and healthy plasma samples.

FIG. 11 presents a heatmap showing hierarchical clustering of esophageal adenocarcinoma (EAC) and healthy plasma samples.

FIG. 12 presents a heatmap showing hierarchical clustering of pancreatic ductal adenocarcinoma (PDAC) and healthy plasma samples.

FIG. 13 is a boxplot showing a comparison of several machine learning classifiers.

FIG. 14 presents colorectal cancer (CRC) prediction accuracy using various number of DMRs identified from CRC versus healthy plasma sample analysis.

FIG. 15 presents hepatocellular carcinoma (HCC) prediction accuracy using various number of DMRs identified from HCC versus healthy plasma sample analysis.

FIG. 16 presents esophageal squamous cell carcinoma (ESCC) prediction accuracy using various number of DMRs identified from ESCC versus healthy plasma sample analysis.

FIG. 17 presents gastric cancer (GC) prediction accuracy using various number of DMRs identified from GC versus healthy plasma sample analysis.

FIG. 18 presents esophageal adenocarcinoma (EAC) prediction accuracy using various number of DMRs identified from EAC versus healthy plasma sample analysis.

FIG. 19 presents pancreatic ductal adenocarcinoma (PDAC) prediction accuracy using various number of DMRs identified from PDAC versus healthy plasma sample analysis.

FIG. 20 presents pan-gastrointestinal (panGI) prediction accuracy using various number of DMRs identified from panGI versus healthy plasma sample analysis.

FIG. 21 presents multi-class (top) prediction accuracy using various number of gastrointestinal cancer specific DMRs.

FIG. 22 presents multi-class (sec) prediction accuracy using various number of gastrointestinal cancer specific DMRs.

FIG. 23 presents coverage distribution of the GI targeted bisulfite sequencing panel (gitBS) performed on 300 plasma samples.

FIGS. 24A-24B present methylation ratio distribution of the GI targeted bisulfite sequencing panel (gitBS) performed on normal plasma samples (FIG. 24A) and GI cancer plasma samples (FIG. 24B).

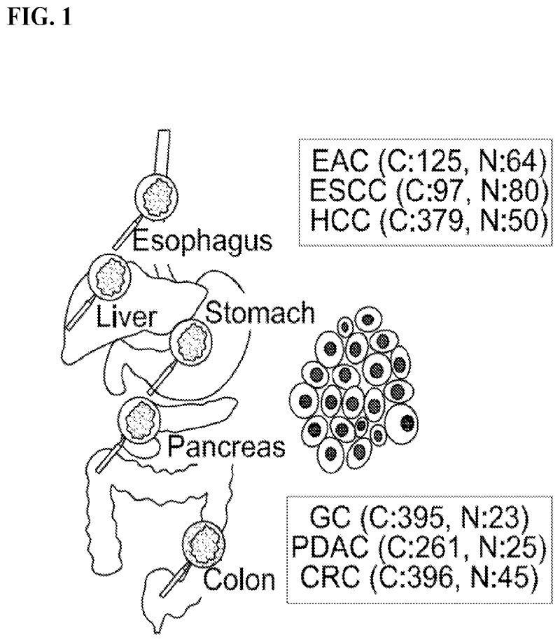

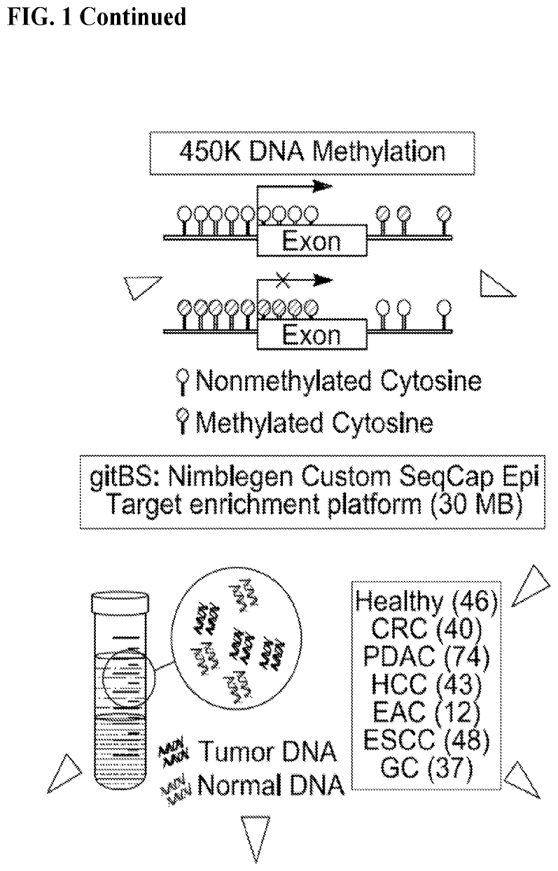

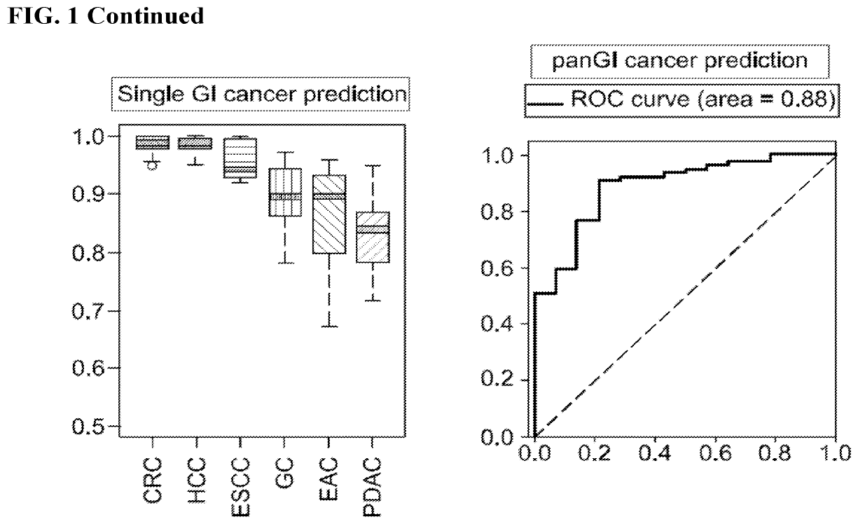

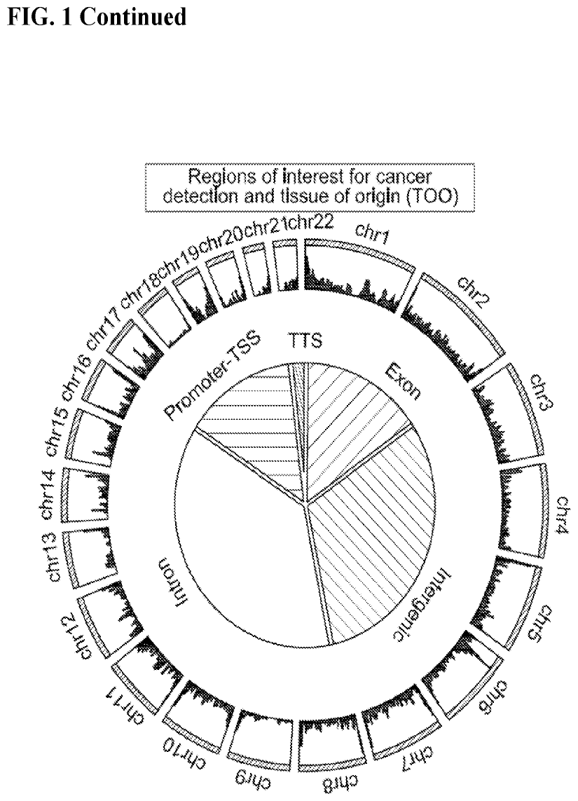

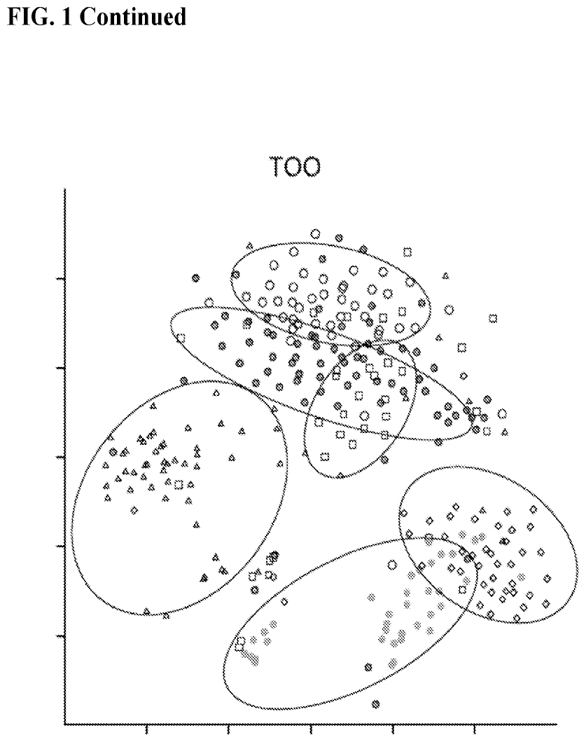

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

The singular forms “a”, “an” and “the” are intended to include the plural forms as well, unless the context clearly indicates otherwise.

The term “cancer” refers to all types of cancer, neoplasm or malignant tumors found in mammals (e.g. humans), including leukemias, lymphomas, carcinomas and sarcomas.

The term “carcinoma” refers to a malignant new growth made up of epithelial cells tending to infiltrate the surrounding tissues and give rise to metastases.

“Gastrointestinal cancer” or “GI cancer” refers to malignant conditions of the gastrointestinal tract (GI tract) and accessory organs of digestion, including the esophagus, stomach, biliary system, pancreas, small intestine, large intestine, rectum, and anus. The symptoms relate to the organ affected and can include obstruction (leading to difficulty swallowing or defecating), abnormal bleeding or other associated problems. Risk factors for an individual to develop gastrointestinal cancers include obesity, diet, family history, tobacco use, alcohol use, age, gender, and physical activity. “Pan-gastrointestinal” or “panGI” detection refers to detecting any one of a number of cancers of the gastrointestinal tract. Exemplary gastrointestinal cancers include colorectal cancer, hepatic cancer (e.g., hepatocellular carcinoma, esophageal cancers (e.g., esophageal adenocarcinoma, esophageal squamous cell carcinoma), and pancreatic cancer (e.g., pancreatic ductal adenocarcinoma).

“Colorectal cancer” or “CRC” (also known as colon cancer or rectal cancer) refers to cancer that develops in the colon or rectum. Risk factors for an individual to develop colorectal cancer include obesity, diet, family history, tobacco use, alcohol use, age, physical activity, diabetes, and diseases such as Barrett's esophagus, Lye, Achalasia, human papillomavirus infection, inflammatory bowel disease, Lynch syndrome, or familial adenomatous polyposis.

“Gastric cancer” or “stomach cancer” refers to a cancer that develops in the lining of the stomach. Most cases of stomach cancers are gastric carcinomas, which can be divided into a number of subtypes including gastric adenocarcinomas. Lymphomas and mesenchymal tumors may also develop in the stomach. Risk factors for an individual to develop gastric cancer (GC) include obesity, diet, family history, tobacco use, alcohol use, age, gender, physical activity, infection with Helicobacter pylori, long-term stomach inflammation (gastritis), stomach polyps, pernicious anemia, and Menetrier disease (hypertrophic gastropathy).

“Hepatocellular carcinoma” or “HCC” refers to the most common type of primary liver cancer in adults, and is the most common cause of death in people with cirrhosis. It occurs in the setting of chronic liver inflammation, and is most closely linked to chronic viral hepatitis infection (hepatitis B or C) or exposure to toxins. Certain diseases, such as hemochromatosis and alpha 1-antitrypsin deficiency, increase the risk of developing hepatocellular carcinoma. Metabolic syndrome and nonalcoholic steatohepatitis are also recognized as risk factors for hepatocellular carcinoma. Risk factors for an individual to develop hepatocellular carcinoma include chronic viral hepatitis, cirrhosis, non-alcoholic fatty liver disease, primary biliary cirrhosis, alcohol use, tobacco use, obesity, and type 2 diabetes.

“Esophageal cancer” refers to a tumor or cancer arising in the epithelial cells lining the esophagus and can be divided into two subtypes: esophageal squamous cell carcinoma and esophageal adenocarcinoma.

“Esophageal squamous cell carcinoma” or “ESCC” refers to an esophageal cancer that can affect any part of the esophagus, but is usually located in the upper or middle third.

“Esophageal adenocarcinoma” or “EAC” refers to esophageal cancer affecting the glandular cells of the lower esophagus at the junction with the stomach.

“Pancreatic ductal adenocarcinoma” or “PDAC” refers to a tumor arising in the pancreatic ductal epithelium. This cancer originates in the ducts that carry secretions away from the pancreas, and results in pancreatic cancer. Risk factors for developing pancreatic ductal adenocarcinoma include obesity, diet, family history, tobacco use, alcohol use, age, gender, physical activity, diabetes, family history, other inherited diseases (e.g. hereditary pancreatitis, Lynch syndrome, hereditary breast, or ovarian cancer syndrome), chronic pancreatitis, hepatitis B infection, and cirrhosis. PDAC is the most common type of pancreatic cancer.

The term “diagnosis” refers to the identification of a cancer. In embodiments, “diagnosis” refers to the process of determining or identifying whether a patient has cancer based on the levels of methylated CpG sites within a plurality of gene regions in a DNA sample obtained from the patient. The terms “confirmatory diagnostic procedure” or “confirmatory diagnosis procedure” refer to a process of confirming a diagnosis.

The term “in vitro” refers to assays, studies, or methods (e.g., detecting levels of methylated CpG sites within a plurality of gene regions) that are performed outside of a patient (e.g., outside the body of a human patient). Assays, studies, or methods performed on a DNA sample or biological fluid (e.g., blood, plasma, serum) obtained from a patient are in vitro because they are performed on a DNA sample or biological fluid that has been taken from the body of the patient.

“Patient” or “subject” refers to a living organism suffering from or prone to a disease (i.e., cancer) that can be treated as described herein. Non-limiting examples include humans, other mammals, bovines, rats, mice, dogs, cats, monkeys, goat, sheep, cows, and other non-mammalian animals. In embodiments, a patient is human. In embodiments, a patient is human having cancer. In embodiments, a patient is healthy human (e.g., a patient that does not have cancer). In embodiments, a patient is a human at risk of developing cancer.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, the control is a level of DNA methylation against which another level of DNA methylation (e.g. the DNA methylation level of a gene region disclosed herein) is compared, e.g., to make a diagnostic (e.g., predictive and/or prognostic) and/or therapeutic determination. In embodiments, the control is a level of methylated CpG sites against which another level of methylated CpG sites (e.g. the level of methylated CpG sites in a gene region disclosed herein) is compared, e.g., to make a diagnostic (e.g., predictive and/or prognostic) and/or therapeutic determination. In embodiments, a control is a healthy patient or a population of healthy patients. In embodiments, a “healthy patient” is a patient that does not have cancer. In embodiments, a “healthy patient” is a patient that does not have a gastrointestinal cancer. The term “standard control” in the context of measuring DNA methylation levels in a biological sample from a subject suffering from cancer refers to the detected levels of DNA methylation in a biological sample from a subject not suffering from cancer. In embodiments, the term “standard control” in the context of measuring DNA methylation levels in a biological sample from a subject suffering from cancer refers to the detected levels of DNA methylation in a biological sample from healthy tissue (i.e., tissue that does not have cancerous cells). In embodiments, a control is a pre-assigned value, e.g., a cut-off value which was previously determined to significantly separate tissue origins based on DMRs. In embodiments, the cut-off value is the median or mean (preferably median) DNA methylation level in the reference population. A control can also be obtained from the same individual, e.g., from an earlier-obtained sample, prior to disease, or prior to treatment. One of skill will recognize that controls can be designed for assessment of any number of parameters. In embodiments, a control is a negative control. In embodiments, a control comprises the average amount of DNA methylation (e.g., methylated CpG sites) in a population of subjects (e.g., with a gastrointestinal cancer) or in a healthy population. In embodiments, the control comprises an average amount (e.g. amount of DNA methylation) in a population in which the number of subjects (n) is 5 or more, 20 or more, 50 or more, 100 or more, 1,000 or more, and the like. In embodiments, the control is a standard control. In embodiments, a standard control is a level of DNA methylation (e.g., methylated CpG sites) of the gene region that has been correlated with a particular gastrointestinal cancer (e.g., colorectal cancer, hepatic cancer, esophageal cancer, pancreatic cancer). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect, and human cells. Cells may be useful when they are naturally nonadherent or have been treated not to adhere to surfaces, for example by trypsinization.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

The terms “DNA” or “deoxyribonucleic acid” refer to a molecule composed of two polynucleotide chains that coil around each other to form a double helix carrying genetic instructions for the development, functioning, growth and reproduction of all known organisms and many viruses. DNA and ribonucleic acid (RNA) are nucleic acids. Alongside proteins, lipids and complex carbohydrates (polysaccharides), nucleic acids are one of the four major types of macromolecules that are essential for all known forms of life. The two DNA strands are known as polynucleotides as they are composed of simpler monomeric units called nucleotides. Each nucleotide is composed of one of four nitrogen-containing nucleobases (cytosine (C), guanine (G), adenine (A) or thymine (T)), a sugar called deoxyribose, and a phosphate group. The nucleotides are joined to one another in a chain by covalent bonds (known as the phosphodiester linkage) between the sugar of one nucleotide and the phosphate of the next, resulting in an alternating sugar-phosphate backbone. The nitrogenous bases of the two separate polynucleotide strands are bound together, according to base pairing rules (A with T and C with G), with hydrogen bonds to make double-stranded DNA. The complementary nitrogenous bases are divided into two groups, pyrimidines and purines. In DNA, the pyrimidines are thymine and cytosine; the purines are adenine and guanine.

The term “DNA fraction” refers to DNA or portion of DNA partitioned from other molecules of a biological sample (e.g., biological fluid, such as blood, plasma, or serum).

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. The complementarity of sequences may be partial, in which only some of the nucleic acids match according to base pairing, or complete, where all the nucleic acids match according to base pairing. Thus, two sequences that are complementary to each other, may have a specified percentage of nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region).

The terms “biological fluids” or “biological fluid” refer to liquids within the human body. Such liquids can be blood, serum, plasma, saliva, ascites fluid, peritoneal fluid, and urine. In embodiments, the biological fluid is blood. In embodiments, the biological fluid is serum. In embodiments, the biological fluid is plasma. In embodiments, the biological fluid is saliva. In embodiments, the biological fluid is ascites fluid. In embodiments, the biological fluid is peritoneal fluid. In embodiments, the biological fluid is urine.

The terms “CpG sites” or “CG sites” as used herein refer to regions of DNA where a cytosine nucleotide is followed by a guanine nucleotide in the linear sequence of bases along its 5′-3′ direction. CpG sites occur with high frequency in genomic regions called CpG islands (or CG islands). Cytosines in CpG dinucleotides can be methylated to form 5-methylcytosines. Enzymes that add a methyl group are called DNA methyltransferases. In mammals, 70% to 80% of CpG cytosines are methylated. Methylating the cytosine within a gene can change its expression. In humans, DNA methylation occurs at the 5′ position of the pyrimidine ring of the cytosine residues within CpG sites to form 5-methylcytosines. The presence of multiple methylated CpG sites in CpG islands of promoters causes stable silencing of genes. In humans, about 70% of promoters located near the transcription start site of a gene (proximal promoters) contain a CpG island.

The terms “DNA methylation” refer to the addition of a methyl group on a biological process by which methyl groups are added to the DNA molecule. Methylation can change the activity of a DNA segment without changing the sequence. When located in a gene promoter, DNA methylation typically acts to repress gene transcription. In mammals, DNA methylation is essential for normal development and is associated with a number of key processes including genomic imprinting, X-chromosome inactivation, repression of transposable elements, aging, and carcinogenesis. DNA methylation in vertebrates typically occurs at CpG sites (cytosine-phosphate-guanine sites—that is, where a cytosine is directly followed by a guanine in the DNA sequence). This methylation results in the conversion of the cytosine to 5-methylcytosine. The formation of Me-CpG is catalyzed by the enzyme DNA methyltransferase. In mammals, DNA methylation is common in body cells, and methylation of CpG sites seems to be the default. Human DNA has about 80-90% of CpG sites methylated, but there are certain areas, known as CpG islands, that are CG-rich (high cytosine and guanine content, made up of about 65% CG residues), wherein none is methylated.

The terms “differentially methylated regions” or “DMRs” refer to genomic (gene) regions with different DNA methylation status across different biological samples and regarded as possible functional regions involved in gene transcriptional regulation. The biological samples can be different cells, tissues, or biological fluids within the same individual; the same cell, tissue or biological fluids at different times; or cells, tissues, or biological fluids from different individuals, even different alleles in the same cell. There are several different types of DMRs. These include tissue-specific DMR (tDMR), cancer-specific DMR (cDMR), development stages (dDMRs), reprogramming-specific DMR (rDMR), allele-specific DMR (AMR), and aging-specific DMR (aDMR). DNA methylation is associated with cell differentiation and proliferation. The gene regions in each of the tables can alternatively be referred to as the DMRs. In embodiments, the DMRs refer to gene regions with an elevated DNA methylation status in biological fluids of patients with cancer when compared to a standard control (e.g., biological fluids of people without cancer).

The terms “degree of methylation” or “degree of methylation of CpG sites” refer to the detected level of methylation of a specific DNA sequence (e.g. chromosome, gene, or non-coding DNA region), which correspond to the number of methylated CpG sites in the DNA sequence being analyzed. “DNA methylation level” or “methylation level” refers to the quantity of methylation of CpG sites in a gene region as described herein. The methylation level of CpG sites can be expressed as a relative or absolute value, additionally but not necessarily normalized to a standard or a reference sample or control. The value can also be expressed as a percentage or a proportion of a reference sample or control.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The term “gene region” is any portion of a full length gene, including non-coding regions, and can be defined by a beginning and end nucleotide of a DNA sequence. For example, Table MCC lists 382 gene regions, the first entry is a gene region from nucleotide 93905177 to nucleotide 93905542 of chromosome 5. The term “gene region” can alternatively be referred to as “DMR” when the gene region has differentially methylated regions (e.g., elevated DNA methylation) in biological fluids of patients with cancer when compared to a standard control (e.g., biological fluids of people without cancer). With respect to the tables herein, the term “gene region” does not include “Adjusted p-value” and “Freq” or “frequency” as those columns appear in the tables herein.

The term “aberrant” as used herein refers to different from normal. When used to describe DNA methylation, aberrant refers to methylation that is greater or less than a normal control or the average of normal non-diseased control samples. In embodiments, aberrant refers to methylation that is greater than a normal control or the average of normal non-diseased control samples. Aberrant activity may refer to an amount of activity that results in a disease, wherein returning the aberrant activity to a normal or non-disease-associated amount (e.g. by administering a compound or using a method as described herein), results in reduction of the disease or one or more disease symptoms.

The term “cell-free nucleic acid” refers to nucleic acid (e.g., DNA) present in a sample from a subject or portion thereof that can be isolated or otherwise manipulated without applying a lysis step to the sample as originally collected (e.g., as in extraction from cells or viruses). Cell-free nucleic acid (e.g., DNA) are thus unencapsulated or “free” from the cells or viruses from which they originate, even before a sample of the subject is collected. Cell-free nucleic acid (e.g., DNA) may be produced as a byproduct of cell death (e.g. apoptosis or necrosis) or cell shedding, releasing nucleic acids into surrounding biological fluids or into circulation. Accordingly, cell-free nucleic acid (e.g., DNA) may be isolated from a non-cellular fraction of blood (e.g. serum or plasma), from other biological fluids (e.g. urine), or from non-cellular fractions of other types of samples. In embodiments, the cell-free nucleic acid is cell-free DNA.

Methods for extracting DNA for a substantially cell-free sample of blood plasma or blood serum to obtain cell-free DNA is known in the art and described herein. In embodiments, “substantially” is at least 50% (e.g., a substantially cell-free DNA sample is a sample in which at least 50% of the DNA is cell-free DNA). In embodiments, “substantially” is at least 60%. In embodiments, “substantially” is at least 70%. In embodiments, “substantially” is at least 80%. In embodiments, “substantially” is at least 90%. In embodiments, “substantially” is at least 95%. In embodiments, “substantially” is at least 98%. In embodiments, “substantially” is at least 99%. In embodiments, “substantially” is 100%.

Methods for extracting DNA for a cell-free sample of blood, plasma, or serum to obtain cell-free DNA is known in the art. In embodiments, a fraction of DNA is produced by treating the cell-free DNA with sodium bisulfite to produce either a set of uracil modified cell-free DNA and a set of methylated cfDNA and then selectively amplifying only methylated cell-free DNA with at least two methylation biomarkers wherein the DNA fraction comprises a plurality of genetic loci of the cell-free DNA. In embodiments, the cell-free DNA is quantified and analyzed for methylation as a plurality of genetic loci. Sodium bisulfite treatment refers to a reaction that protects methylated cytosines from conversion, whereas unmethylated cytosines are converted into uracil. In embodiment, after PCR the converted uracils are recognized as thymines, whereas the methylated cytosines will appear as cytosines. In embodiments, methylated cell-free DNA is amplified by use of a polymerase chain reaction (PCR). PCR is well-known in the art and refers to a method to rapidly make multiple copies of specific DNA samples from a mixture of DNA molecules. In embodiments, the methylated cell-free DNA is quantified and analyzed by quantitative PCR (qPCR). qPCR refers to a method to determine absolute or relative quantities of a known sequence in a sample. In embodiments, the quantified sequence is analyzed to determine the methylation levels of the cell-free DNA in the sample.

### Methods

The methods provided herein, including embodiments thereof, allow for the detection of a level of DNA methylation in a subject at risk of developing a cancer, wherein the methods include determining the degree of methylation of CpG sites within a plurality of gene regions in a DNA sample from said subject, wherein the plurality of gene regions includes different gene regions. The methods provided herein, including embodiments thereof, allow for the treatment of cancer by detecting an increased level of methylated CpG sites, relative to a standard control, within a plurality of gene regions in a DNA sample obtained from the patient; and treating the patient for cancer. The methods provided herein, including embodiments thereof, allow for diagnosing cancer in a patient by detecting a level of methylated CpG sites within a plurality of gene regions in a DNA sample from the patient, and diagnosing the patient with cancer when the DNA sample has an increased level of methylated CpG sites, relative to a standard control, within the plurality of gene regions. The methods provided herein, including embodiments thereof, allow for monitoring risk for developing cancer in a patient or monitoring treatment in a patient having cancer by detecting a level of methylated CpG sites within a plurality of gene regions in a DNA sample from the patient at a first time point; detecting a level of methylated CpG sites within the plurality of gene regions in a DNA sample from the patient at a second time point, wherein the second time point is later than the first time point; and comparing the level of methylated CpG sites at the second time point to the level of methylated CpG sites at the first time point, thereby monitoring risk or monitoring treatment. The methods provided herein, including embodiments thereof, allow for the preparation and use of a DNA fraction from a subject. The DNA fraction may be prepared from a biological fluid of the subject. Thus, in another aspect is provided a method for preparing a DNA fraction from a subject at risk of developing a gastrointestinal cancer, the method including: (a) extracting DNA from a substantially cell-free sample of biological fluid of the subject to obtain extracellular DNA; and, (b) determining a level of DNA methylation in a gene region of a subject at risk according to including any of the methods disclosed herein including embodiments thereof. In embodiments, the gene regions are provided in Table PGI, Table CRC, Table HCC, Table ESCC, Table G, Table EAC, Table PDAC, or Table MCC of the present specification. “PGI” is pan-gastrointestinal cancers. “MCC” is multi-Cancer_classification.

### Gastrointestinal Cancer

Provided here is a method of detecting a level of DNA methylation in a subject at risk of developing a gastrointestinal cancer, the method including: determining the degree of methylation of CpG sites within a plurality of gene regions in a DNA sample from the subject, wherein the plurality of gene regions includes at least 50 different gene regions in Table PGI. In embodiments, the specific type of gastrointestinal cancer is not identified. In embodiments, the specific type of gastrointestinal cancer is not known. In embodiments, the gastrointestinal cancer is likely to be colorectal cancer, liver cancer (e.g., hepatocellular carcinoma), esophageal cancer (e.g., esophageal squamous cell carcinoma, esophageal adenocarcinoma), or pancreatic cancer (e.g., pancreatic ductal adenocarcinoma). In embodiments, the gastrointestinal cancer is Stage I, Stage II, or Stage III. In embodiments, the gastrointestinal cancer is Stage I. In embodiments, the gastrointestinal cancer is Stage II. In embodiments, the gastrointestinal cancer is Stage III. In embodiments, an increased level of methylation of CpG sites relative to a standard control indicates a higher risk of gastrointestinal cancer.

Provided herein is a method of treating a gastrointestinal cancer in a patient in need thereof comprising: (a) detecting an increased level of methylated CpG sites, relative to a standard control, within a plurality of gene regions in a DNA sample obtained from the patient, wherein the plurality of gene regions comprise at least 50 different gene regions in Table PGI; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the specific type of gastrointestinal cancer is not identified. In embodiments, the specific type of gastrointestinal cancer is not known. In embodiments, the gastrointestinal cancer is likely to be colorectal cancer, liver cancer (e.g., hepatocellular carcinoma), esophageal cancer (e.g., esophageal squamous cell carcinoma, esophageal adenocarcinoma), or pancreatic cancer (e.g., pancreatic ductal adenocarcinoma). In embodiments, the gastrointestinal cancer is Stage I, Stage II, or Stage III. In embodiments, the gastrointestinal cancer is Stage I. In embodiments, the gastrointestinal cancer is Stage II. In embodiments, the gastrointestinal cancer is Stage III.

Provided herein is a method of diagnosing a gastrointestinal cancer in a patient comprising: (a) detecting a level of methylated CpG sites within a plurality of gene regions in a DNA sample from the patient, wherein the plurality of gene regions comprise at least 50 different gene regions in Table PGI; and (b) diagnosing the patient with a gastrointestinal cancer when the DNA sample has an increased level of methylated CpG sites, relative to a standard control, within the plurality of gene regions. In embodiments, the specific type of gastrointestinal cancer is not identified. In embodiments, the specific type of gastrointestinal cancer is not known. In embodiments, the gastrointestinal cancer is likely to be colorectal cancer, liver cancer (e.g., hepatocellular carcinoma), esophageal cancer (e.g., esophageal squamous cell carcinoma, esophageal adenocarcinoma), or pancreatic cancer (e.g., pancreatic ductal adenocarcinoma). In embodiments, the gastrointestinal cancer is Stage I, Stage II, or Stage III. In embodiments, the gastrointestinal cancer is Stage I. In embodiments, the gastrointestinal cancer is Stage II. In embodiments, the gastrointestinal cancer is Stage III. In embodiments, the method further comprises treating the patient for cancer. In embodiments, the method further comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having a gastrointestinal cancer or monitoring risk for developing a gastrointestinal cancer in a patient comprising: (a) detecting a level of methylated CpG sites within a plurality of gene regions in a DNA sample from the patient at a first time point, wherein the plurality of gene regions comprise at least 50 different gene regions in Table PGI; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing a gastrointestinal cancer. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing a gastrointestinal cancer or does not have a gastrointestinal cancer. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing a gastrointestinal cancer or may have a gastrointestinal cancer. In embodiments, the specific type of gastrointestinal cancer is not identified. In embodiments, the specific type of gastrointestinal cancer is not known. In embodiments, the gastrointestinal cancer is likely to be colorectal cancer, liver cancer (e.g., hepatocellular carcinoma), esophageal cancer (e.g., esophageal squamous cell carcinoma, esophageal adenocarcinoma), or pancreatic cancer (e.g., pancreatic ductal adenocarcinoma). In embodiments, the gastrointestinal cancer is Stage I, Stage II, or Stage III. In embodiments, the gastrointestinal cancer is Stage I. In embodiments, the gastrointestinal cancer is Stage II. In embodiments, the gastrointestinal cancer is Stage III. In embodiments, the method further comprises treating the patient for cancer. In embodiments, the method further comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 75 different gene regions in Table PGI. In embodiments, the plurality of gene regions includes at least 100 different gene regions in Table PGI. In embodiments, the plurality of gene regions includes at least 110 different gene regions in Table PGI. In embodiments, the plurality of gene regions includes at least 120 different gene regions in Table PGI. In embodiments, the plurality of gene regions includes at least 130 different gene regions in Table PGI. In embodiments, the plurality of gene regions includes at least 140 different gene regions in Table PGI. In embodiments, the plurality of gene regions includes at least 150 different gene regions in Table PGI. In embodiments, the plurality of gene regions includes at least 160 different gene regions in Table PGI. In embodiments, the plurality of gene regions includes at least 170 different gene regions in Table PGI. In embodiments, the plurality of gene regions includes at least 180 different gene regions in Table PGI. In embodiments, the plurality of gene regions includes at least 190 different gene regions in Table PGI. In embodiments, the plurality of gene regions includes at least 200 different gene regions in Table PGI. In embodiments, the plurality of gene regions includes at least 225 different gene regions in Table PGI. In embodiments, the plurality of gene regions includes at least 250 different gene regions in Table PGI. In embodiments, the plurality of gene regions includes at least 275 different gene regions in Table PGI. In embodiments, the plurality of gene regions includes 285 different gene regions in Table PGI. In embodiments, the plurality of gene regions consists of the 285 gene regions in Table PGI.

In embodiments, the plurality of gene regions includes the first 50 gene regions in Table PGI. In embodiments, the plurality of gene regions includes the first 60 gene regions in Table PGI. In embodiments, the plurality of gene regions includes the first 70 gene regions in Table PGI. In embodiments, the plurality of gene regions includes the first 80 gene regions in Table PGI. In embodiments, the plurality of gene regions includes the first 90 gene regions in Table PGI. In embodiments, the plurality of gene regions includes the first 100 gene regions in Table PGI. In embodiments, the plurality of gene regions includes the first 110 gene regions in Table PGI. In embodiments, the plurality of gene regions includes the first 120 gene regions in Table PGI. In embodiments, the plurality of gene regions includes the first 130 gene regions in Table PGI. In embodiments, the plurality of gene regions includes the first 140 gene regions in Table PGI. In embodiments, the plurality of gene regions includes the first 150 gene regions in Table PGI. In embodiments, the plurality of gene regions includes the first 160 gene regions in Table PGI. In embodiments, the plurality of gene regions includes the first 170 gene regions in Table PGI. In embodiments, the plurality of gene regions includes the first 180 gene regions in Table PGI. In embodiments, the plurality of gene regions includes the first 190 gene regions in Table PGI. In embodiments, the plurality of gene regions includes the first 200 gene regions in Table PGI. In embodiments, the plurality of gene regions includes the first 225 gene regions in Table PGI. In embodiments, the plurality of gene regions includes the first 250 gene regions in Table PGI. In embodiments, the plurality of gene regions includes the first 275 gene regions in Table PGI.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is a fine needle aspiration, an endoscopy, or a biopsy collection. In embodiments, the confirmatory diagnostic procedure is a fine needle aspiration, an endoscopy, or a biopsy. In embodiments, the confirmatory diagnostic procedure is an X-Ray, a computed tomography scan (CT scan), a magnetic resonance imaging scan (MRI scan), a positron emission tomography scan (PET scan), a blood test, or a fecal test.

In embodiments, the method further includes treating the subject for a gastrointestinal cancer. In embodiments, treatment for a gastrointestinal cancer includes surgery, systemic chemotherapy, radiotherapy or targeted therapy. In embodiments, treatment for a gastrointestinal cancer comprises surgery, chemotherapy, radiotherapy, targeted therapy, or a combination of two or more thereof.

TABLE PGI

| Gene | | | | Gene | Adjusted | |
|---|---|---|---|---|---|---|
| Region No. | Chr | Start | End | Name | p-value | Freq |
| 1 | chr9 | 140772527 | 140772595 | CACNA1B | 1.1E−11 | 418 |
| 2 | chr7 | 100942951 | 100943665 | LOC101927746 | 9.3E−42 | 413 |
| 3 | chr12 | 312591 | 312753 | LOC101929384 | 2E−45 | 408 |
| 4 | chr3 | 156009098 | 156009319 | KCNAB1 | 1.6E−18 | 404 |
| 5 | chr4 | 8582798 | 8583233 | GPR78 | 5.1E−28 | 402 |
| 6 | chr7 | 4832002 | 4832627 | MIR4656 | 2.5E−61 | 401 |
| 7 | chr1 | 16085208 | 16085925 | FBLIM1 | 8.8E−74 | 399 |
| 8 | chr20 | 5297480 | 5297906 | PROKR2 | 3.8E−71 | 396 |
| 9 | chr1 | 112058046 | 112058721 | ADORA3 | 9.9E−78 | 391 |
| 10 | chr10 | 108923670 | 108924867 | SORCS1 | 5.9E−143 | 389 |
| 11 | chr9 | 122131297 | 122131749 | BRINP1 | 9E−54 | 386 |
| 12 | chr11 | 30037608 | 30038013 | KCNA4 | 4.8E−23 | 384 |
| 13 | chr1 | 107348218 | 107348586 | PRMT6 | 0.000095 | 381 |
| 14 | chr5 | 2751128 | 2757191 | C5orf38 | 1.3E−152 | 379 |
| 15 | chr12 | 125139527 | 125140249 | NCOR2 | 0.0038 | 359 |
| 16 | chr7 | 155151059 | 155151576 | BLACE | 2.7E−27 | 357 |
| 17 | chr15 | 96895376 | 96895791 | NR2F2 | 5.8E−21 | 341 |
| 18 | chr5 | 93905177 | 93905542 | KIAA0825 | 1.1E−73 | 334 |
| 19 | chr4 | 158141284 | 158141917 | GRIA2 | 5.7E−62 | 319 |
| 20 | chr19 | 53561167 | 53561775 | ERVV-2 | 1.3E−57 | 316 |
| 21 | chr2 | 74726477 | 74727000 | LBX2 | 3.6E−46 | 290 |
| 22 | chr19 | 56988313 | 56989846 | ZNF667-AS1 | 2.7E−236 | 279 |
| 23 | chr19 | 36912350 | 36912880 | LOC644189 | 7E−91 | 276 |
| 24 | chr8 | 41165745 | 41167139 | SFRP1 | 7E−164 | 274 |
| 25 | chr16 | 87447837 | 87448079 | MAP1LC3B | 1.8E−41 | 271 |
| 26 | chr22 | 32753597 | 32754024 | RFPL3 | 0.55 | 267 |
| 27 | chr16 | 32896382 | 32896472 | SLC6A10P | 0.013 | 255 |
| 28 | chr3 | 147136946 | 147137256 | LOC440982 | 3.2E−16 | 250 |
| 29 | chr1 | 237206266 | 237206721 | RYR2 | 3.4E−55 | 236 |
| 30 | chr4 | 174429648 | 174430640 | HAND2 | 1.5E−68 | 227 |
| 31 | chr18 | 31739158 | 31739455 | NOL4 | 9.6E−46 | 221 |
| 32 | chr7 | 155242318 | 155242925 | EN2 | 1.1E−58 | 214 |
| 33 | chr7 | 155250353 | 155250415 | EN2 | 1 | 209 |
| 34 | chr10 | 50976347 | 50977132 | OGDHL | 2.4E−81 | 192 |
| 35 | chr12 | 125139647 | 125140269 | NCOR2 | 0.0017 | 189 |
| 36 | chr8 | 65282005 | 65282932 | LOC102724623 | 2.4E−121 | 184 |
| 37 | chr6 | 50818180 | 50818424 | TFAP2B | 4.3E−100 | 183 |
| 38 | chr8 | 132052036 | 132054784 | ADCY8 | 5.2E−216 | 181 |
| 39 | chr4 | 165304258 | 165305137 | MARCH1 | 2.6E−88 | 181 |
| 40 | chr19 | 58545029 | 58545559 | ZSCAN1 | 2.4E−140 | 181 |
| 41 | chr6 | 58147126 | 58149415 | LINC00680 | 1.7E−97 | 179 |
| 42 | chr1 | 6268887 | 6269008 | RNF207 | 0.00035 | 177 |
| 43 | chr12 | 128752541 | 128753150 | TMEM132C | 6.3E−65 | 176 |
| 44 | chr19 | 38308083 | 38308175 | LOC644554 | 1.7E−17 | 176 |
| 45 | chr1 | 50881119 | 50882140 | DMRTA2 | 1.4E−33 | 176 |
| 46 | chr20 | 61788160 | 61788642 | MIR124-3 | 4.5E−18 | 176 |
| 47 | chr18 | 908970 | 909154 | ADCYAP1 | 1.7E−18 | 176 |
| 48 | chr16 | 32896752 | 32896840 | SLC6A10P | 1 | 171 |
| 49 | chr13 | 112723041 | 112724206 | SOX1 | 2.1E−56 | 168 |
| 50 | chr2 | 124782229 | 124783355 | CNTNAP5 | 2E−81 | 168 |
| 51 | chr13 | 28498102 | 28499045 | PDX1-AS1 | 0.0000017 | 168 |
| 52 | chr19 | 37282439 | 37282687 | ZNF790-AS1 | 0.00000014 | 168 |
| 53 | chr5 | 178367536 | 178368804 | ZNF454 | 6.3E−99 | 165 |
| 54 | chr4 | 13524753 | 13526008 | LINC01097 | 2E−62 | 163 |
| 55 | chr10 | 118031745 | 118033581 | GFRA1 | 4.1E−45 | 162 |
| 56 | chr2 | 5832525 | 5834007 | SOX11 | 1.9E−195 | 161 |
| 57 | chr19 | 30017510 | 30021485 | VSTM2B | 8.5E−90 | 160 |
| 58 | chr4 | 134071516 | 134074046 | PCDH10 | 9.4E−218 | 158 |
| 59 | chr8 | 70946912 | 70947440 | PRDM14 | 8.8E−32 | 157 |
| 60 | chr8 | 120685162 | 120685317 | ENPP2 | 0.032 | 150 |
| 61 | chr7 | 57484714 | 57484819 | MIR3147 | 0.043 | 149 |
| 62 | chr20 | 21695247 | 21695306 | PAX1 | 3.6E−14 | 148 |
| 63 | chr8 | 53851723 | 53853197 | NPBWR1 | 6.5E−128 | 146 |
| 64 | chr19 | 54445370 | 54445617 | CACNG8 | 2.5E−11 | 146 |
| 65 | chr2 | 115419825 | 115420075 | DPP10-AS3 | 4E−26 | 145 |
| 66 | chr17 | 79615325 | 79615612 | TSPAN10 | 1.6E−25 | 141 |
| 67 | chr16 | 214606 | 216803 | HBM | 0.00028 | 139 |
| 68 | chr9 | 104499982 | 104500205 | GRIN3A | 2.2E−25 | 133 |
| 69 | chr1 | 119532735 | 119532899 | TBX15 | 0.0000058 | 133 |
| 70 | chr5 | 92931643 | 92932639 | MIR548AO | 2.9E−10 | 131 |
| 71 | chr12 | 175962 | 176081 | IQSEC3 | 2.9E−44 | 129 |
| 72 | chr20 | 56283770 | 56284043 | PMEPA1 | 7E−11 | 127 |
| 73 | chr20 | 37356055 | 37357918 | SLC32A1 | 1.2E−132 | 124 |
| 74 | chr19 | 58570393 | 58570646 | ZNF135 | 3.6E−46 | 124 |
| 75 | chr16 | 48844551 | 48845153 | N4BP1 | 7.4E−82 | 123 |
| 76 | chr12 | 43944719 | 43946284 | ADAMTS20 | 1.7E−154 | 120 |
| 77 | chr6 | 55443608 | 55443781 | HMGCLL1 | 9.5E−09 | 115 |
| 78 | chr7 | 70597033 | 70598501 | WBSCR17 | 8.7E−118 | 113 |
| 79 | chr7 | 20823895 | 20824488 | SP8 | 4.2E−89 | 112 |
| 80 | chr4 | 8012162 | 8012350 | MIR95 | 1 | 112 |
| 81 | chr18 | 44773291 | 44775576 | SKOR2 | 4E−205 | 102 |
| 82 | chr7 | 158751060 | 158751160 | LINC00689 | 0.000074 | 99 |
| 83 | chr5 | 92930812 | 92931061 | MIR548AO | 0.000000012 | 99 |
| 84 | chr5 | 76934957 | 76935276 | OTP | 4.4E−32 | 90 |
| 85 | chr1 | 1244840 | 1245076 | PUSL1 | 8.8E−09 | 89 |
| 86 | chr19 | 51107536 | 51107741 | SNAR-F | 1.4E−23 | 88 |
| 87 | chr7 | 2774325 | 2774779 | GNA12 | 8.3E−32 | 86 |
| 88 | chr22 | 20792363 | 20792784 | SCARF2 | 1.3E−38 | 84 |
| 89 | chr13 | 53775090 | 53775595 | LINC01065 | 1.4E−135 | 82 |
| 90 | chr16 | 86066512 | 86066907 | MIR6774 | 9.9E−10 | 81 |
| 91 | chr13 | 112978530 | 112978862 | LINC01044 | 1.1E−26 | 80 |
| 92 | chr5 | 172671531 | 172672649 | NKX2-5 | 8.8E−43 | 80 |
| 93 | chr20 | 61809243 | 61809785 | MIR124-3 | 5.9E−83 | 79 |
| 94 | chr17 | 1551612 | 1551780 | RILP | 1.7E−13 | 78 |
| 95 | chr19 | 52957112 | 52957231 | ZNF578 | 0.00000023 | 76 |
| 96 | chr16 | 51183896 | 51185739 | SALL1 | 8E−132 | 71 |
| 97 | chr19 | 57154160 | 57155084 | SMIM17 | 5.8E−46 | 69 |
| 98 | chr8 | 57358126 | 57359512 | PENK | 6.1E−164 | 69 |
| 99 | chr1 | 248551109 | 248551544 | OR2T6 | 0.0000012 | 65 |
| 100 | chr2 | 63274711 | 63275273 | LOC100132215 | 1.2E−33 | 65 |
| 101 | chr10 | 50819078 | 50820313 | SLC18A3 | 1.2E−133 | 59 |
| 102 | chr20 | 57443951 | 57444145 | GNAS | 0.0099 | 57 |
| 103 | chr13 | 112575679 | 112576115 | LINC00354 | 1 | 56 |
| 104 | chr22 | 48945886 | 48946735 | LOC284933 | 5.1E−12 | 55 |
| 105 | chr1 | 9380774 | 9381262 | SPSB1 | 1 | 55 |
| 106 | chr3 | 171528450 | 171528601 | PLD1 | 1 | 54 |
| 107 | chr19 | 39755028 | 39755922 | IFNL2 | 5.3E−101 | 54 |
| 108 | chr1 | 78956690 | 78956865 | PTGFR | 2.6E−37 | 54 |
| 109 | chr8 | 1292265 | 1292374 | LOC286083 | 0.00000059 | 53 |
| 110 | chr2 | 233284689 | 233284880 | ALPPL2 | 1 | 53 |
| 111 | chr7 | 56355481 | 56355797 | LOC650226 | 8.1E−65 | 53 |
| 112 | chr10 | 119494424 | 119495015 | EMX2OS | 1.1E−46 | 52 |
| 113 | chr19 | 9473589 | 9474000 | ZNF177 | 3.7E−40 | 52 |
| 114 | chr14 | 103415848 | 103416095 | AMN | 0.00078 | 50 |
| 115 | chr19 | 14584258 | 14584325 | PTGER1 | 4.8E−11 | 50 |
| 116 | chr7 | 62574764 | 62574921 | ZNF733P | 1.7E−10 | 50 |
| 117 | chr16 | 764994 | 765059 | METRN | 1 | 47 |
| 118 | chr15 | 95870027 | 95870409 | LINC01197 | 1.5E−49 | 47 |
| 119 | chr8 | 49340828 | 49341091 | LOC101929268 | 3.4E−13 | 46 |
| 120 | chr17 | 5000871 | 5001123 | USP6 | 6.4E−60 | 46 |
| 121 | chr3 | 138763524 | 138763648 | PRR23C | 0.025 | 44 |
| 122 | chr11 | 132953090 | 132953393 | OPCML | 4.1E−33 | 43 |
| 123 | chr3 | 194097043 | 194097329 | LRRC15 | 0.1 | 43 |
| 124 | chr8 | 97162411 | 97162548 | GDF6 | 6.5E−10 | 43 |
| 125 | chr9 | 115142290 | 115142368 | HSDL2 | 1 | 42 |
| 126 | chr13 | 70681585 | 70682357 | ATXN8OS | 2.2E−51 | 39 |
| 127 | chr9 | 71788589 | 71789667 | TJP2 | 5.9E−120 | 38 |
| 128 | chr5 | 76249438 | 76249789 | CRHBP | 1.4E−54 | 36 |
| 129 | chr9 | 842097 | 842188 | DMRT1 | 0.00000005 | 36 |
| 130 | chr8 | 105431160 | 105432052 | DPYS | 0.000000058 | 33 |
| 131 | chr2 | 202900352 | 202900702 | FZD7 | 2.6E−16 | 32 |
| 132 | chr7 | 63642030 | 63642712 | ZNF735 | 3.8E−09 | 32 |
| 133 | chr6 | 100906564 | 100906629 | SIM1 | 3.2E−15 | 31 |
| 134 | chr11 | 64646155 | 64646271 | EHD1 | 1 | 31 |
| 135 | chr14 | 86000161 | 86000222 | FLRT2 | 1.5E−10 | 31 |
| 136 | chr7 | 103630754 | 103631004 | RELN | 1 | 30 |
| 137 | chr16 | 2042574 | 2042815 | SYNGR3 | 2E−21 | 29 |
| 138 | chr18 | 24765323 | 24765370 | CHST9 | 1 | 29 |
| 139 | chr8 | 99439920 | 99440255 | KCNS2 | 2.1E−39 | 28 |
| 140 | chr7 | 32467625 | 32467947 | LOC100130673 | 2.9E−29 | 27 |
| 141 | chr7 | 1563405 | 1563846 | MAFK | 2.3E−10 | 26 |
| 142 | chr1 | 3120942 | 3121391 | MIR4251 | 0.0009 | 24 |
| 143 | chr10 | 23461316 | 23463617 | PTF1A | 5.9E−163 | 23 |
| 144 | chr7 | 24323193 | 24325522 | NPY | 2.3E−106 | 22 |
| 145 | chr7 | 101961700 | 101962042 | MIR4285 | 1.8E−28 | 21 |
| 146 | chr] | 2949457 | 2949824 | ACTRT2 | 7.8E−30 | 21 |
| 147 | chr11 | 83392903 | 83393677 | DLG2 | 0.0000035 | 21 |
| 148 | chr8 | 11607240 | 11607395 | C8orf49 | 1 | 20 |
| 149 | chr1 | 21864514 | 21864996 | ALPL | 2.5E−19 | 20 |
| 150 | chr13 | 28500948 | 28503530 | PDX1-AS1 | 4.3E−128 | 20 |
| 151 | chr14 | 99786830 | 99786937 | BCL11B | 1 | 20 |
| 152 | chr3 | 26664476 | 26666187 | LRRC3B | 1.5E−49 | 19 |
| 153 | chr13 | 95364026 | 95364786 | SOX21 | 2.3E−94 | 19 |
| 154 | chr5 | 178421407 | 178422336 | GRM6 | 2E−164 | 18 |
| 155 | chr7 | 44185046 | 44185523 | MYL7 | 1.8E−18 | 18 |
| 156 | chr19 | 58545562 | 58545986 | ZSCAN1 | 9.9E−24 | 18 |
| 157 | chr12 | 11653268 | 11653352 | LINC01252 | 0.000000006 | 17 |
| 158 | chr7 | 1979883 | 1980131 | MAD1L1 | 0.00021 | 17 |
| 159 | chr19 | 58094931 | 58095898 | ZIK1 | 2.3E−72 | 17 |
| 160 | chr4 | 5891985 | 5892089 | CRMP1 | 0.000000003 | 17 |
| 161 | chr1 | 3210043 | 3210544 | ARHGEF16 | 0.0012 | 16 |
| 162 | chr14 | 57283790 | 57284277 | OTX2-AS1 | 3.7E−38 | 16 |
| 163 | chr19 | 58399818 | 58400409 | ZNF814 | 1E−36 | 16 |
| 164 | chr2 | 105275648 | 105276050 | LINC01114 | 2.7E−52 | 15 |
| 165 | chr14 | 101487395 | 101488558 | MIR379 | 0.000000022 | 14 |
| 166 | chr19 | 52222262 | 52223082 | HAS1 | 1.7E−83 | 14 |
| 167 | chr12 | 103350040 | 103350406 | ASCL1 | 2.8E−48 | 13 |
| 168 | chr19 | 18543915 | 18544445 | ISYNA1 | 8.8E−37 | 13 |
| 169 | chr3 | 147108577 | 147111786 | ZIC4 | 1.5E−203 | 12 |
| 170 | chr22 | 38221446 | 38221545 | GALR3 | 0.037 | 11 |
| 171 | chr8 | 97156853 | 97158021 | GDF6 | 1.8E−136 | 11 |
| 172 | chr10 | 1094759 | 1094846 | IDI1 | 0.008 | 10 |
| 173 | chr10 | 131265375 | 131265496 | MGMT | 0.0093 | 10 |
| 174 | chr4 | 13545251 | 13545612 | NKX3-2 | 7.9E−39 | 10 |
| 175 | chr2 | 177024366 | 177024783 | HOXD3 | 1E−24 | 10 |
| 176 | chr7 | 98972247 | 98972345 | ARPC1B | 0.031 | 10 |
| 177 | chr10 | 1260127 | 1260369 | LINC00200 | 0.000095 | 9 |
| 178 | chr9 | 19788215 | 19789383 | SLC24A2 | 1.3E−84 | 8 |
| 179 | chr10 | 63212495 | 63213003 | TMEM26-AS1 | 4.7E−28 | 8 |
| 180 | chr13 | 113764079 | 113764819 | F7 | 1.5E−16 | 7 |
| 181 | chr13 | 114965052 | 114965100 | CDC16 | 0.01 | 7 |
| 182 | chr19 | 38042033 | 38042418 | ZNF540 | 1.4E−33 | 7 |
| 183 | chr7 | 50343650 | 50344001 | IKZF1 | 1E−23 | 7 |
| 184 | chr19 | 58520728 | 58521646 | ZNF606 | 4.5E−82 | 7 |
| 185 | chr15 | 66914678 | 66914731 | LINC01169 | 0.000024 | 7 |
| 186 | chr10 | 7139067 | 7139412 | SFMBT2 | 0.000005 | 7 |
| 187 | chr17 | 80662269 | 80662763 | RAB40B | 0.0000074 | 7 |
| 188 | chr14 | 103605146 | 103605483 | TNFAIP2 | 0.037 | 6 |
| 189 | chr14 | 103740135 | 103740268 | EIF5 | 0.0019 | 6 |
| 190 | chr7 | 150038187 | 150038930 | RARRES2 | 1.4E−19 | 6 |
| 191 | chr1 | 205312383 | 205312981 | KLHDC8A | 9.2E−48 | 6 |
| 192 | chr16 | 67034939 | 67035037 | CES4A | 1 | 6 |
| 193 | chr4 | 7045265 | 7045311 | TADA2B | 0.0018 | 6 |
| 194 | chr10 | 123923232 | 123923630 | TACC2 | 6.5E−41 | 5 |
| 195 | chr5 | 180591445 | 180591786 | OR2V2 | 2.1E−12 | 5 |
| 196 | chr15 | 24722579 | 24723145 | PWRN3 | 2.1E−14 | 5 |
| 197 | chr11 | 2481009 | 2482442 | KCNQ1 | 5.4E−19 | 5 |
| 198 | chr22 | 30476027 | 30476089 | HORMAD2 | 1 | 5 |
| 199 | chr21 | 45770244 | 45770310 | TRPM2 | 9.1E−10 | 5 |
| 200 | chr17 | 72848047 | 72848236 | GRIN2C | 0.0045 | 5 |
| 201 | chr17 | 79008901 | 79008969 | BAIAP2 | 1 | 5 |
| 202 | chr9 | 94185994 | 94186209 | NFIL3 | 1 | 5 |
| 203 | chr5 | 1201660 | 1201805 | SLC6A19 | 0.000046 | 4 |
| 204 | chr5 | 140777343 | 140778048 | PCDHGB5 | 1.4E−28 | 4 |
| 205 | chr17 | 15689705 | 15689796 | MEIS3P1 | 1 | 4 |
| 206 | chr1 | 167486793 | 167487885 | CD247 | 1.4E−63 | 4 |
| 207 | chr5 | 180100616 | 180101412 | FLT4 | 2.8E−59 | 4 |
| 208 | chr19 | 22966485 | 22967051 | ZNF99 | 7E−36 | 4 |
| 209 | chr8 | 494817 | 494884 | TDRP | 0.0028 | 4 |
| 210 | chr13 | 58208641 | 58208747 | PCDH17 | 0.00000001 | 4 |
| 211 | chr3 | 128209603 | 128210307 | GATA2-AS1 | 4.1E−15 | 3 |
| 212 | chr2 | 132056778 | 132056928 | LOC440910 | 0.37 | 3 |
| 213 | chr12 | 133049696 | 133050826 | FBRSL1 | 4.4E−13 | 3 |
| 214 | chr3 | 150238117 | 150238551 | SERP1 | 3.6E−14 | 3 |
| 215 | chr3 | 170302816 | 170303891 | SLC7A14 | 1.3E−61 | 3 |
| 216 | chr6 | 3259103 | 3259165 | PSMG4 | 0.019 | 3 |
| 217 | chr5 | 3595980 | 3596030 | IRX1 | 1.9E−12 | 3 |
| 218 | chr5 | 37836772 | 37836838 | GDNF | 5.9E−10 | 3 |
| 219 | chr22 | 42311089 | 42311132 | SHISA8 | 1 | 3 |
| 220 | chr17 | 46604816 | 46604998 | HOXB1 | 1 | 3 |
| 221 | chr10 | 50817144 | 50817275 | CHAT | 3.9E−09 | 3 |
| 222 | chr18 | 67068677 | 67069273 | DOK6 | 2.7E−58 | 3 |
| 223 | chr7 | 84815360 | 84815805 | SEMA3D | 1.3E−16 | 3 |
| 224 | chr16 | 88228256 | 88228588 | LOC101928880 | 0.031 | 3 |
| 225 | chr9 | 96622911 | 96623196 | MIR4291 | 0.00045 | 3 |
| 226 | chr8 | 11416318 | 11416454 | LINC00208 | 0.014 | 2 |
| 227 | chr12 | 124393534 | 124394014 | CCDC92 | 0.000039 | 2 |
| 228 | chr10 | 126254543 | 126254769 | LHPP | 0.046 | 2 |
| 229 | chr9 | 133779739 | 133780018 | QRFP | 0.00000045 | 2 |
| 230 | chr22 | 17488797 | 17488993 | GAB4 | 0.067 | 2 |
| 231 | chr3 | 187387984 | 187388267 | SST | 1.1E−19 | 2 |
| 232 | chr9 | 34577906 | 34578054 | CNTFR-AS1 | 1 | 2 |
| 233 | chr1 | 36042841 | 36043373 | TFAP2E | 2.8E−63 | 2 |
| 234 | chr10 | 36476957 | 36477264 | PCAT5 | 0.33 | 2 |
| 235 | chr17 | 42248061 | 42248358 | ASB16 | 0.00094 | 2 |
| 236 | chr20 | 44880167 | 44880263 | CDH22 | 0.98 | 2 |
| 237 | chr5 | 50673176 | 50673303 | ISL1 | 0.000000019 | 2 |
| 238 | chr18 | 5891167 | 5891444 | TMEM200C | 3.9E−55 | 2 |
| 239 | chr20 | 61273694 | 61273818 | SLCO4A1 | 1 | 2 |
| 240 | chr2 | 90413838 | 90414075 | MIR4436A | 1.3E−27 | 2 |
| 241 | chr7 | 99817648 | 99818408 | PVRIG | 5.2E−72 | 2 |
| 242 | chr13 | 100069021 | 100069251 | MIR548AN | 9.7E−17 | 1 |
| 243 | chr14 | 100438283 | 100438345 | EVL | 0.0000011 | 1 |
| 244 | chr7 | 101961741 | 101962354 | MIR4285 | 4.4E−75 | 1 |
| 245 | chr14 | 103593247 | 103593593 | TNFAIP2 | 4.2E−51 | 1 |
| 246 | chr1 | 10764635 | 10764790 | CASZ1 | 2.9E−09 | 1 |
| 247 | chr10 | 109674693 | 109675094 | LINC01435 | 9.4E−09 | 1 |
| 248 | chr6 | 116692093 | 116692175 | DSE | 1 | 1 |
| 249 | chr16 | 1171931 | 1172126 | C1QTNF8 | 1 | 1 |
| 250 | chr12 | 132177099 | 132177517 | SFSWAP | 0.000000036 | 1 |
| 251 | chr11 | 134281625 | 134281685 | B3GAT1 | 0.023 | 1 |
| 252 | chr11 | 1424244 | 1424684 | BRSK2 | 0.00002 | 1 |
| 253 | chr8 | 144236492 | 144236682 | LY6H | 0.0000012 | 1 |
| 254 | chr2 | 154727822 | 154729150 | GALNT13 | 2.7E−157 | 1 |
| 255 | chr4 | 156129443 | 156129588 | NPY2R | 4.9E−16 | 1 |
| 256 | chr1 | 17020797 | 17020930 | | 0.000018 | 1 |
| 257 | chr5 | 176559237 | 176559421 | NSD1 | 1.1E−09 | 1 |
| 258 | chr5 | 178801251 | 178801695 | ADAMTS2 | 9.7E−13 | 1 |
| 259 | chr10 | 1850245 | 1850362 | ADARB2 | 0.053 | 1 |
| 260 | chr6 | 21801247 | 21801614 | CASC15 | 0.046 | 1 |
| 261 | chr2 | 219264602 | 219264771 | CTDSP1 | 7.7E−29 | 1 |
| 262 | chr1 | 228400272 | 228400693 | C1orf145 | 0.095 | 1 |
| 263 | chr2 | 240168706 | 240169346 | MGC16025 | 1.9E−36 | 1 |
| 264 | chr16 | 30389555 | 30389643 | ZNF48 | 1 | 1 |
| 265 | chr11 | 31827820 | 31827870 | PAX6 | 8.9E−09 | 1 |
| 266 | chr1 | 33367000 | 33367057 | TMEM54 | 0.0026 | 1 |
| 267 | chr17 | 35299347 | 35300455 | LHX1 | 9.4E−93 | 1 |
| 268 | chr2 | 38301293 | 38302642 | CYP1B1 | 4.1E−102 | 1 |
| 269 | chr21 | 38362713 | 38362815 | HLCS | 1 | 1 |
| 270 | chr6 | 41604583 | 41605020 | MDFI | 2E−18 | 1 |
| 271 | chr22 | 43807648 | 43807718 | MPPED1 | 1 | 1 |
| 272 | chr15 | 45408769 | 45409558 | DUOXA2 | 3.3E−36 | 1 |
| 273 | chr21 | 46001549 | 46002061 | KRTAP10-5 | 1 | 1 |
| 274 | chr14 | 48143369 | 48145724 | MDGA2 | 1.2E−274 | 1 |
| 275 | chr1 | 51984412 | 51984460 | EPS15 | 1 | 1 |
| 276 | chr12 | 52685008 | 52685334 | KRT81 | 6.4E−19 | 1 |
| 277 | chr8 | 55380105 | 55380185 | SOX17 | 0.064 | 1 |
| 278 | chr8 | 55533954 | 55534032 | RP1 | 0.065 | 1 |
| 279 | chr7 | 56605646 | 56606225 | LOC101928401 | 0.000000012 | 1 |
| 280 | chr7 | 63154756 | 63154933 | MIR4283-1 | 1.8E−12 | 1 |
| 281 | chr19 | 719203 | 719302 | PALM | 7.1E−12 | 1 |
| 282 | chr10 | 72268796 | 72269050 | PALD1 | 0.0000032 | 1 |
| 283 | chr17 | 77813332 | 77813460 | CBX4 | 1 | 1 |
| 284 | chr6 | 78172191 | 78173170 | HTR1B | 2.5E−97 | 1 |
| 285 | chr16 | 88717488 | 88717659 | CYBA | 0.000016 | 1 |

### Colorectal Cancer

In another aspect is provided a method of detecting a level of DNA methylation in a subject at risk of developing a colorectal cancer (CRC), the method including: determining the degree of methylation of CpG sites within a plurality of gene regions in a DNA sample from the subject, wherein the plurality of gene regions includes at least 5 different gene regions in Table CRC. In embodiments, an increased level of methylated CpG sites relative to a standard control indicates a higher risk of colorectal cancer.

Provided herein is a method of treating colorectal cancer in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table CRC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the colorectal cancer is Stage I, Stage II, or Stage III. In embodiments, the colorectal cancer is Stage I. In embodiments, the colorectal cancer is Stage II. In embodiments, the colorectal cancer is Stage III. In embodiments, the method comprises administering to the patient an effective amount of radiotherapy, administering to the patient an effective amount of chemotherapy, administering to the patient an effective amount of targeted therapy, or a combination of two or more thereof. In embodiments, the method comprises administering to the patient an effective amount of chemotherapy. In embodiments, the method comprises surgically removing the cancer from the patient and administering to the patient an effective amount of chemotherapy.

Provided herein is a method of diagnosing colorectal cancer in a patient comprising: (a) detecting a level of methylated CpG sites within a plurality of gene regions in a DNA sample from the patient, wherein the plurality of gene regions comprise at least 5 different gene regions in Table CRC; and (b) diagnosing the patient with colorectal cancer when the DNA sample has an increased level of methylated CpG sites, relative to a standard control, within the plurality of gene regions. In embodiments, the colorectal cancer is Stage I, Stage II, or Stage III. In embodiments, the colorectal cancer is Stage I. In embodiments, the colorectal cancer is Stage II. In embodiments, the colorectal cancer is Stage III. In embodiments, the method further comprises treating the patient for cancer. In embodiments, the method further comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having colorectal cancer or monitoring risk for developing colorectal cancer in a patient comprising: (a) detecting a level of methylated CpG sites within a plurality of gene regions in a DNA sample from the patient at a first time point, wherein the plurality of gene regions comprise at least 5 different gene regions in Table CRC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing colorectal cancer. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing colorectal cancer or does not have colorectal cancer. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing colorectal cancer or may have colorectal cancer. In embodiments, the colorectal cancer is Stage I, Stage II, or Stage III. In embodiments, the colorectal cancer is Stage I. In embodiments, the colorectal cancer is Stage II. In embodiments, the colorectal cancer is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table CRC. In embodiments, the plurality of gene regions includes at least 2 DMRs (i.e., gene regions) in Table CRC. In embodiments, the plurality of gene regions includes at least 3 DMRs in Table CRC. In embodiments, the plurality of gene regions includes at least 4 DMRs in Table CRC. In embodiments, the plurality of gene regions includes at least 5 DMRs in Table CRC. In embodiments, the plurality of gene regions includes at least 6 DMRs in Table CRC. In embodiments, the plurality of gene regions includes at least 7 DMRs in Table CRC. In embodiments, the plurality of gene regions includes at least 8 DMRs in Table CRC. In embodiments, the plurality of gene regions includes at least 9 DMRs in Table CRC. In embodiments, the plurality of gene regions includes at least 10 DMRs in Table CRC. In embodiments, the plurality of gene regions includes at least 15 DMRs in Table CRC. In embodiments, the plurality of gene regions includes at least 20 DMRs in Table CRC. In embodiments, the plurality of gene regions includes at least 25 DMRs in Table CRC. In embodiments, the plurality of gene regions includes at least 30 DMRs in Table CRC. In embodiments, the plurality of gene regions includes at least 35 DMRs in Table CRC. In embodiments, the plurality of gene regions includes at least 40 DMRs in Table CRC. In embodiments, the plurality of gene regions includes at least 45 DMRs in Table CRC. In embodiments, the plurality of gene regions includes at least 50 DMRs in Table CRC. In embodiments, the plurality of gene regions includes at least 55 DMRs in Table CRC. In embodiments, the plurality of gene regions includes at least 60 DMRs in Table CRC. In embodiments, the plurality of gene regions includes at least 65 DMRs in Table CRC. In embodiments, the plurality of gene regions includes at least 70 DMRs in Table CRC. In embodiments, the plurality of gene regions includes at least 75 DMRs in Table CRC. In embodiments, the plurality of gene regions includes at least 80 DMRs in Table CRC. In embodiments, the plurality of gene regions includes at least 85 DMRs in Table CRC. In embodiments, the plurality of gene regions includes at least 90 DMRs in Table CRC. In embodiments, the plurality of gene regions includes at least 95 DMRs in Table CRC. In embodiments, the plurality of gene regions includes at least 110 DMRs in Table CRC. In embodiments, the plurality of gene regions includes at least 120 DMRs in Table CRC. In embodiments, the plurality of gene regions includes at least 130 DMRs in Table CRC. In embodiments, the plurality of gene regions includes at least 140 DMRs in Table CRC. In embodiments, the plurality of gene regions includes at least 150 DMRs in Table CRC. In embodiments, the plurality of gene regions includes at least 160 DMRs in Table CRC. In embodiments, the plurality of gene regions includes at least 170 DMRs in Table CRC. In embodiments, the plurality of gene regions includes at least 180 DMRs in Table CRC. In embodiments, the plurality of gene regions includes at least 190 DMRs in Table CRC. In embodiments, the plurality of gene regions includes at least 200 DMRs in Table CRC. In embodiments, the plurality of gene regions includes at least 225 DMRs in Table CRC. In embodiments, the plurality of gene regions includes at least 250 DMRs in Table CRC. In embodiments, the plurality of gene regions includes at least 275 DMRs in Table CRC. In embodiments, the plurality of gene regions includes at least 300 DMRs in Table CRC. In embodiments, the plurality of gene regions includes at least 325 DMRs in Table CRC. In embodiments, the plurality of gene regions includes at least 350 DMRs in Table CRC. In embodiments, the plurality of gene regions includes at least 375 DMRs in Table CRC. In embodiments, the plurality of gene regions includes at least 400 DMRs in Table CRC. In embodiments, the plurality of gene regions includes at least 425 DMRs in Table CRC. In embodiments, the plurality of gene regions includes at least 450 DMRs in Table CRC. In embodiments, the plurality of gene regions includes at least 475 DMRs in Table CRC. In embodiments, the plurality of gene regions includes at least 500 DMRs in Table CRC. In embodiments, the plurality of gene regions includes at least 525 DMRs in Table CRC.

In embodiments, the plurality of gene regions includes the first DMR (i.e., gene region) in Table CRC. In embodiments, the plurality of gene regions includes the first 2 DMRs (i.e., gene regions) in Table CRC. In embodiments, the plurality of gene regions includes the first 3 DMRs in Table CRC. In embodiments, the plurality of gene regions includes the first 4 DMRs in Table CRC. In embodiments, the plurality of gene regions includes the first 5 DMRs in Table CRC. In embodiments, the plurality of gene regions includes the first 6 DMRs in Table CRC. In embodiments, the plurality of gene regions includes the first 7 DMRs in Table CRC. In embodiments, the plurality of gene regions includes the first 8 DMRs in Table CRC. In embodiments, the plurality of gene regions includes the first 9 DMRs in Table CRC. In embodiments, the plurality of gene regions includes the first 10 DMRs in Table CRC. In embodiments, the plurality of gene regions includes the first 11 DMRs in Table CRC. In embodiments, the plurality of gene regions includes the first 12 DMRs in Table CRC. In embodiments, the plurality of gene regions includes the first 13 DMRs in Table CRC. In embodiments, the plurality of gene regions includes the first 14 DMRs in Table CRC. In embodiments, the plurality of gene regions includes the first 15 DMRs in Table CRC. In embodiments, the plurality of gene regions includes the first 16 DMRs in Table CRC. In embodiments, the plurality of gene regions includes the first 17 DMRs in Table CRC. In embodiments, the plurality of gene regions includes the first 18 DMRs in Table CRC. In embodiments, the plurality of gene regions includes the first 19 DMRs in Table CRC. In embodiments, the plurality of gene regions includes the first 20 DMRs in Table CRC. In embodiments, the plurality of gene regions includes the first 21 DMRs in Table CRC. In embodiments, the plurality of gene regions includes the first 22 DMRs in Table CRC. In embodiments, the plurality of gene regions includes the first 23 DMRs in Table CRC. In embodiments, the plurality of gene regions includes the first 24 DMRs in Table CRC. In embodiments, the plurality of gene regions includes the first 25 DMRs in Table CRC. In embodiments, the plurality of gene regions includes the first 30 DMRs in Table CRC. In embodiments, the plurality of gene regions includes the first 35 DMRs in Table CRC. In embodiments, the plurality of gene regions includes the first 40 DMRs in Table CRC. In embodiments, the plurality of gene regions includes the first 45 DMRs in Table CRC. In embodiments, the plurality of gene regions includes the first 50 DMRs in Table CRC. In embodiments, the plurality of gene regions includes the first 55 DMRs in Table CRC. In embodiments, the plurality of gene regions includes the first 60 DMRs in Table CRC. In embodiments, the plurality of gene regions includes the first 65 DMRs in Table CRC. In embodiments, the plurality of gene regions includes the first 70 DMRs in Table CRC. In embodiments, the plurality of gene regions includes the first 75 DMRs in Table CRC. In embodiments, the plurality of gene regions includes the first 80 DMRs in Table CRC. In embodiments, the plurality of gene regions includes the first 85 DMRs in Table CRC. In embodiments, the plurality of gene regions includes the first 90 DMRs in Table CRC. In embodiments, the plurality of gene regions includes the first 95 DMRs in Table CRC. In embodiments, the plurality of gene regions includes the first 110 DMRs in Table CRC. In embodiments, the plurality of gene regions includes the first 120 DMRs in Table CRC. In embodiments, the plurality of gene regions includes the first 130 DMRs in Table CRC. In embodiments, the plurality of gene regions includes the first 140 DMRs in Table CRC. In embodiments, the plurality of gene regions includes the first 150 DMRs in Table CRC. In embodiments, the plurality of gene regions includes the first 160 DMRs in Table CRC. In embodiments, the plurality of gene regions includes the first 170 DMRs in Table CRC. In embodiments, the plurality of gene regions includes the first 180 DMRs in Table CRC. In embodiments, the plurality of gene regions includes the first 190 DMRs in Table CRC. In embodiments, the plurality of gene regions includes the first 200 DMRs in Table CRC. In embodiments, the plurality of gene regions includes the first 225 DMRs in Table CRC. In embodiments, the plurality of gene regions includes the first 250 DMRs in Table CRC. In embodiments, the plurality of gene regions includes the first 275 DMRs in Table CRC. In embodiments, the plurality of gene regions includes the first 300 DMRs in Table CRC. In embodiments, the plurality of gene regions includes the first 325 DMRs in Table CRC. In embodiments, the plurality of gene regions includes the first 350 DMRs in Table CRC. In embodiments, the plurality of gene regions includes the first 375 DMRs in Table CRC. In embodiments, the plurality of gene regions includes the first 400 DMRs in Table CRC. In embodiments, the plurality of gene regions includes the first 425 DMRs in Table CRC. In embodiments, the plurality of gene regions includes the first 450 DMRs in Table CRC. In embodiments, the plurality of gene regions includes the first 475 DMRs in Table CRC. In embodiments, the plurality of gene regions includes the first 500 DMRs in Table CRC. In embodiments, the plurality of gene regions includes the first 525 DMRs in Table CRC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is a fine needle aspiration, an endoscopy, or a tissue biopsy. In embodiments, the confirmatory diagnostic procedure is a fine needle aspiration, an endoscopy, or a biopsy. In embodiments, the confirmatory diagnostic procedure is a fecal DNA test or a carcinoembryonic antigen test.

In embodiments, the method further includes treating the subject for colorectal cancer. In embodiments, treating includes surgery, ablation, embolization, or radiotherapy. In embodiments, treating includes chemotherapy, targeted therapy, or immunotherapy. In embodiments, treating includes chemotherapy, targeted therapy, immunotherapy, or a combination of two or more thereof.

TABLE CRC

| Gene | | | | Gene | Adjusted | |
|---|---|---|---|---|---|---|
| Region No. | Chr | Start | End | Name | p-value | Freq |
| 1 | chr5 | 76924167 | 76924428 | OTP | 1.9E−30 | 418 |
| 2 | chr10 | 1460306 | 1460720 | ADARB2-AS1 | 0.00000001 | 416 |
| 3 | chr4 | 165304258 | 165305137 | MARCH1 | 2.6E−88 | 416 |
| 4 | chr19 | 18760598 | 18760955 | KLHL26 | 1.1E−36 | 415 |
| 5 | chr4 | 5894295 | 5894780 | CRMP1 | 4.1E−138 | 415 |
| 6 | chr8 | 41165745 | 41167139 | SFRP1 | 7E−164 | 413 |
| 7 | chr11 | 315696 | 316680 | IFITM1 | 2.7E−287 | 411 |
| 8 | chr5 | 2751128 | 2757191 | C5orf38 | 1.3E−152 | 409 |
| 9 | chr5 | 176046720 | 176047338 | MIR4281 | 1.2E−26 | 408 |
| 10 | chr15 | 37172579 | 37172784 | LOC145845 | 1.9E−35 | 408 |
| 11 | chr16 | 48844551 | 48845153 | N4BP1 | 7.4E−82 | 407 |
| 12 | chr19 | 58446168 | 58446960 | ZNF418 | 3.6E−59 | 407 |
| 13 | chr13 | 53775090 | 53775595 | LINC01065 | 1.4E−135 | 406 |
| 14 | chr2 | 5832525 | 5834007 | SOX11 | 1.9E−195 | 406 |
| 15 | chr15 | 95870027 | 95870409 | LINC01197 | 1.5E−49 | 406 |
| 16 | chr9 | 125391116 | 125391618 | OR1B1 | 0.0000065 | 405 |
| 17 | chr10 | 44198164 | 44198369 | ZNF32 | 0.00029 | 403 |
| 18 | chr6 | 50818180 | 50818424 | TFAP2B | 4.3E−100 | 403 |
| 19 | chr14 | 106235606 | 106235783 | MIR4507 | 0.0065 | 401 |
| 20 | chr4 | 13524753 | 13526008 | LINC01097 | 2E−62 | 401 |
| 21 | chr5 | 140531412 | 140531466 | PCDHB6 | 4.9E−15 | 400 |
| 22 | chr19 | 14591194 | 14591301 | PTGER1 | 0.007 | 400 |
| 23 | chr11 | 131780275 | 131781293 | NTM | 2.5E−155 | 399 |
| 24 | chr8 | 35092679 | 35093462 | UNC5D | 5.2E−117 | 399 |
| 25 | chr12 | 130647670 | 130648342 | FZD10 | 1.9E−85 | 398 |
| 26 | chr19 | 34113303 | 34113780 | CHST8 | 2.5E−69 | 398 |
| 27 | chr19 | 39755028 | 39755922 | IFNL2 | 5.3E−101 | 398 |
| 28 | chr1 | 167486793 | 167487885 | CD247 | 1.4E−63 | 396 |
| 29 | chr1 | 50882599 | 50886519 | DMRTA2 | 0.043 | 396 |
| 30 | chr8 | 144155053 | 144155326 | C8orf31 | 7.7E−16 | 395 |
| 31 | chr13 | 58206330 | 58207289 | PCDH17 | 7.7E−97 | 395 |
| 32 | chr19 | 30016974 | 30017506 | VSTM2B | 6.8E−96 | 394 |
| 33 | chr5 | 3596042 | 3603149 | IRX1 | 6.2E−139 | 394 |
| 34 | chr4 | 5570160 | 5570245 | LINC01587 | 1 | 393 |
| 35 | chr14 | 105318120 | 105318595 | CEP170B | 0.00037 | 392 |
| 36 | chr2 | 90016149 | 90016431 | MIR4436A | 4.1E−41 | 392 |
| 37 | chr5 | 93905177 | 93905542 | KIAA0825 | 1.1E−73 | 392 |
| 38 | chr7 | 99817648 | 99818408 | PVRIG | 5.2E−72 | 392 |
| 39 | chr1 | 148901738 | 148902370 | LOC101060524 | 9.8E−118 | 390 |
| 40 | chr6 | 26689679 | 26690160 | ZNF322 | 2.7E−45 | 390 |
| 41 | chr19 | 37282439 | 37282687 | ZNF790-AS1 | 0.00000014 | 390 |
| 42 | chr4 | 7483396 | 7483618 | MIR4274 | 0.0000039 | 389 |
| 43 | chr13 | 112978530 | 112978862 | LINC01044 | 1.1E−26 | 387 |
| 44 | chr8 | 11607240 | 11607395 | C8orf49 | 1 | 387 |
| 45 | chr17 | 25798664 | 25799447 | KSR1 | 0.000000016 | 386 |
| 46 | chr3 | 75955728 | 75956395 | ZNF717 | 8.7E−50 | 385 |
| 47 | chr4 | 134071516 | 134074046 | PCDH10 | 9.4E−218 | 382 |
| 48 | chr3 | 13974336 | 13974661 | FGD5P1 | 1.1E−46 | 382 |
| 49 | chr11 | 64034860 | 64034997 | PLCB3 | 7.2E−26 | 379 |
| 50 | chr20 | 25063816 | 25064630 | VSX1 | 2.9E−69 | 378 |
| 51 | chr4 | 298753 | 299338 | ZNF732 | 3.2E−65 | 375 |
| 52 | chr12 | 43944719 | 43946284 | ADAMTS20 | 1.7E−154 | 369 |
| 53 | chr16 | 87447837 | 87448079 | MAP1LC3B | 1.8E−41 | 369 |
| 54 | chr8 | 494817 | 494884 | TDRP | 0.0028 | 364 |
| 55 | chr6 | 170730605 | 170730682 | MIR4644 | 0.025 | 357 |
| 56 | chr3 | 147108577 | 147111786 | ZIC4 | 1.5E−203 | 354 |
| 57 | chr16 | 55689441 | 55690964 | SLC6A2 | 2.7E−147 | 346 |
| 58 | chr19 | 22989927 | 22990607 | ZNF99 | 1.7E−83 | 340 |
| 59 | chr4 | 147561142 | 147561866 | POU4F2 | 7.6E−68 | 336 |
| 60 | chr15 | 31175180 | 31175434 | FAN1 | 2.5E−13 | 334 |
| 61 | chr1 | 149137698 | 149138116 | | 6.5E−63 | 332 |
| 62 | chr5 | 2750524 | 2751117 | IRX2 | 8.2E−69 | 317 |
| 63 | chr17 | 77777037 | 77777782 | CBX8 | 1.4E−40 | 316 |
| 64 | chr19 | 22805734 | 22806241 | ZNF492 | 6E−76 | 311 |
| 65 | chr1 | 230882247 | 230883682 | CAPN9 | 1.3E−12 | 305 |
| 66 | chr8 | 142613052 | 142613443 | MROH5 | 2.9E−12 | 295 |
| 67 | chr14 | 99655528 | 99655861 | BCL11B | 1.5E−21 | 291 |
| 68 | chr2 | 154727822 | 154729150 | GALNT13 | 2.7E−157 | 286 |
| 69 | chr5 | 32712092 | 32712166 | NPR3 | 6.4E−26 | 286 |
| 70 | chr17 | 39254214 | 39254900 | KRTAP4-8 | 2.2E−11 | 284 |
| 71 | chr1 | 2949457 | 2949824 | ACTRT2 | 7.8E−30 | 280 |
| 72 | chr6 | 170373633 | 170374061 | LOC102724511 | 3.5E−18 | 278 |
| 73 | chr7 | 50632993 | 50634081 | DDC | 0.00036 | 278 |
| 74 | chr6 | 110300487 | 110301094 | GPR6 | 2.5E−45 | 275 |
| 75 | chr5 | 170743598 | 170743662 | TLX3 | 1.8E−17 | 261 |
| 76 | chr18 | 44773291 | 44775576 | SKOR2 | 4E−205 | 254 |
| 77 | chr10 | 106400989 | 106402192 | SORCS3 | 9.8E−153 | 245 |
| 78 | chr4 | 1399785 | 1400798 | NKX1-1 | 5.4E−175 | 236 |
| 79 | chr6 | 43612626 | 43612953 | RSPH9 | 1.8E−31 | 232 |
| 80 | chr10 | 23461316 | 23463617 | PTF1A | 5.9E−163 | 223 |
| 81 | chr12 | 29935995 | 29936081 | TMTC1 | 2.2E−21 | 220 |
| 82 | chr6 | 105388312 | 105388550 | LINC00577 | 2.4E−19 | 219 |
| 83 | chr2 | 239756351 | 239757924 | TWIST2 | 7.5E−148 | 218 |
| 84 | chr2 | 121223490 | 121224836 | LINC01101 | 1.3E−15 | 217 |
| 85 | chr17 | 59534376 | 59535253 | TBX4 | 3.4E−43 | 216 |
| 86 | chr12 | 127212288 | 127212578 | LINC00943 | 1E−22 | 214 |
| 87 | chr11 | 45718064 | 45718522 | MIR7154 | 0.00000015 | 212 |
| 88 | chr2 | 225265848 | 225266580 | FAM124B | 5.2E−42 | 211 |
| 89 | chr5 | 140595313 | 140595467 | PCDHB13 | 5.2E−26 | 210 |
| 90 | chr13 | 95364026 | 95364786 | SOX21 | 2.3E−94 | 210 |
| 91 | chr21 | 38068193 | 38070395 | SIM2 | 6.3E−115 | 207 |
| 92 | chr17 | 80163090 | 80163377 | CCDC57 | 1 | 206 |
| 93 | chr13 | 112710922 | 112711829 | SOX1 | 1.2E−42 | 205 |
| 94 | chr14 | 101489246 | 101489649 | MIR411 | 5.1E−09 | 204 |
| 95 | chr10 | 102419094 | 102419667 | PAX2 | 8.5E−39 | 204 |
| 96 | chr1 | 117753496 | 117753965 | VTCN1 | 0.000000099 | 204 |
| 97 | chr6 | 158080653 | 158080862 | MIR3692 | 2.3E−09 | 204 |
| 98 | chr20 | 21695247 | 21695306 | PAX1 | 3.6E−14 | 204 |
| 99 | chr19 | 58545029 | 58545559 | ZSCAN1 | 2.4E−140 | 204 |
| 100 | chr5 | 170735030 | 170736513 | TLX3 | 2.7E−59 | 202 |
| 101 | chr1 | 23953479 | 23953934 | MDS2 | 5.7E−13 | 202 |
| 102 | chr1 | 159046377 | 159047177 | AIM2 | 1.5E−15 | 201 |
| 103 | chr4 | 158141284 | 158141917 | GRIA2 | 5.7E−62 | 200 |
| 104 | chr5 | 178367536 | 178368804 | ZNF454 | 6.3E−99 | 200 |
| 105 | chr19 | 6753155 | 6753298 | TRIP10 | 3.6E−11 | 200 |
| 106 | chr10 | 1155969 | 1156615 | LINC00200 | 1.2E−27 | 199 |
| 107 | chr11 | 2481009 | 2482442 | KCNQ1 | 5.4E−19 | 199 |
| 108 | chr20 | 57089251 | 57090236 | APCDD1L | 1.3E−74 | 199 |
| 109 | chr12 | 63543712 | 63544966 | AVPR1A | 5.7E−97 | 199 |
| 110 | chr16 | 86066512 | 86066907 | MIR6774 | 9.9E−10 | 199 |
| 111 | chr12 | 128752541 | 128753150 | TMEM132C | 6.3E−65 | 198 |
| 112 | chr18 | 73628004 | 73628378 | LOC339298 | 1.1E−60 | 198 |
| 113 | chr18 | 77157010 | 77158037 | NFATC1 | 1.4E−54 | 198 |
| 114 | chr7 | 153583317 | 153584340 | DPP6 | 2.7E−90 | 197 |
| 115 | chr12 | 130526741 | 130527116 | LOC100190940 | 5.3E−31 | 196 |
| 116 | chr5 | 140213923 | 140214585 | PCDHA7 | 3.8E−32 | 196 |
| 117 | chr20 | 55201041 | 55201481 | TFAP2C | 5.6E−20 | 196 |
| 118 | chr16 | 89674874 | 89675253 | DPEP1 | 0.00066 | 196 |
| 119 | chr10 | 101291127 | 101293384 | NKX2-3 | 3.2E−20 | 195 |
| 120 | chr10 | 109674693 | 109675094 | LINC01435 | 9.4E−09 | 195 |
| 121 | chr3 | 6903030 | 6903205 | GRM7 | 2.2E−41 | 195 |
| 122 | chr5 | 140305727 | 140307192 | PCDHAC1 | 6.2E−93 | 194 |
| 123 | chr17 | 7832993 | 7833294 | KCNAB3 | 7.3E−26 | 194 |
| 124 | chr7 | 130418149 | 130418261 | KLF14 | 4.9E−09 | 193 |
| 125 | chr2 | 242927741 | 242928044 | LOC102723927 | 4.2E−27 | 193 |
| 126 | chr18 | 70533842 | 70535309 | NETO1 | 1.5E−187 | 192 |
| 127 | chr8 | 132052036 | 132054784 | ADCY8 | 5.2E−216 | 191 |
| 128 | chr1 | 17022838 | 17023402 | | 0.000067 | 191 |
| 129 | chr4 | 8873450 | 8876356 | HMX1 | 2.2E−27 | 191 |
| 130 | chr3 | 194785870 | 194786337 | XXYLT1-AS1 | 2.7E−21 | 190 |
| 131 | chr11 | 20626708 | 20627322 | SLC6A5 | 7.5E−18 | 190 |
| 132 | chr20 | 21686714 | 21687523 | PAX1 | 1.1E−72 | 190 |
| 133 | chr5 | 2748307 | 2749889 | IRX2 | 4.4E−146 | 190 |
| 134 | chr10 | 38893389 | 38893461 | ACTR3BP5 | 0.0000026 | 190 |
| 135 | chr6 | 7051353 | 7051576 | RREB1 | 0.069 | 190 |
| 136 | chr11 | 20618869 | 20619104 | SLC6A5 | 1.6E−26 | 189 |
| 137 | chr6 | 78172191 | 78173170 | HTR1B | 2.5E−97 | 189 |
| 138 | chr2 | 87883776 | 87883951 | MIR4435-1 | 5.4E−70 | 189 |
| 139 | chr12 | 126675956 | 126676034 | LOC101927464 | 1.9E−16 | 188 |
| 140 | chr7 | 158816342 | 158816506 | LINC00689 | 2.7E−16 | 188 |
| 141 | chr19 | 475077 | 475675 | ODF3L2 | 4.7E−11 | 188 |
| 142 | chr10 | 7311326 | 7311587 | SFMBT2 | 5.9E−21 | 188 |
| 143 | chr6 | 170413895 | 170414149 | LOC102724511 | 1 | 187 |
| 144 | chr18 | 44790493 | 44790613 | SKOR2 | 9.1E−11 | 187 |
| 145 | chr17 | 5000871 | 5001123 | USP6 | 6.4E−60 | 187 |
| 146 | chr8 | 1292265 | 1292374 | LOC286083 | 0.00000059 | 186 |
| 147 | chr19 | 58629190 | 58629803 | ZSCAN18 | 7.3E−54 | 186 |
| 148 | chr8 | 70041770 | 70041817 | LINC01592 | 1 | 186 |
| 149 | chr6 | 19691925 | 19692300 | ID4 | 1.5E−49 | 185 |
| 150 | chr3 | 48310428 | 48311036 | ZNF589 | 1.1E−33 | 185 |
| 151 | chr18 | 55019953 | 55021680 | ST8SIA3 | 0.84 | 185 |
| 152 | chr7 | 8481842 | 8482826 | NXPH1 | 2.4E−78 | 185 |
| 153 | chr4 | 1397339 | 1397392 | NKX1-1 | 6.2E−14 | 184 |
| 154 | chr3 | 147127952 | 147128456 | ZIC1 | 4.5E−69 | 184 |
| 155 | chr1 | 169429687 | 169430111 | CCDC181 | 1E−34 | 184 |
| 156 | chr10 | 50605228 | 50605945 | DRGX | 1.1E−57 | 184 |
| 157 | chr16 | 51186291 | 51188813 | SALL1 | 1E−71 | 184 |
| 158 | chr16 | 62068900 | 62070319 | CDH8 | 8.9E−118 | 184 |
| 159 | chr7 | 151553549 | 151553813 | PRKAG2-AS1 | 0.00000068 | 183 |
| 160 | chr1 | 17025587 | 17026500 | | 7.9E−89 | 183 |
| 161 | chr2 | 112123586 | 112124284 | | 1.5E−84 | 182 |
| 162 | chr22 | 17600837 | 17600960 | CECR6 | 5E−21 | 182 |
| 163 | chr18 | 31739158 | 31739455 | NOL4 | 9.6E−46 | 182 |
| 164 | chr19 | 38345789 | 38345949 | LOC100631378 | 4.5E−38 | 182 |
| 165 | chr1 | 119527391 | 119527516 | TBX15 | 2.2E−16 | 181 |
| 166 | chr3 | 147136946 | 147137256 | LOC440982 | 3.2E−16 | 181 |
| 167 | chr19 | 36735979 | 36736818 | ZNF565 | 2.5E−71 | 181 |
| 168 | chr7 | 63642030 | 63642712 | ZNF735 | 3.8E−09 | 181 |
| 169 | chr14 | 29237012 | 29237819 | FOXG1 | 3.3E−63 | 180 |
| 170 | chr19 | 52956621 | 52957010 | ZNF578 | 8.9E−42 | 180 |
| 171 | chr2 | 100937916 | 100938596 | LONRF2 | 9.5E−193 | 179 |
| 172 | chr11 | 64635577 | 64635975 | EHD1 | 1.7E−20 | 179 |
| 173 | chr11 | 69974619 | 69974828 | ANO1 | 0.001 | 179 |
| 174 | chr5 | 140188313 | 140188472 | PCDHA4 | 4.5E−16 | 178 |
| 175 | chr5 | 32712589 | 32713895 | NPR3 | 1.6E−117 | 178 |
| 176 | chr4 | 7483771 | 7483929 | MIR4274 | 0.00093 | 178 |
| 177 | chr17 | 56401783 | 56401952 | BZRAP1-AS1 | 4.4E−52 | 177 |
| 178 | chr18 | 76740400 | 76741361 | SALL3 | 7.4E−123 | 177 |
| 179 | chr4 | 37245250 | 37245351 | NWD2 | 1 | 176 |
| 180 | chr12 | 772457 | 772731 | NINJ2 | 0.00000057 | 176 |
| 181 | chr10 | 108923670 | 108924867 | SORCS1 | 5.9E−143 | 175 |
| 182 | chr7 | 31091910 | 31092310 | ADCYAP1R1 | 2.3E−118 | 175 |
| 183 | chr19 | 58629870 | 58630224 | ZSCAN18 | 0.0000025 | 175 |
| 184 | chr4 | 174429648 | 174430640 | HAND2 | 1.5E−68 | 174 |
| 185 | chr10 | 43891546 | 43891653 | HNRNPF | 3.1E−18 | 174 |
| 186 | chr16 | 46462413 | 46462715 | ANKRD26P1 | 2.9E−55 | 174 |
| 187 | chr1 | 53992314 | 53992630 | DMRTB1 | 2.1E−10 | 174 |
| 188 | chr16 | 1052397 | 1052904 | SOX8 | 3.5E−17 | 173 |
| 189 | chr3 | 31494123 | 31494215 | STT3B | 0.00002 | 173 |
| 190 | chr22 | 45132534 | 45133466 | ARHGAP8 | 1.9E−38 | 173 |
| 191 | chr10 | 119001477 | 119001615 | SLC18A2 | 1.7E−10 | 172 |
| 192 | chr11 | 1892037 | 1892423 | LSP1 | 2E−40 | 172 |
| 193 | chr19 | 30866358 | 30866489 | ZNF536 | 0.000059 | 172 |
| 194 | chr4 | 107146 | 107967 | ZNF718 | 1.3E−127 | 170 |
| 195 | chr9 | 96716761 | 96717050 | BARX1 | 1.5E−09 | 170 |
| 196 | chr6 | 1515470 | 1515607 | FOXCUT | 0.0089 | 169 |
| 197 | chr6 | 170553122 | 170553880 | LOC154449 | 6.2E−32 | 168 |
| 198 | chr6 | 58147126 | 58149415 | LINC00680 | 1.7E−97 | 168 |
| 199 | chr19 | 58951882 | 58952275 | ZNF132 | 1.7E−19 | 168 |
| 200 | chr12 | 133049696 | 133050826 | FBRSL1 | 4.4E−13 | 167 |
| 201 | chr2 | 240168706 | 240169346 | MGC16025 | 1.9E−36 | 167 |
| 202 | chr10 | 126254543 | 126254769 | LHPP | 0.046 | 166 |
| 203 | chr19 | 2251000 | 2251061 | MIR4321 | 0.000016 | 166 |
| 204 | chr17 | 25289487 | 25289553 | MIR4522 | 0.00042 | 166 |
| 205 | chr5 | 178016539 | 178017711 | COL23A1 | 1.9E−134 | 165 |
| 206 | chr5 | 145719093 | 145720027 | POU4F3 | 5.6E−64 | 164 |
| 207 | chr20 | 37356055 | 37357918 | SLC32A1 | 1.2E−132 | 163 |
| 208 | chr20 | 50721104 | 50721611 | ZFP64 | 4.4E−36 | 163 |
| 209 | chr7 | 8474829 | 8474939 | NXPH1 | 3.8E−10 | 161 |
| 210 | chr1 | 119522232 | 119522972 | TBX15 | 1.4E−55 | 160 |
| 211 | chr3 | 68981401 | 68982141 | FAM19A4 | 2.4E−65 | 160 |
| 212 | chr5 | 140346515 | 140346985 | PCDHAC2 | 3E−61 | 158 |
| 213 | chr15 | 45408769 | 45409558 | DUOXA2 | 3.3E−36 | 158 |
| 214 | chr7 | 70597033 | 70598501 | WBSCR17 | 8.7E−118 | 158 |
| 215 | chr2 | 168675039 | 168675400 | B3GALT1 | 0.00089 | 157 |
| 216 | chr1 | 17020797 | 17020930 | | 0.000018 | 157 |
| 217 | chr4 | 206281 | 207007 | ZNF876P | 6.9E−92 | 156 |
| 218 | chr15 | 68118223 | 68118660 | SKOR1 | 3.1E−21 | 156 |
| 219 | chr13 | 109147979 | 109149018 | MYO16 | 4.6E−154 | 155 |
| 220 | chr7 | 153584345 | 153585592 | DPP6 | 9.9E−129 | 155 |
| 221 | chr17 | 50235247 | 50236233 | CA10 | 6.9E−82 | 155 |
| 222 | chr13 | 111978726 | 111978866 | TEX29 | 0.89 | 154 |
| 223 | chr11 | 123388711 | 123389533 | GRAMD1B | 0.00000086 | 154 |
| 224 | chr3 | 13515572 | 13515826 | HDAC11-AS1 | 0.00067 | 152 |
| 225 | chr4 | 3877916 | 3878395 | FAM86EP | 3.2E−10 | 152 |
| 226 | chr5 | 140166058 | 140166202 | PCDHA1 | 0.00000043 | 151 |
| 227 | chr5 | 178801251 | 178801695 | ADAMTS2 | 9.7E−13 | 151 |
| 228 | chr19 | 44203509 | 44204090 | IRGC | 6.7E−48 | 151 |
| 229 | chr4 | 8861489 | 8862361 | HMX1 | 3.5E−66 | 151 |
| 230 | chr18 | 56939976 | 56940662 | RAX | 1.2E−79 | 150 |
| 231 | chr2 | 132088520 | 132088963 | WTH3DI | 1.1E−39 | 148 |
| 232 | chr19 | 22966485 | 22967051 | ZNF99 | 7E−36 | 148 |
| 233 | chr19 | 57375772 | 57376128 | MIMT1 | 4.9E−21 | 147 |
| 234 | chr10 | 50819078 | 50820313 | SLC18A3 | 1.2E−133 | 146 |
| 235 | chr19 | 56988313 | 56989846 | ZNF667-AS1 | 2.7E−236 | 146 |
| 236 | chr6 | 134497378 | 134497871 | SGK1 | 1.8E−52 | 145 |
| 237 | chr14 | 99712233 | 99712886 | BCL11B | 6E−70 | 144 |
| 238 | chr | 112058046 | 112058721 | ADORA3 | 9.9E−78 | 143 |
| 239 | chr6 | 170492207 | 170492448 | LOC102724511 | 1.4E−13 | 143 |
| 240 | chr4 | 30724084 | 30724148 | PCDH7 | 1.1E−14 | 143 |
| 241 | chr7 | 3341533 | 3341570 | SDK1 | 1.6E−12 | 141 |
| 242 | chr18 | 77623664 | 77624111 | KCNG2 | 7.1E−83 | 141 |
| 243 | chr5 | 140176513 | 140176604 | PCDHA2 | 0.75 | 139 |
| 244 | chr2 | 72374364 | 72374568 | CYP26B1 | 8.9E−32 | 138 |
| 245 | chr19 | 1047668 | 1047838 | ABCA7 | 5.8E−09 | 137 |
| 246 | chr13 | 110958726 | 110960077 | COL4A1 | 1.3E−159 | 135 |
| 247 | chr11 | 69633517 | 69634108 | FGF3 | 4.3E−34 | 135 |
| 248 | chr7 | 93657 | 93903 | LOC100507642 | 0.0000015 | 135 |
| 249 | chr16 | 57937508 | 57938003 | CNGB1 | 0.00000016 | 134 |
| 250 | chr16 | 85433603 | 85433775 | MIR5093 | 3.7E−10 | 133 |
| 251 | chr17 | 9129737 | 9129856 | LOC101928266 | 0.000082 | 131 |
| 252 | chr17 | 3289582 | 3289717 | OR1E1 | 5.4E−26 | 130 |
| 253 | chr10 | 133048319 | 133048710 | TCERG1L | 0.028 | 129 |
| 254 | chr5 | 140537550 | 140537668 | PCDHB17P | 0.0000061 | 129 |
| 255 | chr12 | 30354525 | 30354711 | TMTC1 | 0.00096 | 128 |
| 256 | chr4 | 81187466 | 81187605 | FGF5 | 4.9E−11 | 128 |
| 257 | chr11 | 22362772 | 22363504 | SLC17A6 | 2.7E−37 | 127 |
| 258 | chr7 | 32467625 | 32467947 | LOC100130673 | 2.9E−29 | 126 |
| 259 | chr20 | 53092293 | 53093101 | DOK5 | 5.8E−71 | 126 |
| 260 | chr19 | 47951120 | 47951495 | SLC8A2 | 2E−39 | 124 |
| 261 | chr1 | 156878418 | 156878573 | LRRC71 | 0.018 | 120 |
| 262 | chr10 | 94451209 | 94452485 | HHEX | 1.7E−69 | 120 |
| 263 | chr19 | 15121590 | 15122224 | CCDC105 | 5.7E−66 | 119 |
| 264 | chr21 | 45770244 | 45770310 | TRPM2 | 9.1E−10 | 118 |
| 265 | chr20 | 54579077 | 54579729 | CBLN4 | 1.1E−35 | 118 |
| 266 | chr11 | 66188570 | 66188960 | NPAS4 | 9.4E−34 | 118 |
| 267 | chr16 | 86825232 | 86825584 | FOXL1 | 1.3E−14 | 118 |
| 268 | chr7 | 1329253 | 1329513 | UNCX | 0.000052 | 117 |
| 269 | chr11 | 65359673 | 65360327 | KCNK7 | 9.3E−100 | 116 |
| 270 | chr22 | 29876701 | 29876739 | NEFH | 1.4E−22 | 114 |
| 271 | chr20 | 61885447 | 61885809 | NKAIN4 | 2.1E−55 | 113 |
| 272 | chr19 | 719371 | 719517 | PALM | 2.4E−21 | 113 |
| 273 | chr11 | 2308315 | 2308626 | C11orf21 | 0.00033 | 112 |
| 274 | chr21 | 10990592 | 10990663 | TPTE | 0.00000044 | 111 |
| 275 | chr2 | 130750104 | 130750307 | RAB6C-AS1 | 1.6E−42 | 108 |
| 276 | chr6 | 26305227 | 26305787 | HIST1H4H | 1E−25 | 107 |
| 277 | chr19 | 3558248 | 3558433 | MFSD12 | 1 | 105 |
| 278 | chr21 | 45575286 | 45575930 | C21orf33 | 4.8E−47 | 105 |
| 279 | chr4 | 568634 | 568727 | PDE6B | 0.0000018 | 105 |
| 280 | chr12 | 1973599 | 1974324 | LRTM2 | 2.1E−36 | 102 |
| 281 | chr11 | 113953544 | 113953909 | ZBTB16 | 3.3E−29 | 100 |
| 282 | chr17 | 79224936 | 79226031 | C17orf89 | 8.9E−20 | 100 |
| 283 | chr2 | 202900905 | 202901100 | FZD7 | 9.8E−12 | 99 |
| 284 | chr22 | 23915439 | 23915669 | IGLL1 | 4.5E−10 | 98 |
| 285 | chr4 | 13545251 | 13545612 | NKX3-2 | 7.9E−39 | 97 |
| 286 | chr20 | 61162111 | 61162334 | MIR133A2 | 0.000000087 | 97 |
| 287 | chr14 | 106025668 | 106025886 | TMEM121 | 1 | 94 |
| 288 | chr1 | 162351633 | 162352162 | C1orf226 | 0.00000016 | 93 |
| 289 | chr22 | 51042795 | 51042987 | MAPK8IP2 | 5.8E−27 | 93 |
| 290 | chr20 | 59827426 | 59828328 | CDH4 | 5.2E−128 | 93 |
| 291 | chr13 | 112723041 | 112724206 | SOX1 | 2.1E−56 | 92 |
| 292 | chr17 | 15820942 | 15821016 | ADORA2B | 8.9E−15 | 90 |
| 293 | chr5 | 415418 | 415925 | EXOC3-AS1 | 3E−41 | 89 |
| 294 | chr11 | 1316235 | 1316522 | TOLLIP | 0.0000063 | 88 |
| 295 | chr2 | 8422081 | 8422507 | LINC00299 | 6.9E−10 | 86 |
| 296 | chr17 | 48154004 | 48154485 | PDK2 | 7.3E−20 | 85 |
| 297 | chr18 | 908970 | 909154 | ADCYAP1 | 1.7E−18 | 83 |
| 298 | chr22 | 29876775 | 29877285 | NEFH | 5.4E−49 | 82 |
| 299 | chr8 | 25899954 | 25900122 | EBF2 | 0.00000015 | 81 |
| 300 | chr19 | 57617483 | 57617984 | USP29 | 1.8E−58 | 81 |
| 301 | chr10 | 38893450 | 38894256 | ACTR3BP5 | 1 | 80 |
| 302 | chr6 | 50682644 | 50682763 | TFAP2D | 3.1E−13 | 80 |
| 303 | chr5 | 16179209 | 16179553 | MARCH11 | 1.8E−51 | 75 |
| 304 | chr2 | 3833710 | 3833982 | DCDC2C | 2.7E−09 | 75 |
| 305 | chr2 | 207506796 | 207507520 | LOC200726 | 8.9E−36 | 73 |
| 306 | chr3 | 172165390 | 172166691 | GHSR | 0.00002 | 70 |
| 307 | chr5 | 140229522 | 140229708 | PCDHA9 | 0.00000021 | 69 |
| 308 | chr11 | 83392903 | 83393677 | DLG2 | 0.0000035 | 69 |
| 309 | chr6 | 170553884 | 170554906 | LOC154449 | 4.7E−10 | 67 |
| 310 | chr21 | 31311386 | 31312141 | GRIK1 | 1.3E−66 | 67 |
| 311 | chr17 | 35299347 | 35300455 | LHX1 | 9.4E−93 | 67 |
| 312 | chr5 | 140249927 | 140250948 | PCDHA11 | 1.4E−84 | 66 |
| 313 | chr5 | 140237185 | 140237453 | PCDHA10 | 2.7E−32 | 65 |
| 314 | chr2 | 233981748 | 233982007 | INPP5D | 4E−11 | 63 |
| 315 | chr19 | 44331123 | 44331262 | ZNF283 | 0.000062 | 63 |
| 316 | chr16 | 32896382 | 32896472 | SLC6A10P | 0.013 | 61 |
| 317 | chr16 | 33040309 | 33040410 | SLC6A10P | 2.2E−11 | 61 |
| 318 | chr14 | 101512524 | 101512693 | MIR487B | 8.5E−11 | 59 |
| 319 | chr17 | 3289378 | 3289570 | OR1E1 | 0.093 | 59 |
| 320 | chr17 | 46685244 | 46685499 | HOXB7 | 5.1E−19 | 57 |
| 321 | chr14 | 48143369 | 48145724 | MDGA2 | 1.2E−274 | 56 |
| 322 | chr5 | 140723454 | 140724037 | PCDHGA3 | 1.5E−28 | 55 |
| 323 | chr5 | 2740084 | 2741271 | IRX2 | 1.2E−52 | 54 |
| 324 | chr1 | 27849011 | 27849318 | WASF2 | 0.00000016 | 53 |
| 325 | chr13 | 112759584 | 112759956 | LINC00403 | 4.4E−19 | 52 |
| 326 | chr1 | 149138346 | 149138516 | | 1.9E−10 | 51 |
| 327 | chr4 | 5892243 | 5892335 | CRMP1 | 5.8E−12 | 51 |
| 328 | chr5 | 140201457 | 140201541 | PCDHA5 | 3.5E−10 | 50 |
| 329 | chr10 | 23483741 | 23484604 | PTF1A | 5.6E−44 | 50 |
| 330 | chr4 | 111553009 | 111554336 | PITX2 | 3.7E−74 | 49 |
| 331 | chr12 | 128751380 | 128751622 | TMEM132C | 0.00004 | 49 |
| 332 | chr8 | 57358126 | 57359512 | PENK | 6.1E−164 | 49 |
| 333 | chr19 | 3382194 | 3382397 | NFIC | 0.000042 | 47 |
| 334 | chr7 | 103630754 | 103631004 | RELN | 1 | 46 |
| 335 | chr2 | 105472533 | 105473187 | POU3F3 | 5E−41 | 46 |
| 336 | chr19 | 12880706 | 12880846 | HOOK2 | 6.5E−29 | 46 |
| 337 | chr10 | 134599091 | 134599161 | NKX6-2 | 9.1E−20 | 46 |
| 338 | chr15 | 32605344 | 32605617 | GOLGA8K | 6.4E−45 | 46 |
| 339 | chr4 | 156129443 | 156129588 | NPY2R | 4.9E−16 | 45 |
| 340 | chr4 | 6450857 | 6451235 | PPP2R2C | 0.00083 | 45 |
| 341 | chr3 | 10858358 | 10858432 | SLC6A11 | 0.00000011 | 44 |
| 342 | chr16 | 1349677 | 1350019 | UBE2I | 1 | 44 |
| 343 | chr10 | 7453773 | 7455338 | SFMBT2 | 1.1E−109 | 44 |
| 344 | chr22 | 40081927 | 40082386 | CACNA1I | 6.6E−53 | 43 |
| 345 | chr19 | 58520728 | 58521646 | ZNF606 | 4.5E−82 | 43 |
| 346 | chr5 | 63256548 | 63257483 | HTR1A | 5E−57 | 43 |
| 347 | chr5 | 140573450 | 140574308 | PCDHB10 | 3.5E−67 | 42 |
| 348 | chr19 | 52222262 | 52223082 | HAS1 | 1.7E−83 | 41 |
| 349 | chr2 | 163695679 | 163696233 | KCNH7 | 8.8E−26 | 40 |
| 350 | chr19 | 18760932 | 18761109 | KLHL26 | 0.0000025 | 40 |
| 351 | chr19 | 4280099 | 4280258 | SHD | 1.5E−14 | 39 |
| 352 | chr17 | 78417763 | 78418214 | MIR4730 | 1E−24 | 39 |
| 353 | chr2 | 90413838 | 90414075 | MIR4436A | 1.3E−27 | 39 |
| 354 | chr12 | 54348949 | 54349679 | HOXC12 | 3.9E−74 | 38 |
| 355 | chr10 | 61900254 | 61901074 | ANK3 | 4.6E−09 | 38 |
| 356 | chr17 | 5000459 | 5000525 | ZFP3 | 2.5E−21 | 37 |
| 357 | chr1 | 50881119 | 50882140 | DMRTA2 | 1.4E−33 | 37 |
| 358 | chr1 | 119549073 | 119551422 | TBX15 | 8.2E−127 | 36 |
| 359 | chr5 | 140568074 | 140569215 | PCDHB9 | 3.9E−69 | 36 |
| 360 | chr7 | 1714908 | 1715440 | ELFN1 | 2.9E−18 | 36 |
| 361 | chr12 | 54354501 | 54355620 | HOXC12 | 1.2E−100 | 35 |
| 362 | chr5 | 180076480 | 180076558 | FLT4 | 0.0000029 | 34 |
| 363 | chr22 | 25678673 | 25679272 | IGLL3P | 1.6E−80 | 34 |
| 364 | chr2 | 865001 | 865075 | LINC01115 | 0.00000086 | 34 |
| 365 | chr5 | 140227983 | 140228669 | PCDHA9 | 2.4E−28 | 33 |
| 366 | chr7 | 157477410 | 157478936 | MIR153-2 | 1.6E−160 | 33 |
| 367 | chr1 | 237206266 | 237206721 | RYR2 | 3.4E−55 | 33 |
| 368 | chr4 | 3443962 | 3444348 | HGFAC | 0.000078 | 33 |
| 369 | chr5 | 1881769 | 1886259 | IRX4 | 6.9E−26 | 31 |
| 370 | chr12 | 75602890 | 75603401 | KCNC2 | 2.2E−35 | 31 |
| 371 | chr8 | 55370442 | 55372108 | SOX17 | 1.1E−157 | 30 |
| 372 | chr19 | 51107536 | 51107741 | SNAR-F | 1.4E−23 | 29 |
| 373 | chr18 | 906366 | 906453 | ADCYAP1 | 6.1E−12 | 29 |
| 374 | chr8 | 144154713 | 144154850 | C8orf31 | 0.000000014 | 28 |
| 375 | chr22 | 19137546 | 19138349 | GSC2 | 9.8E−88 | 28 |
| 376 | chr19 | 38308083 | 38308175 | LOC644554 | 1.7E−17 | 27 |
| 377 | chr7 | 105319306 | 105319905 | ATXN7L1 | 4.2E−36 | 26 |
| 378 | chr5 | 140581175 | 140581476 | PCDHB11 | 4.9E−34 | 26 |
| 379 | chr8 | 145938761 | 145939071 | ARHGAP39 | 1.1E−41 | 26 |
| 380 | chr5 | 140255008 | 140255417 | PCDHA12 | 2.4E−22 | 25 |
| 381 | chr10 | 1850245 | 1850362 | ADARB2 | 0.053 | 25 |
| 382 | chr12 | 85306189 | 85306621 | SLC6A15 | 2.1E−37 | 24 |
| 383 | chr19 | 9473589 | 9474000 | ZNF177 | 3.7E−40 | 24 |
| 384 | chr13 | 95354189 | 95355242 | LOC101927248 | 5.2E−47 | 24 |
| 385 | chr20 | 62092340 | 62092502 | KCNQ2 | 0.00017 | 23 |
| 386 | chr20 | 17206672 | 17206744 | PCSK2 | 0.00000056 | 22 |
| 387 | chr3 | 239204 | 239820 | CHL1 | 2.1E−47 | 22 |
| 388 | chr18 | 31020519 | 31020681 | CCDC178 | 3E−42 | 22 |
| 389 | chr1 | 6196843 | 6197229 | CHD5 | 0.0053 | 22 |
| 390 | chr17 | 25289683 | 25290204 | MIR4522 | 1 | 21 |
| 391 | chr7 | 101961700 | 101962042 | MIR4285 | 1.8E−28 | 20 |
| 392 | chr5 | 140167107 | 140167575 | PCDHA1 | 4.7E−53 | 20 |
| 393 | chr22 | 20792363 | 20792784 | SCARF2 | 1.3E−38 | 20 |
| 394 | chr1 | 228558872 | 228559385 | MIR6742 | 2E−19 | 20 |
| 395 | chr7 | 62574764 | 62574921 | ZNF733P | 1.7E−10 | 20 |
| 396 | chr15 | 98195864 | 98196390 | LOC101927310 | 3.8E−27 | 20 |
| 397 | chr7 | 100942951 | 100943665 | LOC101927746 | 9.3E−42 | 19 |
| 398 | chr1 | 26219044 | 26219341 | STMN1 | 0.000015 | 19 |
| 399 | chr10 | 117817441 | 117817914 | GFRA1 | 0.21 | 18 |
| 400 | chr4 | 153871339 | 153871959 | FHDC1 | 0.0011 | 18 |
| 401 | chr1 | 17012572 | 17012827 | | 1.2E−13 | 17 |
| 402 | chr2 | 503091 | 503140 | TMEM18 | 0.036 | 17 |
| 403 | chr4 | 5891985 | 5892089 | CRMP1 | 0.000000003 | 17 |
| 404 | chr14 | 65008148 | 65008905 | HSPA2 | 1.2E−45 | 17 |
| 405 | chr16 | 84552750 | 84553106 | TLDC1 | 3.2E−15 | 17 |
| 406 | chr8 | 117950453 | 117950884 | AARD | 1.8E−58 | 16 |
| 407 | chr5 | 178421407 | 178422336 | GRM6 | 2E−164 | 16 |
| 408 | chr7 | 45614067 | 45614108 | ADCY1 | 0.21 | 16 |
| 409 | chr18 | 55095098 | 55096420 | ONECUT2 | 3.6E−18 | 16 |
| 410 | chr12 | 57618686 | 57618783 | SHMT2 | 0.000000005 | 16 |
| 411 | chr11 | 60869938 | 60870250 | CD5 | 4.8E−10 | 15 |
| 412 | chr2 | 11809879 | 11809946 | NTSR2 | 0.000013 | 14 |
| 413 | chr6 | 166076755 | 166076790 | PDE10A | 7.7E−10 | 14 |
| 414 | chr1 | 209405127 | 209405216 | MIR205HG | 1.2E−16 | 14 |
| 415 | chr6 | 12296267 | 12296591 | EDN1 | 0.0007 | 13 |
| 416 | chr3 | 196757250 | 196757477 | MFI2 | 0.00014 | 13 |
| 417 | chr6 | 99235956 | 99236151 | POU3F2 | 0.0081 | 13 |
| 418 | chr6 | 21801247 | 21801614 | CASC15 | 0.046 | 12 |
| 419 | chr2 | 241858321 | 241858520 | C2orf54 | 2.4E−10 | 12 |
| 420 | chr15 | 27018354 | 27018756 | GABRB3 | 3.7E−73 | 12 |
| 421 | chr2 | 220361634 | 220362002 | LOC100996693 | 1.9E−30 | 11 |
| 422 | chr2 | 241081083 | 241081490 | OTOS | 0.00016 | 10 |
| 423 | chr22 | 42316084 | 42316198 | MIR378I | 3E−16 | 10 |
| 424 | chr2 | 1358307 | 1358515 | TPO | 0.0013 | 9 |
| 425 | chr5 | 140242203 | 140242951 | PCDHA11 | 5.5E−46 | 9 |
| 426 | chr2 | 31805971 | 31806049 | SRD5A2 | 2E−20 | 9 |
| 427 | chr5 | 45695921 | 45696312 | HCN1 | 2.2E−53 | 9 |
| 428 | chr17 | 5019287 | 5020003 | USP6 | 1.5E−52 | 9 |
| 429 | chr20 | 56283770 | 56284043 | PMEPA1 | 7E−11 | 9 |
| 430 | chr12 | 127256640 | 127256977 | LINC00944 | 1.5E−10 | 8 |
| 431 | chr3 | 169539774 | 169540529 | LRRIQ4 | 4.8E−31 | 8 |
| 432 | chr11 | 2720329 | 2721961 | KCNQ1OT1 | 7E−55 | 8 |
| 433 | chr19 | 33726527 | 33726834 | SLC7A10 | 5.9E−26 | 8 |
| 434 | chr16 | 66400388 | 66400700 | CDH5 | 4.8E−13 | 8 |
| 435 | chr16 | 85133065 | 85133390 | FAM92B | 0.00021 | 8 |
| 436 | chr10 | 120354405 | 120355201 | PRLHR | 4.5E−18 | 7 |
| 437 | chr12 | 130822255 | 130822382 | PIWIL1 | 0.89 | 7 |
| 438 | chr2 | 242833078 | 242833374 | LINC01237 | 1.7E−11 | 7 |
| 439 | chr10 | 47083282 | 47083632 | NPY4R | 3.4E−26 | 7 |
| 440 | chr20 | 62398583 | 62398727 | SLC2A4RG | 3.2E−12 | 7 |
| 441 | chr19 | 20843919 | 20844519 | ZNF626 | 3.8E−25 | 6 |
| 442 | chr2 | 233350491 | 233351425 | ECEL1 | 4.5E−125 | 6 |
| 443 | chr12 | 29937346 | 29937408 | TMTC1 | 3.5E−18 | 6 |
| 444 | chr17 | 3289714 | 3290120 | OR1E1 | 1.7E−42 | 6 |
| 445 | chr5 | 76934957 | 76935276 | OTP | 4.4E−32 | 6 |
| 446 | chr2 | 105459338 | 105459379 | LINC01158 | 7.1E−17 | 5 |
| 447 | chr19 | 15288798 | 15288867 | MIR6795 | 1.6E−10 | 5 |
| 448 | chr3 | 26664476 | 26666187 | LRRC3B | 1.5E−49 | 5 |
| 449 | chr12 | 94954627 | 94954828 | MIR5700 | 1 | 5 |
| 450 | chr4 | 174439903 | 174440378 | HAND2 | 7E−29 | 4 |
| 451 | chr3 | 179754308 | 179755306 | PEX5L | 8.8E−82 | 4 |
| 452 | chr4 | 190944450 | 190944779 | | 5.3E−11 | 4 |
| 453 | chr8 | 2092991 | 2093307 | MIR7160 | 4.4E−17 | 4 |
| 454 | chr12 | 81471418 | 81472263 | ACSS3 | 1.4E−58 | 4 |
| 455 | chr6 | 100896945 | 100897620 | SIM1 | 6.6E−40 | 3 |
| 456 | chr13 | 112720926 | 112720970 | SOX1 | 0.000059 | 3 |
| 457 | chr13 | 112720978 | 112721028 | SOX1 | 0.012 | 3 |
| 458 | chr12 | 123560320 | 123560680 | LOC100507091 | 2.7E−16 | 3 |
| 459 | chr4 | 126237764 | 126239021 | FAT4 | 4.4E−99 | 3 |
| 460 | chr2 | 132587031 | 132587694 | C2orf27B | 9.8E−92 | 3 |
| 461 | chr9 | 138606644 | 138606933 | KCNT1 | 0.0000018 | 3 |
| 462 | chr2 | 171676467 | 171677023 | GAD1 | 4.1E−20 | 3 |
| 463 | chr19 | 19650954 | 19651206 | CILP2 | 6.2E−44 | 3 |
| 464 | chr20 | 23015873 | 23016284 | SSTR4 | 1.8E−77 | 3 |
| 465 | chr19 | 36247321 | 36247963 | HSPB6 | 9.7E−55 | 3 |
| 466 | chr10 | 3823892 | 3824178 | KLF6 | 1.1E−27 | 3 |
| 467 | chr19 | 49575902 | 49575939 | KCNA7 | 1.7E−15 | 3 |
| 468 | chr20 | 61992155 | 61992856 | CHRNA4 | 2.9E−73 | 3 |
| 469 | chr4 | 8582798 | 8583233 | GPR78 | 5.1E−28 | 3 |
| 470 | chr15 | 93122815 | 93123359 | LINC00930 | 0.0000058 | 3 |
| 471 | chr6 | 100441820 | 100441896 | MCHR2-AS1 | 1.7E−25 | 2 |
| 472 | chr10 | 131770707 | 131771555 | EBF3 | 8E−131 | 2 |
| 473 | chr8 | 141599156 | 141599489 | AGO2 | 3.8E−26 | 2 |
| 474 | chr5 | 1430646 | 1431099 | SLC6A3 | 1 | 2 |
| 475 | chr4 | 190962118 | 190962463 | | 7.5E−17 | 2 |
| 476 | chr17 | 26864305 | 26864653 | FOXN1 | 0.058 | 2 |
| 477 | chr2 | 27070467 | 27070520 | DPYSL5 | 2.9E−11 | 2 |
| 478 | chr16 | 30485516 | 30485810 | ITGAL | 3.1E−36 | 2 |
| 479 | chr22 | 32753597 | 32754024 | RFPL3 | 0.55 | 2 |
| 480 | chr17 | 33842094 | 33842307 | SLFN12L | 1.2E−14 | 2 |
| 481 | chr10 | 47653782 | 47654043 | ANTXRL | 4.9E−20 | 2 |
| 482 | chr14 | 69256533 | 69257115 | ZFP36L1 | 8.1E−105 | 2 |
| 483 | chr6 | 72129888 | 72129924 | LINC00472 | 1E−16 | 2 |
| 484 | chr1 | 75595918 | 75596017 | LHX8 | 3.5E−11 | 2 |
| 485 | chr16 | 89888643 | 89888937 | FANCA | 8.6E−09 | 2 |
| 486 | chr15 | 89922050 | 89922792 | MIR9-3HG | 1.2E−57 | 2 |
| 487 | chr6 | 100066597 | 100066795 | PRDM13 | 1.1E−15 | 1 |
| 488 | chr14 | 103740306 | 103741026 | EIF5 | 6.2E−23 | 1 |
| 489 | chr13 | 114876595 | 114876834 | RASA3 | 4E−14 | 1 |
| 490 | chr9 | 115652625 | 115653447 | SLC46A2 | 2.5E−54 | 1 |
| 491 | chr10 | 118899260 | 118900464 | VAX1 | 3E−55 | 1 |
| 492 | chr6 | 123317044 | 123317899 | CLVS2 | 2E−63 | 1 |
| 493 | chr1 | 1244840 | 1245076 | PUSL1 | 8.8E−09 | 1 |
| 494 | chr5 | 127874530 | 127874609 | FBN2 | 2.3E−12 | 1 |
| 495 | chr9 | 136107471 | 136107874 | OBP2B | 2.3E−11 | 1 |
| 496 | chr9 | 139738782 | 139739504 | C9orf172 | 1.8E−116 | 1 |
| 497 | chr5 | 140810403 | 140811314 | PCDHGA12 | 1.1E−57 | 1 |
| 498 | chr8 | 145955849 | 145955908 | ZNF251 | 1 | 1 |
| 499 | chr1 | 167408458 | 167409113 | CD247 | 1.1E−49 | 1 |
| 500 | chr2 | 1695129 | 1695430 | PXDN | 0.0000018 | 1 |
| 501 | chr20 | 17207378 | 17209657 | PCSK2 | 1.1E−11 | 1 |
| 502 | chr5 | 177398015 | 177398535 | PROP1 | 1.1E−36 | 1 |
| 503 | chr11 | 17741505 | 17742031 | MYOD1 | 2.9E−67 | 1 |
| 504 | chr3 | 183817600 | 183818417 | HTR3E | 0.0000017 | 1 |
| 505 | chr9 | 19934400 | 19934826 | SLC24A2 | 3E−29 | 1 |
| 506 | chr1 | 207842502 | 207843415 | CR1L | 4.9E−85 | 1 |
| 507 | chr20 | 21694312 | 21694738 | PAX1 | 5.1E−22 | 1 |
| 508 | chr6 | 25027421 | 25028183 | FAM65B | 1.1E−13 | 1 |
| 509 | chr15 | 31638051 | 31638347 | KLF13 | 7.4E−12 | 1 |
| 510 | chr16 | 32823336 | 32823505 | SLC6A10P | 0.0029 | 1 |
| 511 | chr16 | 33852632 | 33852701 | LINC00273 | 4.6E−19 | 1 |
| 512 | chr5 | 33936457 | 33937261 | RXFP3 | 3.4E−34 | 1 |
| 513 | chr9 | 34809685 | 34810113 | FAM205BP | 2E−61 | 1 |
| 514 | chr1 | 40235714 | 40236348 | OXCT2 | 2.7E−35 | 1 |
| 515 | chr7 | 4901208 | 4901886 | PAPOLB | 1.9E−46 | 1 |
| 516 | chr22 | 50464637 | 50464834 | IL17REL | 0.035 | 1 |
| 517 | chr10 | 50976347 | 50977132 | OGDHL | 2.4E−81 | 1 |
| 518 | chr19 | 52954595 | 52954903 | ZNF578 | 2.7E−33 | 1 |
| 519 | chr19 | 58908103 | 58908181 | RNF225 | 5.7E−16 | 1 |
| 520 | chr20 | 61002544 | 61003481 | RBBP8NL | 1.2E−10 | 1 |
| 521 | chr19 | 611413 | 611702 | HCN2 | 1E−13 | 1 |
| 522 | chr3 | 62356078 | 62356886 | FEZF2 | 3.9E−21 | 1 |
| 523 | chr15 | 66914678 | 66914731 | LINC01169 | 0.000024 | 1 |
| 524 | chr11 | 7272757 | 7274234 | SYT9 | 6.7E−120 | 1 |
| 525 | chr7 | 8483144 | 8483735 | NXPH1 | 4.3E−12 | 1 |
| 526 | chr2 | 85664611 | 85664747 | SH2D6 | 0.00000094 | 1 |
| 527 | chr14 | 86000161 | 86000222 | FLRT2 | 1.5E−10 | 1 |
| 528 | chr1 | 933509 | 933571 | HES4 | 2.9E−16 | 1 |
| 529 | chr10 | 94995 | 95412 | TUBB8 | 3.3E−30 | 1 |

### Hepatocellular Carcinoma

In another aspect is provided a method of detecting a level of DNA methylation in a subject at risk of developing a hepatocellular carcinoma (HCC), the method including: determining the degree of methylation of CpG sites within a plurality of gene regions in a DNA sample from the subject, wherein the plurality of gene regions includes at least 5 different gene regions in Table HCC. In embodiments, an increased level of methylated CpG sites relative to a standard control indicates a higher risk of hepatocellular carcinoma.

Provided herein is a method of treating hepatocellular carcinoma in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table HCC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the hepatocellular carcinoma is Stage I, Stage II, or Stage III. In embodiments, the hepatocellular carcinoma is Stage I. In embodiments, the hepatocellular carcinoma is Stage II. In embodiments, the hepatocellular carcinoma is Stage III.

Provided herein is a method of diagnosing hepatocellular carcinoma in a patient comprising: (a) detecting a level of methylated CpG sites within a plurality of gene regions in a DNA sample from the patient, wherein the plurality of gene regions comprise at least 5 different gene regions in Table HCC; and (b) diagnosing the patient with hepatocellular carcinoma when the DNA sample has an increased level of methylated CpG sites, relative to a standard control, within the plurality of gene regions. In embodiments, the hepatocellular carcinoma is Stage I, Stage II, or Stage III. In embodiments, the hepatocellular carcinoma is Stage I. In embodiments, the hepatocellular carcinoma is Stage II. In embodiments, the hepatocellular carcinoma is Stage III. I In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having hepatocellular carcinoma or monitoring risk for developing hepatocellular carcinoma in a patient comprising: (a) detecting a level of methylated CpG sites within a plurality of gene regions in a DNA sample from the patient at a first time point, wherein the plurality of gene regions comprise at least 5 different gene regions in Table HCC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing hepatocellular carcinoma. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing hepatocellular carcinoma or does not have hepatocellular carcinoma. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing hepatocellular carcinoma or may have hepatocellular carcinoma. In embodiments, the hepatocellular carcinoma is Stage I, Stage II, or Stage III. In embodiments, the hepatocellular carcinoma is Stage I. In embodiments, the hepatocellular carcinoma is Stage II. In embodiments, the hepatocellular carcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table HCC. In embodiments, the plurality of gene regions includes at least 2 DMRs (i.e., gene regions) in Table HCC. In embodiments, the plurality of gene regions includes at least 3 DMRs in Table HCC. In embodiments, the plurality of gene regions includes at least 4 DMRs in Table HCC. In embodiments, the plurality of gene regions includes at least 5 DMRs in Table HCC. In embodiments, the plurality of gene regions includes at least 6 DMRs in Table HCC. In embodiments, the plurality of gene regions includes at least 7 DMRs in Table HCC. In embodiments, the plurality of gene regions includes at least 8 DMRs in Table HCC. In embodiments, the plurality of gene regions includes at least 9 DMRs in Table HCC. In embodiments, the plurality of gene regions includes at least 10 DMRs in Table HCC. In embodiments, the plurality of gene regions includes at least 11 DMRs in Table HCC. In embodiments, the plurality of gene regions includes at least 12 DMRs in Table HCC. In embodiments, the plurality of gene regions includes at least 13 DMRs in Table HCC. In embodiments, the plurality of gene regions includes at least 14 DMRs in Table HCC. In embodiments, the plurality of gene regions includes at least 15 DMRs in Table HCC. In embodiments, the plurality of gene regions includes at least 16 DMRs in Table HCC. In embodiments, the plurality of gene regions includes at least 17 DMRs in Table HCC. In embodiments, the plurality of gene regions includes at least 18 DMRs in Table HCC. In embodiments, the plurality of gene regions includes at least 19 DMRs in Table HCC. In embodiments, the plurality of gene regions includes at least 20 DMRs in Table HCC. In embodiments, the plurality of gene regions includes at least 21 DMRs in Table HCC. In embodiments, the plurality of gene regions includes at least 22 DMRs in Table HCC. In embodiments, the plurality of gene regions includes at least 23 DMRs in Table HCC. In embodiments, the plurality of gene regions includes at least 24 DMRs in Table HCC. In embodiments, the plurality of gene regions includes at least 25 DMRs in Table HCC. In embodiments, the plurality of gene regions includes at least 30 DMRs in Table HCC. In embodiments, the plurality of gene regions includes at least 35 DMRs in Table HCC. In embodiments, the plurality of gene regions includes at least 40 DMRs in Table HCC. In embodiments, the plurality of gene regions includes at least 45 DMRs in Table HCC. In embodiments, the plurality of gene regions includes at least 50 DMRs in Table HCC. In embodiments, the plurality of gene regions includes at least 55 DMRs in Table HCC. In embodiments, the plurality of gene regions includes at least 60 DMRs in Table HCC. In embodiments, the plurality of gene regions includes at least 65 DMRs in Table HCC. In embodiments, the plurality of gene regions includes at least 70 DMRs in Table HCC. In embodiments, the plurality of gene regions includes at least 75 DMRs in Table HCC. In embodiments, the plurality of gene regions includes at least 80 DMRs in Table HCC. In embodiments, the plurality of gene regions includes at least 85 DMRs in Table HCC. In embodiments, the plurality of gene regions includes at least 90 DMRs in Table HCC. In embodiments, the plurality of gene regions includes at least 95 DMRs in Table HCC. In embodiments, the plurality of gene regions includes at least 110 DMRs in Table HCC. In embodiments, the plurality of gene regions includes at least 120 DMRs in Table HCC. In embodiments, the plurality of gene regions includes at least 130 DMRs in Table HCC. In embodiments, the plurality of gene regions includes at least 140 DMRs in Table HCC. In embodiments, the plurality of gene regions includes at least 150 DMRs in Table HCC. In embodiments, the plurality of gene regions includes at least 160 DMRs in Table HCC. In embodiments, the plurality of gene regions includes at least 170 DMRs in Table HCC. In embodiments, the plurality of gene regions includes at least 180 DMRs in Table HCC. In embodiments, the plurality of gene regions includes at least 190 DMRs in Table HCC. In embodiments, the plurality of gene regions includes at least 200 DMRs in Table HCC. In embodiments, the plurality of gene regions includes at least 225 DMRs in Table HCC. In embodiments, the plurality of gene regions includes at least 250 DMRs in Table HCC. In embodiments, the plurality of gene regions includes at least 275 DMRs in Table HCC. In embodiments, the plurality of gene regions includes at least 300 DMRs in Table HCC. In embodiments, the plurality of gene regions includes at least 325 DMRs in Table HCC. In embodiments, the plurality of gene regions includes at least 350 DMRs in Table HCC. In embodiments, the plurality of gene regions includes at least 375 DMRs in Table HCC. In embodiments, the plurality of gene regions includes at least 400 DMRs in Table HCC.

In embodiments, the plurality of gene regions includes the first DMR (i.e., gene region) in Table HCC. In embodiments, the plurality of gene regions includes the first 2 DMRs (i.e., gene regions) in Table HCC. In embodiments, the plurality of gene regions includes the first 3 DMRs in Table HCC. In embodiments, the plurality of gene regions includes the first 4 DMRs in Table HCC. In embodiments, the plurality of gene regions includes the first 5 DMRs in Table HCC. In embodiments, the plurality of gene regions includes the first 6 DMRs in Table HCC. In embodiments, the plurality of gene regions includes the first 7 DMRs in Table HCC. In embodiments, the plurality of gene regions includes the first 8 DMRs in Table HCC. In embodiments, the plurality of gene regions includes the first 9 DMRs in Table HCC. In embodiments, the plurality of gene regions includes the first 10 DMRs in Table HCC. In embodiments, the plurality of gene regions includes the first 11 DMRs in Table HCC. In embodiments, the plurality of gene regions includes the first 12 DMRs in Table HCC. In embodiments, the plurality of gene regions includes the first 13 DMRs in Table HCC. In embodiments, the plurality of gene regions includes the first 14 DMRs in Table HCC. In embodiments, the plurality of gene regions includes the first 15 DMRs in Table HCC. In embodiments, the plurality of gene regions includes the first 16 DMRs in Table HCC. In embodiments, the plurality of gene regions includes the first 17 DMRs in Table HCC. In embodiments, the plurality of gene regions includes the first 18 DMRs in Table HCC. In embodiments, the plurality of gene regions includes the first 19 DMRs in Table HCC. In embodiments, the plurality of gene regions includes the first 20 DMRs in Table HCC. In embodiments, the plurality of gene regions includes the first 21 DMRs in Table HCC. In embodiments, the plurality of gene regions includes the first 22 DMRs in Table HCC. In embodiments, the plurality of gene regions includes the first 23 DMRs in Table HCC. In embodiments, the plurality of gene regions includes the first 24 DMRs in Table HCC. In embodiments, the plurality of gene regions includes the first 25 DMRs in Table HCC. In embodiments, the plurality of gene regions includes the first 30 DMRs in Table HCC. In embodiments, the plurality of gene regions includes the first 35 DMRs in Table HCC. In embodiments, the plurality of gene regions includes the first 40 DMRs in Table HCC. In embodiments, the plurality of gene regions includes the first 45 DMRs in Table HCC. In embodiments, the plurality of gene regions includes the first 50 DMRs in Table HCC. In embodiments, the plurality of gene regions includes the first 55 DMRs in Table HCC. In embodiments, the plurality of gene regions includes the first 60 DMRs in Table HCC. In embodiments, the plurality of gene regions includes the first 65 DMRs in Table HCC. In embodiments, the plurality of gene regions includes the first 70 DMRs in Table HCC. In embodiments, the plurality of gene regions includes the first 75 DMRs in Table HCC. In embodiments, the plurality of gene regions includes the first 80 DMRs in Table HCC. In embodiments, the plurality of gene regions includes the first 85 DMRs in Table HCC. In embodiments, the plurality of gene regions includes the first 90 DMRs in Table HCC. In embodiments, the plurality of gene regions includes the first 95 DMRs in Table HCC. In embodiments, the plurality of gene regions includes the first 110 DMRs in Table HCC. In embodiments, the plurality of gene regions includes the first 120 DMRs in Table HCC. In embodiments, the plurality of gene regions includes the first 130 DMRs in Table HCC. In embodiments, the plurality of gene regions includes the first 140 DMRs in Table HCC. In embodiments, the plurality of gene regions includes the first 150 DMRs in Table HCC. In embodiments, the plurality of gene regions includes the first 160 DMRs in Table HCC. In embodiments, the plurality of gene regions includes the first 170 DMRs in Table HCC. In embodiments, the plurality of gene regions includes the first 180 DMRs in Table HCC. In embodiments, the plurality of gene regions includes the first 190 DMRs in Table HCC. In embodiments, the plurality of gene regions includes the first 200 DMRs in Table HCC. In embodiments, the plurality of gene regions includes the first 225 DMRs in Table HCC. In embodiments, the plurality of gene regions includes the first 250 DMRs in Table HCC. In embodiments, the plurality of gene regions includes the first 275 DMRs in Table HCC. In embodiments, the plurality of gene regions includes the first 300 DMRs in Table HCC. In embodiments, the plurality of gene regions includes the first 325 DMRs in Table HCC. In embodiments, the plurality of gene regions includes the first 350 DMRs in Table HCC. In embodiments, the plurality of gene regions includes the first 375 DMRs in Table HCC. In embodiments, the plurality of gene regions includes the first 400 DMRs in Table HCC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is a tissue biopsy. In embodiments, the confirmatory diagnostic procedure is a biopsy. In embodiments, the confirmatory diagnostic procedure is an ultrasound, a computed tomography scan, a magnetic resonance imaging scan, angiography, or alfa-fetoprotein protein blood test.

In embodiments, the method further includes treating the subject for a hepatocellular carcinoma. In embodiments, treating includes surgery, radiotherapy, chemotherapy, targeted therapy, or immunotherapy. In embodiments, treating includes surgery, radiotherapy, chemotherapy, targeted therapy, immunotherapy, or a combination of two or more thereof. In embodiments, treating includes radiotherapy, chemotherapy, targeted therapy, immunotherapy, or a combination of two or more thereof.

TABLE HCC

| Gene | | | | Gene | Adjusted | |
|---|---|---|---|---|---|---|
| Region No. | Chr | Start | End | Name | p-value | Freq |
| 1 | chr11 | 64034860 | 64034997 | PLCB3 | 7.2E−26 | 418 |
| 2 | chr6 | 42071869 | 42072773 | C6orf132 | 1E−73 | 416 |
| 3 | chr1 | 148901738 | 148902370 | LOC101060524 | 9.8E−118 | 415 |
| 4 | chr2 | 233252812 | 233253144 | ECEL1P2 | 0.015 | 414 |
| 5 | chr4 | 13524753 | 13526008 | LINC01097 | 2E−62 | 413 |
| 6 | chr2 | 90016149 | 90016431 | MIR4436A | 4.1E−41 | 413 |
| 7 | chr14 | 101512524 | 101512693 | MIR487B | 8.5E−11 | 410 |
| 8 | chr7 | 153584345 | 153585592 | DPP6 | 9.9E−129 | 409 |
| 9 | chr4 | 298753 | 299338 | ZNF732 | 3.2E−65 | 409 |
| 10 | chr4 | 7483771 | 7483929 | MIR4274 | 0.00093 | 408 |
| 11 | chr16 | 86066512 | 86066907 | MIR6774 | 9.9E−10 | 407 |
| 12 | chr14 | 105103601 | 105103693 | TMEM179 | 0.00011 | 406 |
| 13 | chr1 | 53992314 | 53992630 | DMRTB1 | 2.1E−10 | 406 |
| 14 | chr13 | 112978530 | 112978862 | LINC01044 | 1.1E−26 | 405 |
| 15 | chr16 | 58019627 | 58019947 | TEPP | 0.00019 | 405 |
| 16 | chr19 | 36912350 | 36912880 | LOC644189 | 7E−91 | 404 |
| 17 | chr13 | 28498102 | 28499045 | PDX1-AS1 | 0.0000017 | 403 |
| 18 | chr11 | 1321811 | 1322152 | TOLLIP | 1.5E−36 | 402 |
| 19 | chr6 | 170730605 | 170730682 | MIR4644 | 0.025 | 402 |
| 20 | chr20 | 37356055 | 37357918 | SLC32A1 | 1.2E−132 | 402 |
| 21 | chr19 | 719203 | 719302 | PALM | 7.1E−12 | 401 |
| 22 | chr17 | 3289582 | 3289717 | OR1E1 | 5.4E−26 | 400 |
| 23 | chr3 | 147136946 | 147137256 | LOC440982 | 3.2E−16 | 399 |
| 24 | chr2 | 5832525 | 5834007 | SOX11 | 1.9E−195 | 399 |
| 25 | chr8 | 143960890 | 143961303 | CYP11B1 | 0.0025 | 398 |
| 26 | chr10 | 135051371 | 135051658 | VENTX | 0.0000011 | 397 |
| 27 | chr8 | 19319176 | 19319381 | CSGALNACT1 | 0.00074 | 397 |
| 28 | chr9 | 125391116 | 125391618 | OR1B1 | 0.0000065 | 396 |
| 29 | chr2 | 112123586 | 112124284 | | 1.5E−84 | 395 |
| 30 | chr3 | 11651407 | 11652034 | VGLL4 | 1.4E−16 | 395 |
| 31 | chr19 | 58545029 | 58545559 | ZSCAN1 | 2.4E−140 | 395 |
| 32 | chr19 | 719371 | 719517 | PALM | 2.4E−21 | 395 |
| 33 | chr5 | 177398015 | 177398535 | PROP1 | 1.1E−36 | 393 |
| 34 | chr8 | 1292265 | 1292374 | LOC286083 | 0.00000059 | 390 |
| 35 | chr20 | 62272671 | 62273058 | STMN3 | 7.9E−36 | 390 |
| 36 | chr17 | 44928287 | 44928439 | WNT9B | 0.00000016 | 388 |
| 37 | chr5 | 2751128 | 2757191 | C5orf38 | 1.3E−152 | 387 |
| 38 | chr14 | 106235606 | 106235783 | MIR4507 | 0.0065 | 385 |
| 39 | chr13 | 28500948 | 28503530 | PDX1-AS1 | 4.3E−128 | 385 |
| 40 | chr6 | 78172191 | 78173170 | HTR1B | 2.5E−97 | 384 |
| 41 | chr16 | 1088290 | 1088515 | SSTR5 | 0.0013 | 383 |
| 42 | chr19 | 58446168 | 58446960 | ZNF418 | 3.6E−59 | 383 |
| 43 | chr1 | 1099206 | 1100655 | MIR200B | 2.3E−62 | 382 |
| 44 | chr17 | 78417763 | 78418214 | MIR4730 | 1E−24 | 382 |
| 45 | chr12 | 133049696 | 133050826 | FBRSL1 | 4.4E−13 | 378 |
| 46 | chr2 | 233284689 | 233284880 | ALPPL2 | 1 | 371 |
| 47 | chr4 | 5570160 | 5570245 | LINC01587 | 1 | 368 |
| 48 | chr5 | 178016539 | 178017711 | COL23A1 | 1.9E−134 | 364 |
| 49 | chr8 | 53851723 | 53853197 | NPBWR1 | 6.5E−128 | 356 |
| 50 | chr3 | 194785870 | 194786337 | XXYLT1-AS1 | 2.7E−21 | 344 |
| 51 | chr1 | 3230469 | 3230688 | ARHGEF16 | 0.0049 | 338 |
| 52 | chr8 | 57358126 | 57359512 | PENK | 6.1E−164 | 338 |
| 53 | chr14 | 73736836 | 73737235 | PAPLN | 8.7E−33 | 327 |
| 54 | chr10 | 104535448 | 104536195 | WBP1L | 1E−24 | 314 |
| 55 | chr7 | 5734902 | 5735261 | RNF216-IT1 | 0.000000028 | 302 |
| 56 | chr16 | 89674874 | 89675253 | DPEP1 | 0.00066 | 300 |
| 57 | chr8 | 97162411 | 97162548 | GDF6 | 6.5E−10 | 296 |
| 58 | chr2 | 225265848 | 225266580 | FAM124B | 5.2E−42 | 291 |
| 59 | chr19 | 14591194 | 14591301 | PTGER1 | 0.007 | 285 |
| 60 | chr14 | 101489246 | 101489649 | MIR411 | 5.1E−09 | 279 |
| 61 | chr19 | 37282439 | 37282687 | ZNF790-AS1 | 0.00000014 | 251 |
| 62 | chr7 | 2149549 | 2150068 | MAD1L1 | 3E−19 | 245 |
| 63 | chr8 | 1449737 | 1450097 | DLGAP2 | 1 | 235 |
| 64 | chr4 | 7483396 | 7483618 | MIR4274 | 0.0000039 | 231 |
| 65 | chr1 | 17020797 | 17020930 | | 0.000018 | 230 |
| 66 | chr1 | 156878418 | 156878573 | LRRC71 | 0.018 | 229 |
| 67 | chr12 | 52685008 | 52685334 | KRT81 | 6.4E−19 | 228 |
| 68 | chr2 | 74726959 | 74727083 | LBX2 | 1.4E−13 | 221 |
| 69 | chr10 | 47653782 | 47654043 | ANTXRL | 4.9E−20 | 218 |
| 70 | chr9 | 116861246 | 116861730 | KIF12 | 9.6E−14 | 215 |
| 71 | chr17 | 3212908 | 3213197 | OR3A4P | 1 | 213 |
| 72 | chr19 | 38308083 | 38308175 | LOC644554 | 1.7E−17 | 211 |
| 73 | chr7 | 1329253 | 1329513 | UNCX | 0.000052 | 204 |
| 74 | chr5 | 140305727 | 140307192 | PCDHAC1 | 6.2E−93 | 203 |
| 75 | chr1 | 153362847 | 153364085 | S100A8 | 4.3E−11 | 200 |
| 76 | chr22 | 46367360 | 46367421 | WNT7B | 0.000000059 | 197 |
| 77 | chr8 | 97156853 | 97158021 | GDF6 | 1.8E−136 | 197 |
| 78 | chr11 | 117069683 | 117070130 | TAGLN | 1.6E−19 | 191 |
| 79 | chr20 | 56283770 | 56284043 | PMEPA1 | 7E−11 | 191 |
| 80 | chr11 | 17741505 | 17742031 | MYOD1 | 2.9E−67 | 190 |
| 81 | chr2 | 864282 | 864406 | LINC01115 | 1.3E−19 | 189 |
| 82 | chr1 | 117753496 | 117753965 | VTCN1 | 0.000000099 | 186 |
| 83 | chr6 | 170413895 | 170414149 | LOC102724511 | 1 | 185 |
| 84 | chr2 | 90413838 | 90414075 | MIR4436A | 1.3E−27 | 185 |
| 85 | chr3 | 192232813 | 192233079 | FGF12 | 8.1E−51 | 184 |
| 86 | chr2 | 202900905 | 202901100 | FZD7 | 9.8E−12 | 184 |
| 87 | chr6 | 26305227 | 26305787 | HIST1H4H | 1E−25 | 183 |
| 88 | chr17 | 48154004 | 48154485 | PDK2 | 7.3E−20 | 183 |
| 89 | chr15 | 96909659 | 96909921 | NR2F2 | 0.0052 | 183 |
| 90 | chr7 | 1270535 | 1270617 | UNCX | 8.6E−09 | 181 |
| 91 | chr17 | 59534376 | 59535253 | TBX4 | 3.4E−43 | 181 |
| 92 | chr11 | 133821131 | 133821312 | IGSF9B | 1 | 180 |
| 93 | chr2 | 242927741 | 242928044 | LOC102723927 | 4.2E−27 | 179 |
| 94 | chr17 | 5000232 | 5000457 | ZFP3 | 1.4E−11 | 179 |
| 95 | chr14 | 105318120 | 105318595 | CEP170B | 0.00037 | 178 |
| 96 | chr10 | 108923670 | 108924867 | SORCS1 | 5.9E−143 | 178 |
| 97 | chr8 | 144155053 | 144155326 | C8orf31 | 7.7E−16 | 178 |
| 98 | chr6 | 166260011 | 166260399 | LINC00602 | 0.22 | 178 |
| 99 | chr17 | 46685244 | 46685499 | HOXB7 | 5.1E−19 | 178 |
| 100 | chr10 | 50819078 | 50820313 | SLC18A3 | 1.2E−133 | 178 |
| 101 | chr2 | 63274711 | 63275273 | LOC100132215 | 1.2E−33 | 178 |
| 102 | chr4 | 165304258 | 165305137 | MARCH1 | 2.6E−88 | 177 |
| 103 | chr10 | 23461316 | 23463617 | PTF1A | 5.9E−163 | 177 |
| 104 | chr1 | 151810557 | 151811034 | LOC100132111 | 6.4E−90 | 176 |
| 105 | chr11 | 24518250 | 24518712 | LUZP2 | 7E−54 | 176 |
| 106 | chr19 | 36735979 | 36736818 | ZNF565 | 2.5E−71 | 176 |
| 107 | chr7 | 101961700 | 101962042 | MIR4285 | 1.8E−28 | 175 |
| 108 | chr1 | 10764635 | 10764790 | CASZ1 | 2.9E−09 | 175 |
| 109 | chr5 | 63256548 | 63257483 | HTR1A | 5E−57 | 175 |
| 110 | chr17 | 70111952 | 70112387 | SOX9-AS1 | 6.5E−15 | 175 |
| 111 | chr1 | 119527391 | 119527516 | TBX15 | 2.2E−16 | 174 |
| 112 | chr17 | 5000871 | 5001123 | USP6 | 6.4E−60 | 174 |
| 113 | chr7 | 5111545 | 5111857 | RBAKDN | 5.3E−12 | 174 |
| 114 | chr4 | 122078292 | 122078409 | TNIP3 | 0.00000006 | 173 |
| 115 | chr9 | 140772527 | 140772595 | CACNA1B | 1.1E−11 | 173 |
| 116 | chr3 | 147108577 | 147111786 | ZIC4 | 1.5E−203 | 173 |
| 117 | chr8 | 38831507 | 38832516 | HTRA4 | 1.6E−120 | 173 |
| 118 | chr13 | 53775090 | 53775595 | LINC01065 | 1.4E−135 | 173 |
| 119 | chr5 | 3386403 | 3386587 | LINC01017 | 1 | 172 |
| 120 | chr7 | 101961741 | 101962354 | MIR4285 | 4.4E−75 | 171 |
| 121 | chr9 | 118046 | 118154 | FOXD4 | 0.0000004 | 171 |
| 122 | chr1 | 217307639 | 217307797 | ESRRG | 0.0000033 | 171 |
| 123 | chr14 | 29237012 | 29237819 | FOXG1 | 3.3E−63 | 171 |
| 124 | chr2 | 865001 | 865075 | LINC01115 | 0.00000086 | 171 |
| 125 | chr1 | 17025587 | 17026500 | | 7.9E−89 | 170 |
| 126 | chr7 | 35294156 | 35299091 | TBX20 | 1.2E−38 | 170 |
| 127 | chr19 | 54481438 | 54481841 | MIR935 | 3.9E−44 | 170 |
| 128 | chr15 | 96959357 | 96959433 | NR2F2 | 0.0000002 | 170 |
| 129 | chr9 | 22005753 | 22006306 | CDKN2B | 9.9E−23 | 169 |
| 130 | chr18 | 55019953 | 55021680 | ST8SIA3 | 0.84 | 169 |
| 131 | chr16 | 86825232 | 86825584 | FOXL1 | 1.3E−14 | 169 |
| 132 | chr2 | 905888 | 906446 | LOC101060385 | 1 | 169 |
| 133 | chr3 | 9987754 | 9988787 | PRRT3-AS1 | 1 | 169 |
| 134 | chr19 | 10403715 | 10404837 | ICAM5 | 0.5 | 168 |
| 135 | chr2 | 74726477 | 74727000 | LBX2 | 3.6E−46 | 168 |
| 136 | chr5 | 132083508 | 132083784 | CCNI2 | 2.9E−11 | 167 |
| 137 | chr20 | 25063816 | 25064630 | VSX1 | 2.9E−69 | 167 |
| 138 | chr17 | 25289683 | 25290204 | MIR4522 | 1 | 167 |
| 139 | chr15 | 66332260 | 66332338 | MIR4311 | 0.00016 | 167 |
| 140 | chr4 | 147561142 | 147561866 | POU4F2 | 7.6E−68 | 166 |
| 141 | chr18 | 19745121 | 19745186 | GATA6-AS1 | 6.1E−09 | 166 |
| 142 | chr3 | 31494123 | 31494215 | STT3B | 0.00002 | 166 |
| 143 | chr13 | 58206330 | 58207289 | PCDH17 | 7.7E−97 | 166 |
| 144 | chr5 | 178421407 | 178422336 | GRM6 | 2E−164 | 165 |
| 145 | chr10 | 22542053 | 22542699 | LOC100130992 | 2.1E−55 | 165 |
| 146 | chr13 | 111978726 | 111978866 | TEX29 | 0.89 | 164 |
| 147 | chr9 | 140128834 | 140129114 | SLC34A3 | 5.6E−12 | 164 |
| 148 | chr7 | 8481842 | 8482826 | NXPH1 | 2.4E−78 | 164 |
| 149 | chr7 | 93657 | 93903 | LOC100507642 | 0.0000015 | 164 |
| 150 | chr2 | 121223490 | 121224836 | LINC01101 | 1.3E−15 | 163 |
| 151 | chr4 | 134071516 | 134074046 | PCDH10 | 9.4E−218 | 163 |
| 152 | chr14 | 26674163 | 26674271 | NOVA1 | 2E−20 | 163 |
| 153 | chr20 | 50721104 | 50721611 | ZFP64 | 4.4E−36 | 163 |
| 154 | chr17 | 5403042 | 5403604 | LOC728392 | 0.0000013 | 163 |
| 155 | chr2 | 114034006 | 114036088 | PAX8 | 1.6E−112 | 162 |
| 156 | chr14 | 23835694 | 23836012 | EFS | 0.00026 | 162 |
| 157 | chr10 | 61900254 | 61901074 | ANK3 | 4.6E−09 | 162 |
| 158 | chr16 | 711211 | 711452 | RHOT2 | 1 | 162 |
| 159 | chr6 | 50818180 | 50818424 | TFAP2B | 4.3E−100 | 161 |
| 160 | chr12 | 54348949 | 54349679 | HOXC12 | 3.9E−74 | 161 |
| 161 | chr2 | 87883776 | 87883951 | MIR4435-1 | 5.4E−70 | 161 |
| 162 | chr10 | 101291127 | 101293384 | NKX2-3 | 3.2E−20 | 160 |
| 163 | chr5 | 140797162 | 140797700 | PCDHGB7 | 4.4E−33 | 160 |
| 164 | chr2 | 176994364 | 176994811 | HOXD8 | 2E−40 | 160 |
| 165 | chr1 | 119549073 | 119551422 | TBX15 | 8.2E−127 | 159 |
| 166 | chr8 | 41165745 | 41167139 | SFRP1 | 7E−164 | 159 |
| 167 | chr20 | 48626382 | 48626670 | SNAI1 | 1.6E−20 | 158 |
| 168 | chr19 | 52956621 | 52957010 | ZNF578 | 8.9E−42 | 158 |
| 169 | chr18 | 44773291 | 44775576 | SKOR2 | 4E−205 | 156 |
| 170 | chr6 | 1515470 | 1515607 | FOXCUT | 0.0089 | 153 |
| 171 | chr8 | 70041770 | 70041817 | LINC01592 | 1 | 152 |
| 172 | chr17 | 77508770 | 77508910 | RBFOX3 | 1 | 152 |
| 173 | chr12 | 100750327 | 100750899 | SLC17A8 | 0.068 | 150 |
| 174 | chr11 | 134281625 | 134281685 | B3GAT1 | 0.023 | 150 |
| 175 | chr3 | 184056350 | 184056670 | FAM131A | 1 | 149 |
| 176 | chr21 | 46896215 | 46896311 | MIR6815 | 1 | 149 |
| 177 | chr1 | 17022838 | 17023402 | | 0.000067 | 148 |
| 178 | chr7 | 57928181 | 57928941 | ZNF716 | 1 | 148 |
| 179 | chr4 | 6754870 | 6755191 | KIAA0232 | 0.00000083 | 148 |
| 180 | chr6 | 84418032 | 84419401 | SNAP91 | 1.5E−39 | 148 |
| 181 | chr14 | 89494301 | 89494437 | TTC8 | 0.0016 | 147 |
| 182 | chr14 | 101923580 | 101925094 | LINC00524 | 1.7E−52 | 145 |
| 183 | chr18 | 55095098 | 55096420 | ONECUT2 | 3.6E−18 | 145 |
| 184 | chr20 | 17206672 | 17206744 | PCSK2 | 0.00000056 | 144 |
| 185 | chr17 | 1901294 | 1901374 | RTN4RL1 | 0.091 | 142 |
| 186 | chr2 | 202900352 | 202900702 | FZD7 | 2.6E−16 | 140 |
| 187 | chr2 | 241294171 | 241294473 | GPC1 | 0.27 | 140 |
| 188 | chr19 | 56988313 | 56989846 | ZNF667-AS1 | 2.7E−236 | 140 |
| 189 | chr5 | 32712092 | 32712166 | NPR3 | 6.4E−26 | 138 |
| 190 | chr8 | 65282005 | 65282932 | LOC102724623 | 2.4E−121 | 135 |
| 191 | chr7 | 73245344 | 73246132 | CLDN4 | 7E−32 | 134 |
| 192 | chr18 | 908970 | 909154 | ADCYAP1 | 1.7E−18 | 133 |
| 193 | chr6 | 25882171 | 25882633 | SLC17A3 | 0.00000012 | 132 |
| 194 | chr16 | 1093345 | 1093682 | SSTR5 | 0.000034 | 129 |
| 195 | chr17 | 3289378 | 3289570 | OR1E1 | 0.093 | 128 |
| 196 | chr22 | 42316084 | 42316198 | MIR378I | 3E−16 | 127 |
| 197 | chr1 | 50882599 | 50886519 | DMRTA2 | 0.043 | 126 |
| 198 | chr11 | 17742046 | 17743840 | MYOD1 | 4.1E−18 | 125 |
| 199 | chr1 | 158066380 | 158066694 | LOC646268 | 1 | 124 |
| 200 | chr12 | 175962 | 176081 | IQSEC3 | 2.9E−44 | 124 |
| 201 | chr2 | 47797414 | 47799268 | KCNK12 | 1 | 123 |
| 202 | chr3 | 196373400 | 196373649 | NRROS | 0.000045 | 122 |
| 203 | chr8 | 54791184 | 54795283 | RGS20 | 8.7E−30 | 122 |
| 204 | chr19 | 827575 | 827995 | AZU1 | 7.6E−12 | 121 |
| 205 | chr11 | 1316235 | 1316522 | TOLLIP | 0.0000063 | 119 |
| 206 | chr9 | 68411147 | 68411576 | MIR4477B | 1 | 119 |
| 207 | chr13 | 114876937 | 114877100 | RASA3 | 3.3E−09 | 118 |
| 208 | chr11 | 65359673 | 65360327 | KCNK7 | 9.3E−100 | 118 |
| 209 | chr9 | 139886483 | 139886538 | C9orf142 | 0.00021 | 116 |
| 210 | chr11 | 83392903 | 83393677 | DLG2 | 0.0000035 | 115 |
| 211 | chr19 | 13207238 | 13207752 | LYL1 | 1.5E−37 | 114 |
| 212 | chr10 | 50605228 | 50605945 | DRGX | 1.1E−57 | 114 |
| 213 | chr19 | 52222262 | 52223082 | HAS1 | 1.7E−83 | 114 |
| 214 | chr2 | 66808599 | 66809107 | LOC100507073 | 4.8E−48 | 112 |
| 215 | chr22 | 28074051 | 28074161 | MN1 | 0.000031 | 111 |
| 216 | chr15 | 20500215 | 20500308 | CHEK2P2 | 0.000000019 | 110 |
| 217 | chr1 | 228651894 | 228652581 | MIR4666A | 2.9E−37 | 110 |
| 218 | chr11 | 315696 | 316680 | IFITM1 | 2.7E−287 | 109 |
| 219 | chr19 | 37287854 | 37287967 | ZNF790-AS1 | 0.00016 | 108 |
| 220 | chr2 | 1358307 | 1358515 | TPO | 0.0013 | 106 |
| 221 | chr10 | 44198164 | 44198369 | ZNF32 | 0.00029 | 105 |
| 222 | chr7 | 96650654 | 96650895 | DLX5 | 3.3E−36 | 105 |
| 223 | chr4 | 106888365 | 106888636 | LOC101929577 | 1 | 104 |
| 224 | chr4 | 111539194 | 111540311 | PITX2 | 5.5E−31 | 104 |
| 225 | chr4 | 8582798 | 8583233 | GPR78 | 5.1E−28 | 104 |
| 226 | chr14 | 23835663 | 23835969 | EFS | 6.1E−13 | 101 |
| 227 | chr6 | 110736231 | 110737053 | DDO | 0.038 | 97 |
| 228 | chr7 | 158224687 | 158225210 | MIR595 | 5.1E−15 | 96 |
| 229 | chr17 | 7832993 | 7833294 | KCNAB3 | 7.3E−26 | 96 |
| 230 | chr5 | 140346515 | 140346985 | PCDHAC2 | 3E−61 | 94 |
| 231 | chr6 | 6614167 | 6614858 | LY86-AS1 | 1.5E−38 | 94 |
| 232 | chr3 | 9177744 | 9178053 | LOC101927416 | 3.4E−15 | 92 |
| 233 | chr2 | 105275648 | 105276050 | LINC01114 | 2.7E−52 | 90 |
| 234 | chr5 | 140430824 | 140431483 | PCDHB1 | 3.2E−20 | 88 |
| 235 | chr2 | 45155998 | 45156907 | SIX3-AS1 | 2.7E−64 | 87 |
| 236 | chr14 | 105945496 | 105945690 | CRIP2 | 0.0023 | 83 |
| 237 | chr7 | 153583317 | 153584340 | DPP6 | 2.7E−90 | 83 |
| 238 | chr22 | 39784943 | 39785145 | TAB1 | 0.000000017 | 83 |
| 239 | chr15 | 68118223 | 68118660 | SKOR1 | 3.1E−21 | 82 |
| 240 | chr11 | 70508058 | 70509170 | SHANK2 | 8.4E−29 | 82 |
| 241 | chr7 | 2677842 | 2678081 | TTYH3 | 0.058 | 80 |
| 242 | chr11 | 33563245 | 33563598 | KIAA1549L | 1 | 79 |
| 243 | chr17 | 72270376 | 72270444 | DNAI2 | 1 | 79 |
| 244 | chr5 | 3596042 | 3603149 | IRX1 | 6.2E−139 | 76 |
| 245 | chr10 | 102893547 | 102895133 | TLX1 | 6.1E−100 | 74 |
| 246 | chr8 | 6484294 | 6484804 | MIR8055 | 1.2E−15 | 73 |
| 247 | chr5 | 140213923 | 140214585 | PCDHA7 | 3.8E−32 | 72 |
| 248 | chr1 | 159046377 | 159047177 | AIM2 | 1.5E−15 | 72 |
| 249 | chr1 | 43472943 | 43473076 | SLC2A1 | 0.058 | 71 |
| 250 | chr12 | 128752541 | 128753150 | TMEM132C | 6.3E−65 | 70 |
| 251 | chr19 | 719101 | 719204 | PALM | 0.0000055 | 70 |
| 252 | chr7 | 50632993 | 50634081 | DDC | 0.00036 | 69 |
| 253 | chr4 | 13545251 | 13545612 | NKX3-2 | 7.9E−39 | 64 |
| 254 | chr6 | 158080653 | 158080862 | MIR3692 | 2.3E−09 | 63 |
| 255 | chr4 | 172734201 | 172734237 | GALNTL6 | 1.8E−18 | 60 |
| 256 | chr4 | 20254562 | 20257031 | SLIT2 | 3.8E−166 | 59 |
| 257 | chr16 | 31548582 | 31548738 | AHSP | 1 | 59 |
| 258 | chr1 | 1244840 | 1245076 | PUSL1 | 8.8E−09 | 57 |
| 259 | chr6 | 70992443 | 70992936 | COL9A1 | 6.8E−35 | 57 |
| 260 | chr12 | 54763057 | 54763367 | GPR84 | 0.029 | 56 |
| 261 | chr1 | 228745309 | 228745412 | MIR7641-2 | 1 | 55 |
| 262 | chr18 | 74962322 | 74963218 | GALR1 | 2.8E−77 | 55 |
| 263 | chr17 | 7906578 | 7906773 | GUCY2D | 0.00069 | 55 |
| 264 | chr5 | 92931643 | 92932639 | MIR548AO | 2.9E−10 | 55 |
| 265 | chr1 | 17012572 | 17012827 | | 1.2E−13 | 54 |
| 266 | chr4 | 206281 | 207007 | ZNF876P | 6.9E−92 | 53 |
| 267 | chr6 | 43612626 | 43612953 | RSPH9 | 1.8E−31 | 53 |
| 268 | chr19 | 53561167 | 53561775 | ERVV-2 | 1.3E−57 | 53 |
| 269 | chr4 | 5891985 | 5892089 | CRMP1 | 0.000000003 | 53 |
| 270 | chr2 | 115419825 | 115420075 | DPP10-AS3 | 4E−26 | 50 |
| 271 | chr2 | 239756351 | 239757924 | TWIST2 | 7.5E−148 | 50 |
| 272 | chr13 | 25946565 | 25946604 | ATP8A2 | 0.00024 | 50 |
| 273 | chr7 | 63154756 | 63154933 | MIR4283-1 | 1.8E−12 | 50 |
| 274 | chr1 | 38461444 | 38462096 | SF3A3 | 1E−33 | 49 |
| 275 | chr11 | 123388711 | 123389533 | GRAMD1B | 0.00000086 | 47 |
| 276 | chr7 | 155151059 | 155151576 | BLACE | 2.7E−27 | 46 |
| 277 | chr10 | 38893450 | 38894256 | ACTR3BP5 | 1 | 44 |
| 278 | chr19 | 719533 | 720333 | PALM | 0.00032 | 42 |
| 279 | chr10 | 36476957 | 36477264 | PCAT5 | 0.33 | 41 |
| 280 | chr10 | 2543890 | 2543986 | LINC00701 | 0.0017 | 40 |
| 281 | chr12 | 312591 | 312753 | LOC101929384 | 2E−45 | 40 |
| 282 | chr6 | 41604583 | 41605020 | MDFI | 2E−18 | 40 |
| 283 | chr8 | 93114293 | 93114360 | RUNX1T1 | 9.5E−10 | 40 |
| 284 | chr6 | 38682849 | 38683236 | DNAH8 | 3.5E−24 | 38 |
| 285 | chr19 | 46915243 | 46916093 | CCDC8 | 1.8E−35 | 38 |
| 286 | chr6 | 170553122 | 170553880 | LOC154449 | 6.2E−32 | 37 |
| 287 | chr6 | 133562460 | 133563564 | EYA4 | 2E−91 | 36 |
| 288 | chr2 | 1719121 | 1719515 | PXDN | 0.018 | 36 |
| 289 | chr11 | 17757186 | 17758226 | KCNC1 | 5.2E−85 | 36 |
| 290 | chr16 | 214606 | 216803 | HBM | 0.00028 | 34 |
| 291 | chr12 | 43944719 | 43946284 | ADAMTS20 | 1.7E−154 | 32 |
| 292 | chr10 | 134225188 | 134225331 | PWWP2B | 1.2E−09 | 31 |
| 293 | chr20 | 54579077 | 54579729 | CBLN4 | 1.1E−35 | 31 |
| 294 | chr7 | 63642030 | 63642712 | ZNF735 | 3.8E−09 | 31 |
| 295 | chr6 | 110300487 | 110301094 | GPR6 | 2.5E−45 | 30 |
| 296 | chr5 | 176559237 | 176559421 | NSD1 | 1.1E−09 | 29 |
| 297 | chr2 | 74742511 | 74743758 | TLX2 | 4.2E−119 | 28 |
| 298 | chr2 | 1203202 | 1204002 | TPO | 0.00082 | 27 |
| 299 | chr8 | 47529009 | 47529429 | LINC00293 | 1.2E−20 | 27 |
| 300 | chr12 | 52963526 | 52963870 | KRT74 | 1 | 27 |
| 301 | chr7 | 2609623 | 2609973 | IQCE | 6.4E−23 | 25 |
| 302 | chr9 | 137111456 | 137111610 | RNU6ATAC | 1 | 24 |
| 303 | chr2 | 168675039 | 168675400 | B3GALT1 | 0.00089 | 24 |
| 304 | chr9 | 68409357 | 68409516 | MIR4477B | 1 | 24 |
| 305 | chr15 | 45408769 | 45409558 | DUOXA2 | 3.3E−36 | 23 |
| 306 | chr19 | 9608672 | 9609495 | ZNF560 | 2.9E−34 | 23 |
| 307 | chr20 | 62092340 | 62092502 | KCNQ2 | 0.00017 | 22 |
| 308 | chr1 | 845447 | 845534 | LOC100130417 | 1.2E−13 | 22 |
| 309 | chr20 | 61788160 | 61788642 | MIR124-3 | 4.5E−18 | 21 |
| 310 | chr7 | 2653570 | 2653873 | TTYH3 | 0.000012 | 20 |
| 311 | chr13 | 109147979 | 109149018 | MYO16 | 4.6E−154 | 19 |
| 312 | chr8 | 57802462 | 57802853 | IMPAD1 | 0.0028 | 19 |
| 313 | chr11 | 69482154 | 69482589 | ORAOV1 | 4.7E−17 | 19 |
| 314 | chr15 | 96904622 | 96905268 | NR2F2 | 1.8E−35 | 19 |
| 315 | chr8 | 142276216 | 142276640 | SLC45A4 | 4E−30 | 18 |
| 316 | chr13 | 95086011 | 95086360 | DCT | 4.7E−09 | 17 |
| 317 | chr5 | 140188207 | 140188264 | PCDHA4 | 2.5E−09 | 16 |
| 318 | chr4 | 174429648 | 174430640 | HAND2 | 1.5E−68 | 16 |
| 319 | chr19 | 55597993 | 55599029 | EPS8L1 | 1.6E−70 | 16 |
| 320 | chr7 | 72440068 | 72440443 | LOC100101148 | 1.5E−10 | 16 |
| 321 | chr10 | 126254543 | 126254769 | LHPP | 0.046 | 15 |
| 322 | chr15 | 66914678 | 66914731 | LINC01169 | 0.000024 | 15 |
| 323 | chr6 | 170553884 | 170554906 | LOC154449 | 4.7E−10 | 14 |
| 324 | chr5 | 175107749 | 175108520 | HRH2 | 8.7E−11 | 13 |
| 325 | chr5 | 178367536 | 178368804 | ZNF454 | 6.3E−99 | 13 |
| 326 | chr19 | 51107536 | 51107741 | SNAR-F | 1.4E−23 | 13 |
| 327 | chr5 | 176046720 | 176047338 | MIR4281 | 1.2E−26 | 12 |
| 328 | chr19 | 57182797 | 57183497 | ZNF835 | 1.8E−80 | 12 |
| 329 | chr15 | 96866536 | 96866786 | NR2F2 | 1.3E−15 | 12 |
| 330 | chr15 | 24722579 | 24723145 | PWRN3 | 2.1E−14 | 11 |
| 331 | chr16 | 32896382 | 32896472 | SLC6A10P | 0.013 | 11 |
| 332 | chr15 | 37172579 | 37172784 | LOC145845 | 1.9E−35 | 10 |
| 333 | chr1 | 226730366 | 226730729 | C1orf95 | 0.077 | 9 |
| 334 | chr6 | 25027421 | 25028183 | FAM65B | 1.1E−13 | 9 |
| 335 | chr2 | 3704470 | 3704864 | ALLC | 0.13 | 9 |
| 336 | chr22 | 51042030 | 51042767 | MAPK8IP2 | 1.3E−15 | 9 |
| 337 | chr19 | 9473589 | 9474000 | ZNF177 | 3.7E−40 | 9 |
| 338 | chr10 | 106400989 | 106402192 | SORCS3 | 9.8E−153 | 8 |
| 339 | chr10 | 131265375 | 131265496 | MGMT | 0.0093 | 8 |
| 340 | chr3 | 13515572 | 13515826 | HDAC11-AS1 | 0.00067 | 8 |
| 341 | chr19 | 30017510 | 30021485 | VSTM2B | 8.5E−90 | 8 |
| 342 | chr7 | 8483144 | 8483735 | NXPH1 | 4.3E−12 | 8 |
| 343 | chr19 | 30866358 | 30866489 | ZNF536 | 0.000059 | 7 |
| 344 | chr3 | 9988376 | 9989677 | PRRT3-AS1 | 2.6E−11 | 7 |
| 345 | chr7 | 24323193 | 24325522 | NPY | 2.3E−106 | 6 |
| 346 | chr19 | 48565035 | 48565323 | CABP5 | 2.5E−27 | 6 |
| 347 | chr8 | 70946912 | 70947440 | PRDM14 | 8.8E−32 | 6 |
| 348 | chr18 | 73628519 | 73628569 | LOC339298 | 3.8E−17 | 6 |
| 349 | chr1 | 161391806 | 161392006 | CFAP126 | 8.5E−12 | 5 |
| 350 | chr2 | 218354129 | 218354315 | DIRC3 | 0.000000026 | 5 |
| 351 | chr7 | 45961198 | 45961575 | IGFBP3 | 0.000000023 | 5 |
| 352 | chr11 | 66314443 | 66314513 | ACTN3 | 1 | 5 |
| 353 | chr5 | 140182566 | 140183013 | PCDHA3 | 7.2E−14 | 4 |
| 354 | chr19 | 1566714 | 1566836 | MEX3D | 1 | 4 |
| 355 | chr1 | 167408458 | 167409113 | CD247 | 1.1E−49 | 4 |
| 356 | chr5 | 170743598 | 170743662 | TLX3 | 1.8E−17 | 4 |
| 357 | chr8 | 1764397 | 1764910 | MIR596 | 1.6E−20 | 4 |
| 358 | chr7 | 31091910 | 31092310 | ADCYAP1R1 | 2.3E−118 | 4 |
| 359 | chr10 | 3977409 | 3977757 | MIR6078 | 2.2E−19 | 4 |
| 360 | chr18 | 70533842 | 70535309 | NETO1 | 1.5E−187 | 4 |
| 361 | chr7 | 157639854 | 157640203 | LOC100506585 | 0.11 | 3 |
| 362 | chr10 | 1850245 | 1850362 | ADARB2 | 0.053 | 3 |
| 363 | chr7 | 32467625 | 32467947 | LOC100130673 | 2.9E−29 | 3 |
| 364 | chr19 | 7852932 | 7853028 | CLEC4GP1 | 0.00046 | 3 |
| 365 | chr19 | 10404842 | 10405414 | ICAM5 | 4.4E−13 | 2 |
| 366 | chr13 | 112723041 | 112724206 | SOX1 | 2.1E−56 | 2 |
| 367 | chr1 | 17199369 | 17199548 | | 1.1E−13 | 2 |
| 368 | chr20 | 21694312 | 21694738 | PAX1 | 5.1E−22 | 2 |
| 369 | chr1 | 22379389 | 22379490 | CDC42 | 9.2E−18 | 2 |
| 370 | chr17 | 25798664 | 25799447 | KSR1 | 0.000000016 | 2 |
| 371 | chr16 | 32823336 | 32823505 | SLC6A10P | 0.0029 | 2 |
| 372 | chr22 | 42896914 | 42896987 | SERHL | 0.00065 | 2 |
| 373 | chr9 | 44842351 | 44842859 | FAM27C | 1.8E−19 | 2 |
| 374 | chr19 | 55889196 | 55889581 | TMEM190 | 0.015 | 2 |
| 375 | chr19 | 58629190 | 58629803 | ZSCAN18 | 7.3E−54 | 2 |
| 376 | chr11 | 60775686 | 60775968 | CD6 | 0.00074 | 2 |
| 377 | chr18 | 77159368 | 77159491 | NFATC1 | 1.1E−09 | 2 |
| 378 | chr17 | 79427821 | 79428094 | MIR3186 | 0.000065 | 2 |
| 379 | chr10 | 89167291 | 89168132 | LINC00864 | 0.000023 | 2 |
| 380 | chr8 | 11607240 | 11607395 | C8orf49 | 1 | 1 |
| 381 | chr7 | 116962696 | 116963587 | WNT2 | 2.1E−77 | 1 |
| 382 | chr1 | 119522232 | 119522972 | TBX15 | 1.4E−55 | 1 |
| 383 | chr8 | 145955929 | 145955988 | ZNF251 | 0.02 | 1 |
| 384 | chr3 | 154146310 | 154146951 | GPR149 | 1.5E−41 | 1 |
| 385 | chr1 | 155290901 | 155291064 | RUSC1 | 2.9E−11 | 1 |
| 386 | chr5 | 178487825 | 178487920 | ZNF354C | 0.00000046 | 1 |
| 387 | chr5 | 179192382 | 179192707 | LTC4S | 0.00000045 | 1 |
| 388 | chr1 | 228400272 | 228400693 | C1orf145 | 0.095 | 1 |
| 389 | chr1 | 230882247 | 230883682 | CAPN9 | 1.3E−12 | 1 |
| 390 | chr2 | 242927372 | 242927735 | LOC102723927 | 4.4E−11 | 1 |
| 391 | chr6 | 26758367 | 26758791 | ZNF322 | 2E−11 | 1 |
| 392 | chr17 | 27920378 | 27920436 | ANKRD13B | 1 | 1 |
| 393 | chr2 | 31457371 | 31457421 | EHD3 | 1.8E−13 | 1 |
| 394 | chr1 | 40235714 | 40236348 | OXCT2 | 2.7E−35 | 1 |
| 395 | chr4 | 44449438 | 44450297 | KCTD8 | 4.9E−108 | 1 |
| 396 | chr1 | 44872995 | 44873957 | RNF220 | 7.8E−68 | 1 |
| 397 | chr19 | 475077 | 475675 | ODF3L2 | 4.7E−11 | 1 |
| 398 | chr17 | 49026916 | 49027266 | TOB1 | 0.42 | 1 |
| 399 | chr6 | 7051353 | 7051576 | RREB1 | 0.069 | 1 |
| 400 | chr2 | 74642004 | 74642776 | C2orf81 | 7E−61 | 1 |
| 401 | chr17 | 75315957 | 75316590 | SEPT9 | 0.000062 | 1 |
| 402 | chr17 | 76354723 | 76355136 | SOCS3 | 1.3E−37 | 1 |
| 403 | chr5 | 76934957 | 76935276 | OTP | 4.4E−32 | 1 |
| 404 | chr17 | 77766909 | 77767043 | CBX8 | 1 | 1 |
| 405 | chr4 | 81187466 | 81187605 | FGF5 | 4.9E−11 | 1 |
| 406 | chr14 | 85997416 | 85998669 | FLRT2 | 1.1E−98 | 1 |
| 407 | chr1 | 933737 | 933974 | HES4 | 1.7E−18 | 1 |

### Esophageal Squamous Cell Carcinoma

In another aspect is provided a method of detecting a level of DNA methylation in a subject at risk of developing a esophageal squamous cell carcinoma, the method including: determining the degree of methylation of CpG sites within a plurality of gene regions in a DNA sample from the subject, wherein the plurality of gene regions includes at least 5 different gene regions in Table ESCC. In embodiments, an increased level of methylated CpG sites relative to a standard control indicates a higher risk of esophageal squamous cell carcinoma.

Provided herein is a method of treating esophageal squamous cell carcinoma in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table ESCC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the esophageal squamous cell carcinoma is Stage I, Stage II, or Stage III. In embodiments, the esophageal squamous cell carcinoma is Stage I. In embodiments, the esophageal squamous cell carcinoma is Stage II. In embodiments, the esophageal squamous cell carcinoma is Stage III.

Provided herein is a method of diagnosing esophageal squamous cell carcinoma in a patient comprising: (a) detecting a level of methylated CpG sites within a plurality of gene regions in a DNA sample from the patient, wherein the plurality of gene regions comprise at least 5 different gene regions in Table ESCC; and (b) diagnosing the patient with esophageal squamous cell carcinoma when the DNA sample has an increased level of methylated CpG sites, relative to a standard control, within the plurality of gene regions. In embodiments, the esophageal squamous cell carcinoma is Stage I, Stage II, or Stage III. In embodiments, the esophageal squamous cell carcinoma is Stage I. In embodiments, the esophageal squamous cell carcinoma is Stage II. In embodiments, the esophageal squamous cell carcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having esophageal squamous cell carcinoma or monitoring risk for developing esophageal squamous cell carcinoma in a patient comprising: (a) detecting a level of methylated CpG sites within a plurality of gene regions in a DNA sample from the patient at a first time point, wherein the plurality of gene regions comprise at least 5 different gene regions in Table ESCC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing esophageal squamous cell carcinoma. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing esophageal squamous cell carcinoma or does not have esophageal squamous cell carcinoma. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing esophageal squamous cell carcinoma or may have esophageal squamous cell carcinoma. In embodiments, the esophageal squamous cell carcinoma is Stage I, Stage II, or Stage III. In embodiments, the esophageal squamous cell carcinoma is Stage I. In embodiments, the esophageal squamous cell carcinoma is Stage II. In embodiments, the esophageal squamous cell carcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table ESCC. In embodiments, the plurality of gene regions includes at least 2 DMRs (i.e., gene regions) in Table ESCC. In embodiments, the plurality of gene regions includes at least 3 DMRs in Table ESCC. In embodiments, the plurality of gene regions includes at least 4 DMRs in Table ESCC. In embodiments, the plurality of gene regions includes at least 5 DMRs in Table ESCC. In embodiments, the plurality of gene regions includes at least 6 DMRs in Table ESCC. In embodiments, the plurality of gene regions includes at least 7 DMRs in Table ESCC. In embodiments, the plurality of gene regions includes at least 8 DMRs in Table ESCC. In embodiments, the plurality of gene regions includes at least 9 DMRs in Table ESCC. In embodiments, the plurality of gene regions includes at least 10 DMRs in Table ESCC. In embodiments, the plurality of gene regions includes at least 15 DMRs in Table ESCC. In embodiments, the plurality of gene regions includes at least 20 DMRs in Table ESCC. In embodiments, the plurality of gene regions includes at least 25 DMRs in Table ESCC. In embodiments, the plurality of gene regions includes at least 30 DMRs in Table ESCC. In embodiments, the plurality of gene regions includes at least 35 DMRs in Table ESCC. In embodiments, the plurality of gene regions includes at least 40 DMRs in Table ESCC. In embodiments, the plurality of gene regions includes at least 45 DMRs in Table ESCC. In embodiments, the plurality of gene regions includes at least 50 DMRs in Table ESCC. In embodiments, the plurality of gene regions includes at least 55 DMRs in Table ESCC. In embodiments, the plurality of gene regions includes at least 60 DMRs in Table ESCC. In embodiments, the plurality of gene regions includes at least 65 DMRs in Table ESCC. In embodiments, the plurality of gene regions includes at least 70 DMRs in Table ESCC. In embodiments, the plurality of gene regions includes at least 75 DMRs in Table ESCC. In embodiments, the plurality of gene regions includes at least 80 DMRs in Table ESCC. In embodiments, the plurality of gene regions includes at least 85 DMRs in Table ESCC. In embodiments, the plurality of gene regions includes at least 90 DMRs in Table ESCC. In embodiments, the plurality of gene regions includes at least 95 DMRs in Table ESCC. In embodiments, the plurality of gene regions includes at least 110 DMRs in Table ESCC. In embodiments, the plurality of gene regions includes at least 120 DMRs in Table ESCC. In embodiments, the plurality of gene regions includes at least 130 DMRs in Table ESCC. In embodiments, the plurality of gene regions includes at least 140 DMRs in Table ESCC. In embodiments, the plurality of gene regions includes at least 150 DMRs in Table ESCC. In embodiments, the plurality of gene regions includes at least 160 DMRs in Table ESCC. In embodiments, the plurality of gene regions includes at least 170 DMRs in Table ESCC. In embodiments, the plurality of gene regions includes at least 180 DMRs in Table ESCC. In embodiments, the plurality of gene regions includes at least 190 DMRs in Table ESCC. In embodiments, the plurality of gene regions includes at least 200 DMRs in Table ESCC. In embodiments, the plurality of gene regions includes at least 225 DMRs in Table ESCC. In embodiments, the plurality of gene regions includes at least 250 DMRs in Table ESCC. In embodiments, the plurality of gene regions includes at least 275 DMRs in Table ESCC. In embodiments, the plurality of gene regions includes at least 300 DMRs in Table ESCC. In embodiments, the plurality of gene regions includes at least 325 DMRs in Table ESCC. In embodiments, the plurality of gene regions includes at least 350 DMRs in Table ESCC. In embodiments, the plurality of gene regions includes at least 375 DMRs in Table ESCC. In embodiments, the plurality of gene regions includes at least 400 DMRs in Table ESCC.

In embodiments, the plurality of gene regions includes the first DMR (i.e., gene region) in Table ESCC. In embodiments, the plurality of gene regions includes the first 2 DMRs (i.e., gene regions) in Table ESCC. In embodiments, the plurality of gene regions includes the first 3 DMRs in Table ESCC. In embodiments, the plurality of gene regions includes the first 4 DMRs in Table ESCC. In embodiments, the plurality of gene regions includes the first 5 DMRs in Table ESCC. In embodiments, the plurality of gene regions includes the first 6 DMRs in Table ESCC. In embodiments, the plurality of gene regions includes the first 7 DMRs in Table ESCC. In embodiments, the plurality of gene regions includes the first 8 DMRs in Table ESCC. In embodiments, the plurality of gene regions includes the first 9 DMRs in Table ESCC. In embodiments, the plurality of gene regions includes the first 10 DMRs in Table ESCC. In embodiments, the plurality of gene regions includes the first 11 DMRs in Table ESCC. In embodiments, the plurality of gene regions includes the first 12 DMRs in Table ESCC. In embodiments, the plurality of gene regions includes the first 13 DMRs in Table ESCC. In embodiments, the plurality of gene regions includes the first 14 DMRs in Table ESCC. In embodiments, the plurality of gene regions includes the first 15 DMRs in Table ESCC. In embodiments, the plurality of gene regions includes the first 16 DMRs in Table ESCC. In embodiments, the plurality of gene regions includes the first 17 DMRs in Table ESCC. In embodiments, the plurality of gene regions includes the first 18 DMRs in Table ESCC. In embodiments, the plurality of gene regions includes the first 19 DMRs in Table ESCC. In embodiments, the plurality of gene regions includes the first 20 DMRs in Table ESCC. In embodiments, the plurality of gene regions includes the first 25 DMRs in Table ESCC. In embodiments, the plurality of gene regions includes the first 30 DMRs in Table ESCC. In embodiments, the plurality of gene regions includes the first 35 DMRs in Table ESCC. In embodiments, the plurality of gene regions includes the first 40 DMRs in Table ESCC. In embodiments, the plurality of gene regions includes the first 45 DMRs in Table ESCC. In embodiments, the plurality of gene regions includes the first 50 DMRs in Table ESCC. In embodiments, the plurality of gene regions includes the first 55 DMRs in Table ESCC. In embodiments, the plurality of gene regions includes the first 60 DMRs in Table ESCC. In embodiments, the plurality of gene regions includes the first 65 DMRs in Table ESCC. In embodiments, the plurality of gene regions includes the first 70 DMRs in Table ESCC. In embodiments, the plurality of gene regions includes the first 75 DMRs in Table ESCC. In embodiments, the plurality of gene regions includes the first 80 DMRs in Table ESCC. In embodiments, the plurality of gene regions includes the first 85 DMRs in Table ESCC. In embodiments, the plurality of gene regions includes the first 90 DMRs in Table ESCC. In embodiments, the plurality of gene regions includes the first 95 DMRs in Table ESCC. In embodiments, the plurality of gene regions includes the first 110 DMRs in Table ESCC. In embodiments, the plurality of gene regions includes the first 120 DMRs in Table ESCC. In embodiments, the plurality of gene regions includes the first 130 DMRs in Table ESCC. In embodiments, the plurality of gene regions includes the first 140 DMRs in Table ESCC. In embodiments, the plurality of gene regions includes the first 150 DMRs in Table ESCC. In embodiments, the plurality of gene regions includes the first 160 DMRs in Table ESCC. In embodiments, the plurality of gene regions includes the first 170 DMRs in Table ESCC. In embodiments, the plurality of gene regions includes the first 180 DMRs in Table ESCC. In embodiments, the plurality of gene regions includes the first 190 DMRs in Table ESCC. In embodiments, the plurality of gene regions includes the first 200 DMRs in Table ESCC. In embodiments, the plurality of gene regions includes the first 225 DMRs in Table ESCC. In embodiments, the plurality of gene regions includes the first 250 DMRs in Table ESCC. In embodiments, the plurality of gene regions includes the first 275 DMRs in Table ESCC. In embodiments, the plurality of gene regions includes the first 300 DMRs in Table ESCC. In embodiments, the plurality of gene regions includes the first 325 DMRs in Table ESCC. In embodiments, the plurality of gene regions includes the first 350 DMRs in Table ESCC. In embodiments, the plurality of gene regions includes the first 375 DMRs in Table ESCC. In embodiments, the plurality of gene regions includes the first 400 DMRs in Table ESCC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is an esophagusgastroduodenoscopy (EGD), an endoscopic ultrasound, a bronchoscopy, or a tissue biopsy. In embodiments, the confirmatory diagnostic procedure is a tumor marker test, a microsatellite instability test, a computed tomography scan, a magnetic resonance imaging scan, or a positron emission tomography scan.

In embodiments, further includes treating the subject for esophageal squamous cell carcinoma. In embodiments, the treating includes surgery, endoscopic therapy, or radiation therapy. In embodiments, the treating includes chemotherapy, targeted therapy, or immunotherapy. In embodiments, the treating includes chemotherapy, targeted therapy, immunotherapy, or a combination of two or more thereof.

TABLE ESCC

| Gene | | | | | | |
|---|---|---|---|---|---|---|
| Region | | | | | Adjusted | |
| No. | Chr | Start | End | Gene Name | p-value | Freq |
| 1 | chr10 | 23461316 | 23463617 | PTF1A | 5.9E−163 | 416 |
| 2 | chr19 | 48565035 | 48565323 | CABP5 | 2.5E−27 | 415 |
| 3 | chr6 | 100906564 | 100906629 | SIM1 | 3.2E−15 | 413 |
| 4 | chr20 | 21695247 | 21695306 | PAX1 | 3.6E−14 | 413 |
| 5 | chr13 | 113968440 | 113968609 | LAMP1 | 0.000000076 | 412 |
| 6 | chr1 | 50882599 | 50886519 | DMRTA2 | 0.043 | 412 |
| 7 | chr12 | 132846038 | 132846261 | LOC100130238 | 0.0072 | 411 |
| 8 | chr1 | 159046377 | 159047177 | AIM2 | 1.5E−15 | 411 |
| 9 | chr1 | 159174283 | 159175211 | ACKR1 | 0.00000034 | 410 |
| 10 | chr17 | 76354723 | 76355136 | SOCS3 | 1.3E−37 | 409 |
| 11 | chr2 | 118593849 | 118594327 | DDX18 | 3.8E−53 | 408 |
| 12 | chr16 | 1171931 | 1172126 | C1QTNF8 | 1 | 407 |
| 13 | chr1 | 44872995 | 44873957 | RNF220 | 7.8E−68 | 407 |
| 14 | chr2 | 90016149 | 90016431 | MIR4436A | 4.1E−41 | 407 |
| 15 | chr12 | 125223501 | 125223749 | SCARB1 | 9.9E−32 | 406 |
| 16 | chr5 | 72705934 | 72706042 | FOXD1 | 1 | 404 |
| 17 | chr11 | 117069683 | 117070130 | TAGLN | 1.6E−19 | 403 |
| 18 | chr8 | 145751955 | 145752311 | LRRC24 | 0.00000036 | 403 |
| 19 | chr21 | 46896215 | 46896311 | MIR6815 | 1 | 402 |
| 20 | chr8 | 99985933 | 99987000 | OSR2 | 5.9E−89 | 402 |
| 21 | chr8 | 132052036 | 132054784 | ADCY8 | 5.2E−216 | 401 |
| 22 | chr19 | 37282439 | 37282687 | ZNF790-AS1 | 0.00000014 | 401 |
| 23 | chr17 | 3675018 | 3675296 | ITGAE | 1 | 400 |
| 24 | chr10 | 369324 | 369404 | | 0.00000015 | 400 |
| 25 | chr20 | 56283770 | 56284043 | PMEPA1 | 7E−11 | 400 |
| 26 | chr19 | 58545029 | 58545559 | ZSCAN1 | 2.4E−140 | 400 |
| 27 | chr6 | 78172191 | 78173170 | HTR1B | 2.5E−97 | 400 |
| 28 | chr19 | 39798183 | 39798881 | LRFN1 | 1.5E−110 | 399 |
| 29 | chr2 | 74742511 | 74743758 | TLX2 | 4.2E−119 | 399 |
| 30 | chr5 | 127872563 | 127872669 | FBN2 | 2.9E−09 | 398 |
| 31 | chr6 | 58147126 | 58149415 | LINC00680 | 1.7E−97 | 398 |
| 32 | chr14 | 48143369 | 48145724 | MDGA2 | 1.2E−274 | 397 |
| 33 | chr8 | 143644935 | 143645493 | ARC | 2.7E−21 | 395 |
| 34 | chr10 | 102893547 | 102895133 | TLX1 | 6.1E−100 | 394 |
| 35 | chr8 | 142394572 | 142394898 | GPR20 | 1 | 394 |
| 36 | chr22 | 23915439 | 23915669 | IGLL1 | 4.5E−10 | 393 |
| 37 | chr14 | 103415848 | 103416095 | AMN | 0.00078 | 392 |
| 38 | chr4 | 134071516 | 134074046 | PCDH10 | 9.4E−218 | 392 |
| 39 | chr11 | 83392903 | 83393677 | DLG2 | 0.0000035 | 392 |
| 40 | chr19 | 58446168 | 58446960 | ZNF418 | 3.6E−59 | 390 |
| 41 | chr20 | 61809243 | 61809785 | MIR124-3 | 5.9E−83 | 389 |
| 42 | chr16 | 1077596 | 1077705 | SSTR5 | 0.00014 | 388 |
| 43 | chr14 | 101487395 | 101488558 | MIR379 | 0.000000022 | 387 |
| 44 | chr15 | 101629342 | 101629581 | CHSY1 | 0.86 | 386 |
| 45 | chr19 | 9473589 | 9474000 | ZNF177 | 3.7E−40 | 385 |
| 46 | chr7 | 157477410 | 157478936 | MIR153-2 | 1.6E−160 | 383 |
| 47 | chr8 | 37082338 | 37082625 | | 1.7E−21 | 382 |
| 48 | chr20 | 37356055 | 37357918 | SLC32A1 | 1.2E−132 | 382 |
| 49 | chr12 | 63543712 | 63544966 | AVPR1A | 5.7E−97 | 381 |
| 50 | chr10 | 128594721 | 128595219 | DOCK1 | 2E−25 | 336 |
| 51 | chr19 | 51107536 | 51107741 | SNAR-F | 1.4E−23 | 303 |
| 52 | chr4 | 8582798 | 8583233 | GPR78 | 5.1E−28 | 284 |
| 53 | chr14 | 104896487 | 104896658 | C14orf180 | 0.00022 | 278 |
| 54 | chr2 | 124782229 | 124783355 | CNTNAP5 | 2E−81 | 267 |
| 55 | chr2 | 9894121 | 9894442 | TAF1B | 0.00078 | 254 |
| 56 | chr16 | 67428314 | 67429097 | TPPP3 | 4.2E−63 | 247 |
| 57 | chr1 | 870922 | 871057 | SAMD11 | 1.2E−16 | 237 |
| 58 | chr13 | 112575679 | 112576115 | LINC00354 | 1 | 236 |
| 59 | chr16 | 55689441 | 55690964 | SLC6A2 | 2.7E−147 | 232 |
| 60 | chr1 | 38461444 | 38462096 | SF3A3 | 1E−33 | 215 |
| 61 | chr19 | 52956621 | 52957010 | ZNF578 | 8.9E−42 | 215 |
| 62 | chr1 | 36042841 | 36043373 | TFAP2E | 2.8E−63 | 213 |
| 63 | chr5 | 2751128 | 2757191 | C5orf38 | 1.3E−152 | 210 |
| 64 | chr7 | 4764575 | 4765382 | FOXK1 | 1.6E−23 | 210 |
| 65 | chr13 | 112978530 | 112978862 | LINC01044 | 1.1E−26 | 208 |
| 66 | chr2 | 90413838 | 90414075 | MIR4436A | 1.3E−27 | 207 |
| 67 | chr4 | 174429648 | 174430640 | HAND2 | 1.5E−68 | 205 |
| 68 | chr4 | 8861489 | 8862361 | HMX1 | 3.5E−66 | 203 |
| 69 | chr4 | 13524753 | 13526008 | LINC01097 | 2E−62 | 201 |
| 70 | chr6 | 33560785 | 33561266 | LINC00336 | 1.8E−43 | 199 |
| 71 | chr2 | 105275648 | 105276050 | LINC01114 | 2.7E−52 | 195 |
| 72 | chr5 | 140305727 | 140307192 | PCDHAC1 | 6.2E−93 | 195 |
| 73 | chr20 | 5297480 | 5297906 | PROKR2 | 3.8E−71 | 193 |
| 74 | chr12 | 125139527 | 125140249 | NCOR2 | 0.0038 | 191 |
| 75 | chr12 | 128752541 | 128753150 | TMEM132C | 6.3E−65 | 191 |
| 76 | chr12 | 43944719 | 43946284 | ADAMTS20 | 1.7E−154 | 191 |
| 77 | chr14 | 101012754 | 101013110 | BEGAIN | 7.3E−30 | 189 |
| 78 | chr14 | 26674163 | 26674271 | NOVA1 | 2E−20 | 189 |
| 79 | chr13 | 112723041 | 112724206 | SOX1 | 2.1E−56 | 188 |
| 80 | chr1 | 243646287 | 243646876 | MIR4677 | 1.9E−38 | 188 |
| 81 | chr5 | 178367536 | 178368804 | ZNF454 | 6.3E−99 | 187 |
| 82 | chr15 | 74426044 | 74427137 | ISLR2 | 1.3E−62 | 187 |
| 83 | chr19 | 30363174 | 30363379 | URI1 | 6.6E−17 | 186 |
| 84 | chr11 | 64479269 | 64479424 | NRXN2 | 0.0000011 | 186 |
| 85 | chr9 | 99838560 | 99840130 | GAS2L1P2 | 8.5E−38 | 186 |
| 86 | chr2 | 66808599 | 66809107 | LOC100507073 | 4.8E−48 | 185 |
| 87 | chr14 | 91836202 | 91836469 | CCDC88C | 0.000037 | 185 |
| 88 | chr7 | 28998417 | 28998540 | TRIL | 0.44 | 184 |
| 89 | chr19 | 53561167 | 53561775 | ERVV-2 | 1.3E−57 | 184 |
| 90 | chr19 | 56988313 | 56989846 | ZNF667-AS1 | 2.7E−236 | 184 |
| 91 | chr8 | 1449737 | 1450097 | DLGAP2 | 1 | 183 |
| 92 | chr16 | 66982589 | 66983023 | CES3 | 2.3E−24 | 183 |
| 93 | chr19 | 1130738 | 1131134 | SBNO2 | 2.8E−20 | 182 |
| 94 | chr3 | 11952643 | 11952992 | TAMM41 | 5.5E−11 | 182 |
| 95 | chr5 | 3596042 | 3603149 | IRX1 | 6.2E−139 | 181 |
| 96 | chr19 | 40421192 | 40421672 | FCGBP | 1E−27 | 181 |
| 97 | chr19 | 58094931 | 58095898 | ZIK1 | 2.3E−72 | 181 |
| 98 | chr10 | 102419094 | 102419667 | PAX2 | 8.5E−39 | 179 |
| 99 | chr19 | 11649285 | 11649840 | CNN1 | 0.0000075 | 179 |
| 100 | chr6 | 159639481 | 159639704 | LOC101929122 | 0.00000032 | 179 |
| 101 | chr7 | 62574764 | 62574921 | ZNF733P | 1.7E−10 | 179 |
| 102 | chr5 | 2748307 | 2749889 | IRX2 | 4.4E−146 | 178 |
| 103 | chr13 | 53420678 | 53422348 | PCDH8 | 1.7E−179 | 177 |
| 104 | chr17 | 80163090 | 80163377 | CCDC57 | 1 | 177 |
| 105 | chr19 | 23253151 | 23254490 | LOC101929144 | 1.8E−65 | 176 |
| 106 | chr1 | 870310 | 870459 | SAMD11 | 0.000022 | 176 |
| 107 | chr2 | 202900352 | 202900702 | FZD7 | 2.6E−16 | 175 |
| 108 | chr12 | 30322944 | 30323207 | TMTC1 | 1.7E−24 | 175 |
| 109 | chr7 | 231221 | 231368 | FAM20C | 1 | 174 |
| 110 | chr2 | 45155998 | 45156907 | SIX3-AS1 | 2.7E−64 | 174 |
| 111 | chr2 | 119614523 | 119615747 | EN1 | 9.5E−78 | 173 |
| 112 | chr6 | 50818180 | 50818424 | TFAP2B | 4.3E−100 | 172 |
| 113 | chr6 | 1410166 | 1410436 | MIR6720 | 0.062 | 170 |
| 114 | chr2 | 1481314 | 1481380 | TPO | 0.0000016 | 170 |
| 115 | chr5 | 3592566 | 3592712 | IRX1 | 0.000000039 | 170 |
| 116 | chr16 | 88263173 | 88263261 | LOC101928880 | 1 | 170 |
| 117 | chr10 | 21799128 | 21799340 | CASC10 | 2.6E−15 | 169 |
| 118 | chr7 | 1266142 | 1266551 | UNCX | 1.9E−09 | 167 |
| 119 | chr2 | 171569945 | 171570045 | LINC01124 | 7.8E−14 | 167 |
| 120 | chr5 | 176816525 | 176816945 | SLC34A1 | 0.11 | 167 |
| 121 | chr7 | 55516723 | 55517117 | LANCL2 | 4.1E−29 | 167 |
| 122 | chr19 | 12305445 | 12306303 | LOC100289333 | 1E−61 | 166 |
| 123 | chr7 | 153584345 | 153585592 | DPP6 | 9.9E−129 | 166 |
| 124 | chr12 | 54348949 | 54349679 | HOXC12 | 3.9E−74 | 166 |
| 125 | chr1 | 119527391 | 119527516 | TBX15 | 2.2E−16 | 165 |
| 126 | chr1 | 10961102 | 10961414 | C1orf127 | 0.00000046 | 164 |
| 127 | chr2 | 131721191 | 131721457 | ARHGEF4 | 1.8E−79 | 164 |
| 128 | chr3 | 196373400 | 196373649 | NRROS | 0.000045 | 164 |
| 129 | chr10 | 50819078 | 50820313 | SLC18A3 | 1.2E−133 | 164 |
| 130 | chr2 | 87883776 | 87883951 | MIR4435-1 | 5.4E−70 | 164 |
| 131 | chr9 | 117266705 | 117266855 | DFNB31 | 0.00000012 | 163 |
| 132 | chr1 | 237206266 | 237206721 | RYR2 | 3.4E−55 | 163 |
| 133 | chr14 | 90991647 | 90992127 | LINC00642 | 5.8E−27 | 163 |
| 134 | chr4 | 107146 | 107967 | ZNF718 | 1.3E−127 | 162 |
| 135 | chr10 | 118031745 | 118033581 | GFRA1 | 4.1E−45 | 162 |
| 136 | chr4 | 298753 | 299338 | ZNF732 | 3.2E−65 | 162 |
| 137 | chr19 | 33726527 | 33726834 | SLC7A10 | 5.9E−26 | 162 |
| 138 | chr17 | 5000871 | 5001123 | USP6 | 6.4E−60 | 161 |
| 139 | chr19 | 50554366 | 50554491 | FLJ26850 | 9.8E−15 | 161 |
| 140 | chr3 | 179754308 | 179755306 | PEX5L | 8.8E−82 | 160 |
| 141 | chr1 | 206316905 | 206317654 | CTSE | 6.5E−10 | 160 |
| 142 | chr7 | 1392549 | 1392744 | MICALL2 | 0.00013 | 159 |
| 143 | chr5 | 4222954 | 4223319 | LOC101929153 | 0.002 | 158 |
| 144 | chr8 | 145003612 | 145003804 | PLEC | 6.4E−22 | 157 |
| 145 | chr7 | 50632993 | 50634081 | DDC | 0.00036 | 157 |
| 146 | chr19 | 36912350 | 36912880 | LOC644189 | 7E−91 | 156 |
| 147 | chr8 | 57358126 | 57359512 | PENK | 6.1E−164 | 156 |
| 148 | chr20 | 58630152 | 58630496 | C20orf197 | 1.4E−14 | 156 |
| 149 | chr4 | 674769 | 675388 | MYL5 | 6.2E−47 | 156 |
| 150 | chr6 | 161187997 | 161188183 | PLG | 9.1E−19 | 155 |
| 151 | chr2 | 19553069 | 19553594 | OSR1 | 2.3E−20 | 155 |
| 152 | chr19 | 58399818 | 58400409 | ZNF814 | 1E−36 | 155 |
| 153 | chr3 | 172165390 | 172166691 | GHSR | 0.00002 | 154 |
| 154 | chr1 | 228763781 | 228764342 | | 3.4E−27 | 154 |
| 155 | chr7 | 21209527 | 21209834 | SP4 | 0.0017 | 153 |
| 156 | chr4 | 188953078 | 188953927 | ZFP42 | 1.1E−36 | 152 |
| 157 | chr1 | 248551109 | 248551544 | OR2T6 | 0.0000012 | 152 |
| 158 | chr4 | 13545251 | 13545612 | NKX3-2 | 7.9E−39 | 151 |
| 159 | chr18 | 19745121 | 19745186 | GATA6-AS1 | 6.1E−09 | 151 |
| 160 | chr22 | 32753597 | 32754024 | RFPL3 | 0.55 | 148 |
| 161 | chr2 | 171676467 | 171677023 | GAD1 | 4.1E−20 | 145 |
| 162 | chr5 | 1973694 | 1974236 | CTD- | 0.000000085 | 145 |
| | | | | 2194D22.4 | | |
| 163 | chr20 | 21694312 | 21694738 | PAX1 | 5.1E−22 | 145 |
| 164 | chr15 | 89922050 | 89922792 | MIR9-3HG | 1.2E−57 | 144 |
| 165 | chr19 | 15090187 | 15090469 | SLC1A6 | 1.2E−46 | 143 |
| 166 | chr2 | 45169678 | 45170464 | SIX3 | 5.5E−41 | 142 |
| 167 | chr19 | 45975734 | 45976395 | FOSB | 2.7E−25 | 141 |
| 168 | chr2 | 131720907 | 131720960 | ARHGEF4 | 1.4E−10 | 140 |
| 169 | chr5 | 172671531 | 172672649 | NKX2-5 | 8.8E−43 | 140 |
| 170 | chr22 | 29866623 | 29866752 | NEFH | 1 | 140 |
| 171 | chr4 | 41634711 | 41635119 | LIMCH1 | 0.000011 | 140 |
| 172 | chr8 | 55370442 | 55372108 | SOX17 | 1.1E−157 | 140 |
| 173 | chr20 | 61471688 | 61471855 | DPH3P1 | 0.033 | 140 |
| 174 | chr19 | 30016974 | 30017506 | VSTM2B | 6.8E−96 | 139 |
| 175 | chr2 | 115419825 | 115420075 | DPP10-AS3 | 4E−26 | 137 |
| 176 | chr19 | 52222262 | 52223082 | HAS1 | 1.7E−83 | 136 |
| 177 | chr2 | 25149911 | 25150130 | ADCY3 | 9.6E−09 | 135 |
| 178 | chr1 | 53992314 | 53992630 | DMRTB1 | 2.1E−10 | 135 |
| 179 | chr11 | 76903112 | 76903288 | MYO7A | 0.0013 | 135 |
| 180 | chr6 | 436438 | 436782 | IRF4 | 1.6E−11 | 134 |
| 181 | chr16 | 67687133 | 67687281 | ACD | 1 | 134 |
| 182 | chr14 | 105995799 | 105996061 | TMEM121 | 5.7E−45 | 133 |
| 183 | chr16 | 214606 | 216803 | HBM | 0.00028 | 132 |
| 184 | chr13 | 58206330 | 58207289 | PCDH17 | 7.7E−97 | 132 |
| 185 | chr7 | 98246419 | 98246483 | NPTX2 | 0.0072 | 131 |
| 186 | chr12 | 131171298 | 131171496 | STX2 | 4.4E−17 | 128 |
| 187 | chr1 | 151810557 | 151811034 | LOC100132111 | 6.4E−90 | 128 |
| 188 | chr18 | 77917520 | 77918370 | PARD6G-AS1 | 2.3E−48 | 128 |
| 189 | chr6 | 105388908 | 105388951 | LINC00577 | 0.000000037 | 125 |
| 190 | chr13 | 105791702 | 105792506 | DAOA | 1.1E−14 | 125 |
| 191 | chr2 | 154727822 | 154729150 | GALNT13 | 2.7E−157 | 125 |
| 192 | chr2 | 242491705 | 242491871 | BOK | 0.00000061 | 125 |
| 193 | chr16 | 86066512 | 86066907 | MIR6774 | 9.9E−10 | 125 |
| 194 | chr8 | 142613052 | 142613443 | MROH5 | 2.9E−12 | 123 |
| 195 | chr17 | 9129737 | 9129856 | LOC101928266 | 0.000082 | 123 |
| 196 | chr4 | 169799502 | 169799589 | PALLD | 0.000025 | 121 |
| 197 | chr15 | 28107317 | 28107443 | OCA2 | 0.0012 | 121 |
| 198 | chr18 | 77398085 | 77398519 | CTDP1 | 3.9E−15 | 120 |
| 199 | chr2 | 112123586 | 112124284 | | 1.5E−84 | 119 |
| 200 | chr12 | 8025364 | 8026003 | SLC2A14 | 8.6E−48 | 119 |
| 201 | chr13 | 109147979 | 109149018 | MYO16 | 4.6E−154 | 118 |
| 202 | chr1 | 38645072 | 38645464 | LINC01343 | 0.00083 | 118 |
| 203 | chr17 | 80292078 | 80292252 | SECTM1 | 0.18 | 118 |
| 204 | chr8 | 97156853 | 97158021 | GDF6 | 1.8E−136 | 117 |
| 205 | chr17 | 32484186 | 32484259 | ASIC2 | 0.00000016 | 116 |
| 206 | chr2 | 130344986 | 130345228 | LOC151121 | 2.5E−29 | 115 |
| 207 | chr3 | 194097043 | 194097329 | LRRC15 | 0.1 | 113 |
| 208 | chr20 | 61877753 | 61877956 | FLJ16779 | 1 | 112 |
| 209 | chr1 | 156094356 | 156095142 | LMNA | 1 | 111 |
| 210 | chr1 | 2863648 | 2863888 | ACTRT2 | 1 | 111 |
| 211 | chr20 | 54579769 | 54580492 | CBLN4 | 2.6E−96 | 111 |
| 212 | chr10 | 14372398 | 14372955 | FRMD4A | 0.001 | 110 |
| 213 | chr4 | 165304258 | 165305137 | MARCH1 | 2.6E−88 | 110 |
| 214 | chr16 | 1093345 | 1093682 | SSTR5 | 0.000034 | 109 |
| 215 | chr9 | 139837729 | 139838191 | FBXW5 | 0.000066 | 107 |
| 216 | chr2 | 200468425 | 200469111 | LOC101927641 | 1.5E−44 | 107 |
| 217 | chr8 | 65282005 | 65282932 | LOC102724623 | 2.4E−121 | 105 |
| 218 | chr1 | 147782027 | 147782547 | MIR5087 | 2.7E−18 | 104 |
| 219 | chr12 | 125139647 | 125140269 | NCOR2 | 0.0017 | 103 |
| 220 | chr10 | 109674693 | 109675094 | LINC01435 | 9.4E−09 | 102 |
| 221 | chr6 | 84417571 | 84417917 | SNAP91 | 6.1E−35 | 101 |
| 222 | chr4 | 70697332 | 70697562 | SULT1E1 | 1.1E−14 | 100 |
| 223 | chr2 | 118617459 | 118618017 | DDX18 | 1.6E−22 | 99 |
| 224 | chr6 | 159654468 | 159655481 | LOC101929122 | 1.4E−48 | 97 |
| 225 | chr3 | 157812169 | 157812814 | SHOX2 | 4.7E−39 | 94 |
| 226 | chr20 | 31618058 | 31618529 | BPIFB6 | 0.000000099 | 92 |
| 227 | chr19 | 37288169 | 37288705 | ZNF790-AS1 | 3.6E−68 | 90 |
| 228 | chr12 | 41086850 | 41087004 | CNTN1 | 0.000000084 | 87 |
| 229 | chr8 | 67873692 | 67873780 | TCF24 | 1.4E−13 | 87 |
| 230 | chr16 | 66612664 | 66613342 | CMTM2 | 9.8E−55 | 86 |
| 231 | chr20 | 26188996 | 26190201 | MIR663AHG | 1.5E−165 | 85 |
| 232 | chr3 | 26664476 | 26666187 | LRRC3B | 1.5E−49 | 82 |
| 233 | chr7 | 4901208 | 4901886 | PAPOLB | 1.9E−46 | 79 |
| 234 | chr5 | 180591445 | 180591786 | OR2V2 | 2.1E−12 | 76 |
| 235 | chr1 | 224804523 | 224804634 | CNIH3 | 0.00071 | 74 |
| 236 | chr16 | 1014739 | 1015103 | LMF1 | 4.1E−34 | 73 |
| 237 | chr7 | 130418149 | 130418261 | KLF14 | 4.9E−09 | 72 |
| 238 | chr18 | 4454952 | 4454987 | DLGAP1 | 2.2E−20 | 71 |
| 239 | chr2 | 91635832 | 91635943 | LOC654342 | 0.00000012 | 71 |
| 240 | chr7 | 117854057 | 117854207 | ANKRD7 | 1 | 70 |
| 241 | chr19 | 38308083 | 38308175 | LOC644554 | 1.7E−17 | 70 |
| 242 | chr16 | 48844551 | 48845153 | N4BP1 | 7.4E−82 | 68 |
| 243 | chr10 | 108923670 | 108924867 | SORCS1 | 5.9E−143 | 67 |
| 244 | chr2 | 121670072 | 121670819 | GLI2 | 2.1E−24 | 67 |
| 245 | chr14 | 103415760 | 103415938 | AMN | 1 | 65 |
| 246 | chr3 | 128209603 | 128210307 | GATA2-AS1 | 4.1E−15 | 63 |
| 247 | chr17 | 73642361 | 73642628 | SMIM6 | 0.00043 | 63 |
| 248 | chr7 | 6703864 | 6704236 | ZNF316 | 1.3E−52 | 62 |
| 249 | chr7 | 30721306 | 30721664 | CRHR2 | 5.5E−37 | 61 |
| 250 | chr1 | 44402352 | 44402522 | ARTN | 0.0000061 | 59 |
| 251 | chr18 | 77548469 | 77548919 | KCNG2 | 2.4E−14 | 58 |
| 252 | chr10 | 22766062 | 22766978 | LOC100499489 | 0.0000046 | 57 |
| 253 | chr7 | 158765970 | 158766499 | LINC00689 | 6.4E−38 | 56 |
| 254 | chr6 | 12292528 | 12292784 | EDN1 | 0.000000029 | 55 |
| 255 | chr11 | 1857513 | 1857939 | SYT8 | 0.00031 | 55 |
| 256 | chr18 | 70533842 | 70535309 | NETO1 | 1.5E−187 | 54 |
| 257 | chr3 | 147108577 | 147111786 | ZIC4 | 1.5E−203 | 53 |
| 258 | chr7 | 100942951 | 100943665 | LOC101927746 | 9.3E−42 | 52 |
| 259 | chr1 | 3139735 | 3140034 | MIR4251 | 0.0000093 | 52 |
| 260 | chr16 | 1217322 | 1217374 | CACNA1H | 9.8E−12 | 50 |
| 261 | chr19 | 12624151 | 12624679 | ZNF709 | 8.2E−26 | 49 |
| 262 | chr2 | 241459501 | 241460162 | ANKMY1 | 1.1E−12 | 49 |
| 263 | chr12 | 130388961 | 130389122 | TMEM132D | 0.2 | 47 |
| 264 | chr1 | 228400272 | 228400693 | C1orf145 | 0.095 | 46 |
| 265 | chr5 | 92930812 | 92931061 | MIR548AO | 0.000000012 | 46 |
| 266 | chr10 | 126254543 | 126254769 | LHPP | 0.046 | 43 |
| 267 | chr5 | 175107749 | 175108520 | HRH2 | 8.7E−11 | 42 |
| 268 | chr10 | 135178458 | 135178918 | MIR3944 | 7.1E−15 | 41 |
| 269 | chr6 | 167069894 | 167070804 | RPS6KA2 | 1.5E−09 | 41 |
| 270 | chr14 | 105251245 | 105251699 | AKT1 | 8.2E−12 | 40 |
| 271 | chr19 | 17404806 | 17405017 | ABHD8 | 0.84 | 40 |
| 272 | chr2 | 202900905 | 202901100 | FZD7 | 9.8E−12 | 40 |
| 273 | chr13 | 70681585 | 70682357 | ATXN8OS | 2.2E−51 | 40 |
| 274 | chr3 | 184056350 | 184056670 | FAM131A | 1 | 39 |
| 275 | chr2 | 242927372 | 242927735 | LOC102723927 | 4.4E−11 | 38 |
| 276 | chr9 | 971867 | 971999 | DMRT3 | 0.0000027 | 37 |
| 277 | chr13 | 114060446 | 114060794 | LOC101928841 | 0.0086 | 35 |
| 278 | chr19 | 57182797 | 57183497 | ZNF835 | 1.8E−80 | 35 |
| 279 | chr12 | 127210739 | 127211525 | LINC00943 | 1E−82 | 32 |
| 280 | chr8 | 55382673 | 55383188 | SOX17 | 8.6E−82 | 32 |
| 281 | chr12 | 107975098 | 107975195 | BTBD11 | 0.00055 | 31 |
| 282 | chr14 | 105048396 | 105048656 | C14orf180 | 0.0012 | 30 |
| 283 | chr10 | 126211559 | 126211820 | LHPP | 0.000017 | 30 |
| 284 | chr12 | 107974803 | 107975132 | BTBD11 | 5.9E−22 | 29 |
| 285 | chr11 | 2481009 | 2482442 | KCNQ1 | 5.4E−19 | 28 |
| 286 | chr17 | 35299347 | 35300455 | LHX1 | 9.4E−93 | 28 |
| 287 | chr16 | 89120615 | 89120850 | ACSF3 | 2.5E−09 | 28 |
| 288 | chr1 | 205818854 | 205819345 | PM20D1 | 1.4E−38 | 27 |
| 289 | chr17 | 72353601 | 72353907 | BTBD17 | 2.5E−17 | 27 |
| 290 | chr1 | 148901738 | 148902370 | LOC101060524 | 9.8E−118 | 26 |
| 291 | chr15 | 45408769 | 45409558 | DUOXA2 | 3.3E−36 | 25 |
| 292 | chr11 | 132952630 | 132952681 | OPCML | 0.0011 | 24 |
| 293 | chr8 | 144437148 | 144437608 | TOP1MT | 3E−19 | 24 |
| 294 | chr8 | 70946912 | 70947440 | PRDM14 | 8.8E−32 | 24 |
| 295 | chr6 | 169425360 | 169425418 | LOC101929504 | 1 | 22 |
| 296 | chr1 | 231299494 | 231299573 | TRIM67 | 0.00014 | 22 |
| 297 | chr5 | 115151235 | 115152712 | CDO1 | 2.1E−94 | 21 |
| 298 | chr13 | 100069021 | 100069251 | MIR548AN | 9.7E−17 | 20 |
| 299 | chr1 | 933737 | 933974 | HES4 | 1.7E−18 | 20 |
| 300 | chr4 | 158141284 | 158141917 | GRIA2 | 5.7E−62 | 19 |
| 301 | chr2 | 177024366 | 177024783 | HOXD3 | 1E−24 | 19 |
| 302 | chr18 | 55019953 | 55021680 | ST8SIA3 | 0.84 | 19 |
| 303 | chr5 | 140871885 | 140872486 | PCDHGC5 | 5.1E−33 | 18 |
| 304 | chr7 | 53286811 | 53287070 | POM121L12 | 9.4E−31 | 18 |
| 305 | chr19 | 56879261 | 56880146 | ZNF542P | 3.1E−64 | 18 |
| 306 | chr1 | 153589743 | 153590365 | S100A14 | 0.000000052 | 17 |
| 307 | chr6 | 27114378 | 27114572 | HIST1H2BK | 1.5E−16 | 17 |
| 308 | chr18 | 67068677 | 67069273 | DOK6 | 2.7E−58 | 17 |
| 309 | chr1 | 50886715 | 50887270 | DMRTA2 | 3.2E−74 | 16 |
| 310 | chr18 | 60263389 | 60263741 | ZCCHC2 | 1.4E−19 | 16 |
| 311 | chr3 | 154146310 | 154146951 | GPR149 | 1.5E−41 | 15 |
| 312 | chr2 | 220174037 | 220174100 | PTPRN | 1.8E−19 | 15 |
| 313 | chr6 | 62995658 | 62996299 | KHDRBS2 | 2.2E−68 | 15 |
| 314 | chr3 | 239997 | 240043 | CHL1 | 6.3E−17 | 14 |
| 315 | chr19 | 46526268 | 46526750 | PGLYRP1 | 6.5E−15 | 14 |
| 316 | chr16 | 51183896 | 51185739 | SALL1 | 8E−132 | 14 |
| 317 | chr1 | 1109468 | 1109757 | TTLL10 | 6.4E−10 | 13 |
| 318 | chr5 | 140871836 | 140871882 | PCDHGC5 | 2.6E−16 | 13 |
| 319 | chr2 | 31457371 | 31457421 | EHD3 | 1.8E−13 | 13 |
| 320 | chr2 | 231855124 | 231855824 | SPATA3-AS1 | 3E−55 | 12 |
| 321 | chr14 | 93153235 | 93153519 | LGMN | 6.1E−27 | 12 |
| 322 | chr1 | 11709190 | 11709276 | FBXO44 | 0.0012 | 11 |
| 323 | chr9 | 140772527 | 140772595 | CACNA1B | 1.1E−11 | 11 |
| 324 | chr2 | 176981980 | 176982522 | HOXD10 | 7.6E−15 | 11 |
| 325 | chr1 | 240256208 | 240257032 | FMN2 | 9.3E−68 | 11 |
| 326 | chr17 | 75369481 | 75370580 | SEPT9 | 1.1E−84 | 11 |
| 327 | chr1 | 119522232 | 119522972 | TBX15 | 1.4E−55 | 10 |
| 328 | chr5 | 3606309 | 3606498 | IRX1 | 0.000000015 | 10 |
| 329 | chr8 | 143546027 | 143546579 | ADGRB1 | 6.7E−24 | 9 |
| 330 | chr11 | 70559082 | 70559473 | SHANK2 | 1 | 9 |
| 331 | chr15 | 96959357 | 96959433 | NR2F2 | 0.0000002 | 9 |
| 332 | chr6 | 123317044 | 123317899 | CLVS2 | 2E−63 | 8 |
| 333 | chr5 | 170743598 | 170743662 | TLX3 | 1.8E−17 | 8 |
| 334 | chr2 | 220313278 | 220313578 | SPEG | 2.4E−59 | 8 |
| 335 | chr19 | 37095856 | 37096662 | ZNF382 | 1.8E−65 | 8 |
| 336 | chr6 | 85482569 | 85483797 | TBX18 | 3E−99 | 8 |
| 337 | chr10 | 135383179 | 135383340 | SPRNP1 | 6.2E−17 | 7 |
| 338 | chr12 | 54763057 | 54763367 | GPR84 | 0.029 | 7 |
| 339 | chr11 | 63768139 | 63768217 | OTUB1 | 1 | 7 |
| 340 | chr8 | 70983045 | 70984036 | PRDM14 | 3.2E−41 | 7 |
| 341 | chr9 | 122131297 | 122131749 | BRINP1 | 9E−54 | 6 |
| 342 | chr11 | 2181458 | 2182795 | INS | 5E−15 | 6 |
| 343 | chr17 | 79615325 | 79615612 | TSPAN10 | 1.6E−25 | 6 |
| 344 | chr1 | 10764635 | 10764790 | CASZ1 | 2.9E−09 | 5 |
| 345 | chr13 | 111819372 | 111819624 | ARHGEF7 | 0.16 | 5 |
| 346 | chr7 | 1234825 | 1235034 | LOC101927021 | 0.00000012 | 5 |
| 347 | chr18 | 12911249 | 12911727 | PTPN2 | 2.1E−40 | 5 |
| 348 | chr8 | 144155053 | 144155326 | C8orf31 | 7.7E−16 | 5 |
| 349 | chr5 | 176046720 | 176047338 | MIR4281 | 1.2E−26 | 5 |
| 350 | chr20 | 61636196 | 61636278 | BHLHE23 | 0.000022 | 5 |
| 351 | chr1 | 85156186 | 85156257 | SSX2IP | 0.00013 | 5 |
| 352 | chr13 | 112984830 | 112985846 | LINC01044 | 1 | 4 |
| 353 | chr8 | 1590944 | 1591041 | DLGAP2-AS1 | 1 | 4 |
| 354 | chr3 | 192126003 | 192126114 | FGF12 | 2E−13 | 4 |
| 355 | chr17 | 41832634 | 41832704 | SOST | 0.53 | 4 |
| 356 | chr17 | 55213562 | 55213720 | AKAP1 | 0.00000001 | 4 |
| 357 | chr19 | 55597993 | 55599029 | EPS8L1 | 1.6E−70 | 4 |
| 358 | chr4 | 85417659 | 85418648 | NKX6-1 | 2.6E−50 | 4 |
| 359 | chr11 | 976859 | 977067 | AP2A2 | 1 | 4 |
| 360 | chr9 | 115652625 | 115653447 | SLC46A2 | 2.5E−54 | 3 |
| 361 | chr12 | 132859655 | 132859824 | LOC100130238 | 0.00000032 | 3 |
| 362 | chr1 | 205818764 | 205819209 | PM20D1 | 0.00047 | 3 |
| 363 | chr20 | 26188638 | 26188976 | MIR663A | 6.4E−13 | 3 |
| 364 | chr15 | 32605344 | 32605617 | GOLGA8K | 6.4E−45 | 3 |
| 365 | chr5 | 54518960 | 54519686 | MCIDAS | 1.2E−44 | 3 |
| 366 | chr19 | 56061179 | 56061515 | SBK3 | 0.000000041 | 3 |
| 367 | chr2 | 63274711 | 63275273 | LOC100132215 | 1.2E−33 | 3 |
| 368 | chr14 | 73736836 | 73737235 | PAPLN | 8.7E−33 | 3 |
| 369 | chr20 | 853374 | 853676 | FAM110A | 0.013 | 3 |
| 370 | chr14 | 104927422 | 104927552 | C14orf180 | 0.0059 | 2 |
| 371 | chr8 | 144303024 | 144303454 | GPIHBP1 | 3.7E−09 | 2 |
| 372 | chr20 | 21686714 | 21687523 | PAX1 | 1.1E−72 | 2 |
| 373 | chr12 | 312591 | 312753 | LOC101929384 | 2E−45 | 2 |
| 374 | chr1 | 42611485 | 42611690 | GUCA2B | 1 | 2 |
| 375 | chr20 | 43378734 | 43379462 | KCNK15 | 5E−59 | 2 |
| 376 | chr21 | 46975852 | 46976310 | SLC19A1 | 3.2E−43 | 2 |
| 377 | chr12 | 57618686 | 57618783 | SHMT2 | 0.000000005 | 2 |
| 378 | chr19 | 58220116 | 58220669 | ZNF154 | 6.9E−40 | 2 |
| 379 | chr15 | 66914678 | 66914731 | LINC01169 | 0.000024 | 2 |
| 380 | chr14 | 76597846 | 76597910 | GPATCH2L | 0.00000061 | 2 |
| 381 | chr10 | 86004394 | 86005121 | RGR | 0.0011 | 2 |
| 382 | chr2 | 90284670 | 90284928 | MIR4436A | 1 | 2 |
| 383 | chr8 | 101821840 | 101822199 | PABPC1 | 4.4E−14 | 1 |
| 384 | chr14 | 105499705 | 105499909 | CDCA4 | 7.7E−37 | 1 |
| 385 | chr11 | 1095098 | 1095324 | MUC2 | 6.5E−09 | 1 |
| 386 | chr12 | 133049696 | 133050826 | FBRSL1 | 4.4E−13 | 1 |
| 387 | chr3 | 13515572 | 13515826 | HDAC11-AS1 | 0.00067 | 1 |
| 388 | chr6 | 167535625 | 167536283 | CCR6 | 3.1E−10 | 1 |
| 389 | chr11 | 19695514 | 19695859 | NAV2 | 0.00043 | 1 |
| 390 | chr1 | 203320137 | 203320661 | FMOD | 3.6E−12 | 1 |
| 391 | chr17 | 2310128 | 2311051 | MNT | 1 | 1 |
| 392 | chr2 | 240168706 | 240169346 | MGC16025 | 1.9E−36 | 1 |
| 393 | chr2 | 241562775 | 241562890 | GPR35 | 6.2E−10 | 1 |
| 394 | chr20 | 25061647 | 25062927 | VSX1 | 8.2E−78 | 1 |
| 395 | chr6 | 27512776 | 27513570 | ZNF184 | 5.4E−27 | 1 |
| 396 | chr11 | 2890555 | 2890941 | KCNQ1DN | 5.7E−37 | 1 |
| 397 | chr22 | 29876775 | 29877285 | NEFH | 5.4E−49 | 1 |
| 398 | chr7 | 37487865 | 37487940 | ELMO1 | 0.0046 | 1 |
| 399 | chr7 | 37488026 | 37488114 | ELMO1 | 0.00003 | 1 |
| 400 | chr8 | 49340828 | 49341091 | LOC101929268 | 3.4E−13 | 1 |
| 401 | chr19 | 58629190 | 58629803 | ZSCAN18 | 7.3E−54 | 1 |
| 402 | chr17 | 80332729 | 80333205 | UTS2R | 4.8E−25 | 1 |
| 403 | chr10 | 94451209 | 94452485 | HHEX | 1.7E−69 | 1 |
| 404 | chr3 | 9988376 | 9989677 | PRRT3-AS1 | 2.6E−11 | 1 |

### Gastric Cancer

In another aspect is provided a method of detecting a level of DNA methylation in a subject at risk of developing a gastric cancer, the method including: determining the degree of methylation of CpG sites within a plurality of gene regions in a DNA sample from the subject, wherein the plurality of gene regions includes at least 5 different gene regions in Table GC. In embodiments, an increased level of methylated CpG sites relative to a standard control indicates a higher risk of gastric cancer.

Provided herein is a method of treating gastric cancer in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table GC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the gastric cancer is Stage I, Stage II, or Stage III. In embodiments, the gastric cancer is Stage I. In embodiments, the gastric cancer is Stage II. In embodiments, the gastric cancer is Stage III.

Provided herein is a method of diagnosing gastric cancer in a patient comprising: (a) detecting a level of methylated CpG sites within a plurality of gene regions in a DNA sample from the patient, wherein the plurality of gene regions comprise at least 5 different gene regions in Table GC; and (b) diagnosing the patient with gastric cancer when the DNA sample has an increased level of methylated CpG sites, relative to a standard control, within the plurality of gene regions. In embodiments, the gastric cancer is Stage I, Stage II, or Stage III. In embodiments, the gastric cancer is Stage I. In embodiments, the gastric cancer is Stage II. In embodiments, the gastric cancer is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having gastric cancer or monitoring risk for developing gastric cancer in a patient comprising: (a) detecting a level of methylated CpG sites within a plurality of gene regions in a DNA sample from the patient at a first time point, wherein the plurality of gene regions comprise at least 5 different gene regions in Table GC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing gastric cancer. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing gastric cancer or does not have gastric cancer. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing gastric cancer or may have gastric cancer. In embodiments, the gastric cancer is Stage I, Stage II, or Stage III. In embodiments, the gastric cancer is Stage I. In embodiments, the gastric cancer is Stage II. In embodiments, the gastric cancer is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table GC. In embodiments, the plurality of gene regions includes at least 2 DMRs (i.e., gene regions) in Table GC. In embodiments, the plurality of gene regions includes at least 3 DMRs in Table GC. In embodiments, the plurality of gene regions includes at least 4 DMRs in Table GC. In embodiments, the plurality of gene regions includes at least 5 DMRs in Table GC. In embodiments, the plurality of gene regions includes at least 6 DMRs in Table GC. In embodiments, the plurality of gene regions includes at least 7 DMRs in Table GC. In embodiments, the plurality of gene regions includes at least 8 DMRs in Table GC. In embodiments, the plurality of gene regions includes at least 9 DMRs in Table GC. In embodiments, the plurality of gene regions includes at least 10 DMRs in Table GC. In embodiments, the plurality of gene regions includes at least 15 DMRs in Table GC. In embodiments, the plurality of gene regions includes at least 20 DMRs in Table GC. In embodiments, the plurality of gene regions includes at least 25 DMRs in Table GC. In embodiments, the plurality of gene regions includes at least 30 DMRs in Table GC. In embodiments, the plurality of gene regions includes at least 35 DMRs in Table GC. In embodiments, the plurality of gene regions includes at least 40 DMRs in Table GC. In embodiments, the plurality of gene regions includes at least 45 DMRs in Table GC. In embodiments, the plurality of gene regions includes at least 50 DMRs in Table GC. In embodiments, the plurality of gene regions includes at least 55 DMRs in Table GC. In embodiments, the plurality of gene regions includes at least 60 DMRs in Table GC. In embodiments, the plurality of gene regions includes at least 65 DMRs in Table GC. In embodiments, the plurality of gene regions includes at least 70 DMRs in Table GC. In embodiments, the plurality of gene regions includes at least 75 DMRs in Table GC. In embodiments, the plurality of gene regions includes at least 80 DMRs in Table GC. In embodiments, the plurality of gene regions includes at least 85 DMRs in Table GC. In embodiments, the plurality of gene regions includes at least 90 DMRs in Table GC. In embodiments, the plurality of gene regions includes at least 95 DMRs in Table GC. In embodiments, the plurality of gene regions includes at least 110 DMRs in Table GC. In embodiments, the plurality of gene regions includes at least 120 DMRs in Table GC. In embodiments, the plurality of gene regions includes at least 130 DMRs in Table GC. In embodiments, the plurality of gene regions includes at least 140 DMRs in Table GC. In embodiments, the plurality of gene regions includes at least 150 DMRs in Table GC. In embodiments, the plurality of gene regions includes at least 160 DMRs in Table GC. In embodiments, the plurality of gene regions includes at least 170 DMRs in Table GC. In embodiments, the plurality of gene regions includes at least 180 DMRs in Table GC. In embodiments, the plurality of gene regions includes at least 190 DMRs in Table GC. In embodiments, the plurality of gene regions includes at least 200 DMRs in Table GC. In embodiments, the plurality of gene regions includes at least 225 DMRs in Table GC. In embodiments, the plurality of gene regions includes at least 250 DMRs in Table GC. In embodiments, the plurality of gene regions includes at least 275 DMRs in Table GC. In embodiments, the plurality of gene regions includes at least 300 DMRs in Table GC. In embodiments, the plurality of gene regions includes at least 320 DMRs in Table GC.

In embodiments, the plurality of gene regions includes the first DMR (i.e., gene region) in Table GC. In embodiments, the plurality of gene regions includes the first 2 DMRs (i.e., gene regions) in Table GC. In embodiments, the plurality of gene regions includes the first 3 DMRs in Table GC. In embodiments, the plurality of gene regions includes the first 4 DMRs in Table GC. In embodiments, the plurality of gene regions includes the first 5 DMRs in Table GC. In embodiments, the plurality of gene regions includes the first 6 DMRs in Table GC. In embodiments, the plurality of gene regions includes the first 7 DMRs in Table GC. In embodiments, the plurality of gene regions includes the first 8 DMRs in Table GC. In embodiments, the plurality of gene regions includes the first 9 DMRs in Table GC. In embodiments, the plurality of gene regions includes the first 10 DMRs in Table GC. In embodiments, the plurality of gene regions includes the first 11 DMRs in Table GC. In embodiments, the plurality of gene regions includes the first 12 DMRs in Table GC. In embodiments, the plurality of gene regions includes the first 13 DMRs in Table GC. In embodiments, the plurality of gene regions includes the first 14 DMRs in Table GC. In embodiments, the plurality of gene regions includes the first 15 DMRs in Table GC. In embodiments, the plurality of gene regions includes the first 16 DMRs in Table GC. In embodiments, the plurality of gene regions includes the first 17 DMRs in Table GC. In embodiments, the plurality of gene regions includes the first 18 DMRs in Table GC. In embodiments, the plurality of gene regions includes the first 19 DMRs in Table GC. In embodiments, the plurality of gene regions includes the first 20 DMRs in Table GC. In embodiments, the plurality of gene regions includes the first 25 DMRs in Table GC. In embodiments, the plurality of gene regions includes the first 30 DMRs in Table GC. In embodiments, the plurality of gene regions includes the first 35 DMRs in Table GC. In embodiments, the plurality of gene regions includes the first 40 DMRs in Table GC. In embodiments, the plurality of gene regions includes the first 45 DMRs in Table GC. In embodiments, the plurality of gene regions includes the first 50 DMRs in Table GC. In embodiments, the plurality of gene regions includes the first 55 DMRs in Table GC. In embodiments, the plurality of gene regions includes the first 60 DMRs in Table GC. In embodiments, the plurality of gene regions includes the first 65 DMRs in Table GC. In embodiments, the plurality of gene regions includes the first 70 DMRs in Table GC. In embodiments, the plurality of gene regions includes the first 75 DMRs in Table GC. In embodiments, the plurality of gene regions includes the first 80 DMRs in Table GC. In embodiments, the plurality of gene regions includes the first 85 DMRs in Table GC. In embodiments, the plurality of gene regions includes the first 90 DMRs in Table GC. In embodiments, the plurality of gene regions includes the first 95 DMRs in Table GC. In embodiments, the plurality of gene regions includes the first 110 DMRs in Table GC. In embodiments, the plurality of gene regions includes the first 120 DMRs in Table GC. In embodiments, the plurality of gene regions includes the first 130 DMRs in Table GC. In embodiments, the plurality of gene regions includes the first 140 DMRs in Table GC. In embodiments, the plurality of gene regions includes the first 150 DMRs in Table GC. In embodiments, the plurality of gene regions includes the first 160 DMRs in Table GC. In embodiments, the plurality of gene regions includes the first 170 DMRs in Table GC. In embodiments, the plurality of gene regions includes the first 180 DMRs in Table GC. In embodiments, the plurality of gene regions includes the first 190 DMRs in Table GC. In embodiments, the plurality of gene regions includes the first 200 DMRs in Table GC. In embodiments, the plurality of gene regions includes the first 225 DMRs in Table GC. In embodiments, the plurality of gene regions includes the first 250 DMRs in Table GC. In embodiments, the plurality of gene regions includes the first 275 DMRs in Table GC. In embodiments, the plurality of gene regions includes the first 300 DMRs in Table GC. In embodiments, the plurality of gene regions includes the first 320 DMRs in Table GC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is a fine needle aspiration, an esophagogastroduodenoscopy, or tissue biopsy. In embodiments, the confirmatory diagnostic procedure is a computed tomography scan, a positron emission tomography scan, a magnetic resonance imaging scan, or fecal occult blood test.

In embodiments, the method further includes treating the subject for gastric cancer. In embodiments, treating includes endoscopic mucosal resection, partial (Distal) Gastrectomy, or total Gastrectomy. In embodiments, treating includes radiotherapy, chemotherapy, targeted therapy, or immunotherapy. In embodiments, treating includes radiotherapy, chemotherapy, targeted therapy, immunotherapy, or a combination of two or more thereof.

TABLE GC

| Gene | | | | | | |
|---|---|---|---|---|---|---|
| Region | | | | | Adjusted | |
| No. | Chr | Start | End | Gene Name | p-value | Freq |
| 1 | chr7 | 62574764 | 62574921 | ZNF733P | 1.7E−10 | 409 |
| 2 | chr20 | 37356055 | 37357918 | SLC32A1 | 1.2E−132 | 407 |
| 3 | chr6 | 41604583 | 41605020 | MDFI | 2E−18 | 407 |
| 4 | chr19 | 35940424 | 35940980 | FFAR2 | 4.9E−15 | 405 |
| 5 | chr2 | 90413838 | 90414075 | MIR4436A | 1.3E−27 | 405 |
| 6 | chr19 | 827575 | 827995 | AZU1 | 7.6E−12 | 403 |
| 7 | chr12 | 128751380 | 128751622 | TMEM132C | 0.00004 | 402 |
| 8 | chr19 | 30866358 | 30866489 | ZNF536 | 0.000059 | 402 |
| 9 | chr2 | 91634830 | 91635236 | LOC654342 | 2.3E−38 | 402 |
| 10 | chr13 | 112723041 | 112724206 | SOX1 | 2.1E−56 | 401 |
| 11 | chr5 | 172671531 | 172672649 | NKX2-5 | 8.8E−43 | 400 |
| 12 | chr1 | 151810557 | 151811034 | LOC100132111 | 6.4E−90 | 399 |
| 13 | chr8 | 41165745 | 41167139 | SFRP1 | 7E−164 | 397 |
| 14 | chr16 | 1171931 | 1172126 | C1QTNF8 | 1 | 396 |
| 15 | chr2 | 5832525 | 5834007 | SOX11 | 1.9E−195 | 396 |
| 16 | chr7 | 8481842 | 8482826 | NXPH1 | 2.4E−78 | 395 |
| 17 | chr12 | 124393534 | 124394014 | CCDC92 | 0.000039 | 392 |
| 18 | chr2 | 74668071 | 74668672 | RTKN | 3.1E−18 | 392 |
| 19 | chr1 | 1244840 | 1245076 | PUSL1 | 8.8E−09 | 390 |
| 20 | chr1 | 153362847 | 153364085 | S100A8 | 4.3E−11 | 390 |
| 21 | chr7 | 103630754 | 103631004 | RELN | 1 | 389 |
| 22 | chr19 | 57276614 | 57277068 | ZIM2-AS1 | 5.4E−41 | 389 |
| 23 | chr2 | 63284081 | 63284165 | OTX1 | 0.00000041 | 379 |
| 24 | chr10 | 7139067 | 7139412 | SFMBT2 | 0.000005 | 367 |
| 25 | chr11 | 31835576 | 31835723 | PAX6 | 0.000097 | 355 |
| 26 | chr20 | 56283770 | 56284043 | PMEPA1 | 7E−11 | 349 |
| 27 | chr19 | 13616986 | 13617209 | CACNA1A | 8.7E−51 | 339 |
| 28 | chr11 | 45718064 | 45718522 | MIR7154 | 0.00000015 | 318 |
| 29 | chr1 | 149137698 | 149138116 | | 6.5E−63 | 289 |
| 30 | chr5 | 145719093 | 145720027 | POU4F3 | 5.6E−64 | 283 |
| 31 | chr11 | 83392903 | 83393677 | DLG2 | 0.0000035 | 279 |
| 32 | chr11 | 27741817 | 27742122 | BDNF | 0.0000021 | 272 |
| 33 | chr11 | 2720261 | 2721440 | KCNQ1OT1 | 6.9E−30 | 267 |
| 34 | chr3 | 26664476 | 26666187 | LRRC3B | 1.5E−49 | 251 |
| 35 | chr10 | 50605228 | 50605945 | DRGX | 1.1E−57 | 250 |
| 36 | chr5 | 76924167 | 76924428 | OTP | 1.9E−30 | 245 |
| 37 | chr18 | 5891167 | 5891444 | TMEM200C | 3.9E−55 | 243 |
| 38 | chr11 | 94278269 | 94278973 | FUT4 | 2.6E−68 | 232 |
| 39 | chr6 | 62995658 | 62996299 | KHDRBS2 | 2.2E−68 | 220 |
| 40 | chr8 | 43132277 | 43132418 | POTEA | 0.000000014 | 219 |
| 41 | chr7 | 56355481 | 56355797 | LOC650226 | 8.1E−65 | 215 |
| 42 | chr3 | 183958943 | 183959882 | MIR1224 | 5.5E−52 | 213 |
| 43 | chr19 | 38308083 | 38308175 | LOC644554 | 1.7E−17 | 213 |
| 44 | chr12 | 54348949 | 54349679 | HOXC12 | 3.9E−74 | 211 |
| 45 | chr13 | 28498102 | 28499045 | PDX1-AS1 | 0.0000017 | 206 |
| 46 | chr4 | 156129443 | 156129588 | NPY2R | 4.9E−16 | 204 |
| 47 | chr7 | 158224687 | 158225210 | MIR595 | 5.1E−15 | 203 |
| 48 | chr5 | 170743598 | 170743662 | TLX3 | 1.8E−17 | 203 |
| 49 | chr7 | 102073609 | 102074185 | ORAI2 | 0.00000029 | 198 |
| 50 | chr3 | 147136946 | 147137256 | LOC440982 | 3.2E−16 | 197 |
| 51 | chr3 | 179754308 | 179755306 | PEX5L | 8.8E−82 | 197 |
| 52 | chr3 | 6903030 | 6903205 | GRM7 | 2.2E−41 | 197 |
| 53 | chr1 | 53308583 | 53308809 | ZYG11A | 0.00000005 | 195 |
| 54 | chr8 | 61835619 | 61835795 | LOC100130298 | 2.9E−09 | 194 |
| 55 | chr12 | 115135925 | 115135961 | TBX3 | 4E−11 | 193 |
| 56 | chr20 | 21695247 | 21695306 | PAX1 | 3.6E−14 | 192 |
| 57 | chr1 | 1099206 | 1100655 | MIR200B | 2.3E−62 | 191 |
| 58 | chr20 | 21694312 | 21694738 | PAX1 | 5.1E−22 | 191 |
| 59 | chr5 | 140261734 | 140262268 | PCDHA13 | 2.2E−13 | 190 |
| 60 | chr10 | 108923670 | 108924867 | SORCS1 | 5.9E−143 | 189 |
| 61 | chr9 | 137393007 | 137393435 | RXRA | 5.2E−17 | 189 |
| 62 | chr12 | 128752541 | 128753150 | TMEM132C | 6.3E−65 | 187 |
| 63 | chr2 | 219246851 | 219247082 | SLC11A1 | 0.00014 | 187 |
| 64 | chr7 | 57484133 | 57484497 | MIR3147 | 6.1E−35 | 187 |
| 65 | chr15 | 24722579 | 24723145 | PWRN3 | 2.1E−14 | 185 |
| 66 | chr1 | 148901738 | 148902370 | LOC101060524 | 9.8E−118 | 182 |
| 67 | chr2 | 232791348 | 232791531 | NPPC | 0.0000041 | 182 |
| 68 | chr7 | 57928181 | 57928941 | ZNF716 | 1 | 181 |
| 69 | chr17 | 4463580 | 4464239 | GGT6 | 1.6E−09 | 180 |
| 70 | chr1 | 10961102 | 10961414 | C1orf127 | 0.00000046 | 176 |
| 71 | chr8 | 132052036 | 132054784 | ADCY8 | 5.2E−216 | 176 |
| 72 | chr10 | 131770707 | 131771555 | EBF3 | 8E−131 | 175 |
| 73 | chr5 | 178016539 | 178017711 | COL23A1 | 1.9E−134 | 173 |
| 74 | chr9 | 122131297 | 122131749 | BRINP1 | 9E−54 | 172 |
| 75 | chr3 | 13974336 | 13974661 | FGD5P1 | 1.1E−46 | 169 |
| 76 | chr16 | 49309030 | 49309352 | CBLN1 | 1.4E−23 | 169 |
| 77 | chr2 | 42719853 | 42720254 | KCNG3 | 2.1E−47 | 168 |
| 78 | chr16 | 48844551 | 48845153 | N4BP1 | 7.4E−82 | 168 |
| 79 | chr7 | 98246419 | 98246483 | NPTX2 | 0.0072 | 168 |
| 80 | chr4 | 5891985 | 5892089 | CRMP1 | 0.000000003 | 167 |
| 81 | chr19 | 2428326 | 2428471 | TIMM13 | 1 | 165 |
| 82 | chr2 | 72374364 | 72374568 | CYP26B1 | 8.9E−32 | 162 |
| 83 | chr6 | 100906564 | 100906629 | SIM1 | 3.2E−15 | 160 |
| 84 | chr3 | 141174173 | 141174534 | RASA2 | 0.0000071 | 159 |
| 85 | chr10 | 36476957 | 36477264 | PCAT5 | 0.33 | 158 |
| 86 | chr20 | 5297480 | 5297906 | PROKR2 | 3.8E−71 | 158 |
| 87 | chr3 | 6903440 | 6903507 | GRM7 | 3.1E−10 | 157 |
| 88 | chr8 | 15094436 | 15094544 | SGCZ | 0.000000041 | 156 |
| 89 | chr20 | 33134749 | 33135071 | MAP1LC3A | 6.3E−09 | 156 |
| 90 | chr10 | 89167291 | 89168132 | LINC00864 | 0.000023 | 156 |
| 91 | chr12 | 43944719 | 43946284 | ADAMTS20 | 1.7E−154 | 155 |
| 92 | chr7 | 56605646 | 56606225 | LOC101928401 | 0.000000012 | 154 |
| 93 | chr3 | 196373400 | 196373649 | NRROS | 0.000045 | 150 |
| 94 | chr5 | 4222954 | 4223319 | LOC101929153 | 0.002 | 149 |
| 95 | chr20 | 62092340 | 62092502 | KCNQ2 | 0.00017 | 147 |
| 96 | chr2 | 45155998 | 45156907 | SIX3-AS1 | 2.7E−64 | 145 |
| 97 | chr5 | 177433790 | 177434106 | FAM153C | 9.1E−16 | 144 |
| 98 | chr13 | 28500948 | 28503530 | PDX1-AS1 | 4.3E−128 | 142 |
| 99 | chr2 | 105472294 | 105472336 | POU3F3 | 1.1E−12 | 137 |
| 100 | chr11 | 2930793 | 2930938 | SLC22A18AS | 1 | 136 |
| 101 | chr1 | 36042437 | 36042509 | TFAP2E | 7.1E−09 | 134 |
| 102 | chr8 | 125741075 | 125741233 | MTSS1 | 0.00000022 | 133 |
| 103 | chr11 | 63768139 | 63768217 | OTUB1 | 1 | 132 |
| 104 | chr5 | 170735030 | 170736513 | TLX3 | 2.7E−59 | 129 |
| 105 | chr12 | 6664346 | 6665439 | IFFO1 | 9.5E−115 | 129 |
| 106 | chr17 | 72848047 | 72848236 | GRIN2C | 0.0045 | 127 |
| 107 | chr4 | 134071516 | 134074046 | PCDH10 | 9.4E−218 | 126 |
| 108 | chr9 | 137809551 | 137810259 | FCN1 | 1 | 126 |
| 109 | chr8 | 67873692 | 67873780 | TCF24 | 1.4E−13 | 125 |
| 110 | chr7 | 45614067 | 45614108 | ADCY1 | 0.21 | 124 |
| 111 | chr7 | 153583317 | 153584340 | DPP6 | 2.7E−90 | 122 |
| 112 | chr9 | 88137539 | 88138100 | AGTPBP1 | 1.3E−30 | 118 |
| 113 | chr2 | 72374558 | 72374612 | CYP26B1 | 0.00024 | 117 |
| 114 | chr12 | 107975098 | 107975195 | BTBD11 | 0.00055 | 113 |
| 115 | chr7 | 63642030 | 63642712 | ZNF735 | 3.8E−09 | 112 |
| 116 | chr1 | 15059591 | 15059936 | KAZN | 1 | 111 |
| 117 | chr7 | 56297353 | 56297661 | NUPR2 | 6.5E−12 | 111 |
| 118 | chr20 | 61809243 | 61809785 | MIR124-3 | 5.9E−83 | 110 |
| 119 | chr1 | 59280126 | 59280619 | LINC01135 | 5.7E−20 | 109 |
| 120 | chr7 | 70596072 | 70597019 | WBSCR17 | 9.7E−127 | 109 |
| 121 | chr5 | 93905177 | 93905542 | KIAA0825 | 1.1E−73 | 108 |
| 122 | chr7 | 155242318 | 155242925 | EN2 | 1.1E−58 | 107 |
| 123 | chr8 | 97156853 | 97158021 | GDF6 | 1.8E−136 | 106 |
| 124 | chr10 | 111216360 | 111217221 | RNU6-53P | 8.2E−63 | 100 |
| 125 | chr7 | 4923117 | 4923615 | RADIL | 0.00000005 | 97 |
| 126 | chr3 | 75955728 | 75956395 | ZNF717 | 8.7E−50 | 97 |
| 127 | chr7 | 1270535 | 1270617 | UNCX | 8.6E−09 | 96 |
| 128 | chr7 | 148959791 | 148960046 | ZNF783 | 0.0019 | 95 |
| 129 | chr18 | 31739158 | 31739455 | NOL4 | 9.6E−46 | 95 |
| 130 | chr5 | 122621239 | 122621491 | CEP120 | 0.00045 | 94 |
| 131 | chr20 | 61340364 | 61340858 | NTSR1 | 1.9E−41 | 94 |
| 132 | chr16 | 86066512 | 86066907 | MIR6774 | 9.9E−10 | 93 |
| 133 | chr2 | 43327840 | 43328443 | LOC102723854 | 2.8E−44 | 90 |
| 134 | chr5 | 178367536 | 178368804 | ZNF454 | 6.3E−99 | 84 |
| 135 | chr1 | 237206266 | 237206721 | RYR2 | 3.4E−55 | 84 |
| 136 | chr7 | 57928469 | 57929147 | ZNF716 | 7.9E−23 | 84 |
| 137 | chr20 | 61808422 | 61809219 | MIR124-3 | 2.1E−51 | 80 |
| 138 | chr8 | 145730875 | 145730928 | GPT | 1 | 79 |
| 139 | chr2 | 31457371 | 31457421 | EHD3 | 1.8E−13 | 78 |
| 140 | chr2 | 113956418 | 113956703 | PSD4 | 4.8E−16 | 77 |
| 141 | chr7 | 57484714 | 57484819 | MIR3147 | 0.043 | 75 |
| 142 | chr1 | 10764635 | 10764790 | CASZ1 | 2.9E−09 | 74 |
| 143 | chr16 | 11769892 | 11770176 | SNN | 0.000000077 | 74 |
| 144 | chr16 | 4730135 | 4730615 | MIR6769A | 1.6E−28 | 72 |
| 145 | chr8 | 494817 | 494884 | TDRP | 0.0028 | 71 |
| 146 | chr17 | 8702665 | 8702908 | MFSD6L | 1.6E−17 | 70 |
| 147 | chr5 | 31798294 | 31799379 | PDZD2 | 0.0024 | 69 |
| 148 | chr20 | 55201884 | 55201971 | TFAP2C | 1.9E−16 | 69 |
| 149 | chr20 | 25228631 | 25228705 | PYGB | 1 | 66 |
| 150 | chr10 | 1231005 | 1231352 | LINC00200 | 0.000074 | 64 |
| 151 | chr10 | 22634217 | 22634256 | SPAG6 | 0.000000024 | 64 |
| 152 | chr18 | 67068677 | 67069273 | DOK6 | 2.7E−58 | 63 |
| 153 | chr3 | 184056350 | 184056670 | FAM131A | 1 | 62 |
| 154 | chr10 | 126211559 | 126211820 | LHPP | 0.000017 | 61 |
| 155 | chr11 | 2828289 | 2828960 | KCNQ1-AS1 | 1.5E−20 | 60 |
| 156 | chr14 | 102027120 | 102027174 | MIR1247 | 1 | 59 |
| 157 | chr16 | 32896382 | 32896472 | SLC6A10P | 0.013 | 59 |
| 158 | chr8 | 54164134 | 54164331 | OPRK1 | 1.5E−13 | 59 |
| 159 | chr8 | 144303024 | 144303454 | GPIHBP1 | 3.7E−09 | 58 |
| 160 | chr7 | 21209527 | 21209834 | SP4 | 0.0017 | 58 |
| 161 | chr16 | 88472496 | 88472807 | ZNF469 | 0.0000023 | 58 |
| 162 | chr14 | 101357519 | 101357769 | MEG8 | 0.0000067 | 54 |
| 163 | chr6 | 85482569 | 85483797 | TBX18 | 3E−99 | 54 |
| 164 | chr11 | 134281625 | 134281685 | B3GAT1 | 0.023 | 51 |
| 165 | chr12 | 127210739 | 127211525 | LINC00943 | 1E−82 | 50 |
| 166 | chr6 | 100915602 | 100915875 | SIM1 | 1.6E−12 | 49 |
| 167 | chr21 | 42218699 | 42218836 | DSCAM | 0.00000023 | 48 |
| 168 | chr2 | 177024366 | 177024783 | HOXD3 | 1E−24 | 47 |
| 169 | chr7 | 70597033 | 70598501 | WBSCR17 | 8.7E−118 | 47 |
| 170 | chr8 | 25899954 | 25900122 | EBF2 | 0.00000015 | 45 |
| 171 | chr8 | 142613052 | 142613443 | MROH5 | 2.9E−12 | 44 |
| 172 | chr17 | 46690862 | 46691020 | HOXB8 | 3E−19 | 41 |
| 173 | chr11 | 6676532 | 6676597 | DCHS1 | 0.00036 | 41 |
| 174 | chr18 | 43913947 | 43913991 | RNF165 | 0.51 | 40 |
| 175 | chr1 | 2949457 | 2949824 | ACTRT2 | 7.8E−30 | 39 |
| 176 | chr2 | 164593036 | 164593216 | FIGN | 1.4E−29 | 38 |
| 177 | chr9 | 138873467 | 138873633 | UBAC1 | 6.7E−14 | 37 |
| 178 | chr19 | 30016974 | 30017506 | VSTM2B | 6.8E−96 | 36 |
| 179 | chr5 | 140305727 | 140307192 | PCDHAC1 | 6.2E−93 | 35 |
| 180 | chr17 | 10632626 | 10632977 | TMEM220- | 1.9E−29 | 34 |
| | | | | AS1 | | |
| 181 | chr6 | 133562460 | 133563564 | EYA4 | 2E−91 | 33 |
| 182 | chr11 | 315696 | 316680 | IFITM1 | 2.7E−287 | 33 |
| 183 | chr4 | 8873450 | 8876356 | HMX1 | 2.2E−27 | 33 |
| 184 | chr18 | 908970 | 909154 | ADCYAP1 | 1.7E−18 | 33 |
| 185 | chr2 | 176936259 | 176936942 | EVX2 | 3E−38 | 32 |
| 186 | chr7 | 32467625 | 32467947 | LOC100130673 | 2.9E−29 | 32 |
| 187 | chr10 | 94451209 | 94452485 | HHEX | 1.7E−69 | 32 |
| 188 | chr9 | 127240369 | 127240588 | NR5A1 | 0.18 | 29 |
| 189 | chr1 | 162351633 | 162352162 | C1orf226 | 0.00000016 | 29 |
| 190 | chr13 | 112710922 | 112711829 | SOX1 | 1.2E−42 | 28 |
| 191 | chr6 | 27512776 | 27513570 | ZNF184 | 5.4E−27 | 28 |
| 192 | chr1 | 110613203 | 110613274 | ALX3 | 5.2E−11 | 25 |
| 193 | chr15 | 44068446 | 44068869 | SERF2 | 0.000000001 | 25 |
| 194 | chr20 | 591013 | 591080 | TCF15 | 2.9E−12 | 25 |
| 195 | chr19 | 853407 | 853494 | ELANE | 1 | 25 |
| 196 | chr4 | 8861489 | 8862361 | HMX1 | 3.5E−66 | 25 |
| 197 | chr10 | 133109760 | 133110646 | TCERG1L | 1.6E−97 | 23 |
| 198 | chr16 | 32213912 | 32214209 | HERC2P4 | 1.4E−10 | 23 |
| 199 | chr16 | 88228256 | 88228588 | LOC101928880 | 0.031 | 23 |
| 200 | chr19 | 30016437 | 30016499 | LOC284395 | 1.5E−15 | 22 |
| 201 | chr15 | 32639196 | 32639273 | GOLGA8K | 1 | 22 |
| 202 | chr15 | 45403319 | 45403540 | DUOX2 | 1 | 22 |
| 203 | chr10 | 23461316 | 23463617 | PTF1A | 5.9E−163 | 21 |
| 204 | chr18 | 44773291 | 44775576 | SKOR2 | 4E−205 | 20 |
| 205 | chr13 | 95364026 | 95364786 | SOX21 | 2.3E−94 | 20 |
| 206 | chr1 | 112058046 | 112058721 | ADORA3 | 9.9E−78 | 19 |
| 207 | chr2 | 239755709 | 239755815 | TWIST2 | 0.000000085 | 19 |
| 208 | chr16 | 29790750 | 29791036 | ZG16 | 0.000016 | 19 |
| 209 | chr1 | 41827046 | 41827105 | FOXO6 | 1 | 19 |
| 210 | chr15 | 90543308 | 90543610 | ZNF710 | 0.000000054 | 19 |
| 211 | chr19 | 1130738 | 1131134 | SBNO2 | 2.8E−20 | 18 |
| 212 | chr9 | 139090953 | 139091044 | LHX3 | 0.00034 | 18 |
| 213 | chr4 | 165304258 | 165305137 | MARCH1 | 2.6E−88 | 18 |
| 214 | chr1 | 36042841 | 36043373 | TFAP2E | 2.8E−63 | 18 |
| 215 | chr7 | 57927855 | 57928416 | ZNF716 | 2.4E−52 | 18 |
| 216 | chr2 | 225265848 | 225266580 | FAM124B | 5.2E−42 | 17 |
| 217 | chr1 | 85156186 | 85156257 | SSX2IP | 0.00013 | 17 |
| 218 | chr6 | 100895159 | 100895296 | SIM1 | 0.000002 | 16 |
| 219 | chr1 | 153606019 | 153606256 | CHTOP | 1 | 16 |
| 220 | chr2 | 38301293 | 38302642 | CYP1B1 | 4.1E−102 | 16 |
| 221 | chr8 | 8749553 | 8750156 | MFHAS1 | 4.1E−12 | 16 |
| 222 | chr16 | 88121641 | 88121781 | LOC400553 | 0.000024 | 16 |
| 223 | chr19 | 30716720 | 30716811 | ZNF536 | 1 | 14 |
| 224 | chr10 | 48998572 | 48998766 | BMS1P5 | 0.001 | 14 |
| 225 | chr16 | 23847106 | 23847165 | PRKCB | 1 | 13 |
| 226 | chr22 | 38221446 | 38221545 | GALR3 | 0.037 | 13 |
| 227 | chr11 | 67134493 | 67134714 | CLCF1 | 1 | 13 |
| 228 | chr9 | 71788589 | 71789667 | TJP2 | 5.9E−120 | 13 |
| 229 | chr19 | 47152127 | 47152511 | DACT3 | 7.5E−48 | 12 |
| 230 | chr11 | 132952630 | 132952681 | OPCML | 0.0011 | 11 |
| 231 | chr8 | 49340828 | 49341091 | LOC101929268 | 3.4E−13 | 11 |
| 232 | chr1 | 11709190 | 11709276 | FBXO44 | 0.0012 | 10 |
| 233 | chr11 | 2720329 | 2721961 | KCNQ1OT1 | 7E−55 | 10 |
| 234 | chr19 | 37288169 | 37288705 | ZNF790-AS1 | 3.6E−68 | 10 |
| 235 | chr7 | 100942951 | 100943665 | LOC101927746 | 9.3E−42 | 9 |
| 236 | chr5 | 92930812 | 92931061 | MIR548AO | 0.000000012 | 9 |
| 237 | chr14 | 19685811 | 19686018 | | 0.04 | 8 |
| 238 | chr3 | 239204 | 239820 | CHL1 | 2.1E−47 | 8 |
| 239 | chr14 | 26674163 | 26674271 | NOVA1 | 2E−20 | 8 |
| 240 | chr2 | 98962893 | 98964186 | CNGA3 | 5.7E−77 | 8 |
| 241 | chr1 | 119522232 | 119522972 | TBX15 | 1.4E−55 | 7 |
| 242 | chr11 | 128736684 | 128737467 | KCNJ1 | 0.00000019 | 7 |
| 243 | chr9 | 139886483 | 139886538 | C9orf142 | 0.00021 | 7 |
| 244 | chr1 | 3210043 | 3210544 | ARHGEF16 | 0.0012 | 7 |
| 245 | chr19 | 54041302 | 54041519 | ZNF331 | 1 | 7 |
| 246 | chr20 | 644763 | 644804 | SRXN1 | 2.8E−11 | 7 |
| 247 | chr11 | 66314443 | 66314513 | ACTN3 | 1 | 7 |
| 248 | chr7 | 19184073 | 19185177 | FERD3L | 9.7E−53 | 6 |
| 249 | chr1 | 2136776 | 2136872 | FAAP20 | 1 | 6 |
| 250 | chr2 | 241772121 | 241772193 | KIF1A | 1 | 6 |
| 251 | chr1 | 243646287 | 243646876 | MIR4677 | 1.9E−38 | 6 |
| 252 | chr14 | 97685059 | 97685145 | LOC101929241 | 0.0000091 | 6 |
| 253 | chr10 | 105004750 | 105005110 | RPEL1 | 1 | 5 |
| 254 | chr3 | 184301210 | 184301607 | EPHB3 | 2.9E−26 | 5 |
| 255 | chr10 | 26506508 | 26507588 | GAD2 | 2.9E−63 | 5 |
| 256 | chr13 | 29106670 | 29106740 | FLT1 | 2E−12 | 5 |
| 257 | chr11 | 3216749 | 3217098 | MRGPRG-AS1 | 1 | 5 |
| 258 | chr5 | 38845781 | 38845843 | OSMR-AS1 | 0.000000015 | 5 |
| 259 | chr16 | 55689441 | 55690964 | SLC6A2 | 2.7E−147 | 5 |
| 260 | chr15 | 89922050 | 89922792 | MIR9-3HG | 1.2E−57 | 5 |
| 261 | chr7 | 101961700 | 101962042 | MIR4285 | 1.8E−28 | 4 |
| 262 | chr12 | 119418924 | 119419143 | SRRM4 | 0.00000046 | 4 |
| 263 | chr8 | 143627374 | 143627680 | ARC | 0.27 | 4 |
| 264 | chr8 | 1908151 | 1908502 | KBTBD11- | 1 | 4 |
| | | | | OT1 | | |
| 265 | chr2 | 219264602 | 219264771 | CTDSP1 | 7.7E−29 | 4 |
| 266 | chr1 | 235105668 | 235105928 | LOC101927851 | 4.3E−14 | 4 |
| 267 | chr1 | 2990215 | 2990607 | PRDM16 | 3.3E−42 | 4 |
| 268 | chr19 | 36912350 | 36912880 | LOC644189 | 7E−91 | 4 |
| 269 | chr8 | 47529009 | 47529429 | LINC00293 | 1.2E−20 | 4 |
| 270 | chr20 | 55500366 | 55501077 | BMP7-AS1 | 6.7E−45 | 4 |
| 271 | chr4 | 7483396 | 7483618 | MIR4274 | 0.0000039 | 4 |
| 272 | chr5 | 140097949 | 140098308 | VTRNA1-2 | 0.00029 | 3 |
| 273 | chr17 | 15689705 | 15689796 | MEIS3P1 | 1 | 3 |
| 274 | chr22 | 19017607 | 19017715 | DGCR10 | 5.8E−10 | 3 |
| 275 | chr2 | 239756351 | 239757924 | TWIST2 | 7.5E−148 | 3 |
| 276 | chr7 | 27169868 | 27170717 | HOXA4 | 3.6E−16 | 3 |
| 277 | chr21 | 36041995 | 36042374 | CLIC6 | 0.00000025 | 3 |
| 278 | chr20 | 45141910 | 45142441 | ZNF334 | 2.1E−39 | 3 |
| 279 | chr7 | 45614115 | 45614160 | ADCY1 | 0.0065 | 3 |
| 280 | chr7 | 73245344 | 73246132 | CLDN4 | 7E−32 | 3 |
| 281 | chr5 | 76934957 | 76935276 | OTP | 4.4E−32 | 3 |
| 282 | chr13 | 113648424 | 113649107 | MCF2L | 3.7E−26 | 2 |
| 283 | chr10 | 134733572 | 134733893 | CFAP46 | 3E−19 | 2 |
| 284 | chr7 | 158065959 | 158066163 | MIR595 | 6.8E−10 | 2 |
| 285 | chr1 | 16847480 | 16847591 | FAM231B | 0.0016 | 2 |
| 286 | chr13 | 20715983 | 20716861 | GJA3 | 6.8E−43 | 2 |
| 287 | chr19 | 22966485 | 22967051 | ZNF99 | 7E−36 | 2 |
| 288 | chr14 | 23876799 | 23877073 | MYH6 | 0.0031 | 2 |
| 289 | chr6 | 392222 | 393517 | IRF4 | 4.6E−91 | 2 |
| 290 | chr19 | 39755028 | 39755922 | IFNL2 | 5.3E−101 | 2 |
| 291 | chr6 | 55443608 | 55443781 | HMGCLL1 | 9.5E−09 | 2 |
| 292 | chr7 | 57472190 | 57472660 | MIR3147 | 3.4E−09 | 2 |
| 293 | chr7 | 57484605 | 57484694 | MIR3147 | 1 | 2 |
| 294 | chr3 | 62358006 | 62358109 | FEZF2 | 6.9E−09 | 2 |
| 295 | chr3 | 9595144 | 9595526 | LHFPL4 | 6.4E−55 | 2 |
| 296 | chr10 | 101874947 | 101875059 | CPN1 | 0.00001 | 1 |
| 297 | chr13 | 111464738 | 111465427 | LINC00346 | 2E−30 | 1 |
| 298 | chr11 | 123065918 | 123066098 | CLMP | 4.9E−26 | 1 |
| 299 | chr12 | 130388691 | 130388945 | TMEM132D | 5E−45 | 1 |
| 300 | chr7 | 153750266 | 153750325 | DPP6 | 1 | 1 |
| 301 | chr5 | 155108451 | 155108512 | SGCD | 1.4E−10 | 1 |
| 302 | chr10 | 23481250 | 23481303 | PTF1A | 3.7E−15 | 1 |
| 303 | chr1 | 33359173 | 33359268 | HPCA | 0.17 | 1 |
| 304 | chr16 | 33778869 | 33779002 | LINC00273 | 1 | 1 |
| 305 | chr14 | 34269691 | 34269768 | EGLN3 | 0.094 | 1 |
| 306 | chr13 | 36052403 | 36053213 | NBEA | 1.3E−27 | 1 |
| 307 | chr15 | 42174719 | 42175003 | SPTBN5 | 4.3E−13 | 1 |
| 308 | chr2 | 43411339 | 43411705 | ZFP36L2 | 5.3E−10 | 1 |
| 309 | chr5 | 45696351 | 45696395 | HCN1 | 3.7E−13 | 1 |
| 310 | chr22 | 46262508 | 46262923 | MIR4762 | 1.8E−45 | 1 |
| 311 | chr20 | 53092293 | 53093101 | DOK5 | 5.8E−71 | 1 |
| 312 | chr13 | 53775090 | 53775595 | LINC01065 | 1.4E−135 | 1 |
| 313 | chr19 | 57182797 | 57183497 | ZNF835 | 1.8E−80 | 1 |
| 314 | chr7 | 57271141 | 57271431 | GUSBP10 | 4.3E−12 | 1 |
| 315 | chr13 | 58206330 | 58207289 | PCDH17 | 7.7E−97 | 1 |
| 316 | chr20 | 61885447 | 61885809 | NKAIN4 | 2.1E−55 | 1 |
| 317 | chr11 | 64480403 | 64480612 | NRXN2 | 6.2E−55 | 1 |
| 318 | chr16 | 67034939 | 67035037 | CES4A | 1 | 1 |
| 319 | chr9 | 70177424 | 70177562 | FOXD4L5 | 0.0027 | 1 |
| 320 | chr4 | 85417659 | 85418648 | NKX6-1 | 2.6E−50 | 1 |
| 321 | chr9 | 96716761 | 96717050 | BARX1 | 1.5E−09 | 1 |

### Esophageal Adenocarcinoma

In another aspect is provided a method of detecting a level of DNA methylation in a subject at risk of developing esophageal adenocarcinoma, the method including: determining the degree of methylation of CpG sites within a plurality of gene regions in a DNA sample from the subject, wherein the plurality of gene regions includes at least 5 different gene regions in Table EAC. In embodiments, an increased level of methylated CpG sites relative to a standard control indicates a higher risk of esophageal adenocarcinoma.

Provided herein is a method of treating esophageal adenocarcinoma in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table EAC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the esophageal adenocarcinoma is Stage I, Stage II, or Stage III. In embodiments, the esophageal adenocarcinoma is Stage I. In embodiments, the esophageal adenocarcinoma is Stage II. In embodiments, the esophageal adenocarcinoma is Stage III.

Provided herein is a method of diagnosing esophageal adenocarcinoma in a patient comprising: (a) detecting a level of methylated CpG sites within a plurality of gene regions in a DNA sample from the patient, wherein the plurality of gene regions comprise at least 5 different gene regions in Table EAC; and (b) diagnosing the patient with esophageal adenocarcinoma when the DNA sample has an increased level of methylated CpG sites, relative to a standard control, within the plurality of gene regions. In embodiments, the esophageal adenocarcinoma is Stage I, Stage II, or Stage III. In embodiments, the esophageal adenocarcinoma is Stage I. In embodiments, the esophageal adenocarcinoma is Stage II. In embodiments, the esophageal adenocarcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having esophageal adenocarcinoma or monitoring risk for developing esophageal adenocarcinoma in a patient comprising: (a) detecting a level of methylated CpG sites within a plurality of gene regions in a DNA sample from the patient at a first time point, wherein the plurality of gene regions comprise at least 5 different gene regions in Table EAC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing esophageal adenocarcinoma. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing esophageal adenocarcinoma or does not have esophageal adenocarcinoma. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing esophageal adenocarcinoma or may have esophageal adenocarcinoma. In embodiments, the esophageal adenocarcinoma is Stage I, Stage II, or Stage III. In embodiments, the esophageal adenocarcinoma is Stage I. In embodiments, the esophageal adenocarcinoma is Stage II. In embodiments, the esophageal adenocarcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table EAC. In embodiments, the plurality of gene regions includes at least 2 DMRs (i.e., gene regions) in Table EAC. In embodiments, the plurality of gene regions includes at least 3 DMRs in Table EAC. In embodiments, the plurality of gene regions includes at least 4 DMRs in Table EAC. In embodiments, the plurality of gene regions includes at least 5 DMRs in Table EAC. In embodiments, the plurality of gene regions includes at least 6 DMRs in Table EAC. In embodiments, the plurality of gene regions includes at least 7 DMRs in Table EAC. In embodiments, the plurality of gene regions includes at least 8 DMRs in Table EAC. In embodiments, the plurality of gene regions includes at least 9 DMRs in Table EAC. In embodiments, the plurality of gene regions includes at least 10 DMRs in Table EAC. In embodiments, the plurality of gene regions includes at least 15 DMRs in Table EAC. In embodiments, the plurality of gene regions includes at least 20 DMRs in Table EAC. In embodiments, the plurality of gene regions includes at least 25 DMRs in Table EAC. In embodiments, the plurality of gene regions includes at least 30 DMRs in Table EAC. In embodiments, the plurality of gene regions includes at least 35 DMRs in Table EAC. In embodiments, the plurality of gene regions includes at least 40 DMRs in Table EAC. In embodiments, the plurality of gene regions includes at least 45 DMRs in Table EAC. In embodiments, the plurality of gene regions includes at least 50 DMRs in Table EAC. In embodiments, the plurality of gene regions includes at least 55 DMRs in Table EAC. In embodiments, the plurality of gene regions includes at least 60 DMRs in Table EAC. In embodiments, the plurality of gene regions includes at least 65 DMRs in Table EAC. In embodiments, the plurality of gene regions includes at least 70 DMRs in Table EAC. In embodiments, the plurality of gene regions includes at least 75 DMRs in Table EAC. In embodiments, the plurality of gene regions includes at least 80 DMRs in Table EAC. In embodiments, the plurality of gene regions includes at least 85 DMRs in Table EAC. In embodiments, the plurality of gene regions includes at least 90 DMRs in Table EAC. In embodiments, the plurality of gene regions includes at least 95 DMRs in Table EAC. In embodiments, the plurality of gene regions includes at least 110 DMRs in Table EAC. In embodiments, the plurality of gene regions includes at least 120 DMRs in Table EAC. In embodiments, the plurality of gene regions includes at least 130 DMRs in Table EAC. In embodiments, the plurality of gene regions includes at least 140 DMRs in Table EAC. In embodiments, the plurality of gene regions includes at least 150 DMRs in Table EAC. In embodiments, the plurality of gene regions includes at least 160 DMRs in Table EAC. In embodiments, the plurality of gene regions includes at least 170 DMRs in Table EAC. In embodiments, the plurality of gene regions includes at least 180 DMRs in Table EAC. In embodiments, the plurality of gene regions includes at least 190 DMRs in Table EAC. In embodiments, the plurality of gene regions includes at least 200 DMRs in Table EAC. In embodiments, the plurality of gene regions includes at least 225 DMRs in Table EAC.

In embodiments, the plurality of gene regions includes the first DMR (i.e., gene region) in Table EAC. In embodiments, the plurality of gene regions includes the first 2 DMRs (i.e., gene regions) in Table EAC. In embodiments, the plurality of gene regions includes the first 3 DMRs in Table EAC. In embodiments, the plurality of gene regions includes the first 4 DMRs in Table EAC. In embodiments, the plurality of gene regions includes the first 5 DMRs in Table EAC. In embodiments, the plurality of gene regions includes the first 6 DMRs in Table EAC. In embodiments, the plurality of gene regions includes the first 7 DMRs in Table EAC. In embodiments, the plurality of gene regions includes the first 8 DMRs in Table EAC. In embodiments, the plurality of gene regions includes the first 9 DMRs in Table EAC. In embodiments, the plurality of gene regions includes the first 10 DMRs in Table EAC. In embodiments, the plurality of gene regions includes the first 11 DMRs in Table EAC. In embodiments, the plurality of gene regions includes the first 12 DMRs in Table EAC. In embodiments, the plurality of gene regions includes the first 13 DMRs in Table EAC. In embodiments, the plurality of gene regions includes the first 14 DMRs in Table EAC. In embodiments, the plurality of gene regions includes the first 15 DMRs in Table EAC. In embodiments, the plurality of gene regions includes the first 16 DMRs in Table EAC. In embodiments, the plurality of gene regions includes the first 17 DMRs in Table EAC. In embodiments, the plurality of gene regions includes the first 18 DMRs in Table EAC. In embodiments, the plurality of gene regions includes the first 19 DMRs in Table EAC. In embodiments, the plurality of gene regions includes the first 20 DMRs in Table EAC. In embodiments, the plurality of gene regions includes the first 25 DMRs in Table EAC. In embodiments, the plurality of gene regions includes the first 30 DMRs in Table EAC. In embodiments, the plurality of gene regions includes the first 35 DMRs in Table EAC. In embodiments, the plurality of gene regions includes the first 40 DMRs in Table EAC. In embodiments, the plurality of gene regions includes the first 45 DMRs in Table EAC. In embodiments, the plurality of gene regions includes the first 50 DMRs in Table EAC. In embodiments, the plurality of gene regions includes the first 55 DMRs in Table EAC. In embodiments, the plurality of gene regions includes the first 60 DMRs in Table EAC. In embodiments, the plurality of gene regions includes the first 65 DMRs in Table EAC. In embodiments, the plurality of gene regions includes the first 70 DMRs in Table EAC. In embodiments, the plurality of gene regions includes the first 75 DMRs in Table EAC. In embodiments, the plurality of gene regions includes the first 80 DMRs in Table EAC. In embodiments, the plurality of gene regions includes the first 85 DMRs in Table EAC. In embodiments, the plurality of gene regions includes the first 90 DMRs in Table EAC. In embodiments, the plurality of gene regions includes the first 95 DMRs in Table EAC. In embodiments, the plurality of gene regions includes the first 110 DMRs in Table EAC. In embodiments, the plurality of gene regions includes the first 120 DMRs in Table EAC. In embodiments, the plurality of gene regions includes the first 130 DMRs in Table EAC. In embodiments, the plurality of gene regions includes the first 140 DMRs in Table EAC. In embodiments, the plurality of gene regions includes the first 150 DMRs in Table EAC. In embodiments, the plurality of gene regions includes the first 160 DMRs in Table EAC. In embodiments, the plurality of gene regions includes the first 170 DMRs in Table EAC. In embodiments, the plurality of gene regions includes the first 180 DMRs in Table EAC. In embodiments, the plurality of gene regions includes the first 190 DMRs in Table EAC. In embodiments, the plurality of gene regions includes the first 200 DMRs in Table EAC. In embodiments, the plurality of gene regions includes the first 225 DMRs in Table EAC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is an esophagusgastroduodenoscopy (EGD), an endoscopic ultrasound, a bronchoscopy, or a tissue biopsy. In embodiments, the confirmatory diagnostic procedure is a tumor marker test, a microsatellite instability test, a computed tomography scan, a magnetic resonance imaging scan, or a positron emission tomography scan.

In embodiments, the method further includes treating the subject for esophageal adenocarcinoma. In embodiments, treating includes surgery, endoscopic therapy, or radiation therapy. In embodiments, the treating includes chemotherapy, targeted therapy, immunotherapy, or a combination of two or more thereof.

TABLE EAC

| | | | | | Adjusted | |
|---|---|---|---|---|---|---|
| No. | Chr | Start | End | Gene Name | p-value | Freq |
| 1 | chr15 | 101629342 | 101629581 | CHSY1 | 0.86 | 416 |
| 2 | chr4 | 5891985 | 5892089 | CRMP1 | 0.000000003 | 408 |
| 3 | chr17 | 41832634 | 41832704 | SOST | 0.53 | 406 |
| 4 | chr21 | 46896215 | 46896311 | MIR6815 | 1 | 406 |
| 5 | chr13 | 112575679 | 112576115 | LINC00354 | 1 | 402 |
| 6 | chr7 | 103629795 | 103629829 | RELN | 0.00000012 | 401 |
| 7 | chr14 | 101012754 | 101013110 | BEGAIN | 7.3E−30 | 396 |
| 8 | chr8 | 67344497 | 67345106 | ADHFE1 | 8.2E−37 | 393 |
| 9 | chr9 | 139880374 | 139880579 | LCNL1 | 0.0000017 | 390 |
| 10 | chr8 | 142394572 | 142394898 | GPR20 | 1 | 390 |
| 11 | chr5 | 140052105 | 140052263 | DND1 | 4.4E−12 | 389 |
| 12 | chr8 | 145730875 | 145730928 | GPT | 1 | 389 |
| 13 | chr10 | 135178458 | 135178918 | MIR3944 | 7.1E−15 | 384 |
| 14 | chr20 | 37356055 | 37357918 | SLC32A1 | 1.2E−132 | 384 |
| 15 | chr10 | 119001477 | 119001615 | SLC18A2 | 1.7E−10 | 381 |
| 16 | chr7 | 30721306 | 30721664 | CRHR2 | 5.5E−37 | 378 |
| 17 | chr22 | 50615177 | 50615336 | PANX2 | 1 | 317 |
| 18 | chr4 | 144620938 | 144622217 | FREM3 | 5E−103 | 315 |
| 19 | chr19 | 56850657 | 56851040 | ZNF542P | 0.00022 | 288 |
| 20 | chr18 | 7117178 | 7117271 | LAMA1 | 1 | 288 |
| 21 | chr8 | 145755611 | 145755904 | C8orf82 | 1 | 248 |
| 22 | chr5 | 136834876 | 136834971 | SPOCK1 | 1 | 240 |
| 23 | chr16 | 68481038 | 68481113 | SMPD3 | 1 | 237 |
| 24 | chr19 | 39055549 | 39055712 | MAP4K1 | 1 | 233 |
| 25 | chr18 | 31020519 | 31020681 | CCDC178 | 3E−42 | 198 |
| 26 | chr9 | 136075475 | 136075811 | OBP2B | 8.5E−09 | 166 |
| 27 | chr11 | 62370462 | 62370627 | MTA2 | 0.47 | 160 |
| 28 | chr9 | 140197441 | 140197553 | NRARP | 1 | 151 |
| 29 | chr22 | 40058678 | 40058999 | CACNA1I | 1.8E−13 | 150 |
| 30 | chr19 | 523537 | 523776 | CDC34 | 0.000035 | 130 |
| 31 | chr18 | 77623664 | 77624111 | KCNG2 | 7.1E−83 | 127 |
| 32 | chr11 | 60534921 | 60535001 | MS4A15 | 0.0017 | 120 |
| 33 | chr19 | 48565035 | 48565323 | CABP5 | 2.5E−27 | 117 |
| 34 | chr5 | 176046720 | 176047338 | MIR4281 | 1.2E−26 | 113 |
| 35 | chr7 | 57484605 | 57484694 | MIR3147 | 1 | 109 |
| 36 | chr6 | 1410166 | 1410436 | MIR6720 | 0.062 | 105 |
| 37 | chr5 | 178986371 | 178987104 | RUFY1 | 1.4E−26 | 105 |
| 38 | chr1 | 77333983 | 77334644 | ST6GALNAC5 | 8.4E−55 | 104 |
| 39 | chr4 | 81187466 | 81187605 | FGF5 | 4.9E−11 | 102 |
| 40 | chr2 | 118593849 | 118594327 | DDX18 | 3.8E−53 | 100 |
| 41 | chr20 | 57042108 | 57042626 | APCDD1L | 5.1E−32 | 99 |
| 42 | chr19 | 11594326 | 11594434 | ELAVL3 | 0.5 | 98 |
| 43 | chr9 | 97094464 | 97095197 | LOC100132077 | 2.1E−38 | 97 |
| 44 | chr7 | 47367672 | 47367832 | TNS3 | 0.00069 | 95 |
| 45 | chr20 | 61002544 | 61003481 | RBBP8NL | 1.2E−10 | 94 |
| 46 | chr10 | 22766062 | 22766978 | LOC100499489 | 0.0000046 | 89 |
| 47 | chr16 | 1171931 | 1172126 | C1QTNF8 | 1 | 87 |
| 48 | chr20 | 47444742 | 47444802 | PREX1 | 1 | 87 |
| 49 | chr14 | 103568641 | 103568719 | EXOC3L4 | 0.000000011 | 86 |
| 50 | chr7 | 103630754 | 103631004 | RELN | 1 | 84 |
| 51 | chr2 | 131674028 | 131674081 | ARHGEF4 | 0.000000084 | 84 |
| 52 | chr8 | 144303024 | 144303454 | GPIHBP1 | 3.7E−09 | 84 |
| 53 | chr12 | 6166094 | 6166299 | VWF | 0.0000002 | 80 |
| 54 | chr15 | 93126770 | 93127164 | LINC00930 | 0.94 | 78 |
| 55 | chr16 | 90143643 | 90143904 | PRDM7 | 2E−12 | 77 |
| 56 | chr19 | 58446168 | 58446960 | ZNF418 | 3.6E−59 | 76 |
| 57 | chr1 | 16085208 | 16085925 | FBLIM1 | 8.8E−74 | 73 |
| 58 | chr7 | 169696 | 169964 | LOC100507642 | 0.008 | 73 |
| 59 | chr2 | 240168706 | 240169346 | MGC16025 | 1.9E−36 | 73 |
| 60 | chr7 | 154862202 | 154862808 | HTR5A | 0.011 | 70 |
| 61 | chr20 | 61471688 | 61471855 | DPH3P1 | 0.033 | 68 |
| 62 | chr11 | 61723273 | 61723429 | BEST1 | 0.00058 | 58 |
| 63 | chr8 | 688402 | 688769 | ERICH1-AS1 | 1.1E−12 | 58 |
| 64 | chr2 | 242927372 | 242927735 | LOC102723927 | 4.4E−11 | 54 |
| 65 | chr3 | 141174173 | 141174534 | RASA2 | 0.0000071 | 53 |
| 66 | chr7 | 37487865 | 37487940 | ELMO1 | 0.0046 | 53 |
| 67 | chr2 | 27070467 | 27070520 | DPYSL5 | 2.9E−11 | 47 |
| 68 | chr17 | 32907996 | 32909030 | TMEM132E | 3.5E−102 | 46 |
| 69 | chr16 | 2391584 | 2391702 | ABCA17P | 1 | 45 |
| 70 | chr19 | 12666316 | 12666454 | ZNF564 | 0.0099 | 44 |
| 71 | chr2 | 5831369 | 5831476 | LINC01248 | 1 | 42 |
| 72 | chr4 | 6107165 | 6107499 | JAKMIP1 | 0.00001 | 42 |
| 73 | chr15 | 66914678 | 66914731 | LINC01169 | 0.000024 | 42 |
| 74 | chr17 | 41833109 | 41833194 | SOST | 1 | 40 |
| 75 | chr6 | 50675387 | 50675836 | TFAP2D | 1.2E−14 | 39 |
| 76 | chr5 | 1295445 | 1295496 | TERT | 2.2E−10 | 38 |
| 77 | chr11 | 1316235 | 1316522 | TOLLIP | 0.0000063 | 38 |
| 78 | chr22 | 43807585 | 43807646 | MPPED1 | 1 | 38 |
| 79 | chr7 | 44144078 | 44144249 | AEBP1 | 1.9E−18 | 38 |
| 80 | chr10 | 105238811 | 105239351 | CALHM3 | 0.00021 | 37 |
| 81 | chr1 | 150522017 | 150522055 | ADAMTSL4 | 1 | 35 |
| 82 | chr1 | 3142901 | 3143056 | MIR4251 | 1 | 35 |
| 83 | chr20 | 33413432 | 33413529 | NCOA6 | 2.2E−12 | 35 |
| 84 | chr19 | 41354451 | 41354666 | CYP2A6 | 5.3E−16 | 34 |
| 85 | chr3 | 32509104 | 32509331 | CMTM6 | 0.47 | 33 |
| 86 | chr17 | 40835731 | 40835939 | CNTNAP1 | 1 | 33 |
| 87 | chr19 | 2888831 | 2889329 | ZNF57 | 9E−12 | 31 |
| 88 | chr5 | 170743598 | 170743662 | TLX3 | 1.8E−17 | 30 |
| 89 | chr16 | 3233911 | 3234030 | OR1F1 | 0.00019 | 29 |
| 90 | chr10 | 135436353 | 135436577 | FRG2B | 0.00072 | 28 |
| 91 | chr10 | 50450715 | 50451086 | C10orf128 | 0.0038 | 28 |
| 92 | chr7 | 120629403 | 120629804 | CPED1 | 0.0018 | 26 |
| 93 | chr7 | 44185732 | 44185983 | MYL7 | 1 | 26 |
| 94 | chr8 | 145751955 | 145752311 | LRRC24 | 0.00000036 | 25 |
| 95 | chr9 | 139886483 | 139886538 | C9orf142 | 0.00021 | 24 |
| 96 | chr17 | 3675018 | 3675296 | ITGAE | 1 | 24 |
| 97 | chr7 | 63361448 | 63361592 | LINC01005 | 1 | 23 |
| 98 | chr13 | 93879543 | 93880836 | GPC6 | 4.1E−137 | 23 |
| 99 | chr3 | 48693932 | 48694016 | CELSR3 | 1 | 21 |
| 100 | chr13 | 111465578 | 111465658 | LINC00346 | 0.057 | 20 |
| 101 | chr5 | 176236974 | 176237045 | UNC5A | 0.000000017 | 20 |
| 102 | chr15 | 29968103 | 29968403 | FAM189A1 | 1 | 20 |
| 103 | chr12 | 3579682 | 3580189 | PRMT8 | 0.19 | 20 |
| 104 | chr4 | 5892243 | 5892335 | CRMP1 | 5.8E−12 | 20 |
| 105 | chr17 | 76121013 | 76121226 | TMC6 | 1 | 20 |
| 106 | chr11 | 76751410 | 76751481 | B3GNT6 | 0.86 | 20 |
| 107 | chr3 | 33260365 | 33260782 | SUSD5 | 5.4E−60 | 19 |
| 108 | chr6 | 27390883 | 27391015 | ZNF391 | 1 | 18 |
| 109 | chr19 | 3585505 | 3585581 | GIPC3 | 1 | 18 |
| 110 | chr1 | 43814168 | 43815386 | CDC20 | 1.6E−36 | 18 |
| 111 | chr22 | 37464961 | 37465138 | KCTD17 | 6.4E−24 | 17 |
| 112 | chr1 | 38461444 | 38462096 | SF3A3 | 1E−33 | 17 |
| 113 | chr19 | 45975734 | 45976395 | FOSB | 2.7E−25 | 16 |
| 114 | chr19 | 6669923 | 6670065 | TNFSF14 | 1 | 16 |
| 115 | chr8 | 145638419 | 145638946 | SLC39A4 | 6.8E−58 | 15 |
| 116 | chr6 | 41376434 | 41377088 | NCR2 | 4.7E−20 | 15 |
| 117 | chr16 | 88472496 | 88472807 | ZNF469 | 0.0000023 | 15 |
| 118 | chr7 | 101961700 | 101962042 | MIR4285 | 1.8E−28 | 14 |
| 119 | chr12 | 562720 | 562926 | B4GALNT3 | 0.15 | 14 |
| 120 | chr8 | 56851845 | 56852430 | LYN | 4.3E−13 | 14 |
| 121 | chr9 | 140033270 | 140033516 | GRIN1 | 3E−15 | 13 |
| 122 | chr12 | 4829052 | 4829303 | LOC101929549 | 0.000016 | 13 |
| 123 | chr19 | 50861655 | 50861825 | NAPSA | 0.000021 | 13 |
| 124 | chr13 | 61987789 | 61987985 | PCDH20 | 1.1E−10 | 12 |
| 125 | chr5 | 63986397 | 63986941 | FAM159B | 5E−52 | 12 |
| 126 | chr16 | 88263173 | 88263261 | LOC101928880 | 1 | 12 |
| 127 | chr2 | 9898066 | 9898391 | TAF1B | 0.000000038 | 12 |
| 128 | chr8 | 143532445 | 143532542 | ADGRB1 | 0.00074 | 10 |
| 129 | chr22 | 42095367 | 42095635 | MEI1 | 2.5E−10 | 10 |
| 130 | chr3 | 23244221 | 23244408 | UBE2E2 | 1 | 9 |
| 131 | chr17 | 48048823 | 48049045 | DLX4 | 5E−11 | 9 |
| 132 | chr5 | 7396238 | 7396286 | ADCY2 | 0.73 | 9 |
| 133 | chr13 | 112984830 | 112985846 | LINC01044 | 1 | 8 |
| 134 | chr17 | 12877226 | 12877772 | ELAC2 | 1.5E−36 | 8 |
| 135 | chr6 | 168502683 | 168502841 | FRMD1 | 1 | 8 |
| 136 | chr1 | 201618256 | 201618335 | NAV1 | 1 | 8 |
| 137 | chr1 | 209405127 | 209405216 | MIR205HG | 1.2E−16 | 8 |
| 138 | chr5 | 2751128 | 2757191 | C5orf38 | 1.3E−152 | 8 |
| 139 | chr16 | 48844551 | 48845153 | N4BP1 | 7.4E−82 | 8 |
| 140 | chr19 | 50861977 | 50862102 | NAPSA | 0.0052 | 8 |
| 141 | chr1 | 75591189 | 75591408 | LHX8 | 1 | 8 |
| 142 | chr14 | 105208235 | 105208483 | SIVA1 | 9.8E−19 | 7 |
| 143 | chr7 | 4848496 | 4849344 | MIR4656 | 0.069 | 7 |
| 144 | chr20 | 57429573 | 57429745 | GNAS | 0.0032 | 7 |
| 145 | chr20 | 61885447 | 61885809 | NKAIN4 | 2.1E−55 | 7 |
| 146 | chr17 | 79109579 | 79109960 | MIR1250 | 8.3E−09 | 7 |
| 147 | chr15 | 93122815 | 93123359 | LINC00930 | 0.0000058 | 7 |
| 148 | chr15 | 31621569 | 31621727 | KLF13 | 1 | 6 |
| 149 | chr11 | 66314443 | 66314513 | ACTN3 | 1 | 6 |
| 150 | chr6 | 158080653 | 158080862 | MIR3692 | 2.3E−09 | 5 |
| 151 | chr7 | 1643473 | 1643975 | TFAMP1 | 3.6E−21 | 5 |
| 152 | chr4 | 172734201 | 172734237 | GALNTL6 | 1.8E−18 | 5 |
| 153 | chr2 | 176981980 | 176982522 | HOXD10 | 7.6E−15 | 5 |
| 154 | chr2 | 240162169 | 240162413 | MGC16025 | 3.6E−10 | 5 |
| 155 | chr9 | 34809685 | 34810113 | FAM205BP | 2E−61 | 5 |
| 156 | chr18 | 74962322 | 74963218 | GALR1 | 2.8E−77 | 5 |
| 157 | chr17 | 751345 | 751538 | NXN | 1 | 5 |
| 158 | chr10 | 104170786 | 104170970 | PSD | 1 | 4 |
| 159 | chr14 | 104896487 | 104896658 | C14orf180 | 0.00022 | 4 |
| 160 | chr10 | 105421059 | 105421194 | SH3PXD2A-AS1 | 1 | 4 |
| 161 | chr1 | 1244840 | 1245076 | PUSL1 | 8.8E−09 | 4 |
| 162 | chr5 | 166405925 | 166405999 | CTB-7E3.1 | 1 | 4 |
| 163 | chr2 | 239072872 | 239072949 | FAM132B | 1 | 4 |
| 164 | chr15 | 45403319 | 45403540 | DUOX2 | 1 | 4 |
| 165 | chr8 | 637561 | 638327 | ERICH1 | 0.018 | 4 |
| 166 | chr5 | 168728192 | 168728274 | SLIT3 | 4E−12 | 3 |
| 167 | chr22 | 19706410 | 19706471 | SEPT5-GP1BB | 1 | 3 |
| 168 | chr10 | 23983436 | 23983738 | KIAA1217 | 1.6E−41 | 3 |
| 169 | chr20 | 34894346 | 34894839 | DLGAP4 | 2E−47 | 3 |
| 170 | chr17 | 35299347 | 35300455 | LHX1 | 9.4E−93 | 3 |
| 171 | chr19 | 40421192 | 40421672 | FCGBP | 1E−27 | 3 |
| 172 | chr6 | 43253057 | 43253134 | SLC22A7 | 1 | 3 |
| 173 | chr21 | 44088962 | 44089089 | PDE9A | 1 | 3 |
| 174 | chr21 | 45770244 | 45770310 | TRPM2 | 9.1E−10 | 3 |
| 175 | chr19 | 46526268 | 46526750 | PGLYRP1 | 6.5E−15 | 3 |
| 176 | chr16 | 72821595 | 72821759 | LINC01572 | 8.6E−16 | 3 |
| 177 | chr7 | 75779489 | 75780140 | SRRM3 | 6.2E−34 | 3 |
| 178 | chr10 | 101281274 | 101281389 | LINC01475 | 0.0003 | 2 |
| 179 | chr16 | 10274586 | 10274739 | GRIN2A | 0.00013 | 2 |
| 180 | chr14 | 105945496 | 105945690 | CRIP2 | 0.0023 | 2 |
| 181 | chr2 | 142887740 | 142887827 | LRP1B | 7.2E−12 | 2 |
| 182 | chr5 | 1444984 | 1445065 | SLC6A3 | 3.7E−09 | 2 |
| 183 | chr5 | 176106939 | 176107301 | TSPAN17 | 2.3E−11 | 2 |
| 184 | chr16 | 1922298 | 1922519 | MEIOB | 1.5E−14 | 2 |
| 185 | chr14 | 23835663 | 23835969 | EFS | 6.1E−13 | 2 |
| 186 | chr12 | 29937010 | 29937069 | TMTC1 | 0.00046 | 2 |
| 187 | chr2 | 31457371 | 31457421 | EHD3 | 1.8E−13 | 2 |
| 188 | chr1 | 6188088 | 6188181 | CHD5 | 1 | 2 |
| 189 | chr6 | 62995658 | 62996299 | KHDRBS2 | 2.2E−68 | 2 |
| 190 | chr9 | 68411147 | 68411576 | MIR4477B | 1 | 2 |
| 191 | chr14 | 103415848 | 103416095 | AMN | 0.00078 | 1 |
| 192 | chr14 | 104808449 | 104808652 | KIF26A | 1 | 1 |
| 193 | chr2 | 105478777 | 105478833 | POU3F3 | 0.045 | 1 |
| 194 | chr13 | 108518347 | 108518419 | FAM155A | 0.000018 | 1 |
| 195 | chr6 | 110300487 | 110301094 | GPR6 | 2.5E−45 | 1 |
| 196 | chr13 | 110958726 | 110960077 | COL4A1 | 1.3E−159 | 1 |
| 197 | chr13 | 112978530 | 112978862 | LINC01044 | 1.1E−26 | 1 |
| 198 | chr6 | 114181256 | 114181622 | MARCKS | 8.4E−18 | 1 |
| 199 | chr13 | 114964065 | 114964710 | CDC16 | 1.1E−27 | 1 |
| 200 | chr2 | 121549563 | 121549996 | GLI2 | 0.000000019 | 1 |
| 201 | chr8 | 1449737 | 1450097 | DLGAP2 | 1 | 1 |
| 202 | chr21 | 14982744 | 14982941 | POTED | 0.6 | 1 |
| 203 | chr8 | 1590944 | 1591041 | DLGAP2-AS1 | 1 | 1 |
| 204 | chr3 | 196757250 | 196757477 | MFI2 | 0.00014 | 1 |
| 205 | chr20 | 20345634 | 20346166 | INSM1 | 5.8E−27 | 1 |
| 206 | chr2 | 21266867 | 21266956 | APOB | 1 | 1 |
| 207 | chr12 | 21926288 | 21926712 | KCNJ8 | 1.3E−12 | 1 |
| 208 | chr14 | 23623479 | 23623706 | SLC7A8 | 1 | 1 |
| 209 | chr20 | 25228631 | 25228705 | PYGB | 1 | 1 |
| 210 | chr2 | 25427063 | 25427168 | POMC | 1 | 1 |
| 211 | chr11 | 2720261 | 2721440 | KCNQ1OT1 | 6.9E−30 | 1 |
| 212 | chr15 | 29980366 | 29980695 | FAM189A1 | 0.19 | 1 |
| 213 | chr19 | 35940424 | 35940980 | FFAR2 | 4.9E−15 | 1 |
| 214 | chr1 | 36042670 | 36042776 | TFAP2E | 9.2E−17 | 1 |
| 215 | chr14 | 38061009 | 38061076 | FOXA1 | 1.7E−09 | 1 |
| 216 | chr6 | 392222 | 393517 | IRF4 | 4.6E−91 | 1 |
| 217 | chr20 | 44880167 | 44880263 | CDH22 | 0.98 | 1 |
| 218 | chr7 | 4764575 | 4765382 | FOXK1 | 1.6E−23 | 1 |
| 219 | chr20 | 48099139 | 48099205 | KCNB1 | 0.000000012 | 1 |
| 220 | chr17 | 5000871 | 5001123 | USP6 | 6.4E−60 | 1 |
| 221 | chr19 | 50554366 | 50554491 | FLJ26850 | 9.8E−15 | 1 |
| 222 | chr17 | 59534376 | 59535253 | TBX4 | 3.4E−43 | 1 |
| 223 | chr11 | 62314325 | 62314664 | AHNAK | 0.000027 | 1 |
| 224 | chr7 | 62574764 | 62574921 | ZNF733P | 1.7E−10 | 1 |
| 225 | chr7 | 63386466 | 63387053 | LINC01005 | 1.8E−14 | 1 |
| 226 | chr11 | 64738922 | 64739097 | C11orf85 | 1 | 1 |
| 227 | chr15 | 65066958 | 65067072 | RBPMS2 | 1 | 1 |
| 228 | chr7 | 6543321 | 6543508 | KDELR2 | 1.1E−17 | 1 |
| 229 | chr7 | 6703583 | 6703647 | ZNF316 | 1.4E−09 | 1 |
| 230 | chr4 | 8872996 | 8873051 | HMX1 | 1 | 1 |
| 231 | chr11 | 92702812 | 92703083 | MTNR1B | 2E−40 | 1 |

### Pancreatic Ductal Adenocarcinoma

In another aspect is provided a method of detecting a level of DNA methylation in a subject at risk of developing pancreatic ductal adenocarcinoma (PDAC), the method including: determining the degree of methylation of CpG sites within a plurality of gene regions in a DNA sample from the subject, wherein the plurality of gene regions includes at least 5 different gene regions in Table PDAC. In embodiments, an increased level of methylated CpG sites relative to a standard control indicates a higher risk of PDAC.

Provided herein is a method of treating pancreatic ductal adenocarcinoma in a patient in need thereof comprising: (a) detecting an increased level of methylated CpG sites, relative to a standard control, within a plurality of gene regions in a DNA sample obtained from the patient, wherein the plurality of gene regions comprise at least 5 different gene regions in Table PDAC; and (b) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the pancreatic ductal adenocarcinoma is Stage I, Stage II, or Stage III. In embodiments, the pancreatic ductal adenocarcinoma is Stage I. In embodiments, the pancreatic ductal adenocarcinoma is Stage II. In embodiments, the pancreatic ductal adenocarcinoma is Stage III.

Provided herein is a method of diagnosing pancreatic ductal adenocarcinoma in a patient comprising: (a) detecting a level of methylated CpG sites within a plurality of gene regions in a DNA sample from the patient, wherein the plurality of gene regions comprise at least 5 different gene regions in Table PDAC; and (b) diagnosing the patient with pancreatic ductal adenocarcinoma when the DNA sample has an increased level of methylated CpG sites, relative to a standard control, within the plurality of gene regions. In embodiments, the pancreatic ductal adenocarcinoma is Stage I, Stage II, or Stage III. In embodiments, the pancreatic ductal adenocarcinoma is Stage I. In embodiments, the pancreatic ductal adenocarcinoma is Stage II. In embodiments, the pancreatic ductal adenocarcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having pancreatic ductal adenocarcinoma or monitoring risk for developing pancreatic ductal adenocarcinoma in a patient comprising: (a) detecting a level of methylated CpG sites within a plurality of gene regions in a DNA sample from the patient at a first time point, wherein the plurality of gene regions comprise at least 5 different gene regions in Table PDAC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing pancreatic ductal adenocarcinoma. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing pancreatic ductal adenocarcinoma or does not have pancreatic ductal adenocarcinoma. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing pancreatic ductal adenocarcinoma or may have pancreatic ductal adenocarcinoma. In embodiments, the pancreatic ductal adenocarcinoma is Stage I, Stage II, or Stage III. In embodiments, the pancreatic ductal adenocarcinoma is Stage I. In embodiments, the pancreatic ductal adenocarcinoma is Stage II. In embodiments, the pancreatic ductal adenocarcinoma is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the plurality of gene regions includes at least 1 DMR (i.e., gene region) in Table PDAC. In embodiments, the plurality of gene regions includes at least 2 DMRs (i.e., gene regions) in Table PDAC. In embodiments, the plurality of gene regions includes at least 3 DMRs in Table PDAC. In embodiments, the plurality of gene regions includes at least 4 DMRs in Table PDAC. In embodiments, the plurality of gene regions includes at least 5 DMRs in Table PDAC. In embodiments, the plurality of gene regions includes at least 6 DMRs in Table PDAC. In embodiments, the plurality of gene regions includes at least 7 DMRs in Table PDAC. In embodiments, the plurality of gene regions includes at least 8 DMRs in Table PDAC. In embodiments, the plurality of gene regions includes at least 9 DMRs in Table PDAC. In embodiments, the plurality of gene regions includes at least 10 DMRs in Table PDAC. In embodiments, the plurality of gene regions includes at least 15 DMRs in Table PDAC. In embodiments, the plurality of gene regions includes at least 20 DMRs in Table PDAC. In embodiments, the plurality of gene regions includes at least 25 DMRs in Table PDAC. In embodiments, the plurality of gene regions includes at least 30 DMRs in Table PDAC. In embodiments, the plurality of gene regions includes at least 35 DMRs in Table PDAC. In embodiments, the plurality of gene regions includes at least 40 DMRs in Table PDAC. In embodiments, the plurality of gene regions includes at least 45 DMRs in Table PDAC. In embodiments, the plurality of gene regions includes at least 50 DMRs in Table PDAC. In embodiments, the plurality of gene regions includes at least 55 DMRs in Table PDAC. In embodiments, the plurality of gene regions includes at least 60 DMRs in Table PDAC. In embodiments, the plurality of gene regions includes at least 65 DMRs in Table PDAC. In embodiments, the plurality of gene regions includes at least 70 DMRs in Table PDAC. In embodiments, the plurality of gene regions includes at least 75 DMRs in Table PDAC. In embodiments, the plurality of gene regions includes at least 80 DMRs in Table PDAC. In embodiments, the plurality of gene regions includes at least 85 DMRs in Table PDAC. In embodiments, the plurality of gene regions includes at least 90 DMRs in Table PDAC. In embodiments, the plurality of gene regions includes at least 95 DMRs in Table PDAC. In embodiments, the plurality of gene regions includes at least 110 DMRs in Table PDAC. In embodiments, the plurality of gene regions includes at least 120 DMRs in Table PDAC. In embodiments, the plurality of gene regions includes at least 130 DMRs in Table PDAC. In embodiments, the plurality of gene regions includes at least 140 DMRs in Table PDAC. In embodiments, the plurality of gene regions includes at least 150 DMRs in Table PDAC. In embodiments, the plurality of gene regions includes at least 160 DMRs in Table PDAC. In embodiments, the plurality of gene regions includes at least 170 DMRs in Table PDAC. In embodiments, the plurality of gene regions includes at least 180 DMRs in Table PDAC. In embodiments, the plurality of gene regions includes at least 190 DMRs in Table PDAC. In embodiments, the plurality of gene regions includes at least 200 DMRs in Table PDAC. In embodiments, the plurality of gene regions includes at least 225 DMRs in Table PDAC. In embodiments, the plurality of gene regions includes at least 250 DMRs in Table PDAC.

In embodiments, the plurality of gene regions includes the first DMR (i.e., gene region) in Table PDAC. In embodiments, the plurality of gene regions includes the first 2 DMRs (i.e., gene regions) in Table PDAC. In embodiments, the plurality of gene regions includes the first 3 DMRs in Table PDAC. In embodiments, the plurality of gene regions includes the first 4 DMRs in Table PDAC. In embodiments, the plurality of gene regions includes the first 5 DMRs in Table PDAC. In embodiments, the plurality of gene regions includes the first 6 DMRs in Table PDAC. In embodiments, the plurality of gene regions includes the first 7 DMRs in Table PDAC. In embodiments, the plurality of gene regions includes the first 8 DMRs in Table PDAC. In embodiments, the plurality of gene regions includes the first 9 DMRs in Table PDAC. In embodiments, the plurality of gene regions includes the first 10 DMRs in Table PDAC. In embodiments, the plurality of gene regions includes the first 11 DMRs in Table PDAC. In embodiments, the plurality of gene regions includes the first 12 DMRs in Table PDAC. In embodiments, the plurality of gene regions includes the first 13 DMRs in Table PDAC. In embodiments, the plurality of gene regions includes the first 14 DMRs in Table PDAC. In embodiments, the plurality of gene regions includes the first 15 DMRs in Table PDAC. In embodiments, the plurality of gene regions includes the first 16 DMRs in Table PDAC. In embodiments, the plurality of gene regions includes the first 17 DMRs in Table PDAC. In embodiments, the plurality of gene regions includes the first 18 DMRs in Table PDAC. In embodiments, the plurality of gene regions includes the first 19 DMRs in Table PDAC. In embodiments, the plurality of gene regions includes the first 20 DMRs in Table PDAC. In embodiments, the plurality of gene regions includes the first 25 DMRs in Table PDAC. In embodiments, the plurality of gene regions includes the first 30 DMRs in Table PDAC. In embodiments, the plurality of gene regions includes the first 35 DMRs in Table PDAC. In embodiments, the plurality of gene regions includes the first 40 DMRs in Table PDAC. In embodiments, the plurality of gene regions includes the first 45 DMRs in Table PDAC. In embodiments, the plurality of gene regions includes the first 50 DMRs in Table PDAC. In embodiments, the plurality of gene regions includes the first 55 DMRs in Table PDAC. In embodiments, the plurality of gene regions includes the first 60 DMRs in Table PDAC. In embodiments, the plurality of gene regions includes the first 65 DMRs in Table PDAC. In embodiments, the plurality of gene regions includes the first 70 DMRs in Table PDAC. In embodiments, the plurality of gene regions includes the first 75 DMRs in Table PDAC. In embodiments, the plurality of gene regions includes the first 80 DMRs in Table PDAC. In embodiments, the plurality of gene regions includes the first 85 DMRs in Table PDAC. In embodiments, the plurality of gene regions includes the first 90 DMRs in Table PDAC. In embodiments, the plurality of gene regions includes the first 95 DMRs in Table PDAC. In embodiments, the plurality of gene regions includes the first 110 DMRs in Table PDAC. In embodiments, the plurality of gene regions includes the first 120 DMRs in Table PDAC. In embodiments, the plurality of gene regions includes the first 130 DMRs in Table PDAC. In embodiments, the plurality of gene regions includes the first 140 DMRs in Table PDAC. In embodiments, the plurality of gene regions includes the first 150 DMRs in Table PDAC. In embodiments, the plurality of gene regions includes the first 160 DMRs in Table PDAC. In embodiments, the plurality of gene regions includes the first 170 DMRs in Table PDAC. In embodiments, the plurality of gene regions includes the first 180 DMRs in Table PDAC. In embodiments, the plurality of gene regions includes the first 190 DMRs in Table PDAC. In embodiments, the plurality of gene regions includes the first 200 DMRs in Table PDAC. In embodiments, the plurality of gene regions includes the first 225 DMRs in Table PDAC. In embodiments, the plurality of gene regions includes the first 250 DMRs in Table PDAC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. In embodiments, the confirmatory diagnostic procedure is an abdominal ultrasound, an endoscopic ultrasound, a fine needle aspiration, or a tissue biopsy. In embodiments, the confirmatory diagnostic procedure is a magnetic resonance imaging scan (MRI scan) (cholangiopancreatography), a computed tomography scan (CT scan), a positron emission tomography scan (PET scan), a Carcinoembryonic Antigen (CEA) test, or a CA19-9 antigen test. In embodiments, the confirmatory diagnostic procedure is a magnetic resonance cholangiopancreatography scan, a computed tomography scan, a positron emission tomography scan, a carcinoembryonic antigen test, or a CA19-9 antigen test.

In embodiments, the method further includes treating the subject for pancreatic ductal adenocarcinoma. In embodiments, treating includes surgery. In embodiments, treating includes radiotherapy, chemotherapy, targeted therapy, or immunotherapy. In embodiments, treating includes radiotherapy, chemotherapy, targeted therapy, immunotherapy, or a combination of two or more thereof.

TABLE PDAC

| Gene | | | | | | |
|---|---|---|---|---|---|---|
| Region | | | | | Adjusted | Freq |
| No. | Chr | Start | End | Gene Name | p-value | (Importance) |
| 1 | chr20 | 50721104 | 50721611 | ZFP64 | 4.4E−36 | 413 |
| 2 | chr12 | 125139647 | 125140269 | NCOR2 | 0.0017 | 410 |
| 3 | chr3 | 147127952 | 147128456 | ZIC1 | 4.5E−69 | 406 |
| 4 | chr16 | 32896752 | 32896840 | SLC6A10P | 1 | 405 |
| 5 | chr10 | 126211559 | 126211820 | LHPP | 0.000017 | 404 |
| 6 | chr10 | 133109760 | 133110646 | TCERG1L | 1.6E−97 | 402 |
| 7 | chr3 | 13974336 | 13974661 | FGD5P1 | 1.1E−46 | 399 |
| 8 | chr15 | 96866536 | 96866786 | NR2F2 | 1.3E−15 | 396 |
| 9 | chr5 | 93905177 | 93905542 | KIAA0825 | 1.1E−73 | 394 |
| 10 | chr12 | 125139527 | 125140249 | NCOR2 | 0.0038 | 392 |
| 11 | chr15 | 95870027 | 95870409 | LINC01197 | 1.5E−49 | 392 |
| 12 | chr19 | 35786422 | 35787037 | MAG | 1 | 388 |
| 13 | chr13 | 28498102 | 28499045 | PDX1-AS1 | 0.0000017 | 382 |
| 14 | chr17 | 7492629 | 7492750 | SOX15 | 2.5E−13 | 375 |
| 15 | chr2 | 28718469 | 28719192 | PLB1 | 0.000000052 | 368 |
| 16 | chr22 | 40081927 | 40082386 | CACNA1I | 6.6E−53 | 351 |
| 17 | chr6 | 392222 | 393517 | IRF4 | 4.6E−91 | 341 |
| 18 | chr7 | 20823895 | 20824488 | SP8 | 4.2E−89 | 333 |
| 19 | chr2 | 175594518 | 175595042 | CHRNA1 | 4.2E−28 | 332 |
| 20 | chr3 | 5137407 | 5137976 | ARL8B | 1.7E−26 | 319 |
| 21 | chr4 | 5891985 | 5892089 | CRMP1 | 0.000000003 | 307 |
| 22 | chr7 | 103630754 | 103631004 | RELN | 1 | 296 |
| 23 | chr1 | 151104366 | 151105046 | SEMA6C | 2.4E−36 | 272 |
| 24 | chr4 | 13545251 | 13545612 | NKX3-2 | 7.9E−39 | 225 |
| 25 | chr19 | 50861655 | 50861825 | NAPSA | 0.000021 | 202 |
| 26 | chr15 | 66914678 | 66914731 | LINC01169 | 0.000024 | 196 |
| 27 | chr5 | 92931643 | 92932639 | MIR548AO | 2.9E−10 | 194 |
| 28 | chr20 | 37356055 | 37357918 | SLC32A1 | 1.2E−132 | 187 |
| 29 | chr3 | 147136946 | 147137256 | LOC440982 | 3.2E−16 | 186 |
| 30 | chr3 | 127347687 | 127347806 | PODXL2 | 1 | 178 |
| 31 | chr9 | 22005753 | 22006306 | CDKN2B | 9.9E−23 | 178 |
| 32 | chr8 | 53851723 | 53853197 | NPBWR1 | 6.5E−128 | 163 |
| 33 | chr8 | 70983045 | 70984036 | PRDM14 | 3.2E−41 | 160 |
| 34 | chr8 | 41165745 | 41167139 | SFRP1 | 7E−164 | 158 |
| 35 | chr7 | 155250353 | 155250415 | EN2 | 1 | 155 |
| 36 | chr5 | 76924167 | 76924428 | OTP | 1.9E−30 | 155 |
| 37 | chr20 | 23066831 | 23067573 | CD93 | 7.6E−12 | 153 |
| 38 | chr16 | 29796434 | 29796710 | KIF22 | 1.2E−15 | 152 |
| 39 | chr8 | 97156853 | 97158021 | GDF6 | 1.8E−136 | 152 |
| 40 | chr1 | 78956690 | 78956865 | PTGFR | 2.6E−37 | 150 |
| 41 | chr3 | 184301210 | 184301607 | EPHB3 | 2.9E−26 | 148 |
| 42 | chr22 | 38221446 | 38221545 | GALR3 | 0.037 | 148 |
| 43 | chr5 | 180100616 | 180101412 | FLT4 | 2.8E−59 | 144 |
| 44 | chr18 | 43913947 | 43913991 | RNF165 | 0.51 | 144 |
| 45 | chr19 | 58545029 | 58545559 | ZSCAN1 | 2.4E−140 | 144 |
| 46 | chr10 | 108923670 | 108924867 | SORCS1 | 5.9E−143 | 143 |
| 47 | chr16 | 67199669 | 67199869 | HSF4 | 0.00000029 | 139 |
| 48 | chr9 | 136075475 | 136075811 | OBP2B | 8.5E−09 | 135 |
| 49 | chr6 | 50818180 | 50818424 | TFAP2B | 4.3E−100 | 135 |
| 50 | chr19 | 39755028 | 39755922 | IFNL2 | 5.3E−101 | 134 |
| 51 | chr16 | 51186291 | 51188813 | SALL1 | 1E−71 | 132 |
| 52 | chr9 | 122131297 | 122131749 | BRINP1 | 9E−54 | 131 |
| 53 | chr19 | 18760598 | 18760955 | KLHL26 | 1.1E−36 | 129 |
| 54 | chr10 | 134843442 | 134843793 | ADGRA1-AS1 | 0.00000026 | 128 |
| 55 | chr7 | 155242318 | 155242925 | EN2 | 1.1E−58 | 128 |
| 56 | chr11 | 134281625 | 134281685 | B3GAT1 | 0.023 | 127 |
| 57 | chr5 | 172671531 | 172672649 | NKX2-5 | 8.8E−43 | 126 |
| 58 | chr6 | 166074314 | 166074364 | PDE10A | 0.00000024 | 125 |
| 59 | chr3 | 156009098 | 156009319 | KCNAB1 | 1.6E−18 | 122 |
| 60 | chr22 | 25678673 | 25679272 | IGLL3P | 1.6E−80 | 116 |
| 61 | chr5 | 16179209 | 16179553 | MARCH11 | 1.8E−51 | 112 |
| 62 | chr7 | 4832002 | 4832627 | MIR4656 | 2.5E−61 | 108 |
| 63 | chr19 | 55597993 | 55599029 | EPS8L1 | 1.6E−70 | 108 |
| 64 | chr1 | 12148201 | 12148489 | TNFRSF8 | 1 | 103 |
| 65 | chr16 | 32896382 | 32896472 | SLC6A10P | 0.013 | 103 |
| 66 | chr6 | 170413895 | 170414149 | LOC102724511 | 1 | 101 |
| 67 | chr1 | 85156186 | 85156257 | SSX2IP | 0.00013 | 100 |
| 68 | chr14 | 103415848 | 103416095 | AMN | 0.00078 | 98 |
| 69 | chr1 | 23279790 | 23279970 | LACTBL1 | 0.000012 | 95 |
| 70 | chr5 | 1201660 | 1201805 | SLC6A19 | 0.000046 | 89 |
| 71 | chr5 | 147862433 | 147862693 | FBXO38 | 0.00044 | 87 |
| 72 | chr8 | 101821840 | 101822199 | PABPC1 | 4.4E−14 | 85 |
| 73 | chr5 | 170735030 | 170736513 | TLX3 | 2.7E−59 | 85 |
| 74 | chr12 | 4140969 | 4141148 | PARP11 | 0.42 | 84 |
| 75 | chr5 | 60922335 | 60922568 | C5orf64 | 0.0000004 | 82 |
| 76 | chr18 | 908970 | 909154 | ADCYAP1 | 1.7E−18 | 81 |
| 77 | chr7 | 32801313 | 32802074 | LINC00997 | 1.7E−11 | 80 |
| 78 | chr16 | 86016293 | 86016452 | MIR6774 | 2.1E−11 | 80 |
| 79 | chr6 | 43612626 | 43612953 | RSPH9 | 1.8E−31 | 75 |
| 80 | chr7 | 98972247 | 98972345 | ARPC1B | 0.031 | 74 |
| 81 | chr3 | 44063543 | 44063686 | MIR138-1 | 1.4E−18 | 73 |
| 82 | chr13 | 36052403 | 36053213 | NBEA | 1.3E−27 | 72 |
| 83 | chr17 | 15689705 | 15689796 | MEIS3P1 | 1 | 70 |
| 84 | chr3 | 179169151 | 179169204 | GNB4 | 0.00000099 | 70 |
| 85 | chr2 | 124782229 | 124783355 | CNTNAP5 | 2E−81 | 69 |
| 86 | chr16 | 214606 | 216803 | HBM | 0.00028 | 69 |
| 87 | chr2 | 43398020 | 43398275 | ZFP36L2 | 0.000000017 | 68 |
| 88 | chr1 | 50881119 | 50882140 | DMRTA2 | 1.4E−33 | 68 |
| 89 | chr1 | 57285033 | 57285466 | C1orf168 | 0.0000027 | 68 |
| 90 | chr10 | 38069616 | 38069756 | ZNF33BP1 | 0.00000094 | 64 |
| 91 | chr6 | 58147126 | 58149415 | LINC00680 | 1.7E−97 | 64 |
| 92 | chr19 | 14584258 | 14584325 | PTGER1 | 4.8E−11 | 62 |
| 93 | chr14 | 97685059 | 97685145 | LOC101929241 | 0.0000091 | 62 |
| 94 | chr11 | 67134493 | 67134714 | CLCF1 | 1 | 59 |
| 95 | chr19 | 51107536 | 51107741 | SNAR-F | 1.4E−23 | 58 |
| 96 | chr17 | 34820329 | 34820801 | TBC1D3B | 1.5E−19 | 57 |
| 97 | chr20 | 44685820 | 44687609 | SLC12A5 | 5.6E−111 | 54 |
| 98 | chr4 | 158141284 | 158141917 | GRIA2 | 5.7E−62 | 53 |
| 99 | chr8 | 145103649 | 145103720 | MIR6846 | 0.0000086 | 52 |
| 100 | chr5 | 136834876 | 136834971 | SPOCK1 | 1 | 51 |
| 101 | chr4 | 165304258 | 165305137 | MARCH1 | 2.6E−88 | 51 |
| 102 | chr22 | 29876701 | 29876739 | NEFH | 1.4E−22 | 51 |
| 103 | chr16 | 67196698 | 67197932 | HSF4 | 1.1E−100 | 47 |
| 104 | chr6 | 166074223 | 166074291 | PDE10A | 4.3E−14 | 45 |
| 105 | chr19 | 15121590 | 15122224 | CCDC105 | 5.7E−66 | 44 |
| 106 | chr5 | 4222954 | 4223319 | LOC101929153 | 0.002 | 44 |
| 107 | chr8 | 143960890 | 143961303 | CYP11B1 | 0.0025 | 43 |
| 108 | chr5 | 172110795 | 172111132 | NEURL1B | 1.1E−47 | 43 |
| 109 | chr20 | 55206573 | 55206645 | TFAP2C | 0.00000016 | 43 |
| 110 | chr11 | 92702812 | 92703083 | MTNR1B | 2E−40 | 43 |
| 111 | chr12 | 25055895 | 25056351 | BCAT1 | 3.4E−44 | 42 |
| 112 | chr1 | 2990108 | 2990171 | PRDM16 | 0.000012 | 40 |
| 113 | chr20 | 61788160 | 61788642 | MIR124-3 | 4.5E−18 | 38 |
| 114 | chr17 | 12877226 | 12877772 | ELAC2 | 1.5E−36 | 37 |
| 115 | chr8 | 140715513 | 140715920 | KCNK9 | 5.9E−57 | 37 |
| 116 | chr8 | 70982620 | 70983018 | PRDM14 | 0.000000064 | 36 |
| 117 | chr6 | 108495504 | 108495979 | NR2E1 | 1.1E−24 | 35 |
| 118 | chr5 | 178367536 | 178368804 | ZNF454 | 6.3E−99 | 34 |
| 119 | chr2 | 43411339 | 43411705 | ZFP36L2 | 5.3E−10 | 32 |
| 120 | chr22 | 16156806 | 16157039 | BMS1P18 | 1 | 30 |
| 121 | chr16 | 1922298 | 1922519 | MEIOB | 1.5E−14 | 28 |
| 122 | chr1 | 211589885 | 211590236 | LINC00467 | 8.1E−31 | 28 |
| 123 | chr9 | 34577906 | 34578054 | CNTFR-AS1 | 1 | 27 |
| 124 | chr17 | 4463580 | 4464239 | GGT6 | 1.6E−09 | 27 |
| 125 | chr8 | 80803608 | 80804203 | LOC101927040 | 9.1E−46 | 27 |
| 126 | chr9 | 99838560 | 99840130 | GAS2L1P2 | 8.5E−38 | 27 |
| 127 | chr3 | 147127578 | 147127935 | ZIC1 | 3.3E−28 | 26 |
| 128 | chr22 | 17488797 | 17488993 | GAB4 | 0.067 | 25 |
| 129 | chr22 | 37464961 | 37465138 | KCTD17 | 6.4E−24 | 25 |
| 130 | chr7 | 101961700 | 101962042 | MIR4285 | 1.8E−28 | 24 |
| 131 | chr14 | 38080380 | 38080646 | TTC6 | 5.5E−31 | 24 |
| 132 | chr5 | 1295363 | 1295444 | TERT | 5.9E−14 | 23 |
| 133 | chr16 | 88504233 | 88504318 | ZNF469 | 0.0031 | 23 |
| 134 | chr2 | 90413838 | 90414075 | MIR4436A | 1.3E−27 | 22 |
| 135 | chr3 | 124860337 | 124861045 | MIR5092 | 1.7E−43 | 21 |
| 136 | chr5 | 175107749 | 175108520 | HRH2 | 8.7E−11 | 21 |
| 137 | chr20 | 21695247 | 21695306 | PAX1 | 3.6E−14 | 21 |
| 138 | chr12 | 115135925 | 115135961 | TBX3 | 4E−11 | 20 |
| 139 | chr2 | 115420089 | 115420215 | DPP10-AS3 | 1 | 20 |
| 140 | chr12 | 312591 | 312753 | LOC101929384 | 2E−45 | 20 |
| 141 | chr4 | 174429648 | 174430640 | HAND2 | 1.5E−68 | 19 |
| 142 | chr5 | 32712589 | 32713895 | NPR3 | 1.6E−117 | 19 |
| 143 | chr3 | 48698862 | 48698933 | CELSR3 | 0.00000013 | 19 |
| 144 | chr3 | 13245874 | 13246211 | IQSEC1 | 0.0000017 | 18 |
| 145 | chr10 | 135178458 | 135178918 | MIR3944 | 7.1E−15 | 18 |
| 146 | chr7 | 62574764 | 62574921 | ZNF733P | 1.7E−10 | 18 |
| 147 | chr14 | 91818586 | 91818752 | CCDC88C | 1 | 18 |
| 148 | chr2 | 149632858 | 149632906 | KIF5C | 1 | 17 |
| 149 | chr3 | 196373400 | 196373649 | NRROS | 0.000045 | 16 |
| 150 | chr7 | 2677842 | 2678081 | TTYH3 | 0.058 | 16 |
| 151 | chr5 | 2751128 | 2757191 | C5orf38 | 1.3E−152 | 16 |
| 152 | chr11 | 66314443 | 66314513 | ACTN3 | 1 | 16 |
| 153 | chr9 | 92291209 | 92291585 | UNQ6494 | 3.4E−15 | 16 |
| 154 | chr13 | 114060446 | 114060794 | LOC101928841 | 0.0086 | 15 |
| 155 | chr11 | 1394693 | 1395391 | BRSK2 | 2.9E−17 | 15 |
| 156 | chr12 | 30322944 | 30323207 | TMTC1 | 1.7E−24 | 15 |
| 157 | chr21 | 47468994 | 47469471 | COL6A2 | 0.00002 | 15 |
| 158 | chr17 | 48858635 | 48858925 | MIR8059 | 1.5E−40 | 15 |
| 159 | chr11 | 6434946 | 6435305 | APBB1 | 0.0000012 | 15 |
| 160 | chr1 | 12203630 | 12203905 | MIR7846 | 0.0000071 | 14 |
| 161 | chr1 | 248551109 | 248551544 | OR2T6 | 0.0000012 | 14 |
| 162 | chr11 | 72533018 | 72533651 | ATG16L2 | 9.7E−33 | 14 |
| 163 | chr15 | 89922050 | 89922792 | MIR9-3HG | 1.2E−57 | 14 |
| 164 | chr1 | 2136776 | 2136872 | FAAP20 | 1 | 13 |
| 165 | chr11 | 2292176 | 2292231 | ASCL2 | 0.37 | 13 |
| 166 | chr14 | 23876799 | 23877073 | MYH6 | 0.0031 | 13 |
| 167 | chr2 | 239756351 | 239757924 | TWIST2 | 7.5E−148 | 13 |
| 168 | chr8 | 70946912 | 70947440 | PRDM14 | 8.8E−32 | 13 |
| 169 | chr1 | 119522232 | 119522972 | TBX15 | 1.4E−55 | 12 |
| 170 | chr9 | 131218649 | 131218710 | ODF2 | 0.00002 | 12 |
| 171 | chr7 | 157477410 | 157478936 | MIR153-2 | 1.6E−160 | 12 |
| 172 | chr2 | 31457371 | 31457421 | EHD3 | 1.8E−13 | 12 |
| 173 | chr20 | 61885447 | 61885809 | NKAIN4 | 2.1E−55 | 12 |
| 174 | chr6 | 110300487 | 110301094 | GPR6 | 2.5E−45 | 11 |
| 175 | chr12 | 2734265 | 2734642 | CACNA1C-AS2 | 0.000029 | 11 |
| 176 | chr11 | 131526606 | 131526947 | NTM-AS1 | 1 | 10 |
| 177 | chr5 | 170743598 | 170743662 | TLX3 | 1.8E−17 | 10 |
| 178 | chr3 | 51741015 | 51741087 | GRM2 | 1 | 10 |
| 179 | chr8 | 65282005 | 65282932 | LOC102724623 | 2.4E−121 | 9 |
| 180 | chr18 | 77218864 | 77219138 | NFATC1 | 1 | 9 |
| 181 | chr11 | 10531381 | 10531779 | MTRNR2L8 | 6.3E−14 | 8 |
| 182 | chr10 | 105344151 | 105344855 | NEURL1-AS1 | 2.3E−61 | 8 |
| 183 | chr1 | 224804523 | 224804634 | CNIH3 | 0.00071 | 8 |
| 184 | chr19 | 37287854 | 37287967 | ZNF790-AS1 | 0.00016 | 8 |
| 185 | chr6 | 41395121 | 41395407 | LINC01276 | 2.5E−09 | 8 |
| 186 | chr5 | 92930812 | 92931061 | MIR548AO | 0.000000012 | 8 |
| 187 | chr3 | 10858358 | 10858432 | SLC6A11 | 0.00000011 | 7 |
| 188 | chr16 | 204220 | 204318 | HBZ | 0.059 | 7 |
| 189 | chr10 | 26855748 | 26856149 | LINC00264 | 2.9E−12 | 7 |
| 190 | chr1 | 43750485 | 43750844 | C1orf210 | 4.9E−10 | 7 |
| 191 | chr2 | 45232447 | 45232534 | SIX2 | 0.0000018 | 7 |
| 192 | chr14 | 48143369 | 48145724 | MDGA2 | 1.2E−274 | 7 |
| 193 | chr1 | 232941599 | 232941809 | MAP10 | 1.2E−12 | 6 |
| 194 | chr6 | 26522244 | 26522317 | HCG11 | 9.4E−10 | 6 |
| 195 | chr6 | 100895620 | 100896203 | SIM1 | 1.1E−21 | 5 |
| 196 | chr7 | 101961741 | 101962354 | MIR4285 | 4.4E−75 | 5 |
| 197 | chr16 | 1171931 | 1172126 | C1QTNF8 | 1 | 5 |
| 198 | chr5 | 140864384 | 140864884 | PCDHGC4 | 2.5E−22 | 5 |
| 199 | chr1 | 16085208 | 16085925 | FBLIM1 | 8.8E−74 | 5 |
| 200 | chr7 | 64343128 | 64343269 | ZNF273 | 1 | 5 |
| 201 | chr16 | 67687133 | 67687281 | ACD | 1 | 5 |
| 202 | chr11 | 1358353 | 1358439 | TOLLIP-AS1 | 0.00094 | 4 |
| 203 | chr7 | 149389572 | 149390059 | KRBA1 | 1.3E−23 | 4 |
| 204 | chr3 | 154146310 | 154146951 | GPR149 | 1.5E−41 | 4 |
| 205 | chr3 | 179754308 | 179755306 | PEX5L | 8.8E−82 | 4 |
| 206 | chr4 | 2065950 | 2066251 | NAT8L | 1.4E−09 | 4 |
| 207 | chr1 | 245772586 | 245772992 | KIF26B | 3.7E−11 | 4 |
| 208 | chr8 | 67873692 | 67873780 | TCF24 | 1.4E−13 | 4 |
| 209 | chr15 | 70740308 | 70740480 | SALRNA3 | 2.2E−18 | 4 |
| 210 | chr10 | 94451209 | 94452485 | HHEX | 1.7E−69 | 4 |
| 211 | chr14 | 103568641 | 103568719 | EXOC3L4 | 0.000000011 | 3 |
| 212 | chr1 | 112058046 | 112058721 | ADORA3 | 9.9E−78 | 3 |
| 213 | chr3 | 171528450 | 171528601 | PLD1 | 1 | 3 |
| 214 | chr17 | 26699228 | 26699301 | SARM1 | 1 | 3 |
| 215 | chr11 | 27741817 | 27742122 | BDNF | 0.0000021 | 3 |
| 216 | chr2 | 38301293 | 38302642 | CYP1B1 | 4.1E−102 | 3 |
| 217 | chr15 | 60884893 | 60884977 | RORA | 0.00000021 | 3 |
| 218 | chr9 | 68411147 | 68411576 | MIR4477B | 1 | 3 |
| 219 | chr13 | 112575679 | 112576115 | LINC00354 | 1 | 2 |
| 220 | chr9 | 132258070 | 132258227 | LINC00963 | 1 | 2 |
| 221 | chr8 | 140715318 | 140715433 | KCNK9 | 0.00000004 | 2 |
| 222 | chr10 | 14372398 | 14372955 | FRMD4A | 0.001 | 2 |
| 223 | chr8 | 145730875 | 145730928 | GPT | 1 | 2 |
| 224 | chr17 | 17626910 | 17627043 | RAI1 | 0.06 | 2 |
| 225 | chr1 | 202129512 | 202129929 | PTPN7 | 0.000024 | 2 |
| 226 | chr19 | 30016974 | 30017506 | VSTM2B | 6.8E−96 | 2 |
| 227 | chr7 | 4901208 | 4901886 | PAPOLB | 1.9E−46 | 2 |
| 228 | chr13 | 53775090 | 53775595 | LINC01065 | 1.4E−135 | 2 |
| 229 | chr7 | 57484605 | 57484694 | MIR3147 | 1 | 2 |
| 230 | chr20 | 61002544 | 61003481 | RBBP8NL | 1.2E−10 | 2 |
| 231 | chr16 | 67428314 | 67429097 | TPPP3 | 4.2E−63 | 2 |
| 232 | chr4 | 674769 | 675388 | MYL5 | 6.2E−47 | 2 |
| 233 | chr14 | 85996310 | 85996698 | FLRT2 | 2.1E−23 | 2 |
| 234 | chr6 | 100906564 | 100906629 | SIM1 | 3.2E−15 | 1 |
| 235 | chr2 | 105480622 | 105480700 | LINC01159 | 0.0000019 | 1 |
| 236 | chr11 | 107799071 | 107799164 | RAB39A | 0.0000078 | 1 |
| 237 | chr13 | 112720926 | 112720970 | SOX1 | 0.000059 | 1 |
| 238 | chr11 | 124790814 | 124791389 | HEPN1 | 3.2E−64 | 1 |
| 239 | chr9 | 127212708 | 127212901 | PSMB7 | 4.9E−16 | 1 |
| 240 | chr11 | 132952630 | 132952681 | OPCML | 0.0011 | 1 |
| 241 | chr9 | 139085334 | 139085378 | LHX3 | 4.2E−09 | 1 |
| 242 | chr8 | 145104789 | 145104875 | MIR6846 | 0.000000058 | 1 |
| 243 | chr17 | 17626841 | 17626913 | RAI1 | 0.00000028 | 1 |
| 244 | chr19 | 18118974 | 18119334 | ARRDC2 | 9.5E−19 | 1 |
| 245 | chr19 | 1852126 | 1852284 | REXO1 | 1 | 1 |
| 246 | chr2 | 20442160 | 20442243 | SDC1 | 1 | 1 |
| 247 | chr2 | 208988834 | 208989405 | CRYGD | 8.4E−33 | 1 |
| 248 | chr2 | 237476333 | 237476544 | ACKR3 | 1.2E−11 | 1 |
| 249 | chr2 | 241459501 | 241460162 | ANKMY1 | 1.1E−12 | 1 |
| 250 | chr2 | 241949234 | 241949400 | SNED1 | 0.036 | 1 |
| 251 | chr1 | 244014027 | 244014122 | AKT3 | 3.4E−15 | 1 |
| 252 | chr12 | 3308835 | 3308996 | TSPAN9 | 3.2E−21 | 1 |
| 253 | chr17 | 35242084 | 35242555 | LHX1 | 0.0037 | 1 |
| 254 | chr7 | 35294156 | 35299091 | TBX20 | 1.2E−38 | 1 |
| 255 | chr19 | 3585505 | 3585581 | GIPC3 | 1 | 1 |
| 256 | chr15 | 41913923 | 41914359 | MGA | 2.4E−34 | 1 |
| 257 | chr20 | 44728216 | 44728399 | NCOA5 | 7.1E−17 | 1 |
| 258 | chr16 | 51168131 | 51169154 | SALL1 | 9.4E−47 | 1 |
| 259 | chr19 | 56728634 | 56729258 | ZSCAN5A | 6E−68 | 1 |
| 260 | chr13 | 58208012 | 58208111 | PCDH17 | 1.3E−10 | 1 |
| 261 | chr17 | 59483153 | 59483406 | C17orf82 | 1 | 1 |
| 262 | chr20 | 61885836 | 61885926 | NKAIN4 | 0.037 | 1 |
| 263 | chr18 | 7117006 | 7117085 | LAMA1 | 1.5E−09 | 1 |
| 264 | chr18 | 77623664 | 77624111 | KCNG2 | 7.1E−83 | 1 |
| 265 | chr1 | 896234 | 896316 | KLHL17 | 0.000024 | 1 |
| 266 | chr12 | 94954627 | 94954828 | MIR5700 | 1 | 1 |
| 267 | chr15 | 96959357 | 96959433 | NR2F2 | 0.0000002 | 1 |

### Gastrointestinal Cancer

In another aspect is provided a method of detecting a level of DNA methylation in a subject at risk of developing a gastrointestinal cancer and determining its likely tissue of origin, the method including: determining the level of methylation of CpG sites within a plurality of gene regions in a DNA sample from the subject, wherein the plurality of gene regions includes at least 50 different gene regions set forth in Table MCC; and wherein the level of methylation of CpG sites identifies the tissue as colorectal, hepatic, esophageal, or pancreatic. In embodiments, an increased level of methylated CpG sites relative to a standard control indicates a higher risk of gastrointestinal cancer. In embodiments, the level of methylation of CpG sites is higher than a DNA sample from a standard control.

Provided herein is a method of treating a gastrointestinal cancer selected from the group consisting of colorectal cancer, hepatic cancer, esophageal cancer, and pancreatic cancer in a patient in need thereof comprising: (a) detecting an increased level of methylated CpG sites, relative to a standard control, within a plurality of gene regions in a DNA sample obtained from the patient, wherein the plurality of gene regions comprise at least 50 different gene regions in Table MCC; and (b) treating the patient for cancer. Provided herein is a method of treating a gastrointestinal cancer selected from the group consisting of colorectal cancer, hepatic cancer, esophageal cancer, and pancreatic cancer in a patient in need thereof comprising: (a) detecting an increased level of methylated CpG sites, relative to a standard control, within a plurality of gene regions in a DNA sample obtained from the patient, wherein the plurality of gene regions comprise at least 50 different gene regions in Table MCC; (b) identifying the tissue of origin based on the plurality of gene regions having the increased levels of methylated CpG sites, thereby identifying the cancer as colorectal cancer, hepatic cancer, esophageal cancer, or pancreatic cancer; and (c) treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the gastrointestinal cancer selected from the group consisting of colorectal cancer, hepatic cancer, esophageal cancer, and pancreatic cancer is Stage I, Stage II, or Stage III. In embodiments, the gastrointestinal cancer selected from the group consisting of colorectal cancer, hepatic cancer, esophageal cancer, and pancreatic cancer is Stage I. In embodiments, the gastrointestinal cancer selected from the group consisting of colorectal cancer, hepatic cancer, esophageal cancer, and pancreatic cancer is Stage II. In embodiments, the gastrointestinal cancer selected from the group consisting of colorectal cancer, hepatic cancer, esophageal cancer, and pancreatic cancer is Stage III.

Provided herein is a method of diagnosing a gastrointestinal cancer selected from the group consisting of colorectal cancer, hepatic cancer, esophageal cancer, and pancreatic cancer in a patient comprising: (a) detecting an elevated level of methylated CpG sites, relative to a standard control, within a plurality of gene regions in a DNA sample from the patient, wherein the plurality of gene regions comprise at least 50 different gene regions in Table MCC; and (b) diagnosing the patient with a gastrointestinal cancer selected from the group consisting of colorectal cancer, hepatic cancer, esophageal cancer, or pancreatic cancer. Provided herein is a method of diagnosing a gastrointestinal cancer selected from the group consisting of colorectal cancer, hepatic cancer, esophageal cancer, and pancreatic cancer in a patient comprising: (a) detecting an elevated level of methylated CpG sites, relative to a standard control, within a plurality of gene regions in a DNA sample from the patient, wherein the plurality of gene regions comprise at least 50 different gene regions in Table MCC; (b) identifying the tissue of origin based on the plurality of gene regions having the increased levels of methylated CpG sites; and (c) diagnosing the patient with colorectal cancer, hepatic cancer, esophageal cancer, or pancreatic cancer based on the tissue of origin. In embodiments, the gastrointestinal cancer selected from the group consisting of colorectal cancer, hepatic cancer, esophageal cancer, and pancreatic cancer is Stage I, Stage II, or Stage III. In embodiments, the gastrointestinal cancer selected from the group consisting of colorectal cancer, hepatic cancer, esophageal cancer, and pancreatic cancer is Stage I. In embodiments, the gastrointestinal cancer selected from the group consisting of colorectal cancer, hepatic cancer, esophageal cancer, and pancreatic cancer is Stage II. In embodiments, the gastrointestinal cancer selected from the group consisting of colorectal cancer, hepatic cancer, esophageal cancer, and pancreatic cancer is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Provided herein is a method of monitoring treatment in a patient having a gastrointestinal cancer selected from the group consisting of colorectal cancer, hepatic cancer, esophageal cancer, and pancreatic cancer or monitoring risk for developing a gastrointestinal cancer selected from the group consisting of colorectal cancer, hepatic cancer, esophageal cancer, and pancreatic cancer in a patient comprising: (a) detecting a level of methylated CpG sites within a plurality of gene regions in a DNA sample from the patient at a first time point, wherein the plurality of gene regions comprise at least 50 different gene regions in Table MCC; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring treatment or monitoring risk. In embodiments, the method comprises monitoring risk for developing a gastrointestinal cancer selected from the group consisting of colorectal cancer, hepatic cancer, esophageal cancer, and pancreatic cancer. In embodiments, the level of methylated CpG sites within the plurality of gene regions at the first time point is substantially the same as a standard control and the level of methylated CpG sites within the plurality of gene regions at the second time point is substantially the same as the level at the first time point, thereby indicating that the patient is likely not at risk for developing gastrointestinal cancer or does not have gastrointestinal cancer. In embodiments, the level of methylated CpG sites within the plurality of gene regions at the first time point is substantially the same as a standard control and the level of methylated CpG sites within the plurality of gene regions at the second time point is elevated when compared to the level at the first time point, thereby indicating that the patient is at risk for developing gastrointestinal cancer or may have gastrointestinal cancer. In embodiments, the method further comprises identifying the tissue of origin based on the plurality of gene regions having the elevated levels of methylated CpG sites, thereby identifying the cancer as colorectal cancer, hepatic cancer, esophageal cancer, or pancreatic cancer. In embodiments, the gastrointestinal cancer selected from the group consisting of colorectal cancer, hepatic cancer, esophageal cancer, and pancreatic cancer is Stage I, Stage II, or Stage III. In embodiments, the gastrointestinal cancer selected from the group consisting of colorectal cancer, hepatic cancer, esophageal cancer, and pancreatic cancer is Stage I. In embodiments, the gastrointestinal cancer selected from the group consisting of colorectal cancer, hepatic cancer, esophageal cancer, and pancreatic cancer is Stage II. In embodiments, the gastrointestinal cancer selected from the group consisting of colorectal cancer, hepatic cancer, esophageal cancer, and pancreatic cancer is Stage III. In embodiments, the method further comprises treating the patient for cancer. In embodiments, treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

In embodiments, the gene regions in Table MCC include different methylated regions which are hyper-methylated in cancer patients when compared to healthy patients (e.g., patients without cancer). In embodiments, some of the differentially methylated regions are unique to individual gastrointestinal cancers which allows for distinguishing between different gastrointestinal cancers (e.g., colorectal cancer, hepatocellular carcinoma, esophageal cancer, pancreatic ductal adenocarcinoma). Thus, in embodiments, the method further comprises identifying the tissue of origin (e.g., colon, liver, esophagus, pancreas) in order to identify the specific gastrointestinal cancer (e.g., colorectal cancer, hepatocellular carcinoma, esophageal cancer, pancreatic ductal adenocarcinoma, respectively). Identifying the tissue of origin as from the colon or rectum indicates that the gastrointestinal cancer is colorectal cancer. Identifying the tissue of origin as from the liver indicates that the gastrointestinal cancer is hepatocellular carcinoma. Identifying the tissue of origin as from the esophagus indicates that the gastrointestinal cancer is esophageal cancer. Identifying the tissue of origin as from the pancreas indicates that the gastrointestinal cancer is pancreatic ductal adenocarcinoma. The tissue of origin can be identified based on the plurality of gene regions having the increased levels of methylated CpG sites. Each tissue (e.g., colon, liver, esophagus, pancreas) will correspond to different gene regions having elevated levels of methylated CpG sites. The differentially methylated regions of the different tissue of origin may or may not be overlapping. In embodiments, the tissue of origin can be identified by comparing the plurality of gene regions having the elevated levels of methylated CpG sites to a control. In embodiments, the control is a population of patients having colorectal cancer, a population of patients having hepatocellular carcinoma, a population of patients having esophageal cancer, a population of patients having pancreatic ductal adenocarcinoma, and a population of healthy patients (i.e., patients that do not have cancer). The control can be prepared as described herein (e.g., clustering data using a t-SNE plot).

In embodiments, the plurality of gene regions includes at least 1 DMR (i.e., gene region) in Table MCC. In embodiments, the plurality of gene regions includes at least 2 DMRs (i.e., gene regions) in Table MCC. In embodiments, the plurality of gene regions includes at least 3 DMRs in Table MCC. In embodiments, the plurality of gene regions includes at least 4 DMRs in Table MCC. In embodiments, the plurality of gene regions includes at least 5 DMRs in Table MCC. In embodiments, the plurality of gene regions includes at least 6 DMRs in Table MCC. In embodiments, the plurality of gene regions includes at least 7 DMRs in Table MCC. In embodiments, the plurality of gene regions includes at least 8 DMRs in Table MCC. In embodiments, the plurality of gene regions includes at least 9 DMRs in Table MCC. In embodiments, the plurality of gene regions includes at least 10 DMRs in Table MCC. In embodiments, the plurality of gene regions includes at least 15 DMRs in Table MCC. In embodiments, the plurality of gene regions includes at least 20 DMRs in Table MCC. In embodiments, the plurality of gene regions includes at least 25 DMRs in Table MCC. In embodiments, the plurality of gene regions includes at least 30 DMRs in Table MCC. In embodiments, the plurality of gene regions includes at least 35 DMRs in Table MCC. In embodiments, the plurality of gene regions includes at least 40 DMRs in Table MCC. In embodiments, the plurality of gene regions includes at least 45 DMRs in Table MCC. In embodiments, the plurality of gene regions includes at least 50 DMRs in Table MCC. In embodiments, the plurality of gene regions includes at least 55 DMRs in Table MCC. In embodiments, the plurality of gene regions includes at least 60 DMRs in Table MCC. In embodiments, the plurality of gene regions includes at least 65 DMRs in Table MCC. In embodiments, the plurality of gene regions includes at least 70 DMRs in Table MCC. In embodiments, the plurality of gene regions includes at least 75 DMRs in Table MCC. In embodiments, the plurality of gene regions includes at least 80 DMRs in Table MCC. In embodiments, the plurality of gene regions includes at least 85 DMRs in Table MCC. In embodiments, the plurality of gene regions includes at least 90 DMRs in Table MCC. In embodiments, the plurality of gene regions includes at least 95 DMRs in Table MCC. In embodiments, the plurality of gene regions includes at least 110 DMRs in Table MCC. In embodiments, the plurality of gene regions includes at least 120 DMRs in Table MCC. In embodiments, the plurality of gene regions includes at least 130 DMRs in Table MCC. In embodiments, the plurality of gene regions includes at least 140 DMRs in Table MCC. In embodiments, the plurality of gene regions includes at least 150 DMRs in Table MCC. In embodiments, the plurality of gene regions includes at least 160 DMRs in Table MCC. In embodiments, the plurality of gene regions includes at least 170 DMRs in Table MCC. In embodiments, the plurality of gene regions includes at least 180 DMRs in Table MCC. In embodiments, the plurality of gene regions includes at least 190 DMRs in Table MCC. In embodiments, the plurality of gene regions includes at least 200 DMRs in Table MCC. In embodiments, the plurality of gene regions includes at least 225 DMRs in Table MCC. In embodiments, the plurality of gene regions includes at least 250 DMRs in Table MCC. In embodiments, the plurality of gene regions includes at least 275 DMRs in Table MCC. In embodiments, the plurality of gene regions includes at least 300 DMRs in Table MCC. In embodiments, the plurality of gene regions includes at least 325 DMRs in Table MCC. In embodiments, the plurality of gene regions includes at least 350 DMRs in Table MCC. In embodiments, the plurality of gene regions includes at least 375 DMRs in Table MCC.

In embodiments, the plurality of gene regions includes the first DMR (i.e., gene region) in Table MCC. In embodiments, the plurality of gene regions includes the first 2 DMRs (i.e., gene regions) in Table MCC. In embodiments, the plurality of gene regions includes the first 3 DMRs in Table MCC. In embodiments, the plurality of gene regions includes the first 4 DMRs in Table MCC. In embodiments, the plurality of gene regions includes the first 5 DMRs in Table MCC. In embodiments, the plurality of gene regions includes the first 6 DMRs in Table MCC. In embodiments, the plurality of gene regions includes the first 7 DMRs in Table MCC. In embodiments, the plurality of gene regions includes the first 8 DMRs in Table MCC. In embodiments, the plurality of gene regions includes the first 9 DMRs in Table MCC. In embodiments, the plurality of gene regions includes the first 10 DMRs in Table MCC. In embodiments, the plurality of gene regions includes the first 15 DMRs in Table MCC. In embodiments, the plurality of gene regions includes the first 20 DMRs in Table MCC. In embodiments, the plurality of gene regions includes the first 25 DMRs in Table MCC. In embodiments, the plurality of gene regions includes the first 30 DMRs in Table MCC. In embodiments, the plurality of gene regions includes the first 35 DMRs in Table MCC. In embodiments, the plurality of gene regions includes the first 40 DMRs in Table MCC. In embodiments, the plurality of gene regions includes the first 45 DMRs in Table MCC. In embodiments, the plurality of gene regions includes the first 50 DMRs in Table MCC. In embodiments, the plurality of gene regions includes the first 55 DMRs in Table MCC. In embodiments, the plurality of gene regions includes the first 60 DMRs in Table MCC. In embodiments, the plurality of gene regions includes the first 65 DMRs in Table MCC. In embodiments, the plurality of gene regions includes the first 70 DMRs in Table MCC. In embodiments, the plurality of gene regions includes the first 75 DMRs in Table MCC. In embodiments, the plurality of gene regions includes the first 80 DMRs in Table MCC. In embodiments, the plurality of gene regions includes the first 85 DMRs in Table MCC. In embodiments, the plurality of gene regions includes the first 90 DMRs in Table MCC. In embodiments, the plurality of gene regions includes the first 95 DMRs in Table MCC. In embodiments, the plurality of gene regions includes the first 110 DMRs in Table MCC. In embodiments, the plurality of gene regions includes the first 120 DMRs in Table MCC. In embodiments, the plurality of gene regions includes the first 130 DMRs in Table MCC. In embodiments, the plurality of gene regions includes the first 140 DMRs in Table MCC. In embodiments, the plurality of gene regions includes the first 150 DMRs in Table MCC. In embodiments, the plurality of gene regions includes the first 160 DMRs in Table MCC. In embodiments, the plurality of gene regions includes the first 170 DMRs in Table MCC. In embodiments, the plurality of gene regions includes the first 180 DMRs in Table MCC. In embodiments, the plurality of gene regions includes the first 190 DMRs in Table MCC. In embodiments, the plurality of gene regions includes the first 200 DMRs in Table MCC. In embodiments, the plurality of gene regions includes the first 225 DMRs in Table MCC. In embodiments, the plurality of gene regions includes the first 250 DMRs in Table MCC. In embodiments, the plurality of gene regions includes the first 275 DMRs in Table MCC. In embodiments, the plurality of gene regions includes the first 300 DMRs in Table MCC. In embodiments, the plurality of gene regions includes the first 325 DMRs in Table MCC. In embodiments, the plurality of gene regions includes the first 350 DMRs in Table MCC. In embodiments, the plurality of gene regions includes the first 375 DMRs in Table MCC.

In embodiments of the methods described herein, the DNA sample is cell-free DNA. In embodiments of the methods described herein, the DNA sample is cell-free-DNA in a biological fluid. In embodiments of the methods described herein, the DNA sample is cell-free-DNA in blood. In embodiments of the methods described herein, the DNA sample is cell-free-DNA in plasma. In embodiments of the methods described herein, the DNA sample is substantially cell-free DNA. In embodiments, the DNA sample from a biological fluid is substantially cell-free DNA. In embodiments, the DNA sample from a biological fluid is cell-free DNA. In embodiments, the biological fluid is plasma.

In embodiments, the method further includes performing a confirmatory diagnostic procedure on the subject. Confirmatory diagnostic procedures for each type of gastrointestinal cancer are described in detail herein.

TABLE MCC

| | | | | | Adjusted | |
|---|---|---|---|---|---|---|
| No. | Chr | Start | End | Gene Name | p-value | Freq |
| 1 | chr5 | 93905177 | 93905542 | KIAA0825 | 6.1E−79 | 423 |
| 2 | chr6 | 170553122 | 170553880 | LOC154449 | 1.9E−32 | 418 |
| 3 | chr1 | 167486793 | 167487885 | CD247 | 3.3E−60 | 417 |
| 4 | chr13 | 112978530 | 112978862 | LINC01044 | 5.8E−10 | 412 |
| 5 | chr19 | 719021 | 719534 | PALM | 2.3E−24 | 412 |
| 6 | chr15 | 101629342 | 101629498 | CHSY1 | 0.000052 | 411 |
| 7 | chr6 | 2891827 | 2892313 | LOC101927730 | 2.4E−60 | 411 |
| 8 | chr2 | 90016149 | 90016457 | MIR4436A | 6.3E−30 | 411 |
| 9 | chr2 | 132202681 | 132202749 | RNU6-81P | 0.000000045 | 409 |
| 10 | chr10 | 369324 | 369404 | | 9.5E−09 | 408 |
| 11 | chr11 | 117069683 | 117070130 | TAGLN | 0.000000032 | 407 |
| 12 | chr12 | 132880235 | 132880460 | GALNT9 | 0.011 | 407 |
| 13 | chr11 | 128693960 | 128694458 | KCNJ1 | 5.6E−91 | 405 |
| 14 | chr1 | 153589743 | 153590365 | S100A14 | 3.5E−17 | 405 |
| 15 | chr1 | 159046208 | 159047271 | AIM2 | 2.1E−12 | 405 |
| 16 | chr6 | 170408207 | 170408314 | LOC102724511 | 0.00064 | 405 |
| 17 | chr8 | 37082396 | 37082577 | | 1.1E−21 | 405 |
| 18 | chr17 | 76354740 | 76355115 | SOCS3 | 3.9E−17 | 405 |
| 19 | chr11 | 64034860 | 64035018 | PLCB3 | 8.7E−29 | 404 |
| 20 | chr11 | 67203157 | 67203797 | PTPRCAP | 7.7E−57 | 404 |
| 21 | chr8 | 19319135 | 19319381 | CSGALNACT1 | 1.5E−15 | 403 |
| 22 | chr11 | 315696 | 316680 | IFITM1 | 2E−259 | 403 |
| 23 | chr8 | 1449737 | 1450097 | DLGAP2 | 0.000037 | 401 |
| 24 | chr4 | 37245350 | 37245479 | NWD2 | 5.5E−15 | 401 |
| 25 | chr14 | 101012875 | 101013110 | BEGAIN | 1E−164 | 400 |
| 26 | chr19 | 48564916 | 48565400 | CABP5 | 9.1E−22 | 400 |
| 27 | chr2 | 242927726 | 242928044 | LOC102723927 | 4.3E−33 | 399 |
| 28 | chr4 | 3566226 | 3566322 | LINC00955 | 3.3E−17 | 398 |
| 29 | chr4 | 7483771 | 7483929 | MIR4274 | 1.9E−10 | 397 |
| 30 | chr17 | 3289582 | 3289717 | OR1E1 | 1.9E−18 | 396 |
| 31 | chr16 | 89674943 | 89675297 | DPEP1 | 0.0000076 | 396 |
| 32 | chr1 | 9775580 | 9776133 | PIK3CD-AS2 | 1.5E−61 | 396 |
| 33 | chr1 | 156094368 | 156095157 | LMNA | 0.008 | 395 |
| 34 | chr21 | 45575286 | 45575930 | C21orf33 | 1.5E−86 | 395 |
| 35 | chr7 | 99817602 | 99818435 | PVRIG | 1.3E−98 | 395 |
| 36 | chr9 | 125391116 | 125391618 | OR1B1 | 0.000006 | 394 |
| 37 | chr5 | 140871895 | 140872219 | PCDHGC5 | 5.5E−65 | 394 |
| 38 | chr16 | 87447837 | 87448079 | MAP1LC3B | 2.9E−38 | 394 |
| 39 | chr11 | 10531471 | 10531779 | MTRNR2L8 | 2.2E−16 | 393 |
| 40 | chr10 | 1155989 | 1156594 | LINC00200 | 2.6E−25 | 393 |
| 41 | chr7 | 155242263 | 155242925 | EN2 | 5.2E−47 | 393 |
| 42 | chr6 | 170373693 | 170374061 | LOC102724511 | 2.4E−20 | 393 |
| 43 | chr6 | 7051353 | 7051554 | RREB1 | 0.0014 | 393 |
| 44 | chr10 | 11085790 | 11086026 | CELF2 | 0.029 | 391 |
| 45 | chr6 | 170730572 | 170731027 | MIR4644 | 0.00002 | 391 |
| 46 | chr22 | 39784831 | 39785135 | TAB1 | 2.7E−13 | 391 |
| 47 | chr6 | 170554663 | 170554906 | LOC154449 | 0.000024 | 390 |
| 48 | chr3 | 31494123 | 31494273 | STT3B | 4.1E−13 | 390 |
| 49 | chr8 | 1764397 | 1764796 | MIR596 | 8.2E−27 | 388 |
| 50 | chr16 | 85478510 | 85479206 | MIR5093 | 3.6E−47 | 387 |
| 51 | chr9 | 91605620 | 91606003 | C9orf47 | 4.5E−35 | 386 |
| 52 | chr1 | 112058046 | 112058721 | ADORA3 | 1.8E−38 | 385 |
| 53 | chr6 | 134497378 | 134497871 | SGK1 | 7.2E−61 | 385 |
| 54 | chr13 | 112575734 | 112575997 | LINC00354 | 7.2E−11 | 381 |
| 55 | chr5 | 549057 | 549217 | MIR4456 | 0.000021 | 381 |
| 56 | chr19 | 58446168 | 58446960 | ZNF418 | 4.1E−36 | 381 |
| 57 | chr8 | 143645555 | 143645722 | ARC | 0.0000011 | 376 |
| 58 | chr1 | 205818764 | 205819356 | PM20D1 | 1E−32 | 376 |
| 59 | chr10 | 134304477 | 134304614 | C10orf91 | 0.000037 | 369 |
| 60 | chr19 | 2607725 | 2608007 | MIR7850 | 1.1E−38 | 364 |
| 61 | chr6 | 170554026 | 170554222 | LOC154449 | 1 | 362 |
| 62 | chr19 | 1130738 | 1131134 | SBNO2 | 4.5E−14 | 355 |
| 63 | chr12 | 127211095 | 127211525 | LINC00943 | 1.7E−57 | 354 |
| 64 | chr17 | 78417763 | 78418214 | MIR4730 | 1.4E−16 | 347 |
| 65 | chr8 | 11607240 | 11607395 | C8orf49 | 1 | 346 |
| 66 | chr20 | 37356084 | 37357918 | SLC32A1 | 8.6E−90 | 340 |
| 67 | chr19 | 3435137 | 3435492 | SMIM24 | 1.1E−52 | 323 |
| 68 | chr14 | 106025695 | 106025886 | TMEM121 | 0.00000004 | 322 |
| 69 | chr2 | 235914654 | 235914887 | SH3BP4 | 0.000014 | 322 |
| 70 | chr8 | 132052209 | 132053847 | ADCY8 | 9.9E−58 | 302 |
| 71 | chr15 | 66332192 | 66332307 | MIR4311 | 1.3E−12 | 299 |
| 72 | chr12 | 130526741 | 130527219 | LOC100190940 | 1.3E−35 | 292 |
| 73 | chr13 | 40761973 | 40762567 | LINC00332 | 3.8E−55 | 291 |
| 74 | chr12 | 123560273 | 123560680 | LOC100507091 | 2.2E−19 | 290 |
| 75 | chr6 | 12292528 | 12292784 | EDN1 | 6.7E−21 | 278 |
| 76 | chr15 | 31175180 | 31175434 | FAN1 | 2.2E−17 | 273 |
| 77 | chr6 | 50818185 | 50818424 | TFAP2B | 1.1E−61 | 262 |
| 78 | chr10 | 1405102 | 1406162 | ADARB2-AS1 | 6.4E−217 | 258 |
| 79 | chr4 | 298742 | 299370 | ZNF732 | 1.9E−54 | 253 |
| 80 | chr6 | 26305227 | 26305876 | HIST1H4H | 1.6E−37 | 249 |
| 81 | chr6 | 78172191 | 78173096 | HTR1B | 1.4E−80 | 243 |
| 82 | chr7 | 5734834 | 5735261 | RNF216-IT1 | 4.2E−18 | 241 |
| 83 | chr2 | 168675039 | 168675400 | B3GALT1 | 0.00019 | 237 |
| 84 | chr8 | 144700415 | 144700607 | TSTA3 | 1.2E−09 | 236 |
| 85 | chr7 | 4832002 | 4832627 | MIR4656 | 1.9E−89 | 236 |
| 86 | chr3 | 48310428 | 48311036 | ZNF589 | 2.4E−49 | 234 |
| 87 | chr1 | 870922 | 871057 | SAMD11 | 1.6E−13 | 234 |
| 88 | chr8 | 1764777 | 1764878 | MIR596 | 1.2E−12 | 230 |
| 89 | chr6 | 170557442 | 170558257 | LOC154449 | 1.2E−51 | 226 |
| 90 | chr5 | 2750626 | 2751117 | IRX2 | 2E−48 | 224 |
| 91 | chr12 | 312594 | 312753 | LOC101929384 | 3E−38 | 222 |
| 92 | chr5 | 415418 | 415931 | EXOC3-AS1 | 1E−46 | 221 |
| 93 | chr19 | 50554366 | 50554491 | FLJ26850 | 1E−12 | 221 |
| 94 | chr12 | 127256640 | 127256977 | LINC00944 | 5.3E−12 | 219 |
| 95 | chr12 | 131171279 | 131171496 | STX2 | 4.1E−35 | 219 |
| 96 | chr4 | 153871339 | 153871959 | FHDC1 | 0.005 | 219 |
| 97 | chr1 | 148901649 | 148902388 | LOC101060524 | 6.4E−86 | 216 |
| 98 | chr4 | 206281 | 206944 | ZNF876P | 2.3E−84 | 216 |
| 99 | chr1 | 158899922 | 158900644 | PYHIN1 | 0.0098 | 215 |
| 100 | chr17 | 9129753 | 9129856 | LOC101928266 | 0.069 | 215 |
| 101 | chr3 | 196705554 | 196706015 | PIGZ | 5.4E−22 | 214 |
| 102 | chr8 | 2092991 | 2093307 | MIR7160 | 6.6E−24 | 214 |
| 103 | chr9 | 44842285 | 44842698 | FAM27C | 6.4E−18 | 214 |
| 104 | chr2 | 5832525 | 5834207 | SOX11 | 5.9E−129 | 213 |
| 105 | chr10 | 1576627 | 1576902 | ADARB2-AS1 | 0.19 | 212 |
| 106 | chr1 | 184633499 | 184633662 | EDEM3 | 2.6E−14 | 212 |
| 107 | chr9 | 138150754 | 138150839 | LOC401557 | 1.9E−13 | 211 |
| 108 | chr8 | 145638392 | 145639314 | SLC39A4 | 6E−117 | 211 |
| 109 | chr12 | 123616622 | 123616976 | PITPNM2 | 0.11 | 210 |
| 110 | chr10 | 2563715 | 2563880 | LINC00701 | 9.8E−11 | 210 |
| 111 | chr7 | 155151059 | 155151576 | BLACE | 2.5E−17 | 209 |
| 112 | chr9 | 138151031 | 138151147 | LOC401557 | 1.1E−15 | 208 |
| 113 | chr17 | 25798664 | 25799212 | KSR1 | 0.000000016 | 206 |
| 114 | chr2 | 130344892 | 130345228 | LOC151121 | 5.8E−28 | 205 |
| 115 | chr8 | 145003483 | 145004084 | PLEC | 5.3E−32 | 205 |
| 116 | chr11 | 1872541 | 1872947 | LSP1 | 1.1E−15 | 202 |
| 117 | chr10 | 95051 | 95429 | TUBB8 | 1.1E−24 | 202 |
| 118 | chr9 | 998920 | 999022 | DMRT3 | 1.2E−11 | 201 |
| 119 | chr1 | 160492736 | 160493089 | SLAMF6 | 3.2E−10 | 200 |
| 120 | chr10 | 3977395 | 3977757 | MIR6078 | 7.9E−25 | 200 |
| 121 | chr7 | 70597766 | 70598501 | WBSCR17 | 1.4E−42 | 200 |
| 122 | chr19 | 58545029 | 58545559 | ZSCAN1 | 2.5E−61 | 199 |
| 123 | chr6 | 6614384 | 6614830 | LY86-AS1 | 3.3E−36 | 199 |
| 124 | chr2 | 105372134 | 105372344 | LINC01114 | 0.0000057 | 196 |
| 125 | chr4 | 165304663 | 165304725 | MARCH1 | 1.1E−29 | 196 |
| 126 | chr19 | 35786456 | 35786698 | MAG | 1.5E−20 | 196 |
| 127 | chr15 | 37172579 | 37172961 | LOC145845 | 1.8E−34 | 194 |
| 128 | chr10 | 36054059 | 36054378 | PCAT5 | 8.1E−39 | 193 |
| 129 | chr19 | 18118303 | 18118626 | ARRDC2 | 0.000084 | 192 |
| 130 | chr10 | 104535448 | 104536195 | WBP1L | 4.6E−20 | 191 |
| 131 | chr11 | 60869600 | 60870250 | CD5 | 9.5E−10 | 191 |
| 132 | chr20 | 62178831 | 62179323 | SRMS | 1.1E−11 | 191 |
| 133 | chr7 | 1266142 | 1266532 | UNCX | 0.000046 | 190 |
| 134 | chr12 | 52994989 | 52995295 | KRT72 | 4.2E−36 | 189 |
| 135 | chr1 | 149137698 | 149138116 | | 1.1E−60 | 188 |
| 136 | chr4 | 8582875 | 8583216 | GPR78 | 1.9E−30 | 188 |
| 137 | chr7 | 75779491 | 75780140 | SRRM3 | 2.6E−30 | 187 |
| 138 | chr19 | 1496679 | 1496805 | REEP6 | 0.000041 | 186 |
| 139 | chr6 | 25027421 | 25028128 | FAM65B | 2.8E−18 | 182 |
| 140 | chr9 | 140772501 | 140772555 | CACNA1B | 0.01 | 181 |
| 141 | chr17 | 57028 | 57313 | LOC100506371 | 1.2E−22 | 181 |
| 142 | chr16 | 771651 | 771729 | FAM173A | 2.4E−14 | 179 |
| 143 | chr15 | 45669970 | 45671148 | GATM | 1.2E−80 | 178 |
| 144 | chr4 | 7483396 | 7483618 | MIR4274 | 0.0000018 | 177 |
| 145 | chr10 | 135051509 | 135051642 | VENTX | 0.029 | 172 |
| 146 | chr10 | 50819087 | 50820320 | SLC18A3 | 2.6E−113 | 172 |
| 147 | chr20 | 26188996 | 26189062 | MIR663A | 0.000082 | 171 |
| 148 | chr2 | 242843594 | 242844819 | LINC01237 | 3.9E−57 | 170 |
| 149 | chr1 | 161441286 | 161441635 | FCGR2A | 0.51 | 168 |
| 150 | chr10 | 133918088 | 133918337 | JAKMIP3 | 0.0000024 | 167 |
| 151 | chr19 | 15391831 | 15392012 | BRD4 | 0.000000045 | 164 |
| 152 | chr7 | 151553549 | 151553813 | PRKAG2-AS1 | 0.00000031 | 163 |
| 153 | chr19 | 52956621 | 52957010 | ZNF578 | 1.6E−18 | 162 |
| 154 | chr11 | 1873744 | 1874017 | LSP1 | 0.06 | 161 |
| 155 | chr20 | 5297722 | 5297906 | PROKR2 | 0.000015 | 161 |
| 156 | chr8 | 141275106 | 141275300 | TRAPPC9 | 0.00003 | 152 |
| 157 | chr2 | 100937909 | 100938596 | LONRF2 | 2.1E−179 | 148 |
| 158 | chr16 | 86066512 | 86066907 | MIR6774 | 0.000000033 | 148 |
| 159 | chr19 | 58549486 | 58549813 | ZSCAN1 | 3.6E−09 | 146 |
| 160 | chr14 | 106026029 | 106026161 | TMEM121 | 0.0000019 | 140 |
| 161 | chr16 | 33039528 | 33040508 | SLC6A10P | 5.5E−96 | 138 |
| 162 | chr12 | 72666548 | 72667095 | TRHDE | 2.1E−70 | 136 |
| 163 | chr20 | 56283770 | 56284016 | PMEPA1 | 3.2E−11 | 135 |
| 164 | chr13 | 95364048 | 95364786 | SOX21 | 5.2E−72 | 134 |
| 165 | chr14 | 105944654 | 105944897 | CRIP2 | 7.2E−40 | 125 |
| 166 | chr4 | 6247085 | 6247744 | WFS1 | 9E−56 | 125 |
| 167 | chr1 | 235105763 | 235105928 | LOC101927851 | 6.8E−32 | 123 |
| 168 | chr9 | 101594116 | 101594307 | GALNT12 | 0.00000053 | 119 |
| 169 | chr14 | 103415848 | 103415938 | AMN | 8.2E−10 | 119 |
| 170 | chr1 | 38461539 | 38462052 | SF3A3 | 3.3E−68 | 117 |
| 171 | chr17 | 10632639 | 10632934 | TMEM220-AS1 | 9.8E−23 | 116 |
| 172 | chr13 | 19918970 | 19919335 | LINC00421 | 3.7E−24 | 115 |
| 173 | chr9 | 132382203 | 132383155 | NTMT1 | 1.5E−82 | 114 |
| 174 | chr16 | 51183896 | 51185814 | SALL1 | 2.1E−126 | 113 |
| 175 | chr20 | 61877854 | 61877956 | FLJ16779 | 0.018 | 113 |
| 176 | chr2 | 8422081 | 8422382 | LINC00299 | 1.7E−20 | 112 |
| 177 | chr14 | 105318120 | 105318552 | CEP170B | 0.00000038 | 110 |
| 178 | chr8 | 142401477 | 142401672 | GPR20 | 1.7E−19 | 109 |
| 179 | chr4 | 2305632 | 2305758 | MXD4 | 0.23 | 109 |
| 180 | chr2 | 112123586 | 112124284 | | 2.7E−43 | 107 |
| 181 | chr7 | 158766060 | 158766499 | LINC00689 | 1.6E−49 | 107 |
| 182 | chr17 | 80840672 | 80841028 | ZNF750 | 9E−21 | 107 |
| 183 | chr8 | 1950728 | 1951319 | KBTBD11 | 5.3E−64 | 106 |
| 184 | chr6 | 27114453 | 27114572 | HIST1H2BK | 2.2E−16 | 104 |
| 185 | chr20 | 25063952 | 25064652 | VSX1 | 7.6E−57 | 103 |
| 186 | chr5 | 140166058 | 140166202 | PCDHA1 | 0.00000007 | 100 |
| 187 | chr10 | 105678023 | 105678128 | OBFC1 | 0.0000002 | 97 |
| 188 | chr16 | 88263173 | 88263261 | LOC101928880 | 0.00014 | 97 |
| 189 | chr17 | 27043962 | 27044677 | RAB34 | 1.8E−80 | 96 |
| 190 | chr12 | 43944719 | 43946284 | ADAMTS20 | 1.1E−111 | 96 |
| 191 | chr10 | 133142601 | 133142686 | TCERG1L | 0.000000034 | 95 |
| 192 | chr5 | 33936457 | 33937593 | RXFP3 | 1.3E−27 | 94 |
| 193 | chr20 | 50108656 | 50109526 | MIR3194 | 6.6E−52 | 91 |
| 194 | chr6 | 15504813 | 15505122 | DTNBP1 | 2E−17 | 90 |
| 195 | chr9 | 34577971 | 34578054 | CNTFR-AS1 | 6.8E−12 | 90 |
| 196 | chr19 | 56988313 | 56989827 | ZNF667-AS1 | 3.3E−129 | 87 |
| 197 | chr1 | 24861601 | 24861919 | LOC100506985 | 6.5E−28 | 85 |
| 198 | chr20 | 48626436 | 48626621 | SNAI1 | 4.8E−20 | 84 |
| 199 | chr3 | 6902823 | 6903618 | GRM7 | 5.5E−94 | 84 |
| 200 | chr1 | 17025888 | 17026534 | | 3.5E−62 | 83 |
| 201 | chr16 | 33852624 | 33852723 | LINC00273 | 5.6E−28 | 83 |
| 202 | chr8 | 143841836 | 143842247 | LYPD2 | 7.3E−28 | 79 |
| 203 | chr19 | 22805744 | 22806540 | ZNF492 | 4.6E−63 | 78 |
| 204 | chr12 | 126675576 | 126676034 | LOC101927464 | 7.8E−38 | 77 |
| 205 | chr16 | 33961213 | 33961464 | LINC00273 | 6.8E−40 | 76 |
| 206 | chr19 | 33726527 | 33726834 | SLC7A10 | 7.3E−21 | 75 |
| 207 | chr2 | 239756518 | 239757924 | TWIST2 | 4.9E−126 | 72 |
| 208 | chr13 | 27927276 | 27927676 | GTF3A | 2E−16 | 69 |
| 209 | chr19 | 38345731 | 38346078 | LOC100631378 | 4.2E−35 | 57 |
| 210 | chr8 | 145955929 | 145955988 | ZNF251 | 0.003 | 55 |
| 211 | chr16 | 15820625 | 15820916 | NDE1 | 7.1E−09 | 55 |
| 212 | chr3 | 196373437 | 196373657 | NRROS | 0.00068 | 53 |
| 213 | chr8 | 494821 | 494884 | TDRP | 1.6E−12 | 52 |
| 214 | chr7 | 6704023 | 6704174 | ZNF316 | 6.9E−12 | 51 |
| 215 | chr1 | 1109468 | 1109757 | TTLL10 | 4.5E−26 | 49 |
| 216 | chr17 | 15820948 | 15821270 | ADORA2B | 3.1E−17 | 48 |
| 217 | chr8 | 55366735 | 55367119 | SOX17 | 0.0000011 | 48 |
| 218 | chr21 | 45149027 | 45149226 | PDXK | 1.6E−10 | 47 |
| 219 | chr12 | 127212444 | 127212519 | LINC00943 | 8E−19 | 45 |
| 220 | chr12 | 133049696 | 133050063 | FBRSL1 | 4.2E−23 | 45 |
| 221 | chr13 | 70681641 | 70682366 | KLHL1 | 3.6E−38 | 45 |
| 222 | chr14 | 105996302 | 105996413 | TMEM121 | 4.3E−23 | 41 |
| 223 | chr11 | 22362862 | 22363249 | SLC17A6 | 7E−30 | 37 |
| 224 | chr7 | 153584345 | 153585592 | DPP6 | 2.5E−104 | 36 |
| 225 | chr2 | 225265848 | 225266563 | FAM124B | 1.4E−20 | 36 |
| 226 | chr19 | 58629900 | 58630005 | ZSCAN18 | 0.000000007 | 36 |
| 227 | chr7 | 94953781 | 94953889 | PON1 | 0.000000018 | 36 |
| 228 | chr9 | 71788570 | 71789667 | TJP2 | 4.6E−108 | 35 |
| 229 | chr10 | 128594721 | 128594812 | DOCK1 | 1.4E−10 | 33 |
| 230 | chr7 | 105319313 | 105319905 | ATXN7L1 | 5.2E−26 | 32 |
| 23 | chr1 | 226730366 | 226730729 | C1orf95 | 0.00000074 | 32 |
| 232 | chr19 | 53561167 | 53561713 | ERVV-2 | 2.6E−36 | 32 |
| 233 | chr9 | 842394 | 843099 | DMRT1 | 7.5E−54 | 31 |
| 234 | chr9 | 136566510 | 136566953 | SARDH | 1E−42 | 30 |
| 235 | chr15 | 29034604 | 29034806 | PDCD6IPP2 | 0.0000035 | 30 |
| 236 | chr3 | 71545400 | 71545754 | MIR1284 | 0.000000046 | 30 |
| 237 | chr14 | 93153235 | 93153550 | LGMN | 4.3E−48 | 30 |
| 238 | chr4 | 62066851 | 62068609 | MIR548AG1 | 4.2E−49 | 28 |
| 239 | chr19 | 18760502 | 18760995 | KLHL26 | 1.7E−21 | 26 |
| 240 | chr2 | 240168506 | 240169412 | MGC16025 | 1.1E−33 | 26 |
| 241 | chr17 | 56401622 | 56401992 | BZRAP1-AS1 | 2.7E−54 | 25 |
| 242 | chr19 | 6659931 | 6660186 | TNFSF14 | 2E−12 | 25 |
| 243 | chr5 | 177398162 | 177398535 | PROP1 | 2.5E−46 | 24 |
| 244 | chr10 | 50323691 | 50323792 | VSTM4 | 4.1E−13 | 24 |
| 245 | chr9 | 998644 | 998958 | DMRT3 | 0.0014 | 24 |
| 246 | chr5 | 176559286 | 176559400 | NSD1 | 2E−13 | 23 |
| 247 | chr19 | 22989927 | 22990607 | ZNF99 | 4.4E−70 | 23 |
| 248 | chr12 | 54763222 | 54763387 | GPR84 | 0.0083 | 23 |
| 249 | chr18 | 77155749 | 77160758 | NFATC1 | 0.00028 | 23 |
| 250 | chr7 | 101961741 | 101962019 | MIR4285 | 2.8E−65 | 22 |
| 251 | chr1 | 203320137 | 203320661 | FMOD | 4.2E−16 | 22 |
| 252 | chr16 | 46462413 | 46462697 | ANKRD26P1 | 1.5E−53 | 21 |
| 253 | chr7 | 54612184 | 54612557 | VSTM2A | 2.6E−23 | 21 |
| 254 | chr14 | 23835562 | 23835859 | EFS | 0.00000022 | 20 |
| 255 | chr16 | 48844551 | 48845153 | N4BP1 | 2.7E−55 | 20 |
| 256 | chr13 | 95354189 | 95354282 | LOC101927248 | 0.000011 | 20 |
| 257 | chr19 | 10404842 | 10405309 | ICAM5 | 2.7E−54 | 19 |
| 258 | chr4 | 568854 | 568909 | PDE6B | 3.6E−10 | 19 |
| 259 | chr16 | 79692311 | 79692516 | MAF | 8.5E−15 | 19 |
| 260 | chr5 | 150521132 | 150521473 | ANXA6 | 0.53 | 17 |
| 261 | chr5 | 42944099 | 42944683 | FLJ32255 | 3.9E−30 | 17 |
| 262 | chr12 | 128752535 | 128753138 | TMEM132C | 1.5E−44 | 16 |
| 263 | chr13 | 109147997 | 109149018 | MYO16 | 1.4E−86 | 15 |
| 264 | chr8 | 38831542 | 38832432 | HTRA4 | 3.2E−76 | 15 |
| 265 | chr6 | 436571 | 436756 | IRF4 | 4.2E−10 | 15 |
| 266 | chr7 | 57928602 | 57928726 | ZNF716 | 0.0000039 | 15 |
| 267 | chr4 | 5894295 | 5894775 | CRMP1 | 3.2E−86 | 15 |
| 268 | chr7 | 63642022 | 63642692 | ZNF735 | 1.4E−65 | 15 |
| 269 | chr9 | 19934400 | 19934565 | SLC24A2 | 1.9E−17 | 14 |
| 270 | chr1 | 237206274 | 237206500 | RYR2 | 1.6E−27 | 14 |
| 271 | chr2 | 242973561 | 242973869 | LOC728323 | 0.016 | 13 |
| 272 | chr6 | 25882171 | 25882633 | SLC17A3 | 2.4E−20 | 13 |
| 273 | chr16 | 3114846 | 3115906 | IL32 | 2.2E−33 | 13 |
| 274 | chr16 | 73516908 | 73517648 | LINC01568 | 2.3E−21 | 13 |
| 275 | chr16 | 771788 | 772078 | FAM173A | 5.3E−38 | 13 |
| 276 | chr5 | 128300670 | 128301083 | SLC27A6 | 3.5E−34 | 12 |
| 277 | chr15 | 27216224 | 27216842 | GABRG3 | 5.9E−86 | 12 |
| 278 | chr20 | 41818612 | 41818643 | PTPRT | 0.024 | 12 |
| 279 | chr5 | 179192382 | 179192752 | LTC4S | 0.0000002 | 11 |
| 280 | chr16 | 2214161 | 2214180 | TRAF7 | 0.013 | 11 |
| 281 | chr2 | 132202426 | 132202537 | LOC401010 | 0.000012 | 10 |
| 282 | chr9 | 13278349 | 13278785 | MPDZ | 7.3E−45 | 10 |
| 283 | chr17 | 48154015 | 48154485 | PDK2 | 7E−14 | 10 |
| 284 | chr7 | 53286811 | 53287070 | POM121L12 | 4.9E−21 | 10 |
| 285 | chr7 | 73623955 | 73624529 | LAT2 | 4.8E−15 | 10 |
| 286 | chr6 | 109776070 | 109776252 | MICAL1 | 6.4E−16 | 9 |
| 287 | chr6 | 144329093 | 144329329 | PLAGL1 | 9.3E−32 | 9 |
| 288 | chr5 | 178421527 | 178422157 | GRM6 | 9E−87 | 9 |
| 289 | chr11 | 33562940 | 33563598 | KIAA1549L | 0.00016 | 9 |
| 290 | chr21 | 45773781 | 45774407 | TRPM2 | 1.7E−16 | 9 |
| 291 | chr13 | 114905582 | 114905873 | RASA3 | 0.0000093 | 8 |
| 292 | chr20 | 31618058 | 31618529 | BPIFB6 | 8.3E−10 | 8 |
| 293 | chr5 | 33937599 | 33938284 | RXFP3 | 4.6E−90 | 8 |
| 294 | chr19 | 39261775 | 39262344 | LGALS7 | 2E−12 | 8 |
| 295 | chr22 | 44258036 | 44258326 | SULT4A1 | 4.6E−39 | 8 |
| 296 | chr17 | 48585254 | 48585434 | MYCBPAP | 0.00054 | 8 |
| 297 | chr11 | 72379866 | 72380940 | PDE2A | 0.000099 | 8 |
| 298 | chr13 | 114964475 | 114964694 | CDC16 | 1.6E−40 | 7 |
| 299 | chr12 | 133050235 | 133050359 | FBRSL1 | 0.000000078 | 7 |
| 300 | chr5 | 140237056 | 140237453 | PCDHA10 | 5.8E−46 | 7 |
| 301 | chr1 | 228772137 | 228772363 | | 6.3E−13 | 7 |
| 302 | chr11 | 69482205 | 69482550 | ORAOV1 | 2.2E−09 | 7 |
| 303 | chr13 | 113764277 | 113764376 | F7 | 2.8E−09 | 6 |
| 304 | chr5 | 140531178 | 140531491 | PCDHB6 | 1.6E−30 | 6 |
| 305 | chr15 | 29213249 | 29213965 | APBA2 | 0.0015 | 6 |
| 306 | chr17 | 33775342 | 33776150 | SLFN13 | 2.2E−44 | 6 |
| 307 | chr13 | 53313142 | 53313686 | LECT1 | 2.7E−25 | 6 |
| 308 | chr9 | 100616468 | 100617303 | FOXE1 | 3.5E−75 | 5 |
| 309 | chr14 | 101012788 | 101012852 | BEGAIN | 8.7E−10 | 5 |
| 310 | chr11 | 107799071 | 107799145 | RAB39A | 4.6E−09 | 5 |
| 311 | chr13 | 110966129 | 110966348 | COL4A2 | 1 | 5 |
| 312 | chr5 | 177433176 | 177433484 | FAM153C | 0.00000031 | 5 |
| 313 | chr8 | 22423399 | 22423478 | SORBS3 | 7.9E−10 | 5 |
| 314 | chr7 | 63642578 | 63643893 | ZNF735 | 2E−13 | 5 |
| 315 | chr2 | 10182524 | 10183208 | KLF11 | 1E−31 | 4 |
| 316 | chr4 | 109708517 | 109708812 | ETNPPL | 0.0013 | 4 |
| 317 | chr5 | 155108451 | 155108512 | SGCD | 8.6E−09 | 4 |
| 318 | chr20 | 21686714 | 21687523 | PAX1 | 5.5E−61 | 4 |
| 319 | chr2 | 233981748 | 233982007 | INPP5D | 8.8E−14 | 4 |
| 320 | chr6 | 116691966 | 116692094 | DSE | 3.2E−38 | 3 |
| 321 | chr5 | 127872601 | 127873556 | FBN2 | 1.7E−84 | 3 |
| 322 | chr3 | 129024511 | 129024861 | H1FX-AS1 | 3.9E−16 | 3 |
| 323 | chr16 | 32822775 | 32823165 | SLC6A10P | 2.5E−72 | 3 |
| 324 | chr19 | 37463400 | 37463629 | ZNF568 | 3.5E−12 | 3 |
| 325 | chr7 | 4848511 | 4848640 | MIR4656 | 0.0000039 | 3 |
| 326 | chr20 | 51648719 | 51648963 | TSHZ2 | 0.00000022 | 3 |
| 327 | chr20 | 61371295 | 61371912 | NTSR1 | 1E−37 | 3 |
| 328 | chr11 | 627289 | 627351 | SCT | 0.00092 | 3 |
| 329 | chr10 | 106401221 | 106401269 | SORCS3 | 3.4E−13 | 2 |
| 330 | chr4 | 1399872 | 1400794 | NKX1-1 | 8.8E−87 | 2 |
| 331 | chr8 | 142613052 | 142613437 | MROH5 | 0.000048 | 2 |
| 332 | chr7 | 149917788 | 149918384 | ACTR3C | 4.1E−25 | 2 |
| 333 | chr5 | 150325904 | 150326033 | ZNF300P1 | 0.000000001 | 2 |
| 334 | chr4 | 187549313 | 187549585 | MTNR1A | 0.047 | 2 |
| 335 | chr2 | 202900530 | 202900642 | FZD7 | 9.3E−12 | 2 |
| 336 | chr19 | 22966554 | 22967007 | ZNF99 | 2.8E−30 | 2 |
| 337 | chr2 | 241459543 | 241460046 | ANKMY1 | 3.2E−33 | 2 |
| 338 | chr17 | 25289742 | 25290204 | MIR4522 | 5.4E−29 | 2 |
| 339 | chr16 | 33070873 | 33071036 | | 8.3E−21 | 2 |
| 340 | chr20 | 33134749 | 33135071 | MAP1LC3A | 1.1E−11 | 2 |
| 341 | chr6 | 37617723 | 37618064 | MDGA1 | 2.2E−31 | 2 |
| 342 | chr5 | 41509744 | 41510165 | PLCXD3 | 3.4E−24 | 2 |
| 343 | chr7 | 5594530 | 5594721 | ACTB | 1.7E−21 | 2 |
| 344 | chr7 | 56355446 | 56355797 | LOC650226 | 5.4E−40 | 2 |
| 345 | chr7 | 56605483 | 56605719 | LOC101928401 | 0.0014 | 2 |
| 346 | chr20 | 57089732 | 57090023 | APCDD1L | 1.6E−35 | 2 |
| 347 | chr13 | 58206330 | 58206472 | PCDH17 | 5.8E−18 | 2 |
| 348 | chr19 | 58629190 | 58629803 | ZSCAN18 | 6.8E−46 | 2 |
| 349 | chr20 | 62079508 | 62079911 | KCNQ2 | 1.7E−18 | 2 |
| 350 | chr8 | 65281959 | 65282932 | LOC102724623 | 2.5E−88 | 2 |
| 351 | chr2 | 66808615 | 66809107 | LOC100507073 | 7.9E−28 | 2 |
| 352 | chr7 | 70596771 | 70597762 | WBSCR17 | 8.1E−97 | 2 |
| 353 | chr2 | 10184491 | 10184638 | KLF11 | 7.8E−11 | 1 |
| 354 | chr6 | 110300487 | 110301094 | GPR6 | 6.8E−38 | 1 |
| 355 | chr10 | 119494392 | 119495015 | EMX2OS | 1.5E−33 | 1 |
| 356 | chr2 | 121670197 | 121670819 | GLI2 | 7.9E−17 | 1 |
| 357 | chr11 | 122847581 | 122847621 | BSX | 7.4E−12 | 1 |
| 358 | chr19 | 12876798 | 12877235 | HOOK2 | 1.6E−15 | 1 |
| 359 | chr12 | 130647670 | 130648336 | FZD10 | 4.2E−71 | 1 |
| 360 | chr9 | 136004665 | 136004977 | RALGDS | 9.6E−12 | 1 |
| 361 | chr2 | 1370349 | 1370660 | TPO | 8.3E−13 | 1 |
| 362 | chr5 | 140346483 | 140346876 | PCDHAC2 | 2.7E−55 | 1 |
| 363 | chr9 | 140356754 | 140356922 | NSMF | 1.5E−10 | 1 |
| 364 | chr5 | 140536896 | 140537360 | PCDHB17P | 3.8E−25 | 1 |
| 365 | chr5 | 140554032 | 140554096 | PCDHB7 | 2.3E−14 | 1 |
| 366 | chr7 | 154795072 | 154795177 | PAXIP1-AS1 | 0.00000021 | 1 |
| 367 | chr17 | 1686633 | 1686905 | SERPINF1 | 9.8E−31 | 1 |
| 368 | chr22 | 17600837 | 17600960 | CECR6 | 2.2E−18 | 1 |
| 369 | chr14 | 21494160 | 21494269 | NDRG2 | 1.2E−10 | 1 |
| 370 | chr20 | 21695251 | 21695308 | PAX1 | 0.000064 | 1 |
| 371 | chr10 | 27235407 | 27235752 | LINC00202-1 | 3E−19 | 1 |
| 372 | chr22 | 38485160 | 38485375 | SLC16A8 | 5.6E−11 | 1 |
| 373 | chr20 | 43247163 | 43247345 | ADA | 0.0000073 | 1 |
| 374 | chr22 | 43738753 | 43738821 | SCUBE1 | 1.1E−32 | 1 |
| 375 | chr20 | 56247249 | 56247617 | PMEPA1 | 2.8E−10 | 1 |
| 376 | chr8 | 57069543 | 57069853 | MOS | 2.9E−16 | 1 |
| 377 | chr16 | 57680024 | 57680206 | ADGRG1 | 0.000012 | 1 |
| 378 | chr7 | 57928343 | 57928395 | ZNF716 | 7.3E−13 | 1 |
| 379 | chr3 | 69591948 | 69592049 | FRMD4B | 9.1E−10 | 1 |
| 380 | chr13 | 79161066 | 79161721 | POU4F1 | 5.4E−16 | 1 |
| 381 | chr12 | 81471418 | 81472263 | ACSS3 | 2.7E−45 | 1 |

### Treatments

In embodiments, the methods described herein comprise treating a patient for cancer. In embodiments, treating a patient for cancer comprises surgically removing the cancer from the patient, administering to the patient an effective amount of radiotherapy, administering to the patient an effective amount of an anti-cancer agent, or a combination of two or more thereof. In embodiments, treating a patient for cancer comprises surgically removing the cancer from the patient, administering to the patient an effective amount of an anti-cancer agent, or a combination thereof. In embodiments, treating a patient for cancer comprises administering to the patient an effective amount of an anti-cancer agent. In embodiments, the anti-cancer agent is radiotherapy, immunotherapy, targeted therapy, chemotherapy, or a combination of two or more thereof. In embodiments, the anti-cancer agent is immunotherapy, targeted therapy, chemotherapy, or a combination of two or more thereof. In embodiments, treating a patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the methods described herein comprise surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof. In embodiments, the methods comprise surgically removing the cancer from the patient. In embodiments, the methods comprise administering to the patient an effective amount of radiotherapy. In embodiments, the methods comprise administering to the patient an effective amount of chemotherapy. In embodiments, the methods comprise administering to the patient an effective amount of targeted therapy. In embodiments, the methods comprise administering to the patient an effective amount of immunotherapy. In embodiments, the methods described herein comprise surgically removing the cancer from the patient and administering to the patient an effective amount of chemotherapy. In embodiments, the methods described herein comprise surgically removing the cancer from the patient, administering to the patient an effective amount of chemotherapy, administering to the patient an effective amount of targeted therapy, and administering to the patient an effective amount of immunotherapy. In embodiments, the methods described herein comprise administering to the patient an effective amount of chemotherapy, administering to the patient an effective amount of targeted therapy, and administering to the patient an effective amount of immunotherapy. In embodiments, the methods described herein comprise administering to the patient an effective amount of chemotherapy and administering to the patient an effective amount of targeted therapy. In embodiments, the methods described herein comprise administering to the patient an effective amount of chemotherapy and administering to the patient an effective amount of immunotherapy. In embodiments, the methods described herein comprise administering to the patient an effective amount of targeted therapy and administering to the patient an effective amount of immunotherapy.

In embodiments of the methods described herein, the chemotherapy is any chemotherapy known in the art. In embodiments, the chemotherapy comprises 5-fluorouracil, leucovorin, oxaliplatin, irinotecan, capecitabine, docetaxel, doxorubicin, or a combination of two or more thereof. In embodiments, the chemotherapy comprises an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof. In embodiments, the alkylating agent is carboplatin, chlorambucil, cyclophosphamide, melphalan, mechlorethamine, procarbazine, or thiotepa. In embodiments, the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine. In embodiments, the anthracycline compound is daunorubicin, doxorubicin, idarubicin, epirubicin, or mitoxantrone. In embodiments, the antitumor antibiotic is actinomycin, bleomycin, mitomycin, or valrubicin. In embodiments, the platinum compound is cisplatin or oxaliplatin. In embodiments, the topoisomerase inhibitor is irinotecan, topotecan, amsacrine, etoposide, teniposide, or eribulin. In embodiments, the vinca alkaloid is vincristine, vinblastine, vinorelbine, or vindesine. In embodiments, the taxane compound is paclitaxel or docetaxel. In embodiments, the epothilone compound is epothilone, ixabepilone, patupilone, or sagopilone.

In embodiments of the methods described herein, the immunotherapy is any immunotherapy known in the art. In embodiments, the immunotherapy is a checkpoint inhibitor. In embodiments, the immunotherapy comprises a PD-1 inhibitor, a PD-L1 inhibitor, a CTLA-4 inhibitor, a LAG-3 inhibitor, or a combination of two or more thereof. In embodiments, the immunotherapy comprises a PD-1 inhibitor. In embodiments, the PD-1 inhibitor is pembrolizumab, nivolumab, cemiplimab, dostarlimab, sparlalizumab, camrelizumab, sintilimab, tiselizumab, or toripalimab. In embodiments, the PD-1 inhibitor is pembrolizumab, nivolumab, cemiplimab, or dostarlimab. In embodiments, the immunotherapy comprises a PD-L1 inhibitor.

In embodiments, the PD-L1 inhibitor is atezolizumab, avelumab, or durvalumab. In embodiments, the immunotherapy comprises a CTLA-4 inhibitor. In embodiments, the CTLA-4 inhibitor is ipilimumab. In embodiments, the immunotherapy comprises a LAG-3 inhibitor. In embodiments, the LAG-3 inhibitor is relatlimab. In embodiments, the immunotherapy comprises pembrolizumab, nivolumab, cemiplimab, dostarlimab, sparlalizumab, camrelizumab, sintilimab, tiselizumab, toripalimab, ipilimumab, atezolizumab, avelumab, durvalumab, relatlimab, or a combination of two or more thereof. In embodiments, the immunotherapy comprises pembrolizumab, nivolumab, cemiplimab, dostarlimab, ipilimumab, atezolizumab, avelumab, durvalumab, relatlimab, or a combination of two or more thereof. In embodiments of the methods described herein, the targeted therapy is any targeted therapy known in the art. In embodiments, the targeted therapy is a multi-kinase inhibitor. In embodiments, the targeted therapy is ramucirumab, trastuzumab, dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib, or a combination of two or more thereof.

In embodiments of the methods described herein, the targeted therapy is any targeted therapy known in the art. In embodiments, the targeted therapy is ramucirumab, trastuzumab, dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, sorafenib, vorinostat, romidepsin, tacedinaline, belinostat, panobinostat, givinostat, entinostat, mocetinostat, resveratrol, quisinostat, abexinostat, azacitidine, decitabine, pinometostat, pargyline, tranylcypromine, 5-chloro-2-(4-nitrophenyl)-3(2H)-isothiazolone (CCT077791), garcinol, or a combination of two or more thereof. In embodiments, the targeted therapy is a multi-kinase inhibitor or an epigenetic inhibitor.

In embodiments, the targeted therapy is a multi-kinase inhibitor. In embodiments, the multi-kinase inhibitor is a therapeutic agent that targets the VEGFNEGFR pathway, the EGFR pathway, the VEGFNEGFR2 pathway, or the HER2 pathway. In embodiments, the multi-kinase inhibitor is a therapeutic agent that targets the VEGFNEGFR pathway. In embodiments, the multi-kinase inhibitor is a therapeutic agent that targets the EGFR pathway. In embodiments, the multi-kinase inhibitor is a therapeutic agent that targets the VEGFNEGFR2 pathway. In embodiments, the multi-kinase inhibitor is a therapeutic agent that targets the HER2 pathway. In embodiments, the multi-kinase inhibitor is ramucirumab, trastuzumab, dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib. In embodiments, the multi-kinase inhibitor is a therapeutic agent that targets the VEGFNEGFR pathway, the EGFR pathway, the VEGF/VEGFR2 pathway, or the HER2 pathway. In embodiments, the multi-kinase inhibitor is a therapeutic agent that targets the VEGF/VEGFR pathway. In embodiments, the multi-kinase inhibitor is a therapeutic agent that targets the EGFR pathway. In embodiments, the multi-kinase inhibitor is a therapeutic agent that targets the VEGF/VEGFR2 pathway. In embodiments, the multi-kinase inhibitor is a therapeutic agent that targets the HER2 pathway.

In embodiments, the targeted therapy is an epigenetic inhibitor. In embodiments, the epigenetic inhibitor is a histone-deacetylase inhibitor, a DNA methyltransferase inhibitor, a histone methyltransferase inhibitor, a histone demethylase inhibitor, a histone acetyltransferase inhibitor, or a combination of two or more thereof. In embodiments, the epigenetic inhibitor is a histone-deacetylase inhibitor. In embodiments, the epigenetic inhibitor is a DNA methyltransferase inhibitor. In embodiments, the epigenetic inhibitor is a histone methyltransferase inhibitor. In embodiments, the epigenetic inhibitor is a histone demethylase inhibitor. In embodiments, the epigenetic inhibitor is a histone acetyltransferase inhibitor. In embodiments, the histone-deacetylase inhibitor is vorinostat, romidepsin, tacedinaline, belinostat, panobinostat, givinostat, entinostat, mocetinostat, resveratrol, quisinostat, or abexinostat. In embodiments, the DNA methyltransferase inhibitor is azacitidine and decitabine. In embodiments, the histone methyltransferase inhibitor is pinometostat. In embodiments, the histone demethylase inhibitor is pargyline or tranylcypromine. In embodiments, the histone acetyltransferase inhibitor is 5-chloro-2-(4-nitrophenyl)-3(2H)-isothiazolone (CCT077791) or garcinol. In embodiments, the epigenetic inhibitor is vorinostat, romidepsin, tacedinaline, belinostat, panobinostat, givinostat, entinostat, mocetinostat, resveratrol, quisinostat, abexinostat, azacitidine, decitabine, pinometostat, pargyline, tranylcypromine, 5-chloro-2-(4-nitrophenyl)-3(2H)-isothiazolone (CCT077791), or garcinol.

“Chemotherapy” is a type of cancer treatment that uses one or more anti-cancer drugs (e.g. chemotherapeutic agents) as part of a standardized chemotherapy regimen. The use of drugs constitutes “systemic therapy” or “systemic chemotherapy” for cancer in that they are introduced into the blood stream and are therefore in principle able to address cancer at any anatomic location in the body. In embodiments of the methods described herein, the chemotherapy is systemic chemotherapy. Systemic therapy is often used in conjunction with other modalities that constitute local therapy (i.e. treatments whose efficacy is confined to the anatomic area where they are applied) for cancer such as radiation therapy, surgery or hyperthermia therapy.

“Radiation therapy” or “radiotherapy” refer to a therapy using ionizing radiation, generally as part of cancer treatment to control or kill malignant cells and normally delivered by a linear accelerator. Radiation therapy may be curative in a number of types of cancer if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and has been used before, during, and after chemotherapy in susceptible cancers. The subspecialty of oncology concerned with radiotherapy is called radiation oncologist.

“Immunotherapy” refers to the treatment of disease by activating or suppressing the immune system. In the context of cancer, a cancer immunotherapy refers to the artificial stimulation of the immune system to treat cancer, improving on the immune system's natural ability to fight the disease. Cancer immunotherapy exploits the fact that cancer cells often have tumor antigens, molecules on their surface that can be detected by the antibody proteins of the immune system, binding to them. The tumor antigens are often proteins or other macromolecules (e.g., carbohydrates). Normal antibodies bind to external pathogens, but the modified immunotherapy antibodies bind to the tumor antigens marking and identifying the cancer cells for the immune system to inhibit or kill.

“Targeted therapy” refers to the use of a drug or drugs or other substances to block the growth and spread of cancer by interfering with specific target molecules or pathways that are involved in the growth, progression, and spread of cancer. In embodiments, targeted therapy is a multi-kinase inhibitor, an epigenetic inhibitor, or a combination thereof. In embodiments, targeted therapy is a multi-kinase inhibitor. In embodiments, targeted therapy is an epigenetic inhibitor.

A “multi-kinase inhibitor” is a small molecule inhibitor of at least one protein kinase, including tyrosine protein kinases and serine/threonine kinases. A multi-kinase inhibitor may include a single kinase inhibitor. Multi-kinase inhibitors may block phosphorylation. Multi-kinases inhibitors may act as covalent modifiers of protein kinases. Multi-kinase inhibitors may bind to the kinase active site or to a secondary or tertiary site inhibiting protein kinase activity. A multi-kinase inhibitor may be an anti-cancer multi-kinase inhibitor. Exemplary anti-cancer multi-kinase inhibitors include ramucirumab, trastuzumab, dasatinib, sunitinib, erlotinib, bevacizumab, vatalanib, vemurafenib, vandetanib, cabozantinib, poatinib, axitinib, ruxolitinib, regorafenib, crizotinib, bosutinib, cetuximab, gefitinib, imatinib, lapatinib, lenvatinib, mubritinib, nilotinib, panitumumab, pazopanib, trastuzumab, or sorafenib. In embodiments, the multi-kinase inhibitor targets the VEGF/VEGFR pathway, the EGFR pathway the VEGF/VEGFR2 pathway, or the HER2 pathway.

An “epigenetic inhibitor” as used herein, refers to an inhibitor of an epigenetic process, such as DNA methylation (a DNA methylation Inhibitor) or modification of histones (a Histone Modification Inhibitor). An epigenetic inhibitor may be a histone-deacetylase (HDAC) inhibitor, a DNA methyltransferase (DNMT) inhibitor, a histone methyltransferase (HMT) inhibitor, a histone demethylase (HDM) inhibitor, or a histone acetyltransferase (HAT). Examples of HDAC inhibitors include vorinostat, romidepsin, CI-994, belinostat, panobinostat, givinostat, entinostat, mocetinostat, SRT501, CUDC-101, JNJ-26481585, or PCI24781. Examples of DNMT inhibitors include azacitidine and decitabine. Examples of HMT inhibitors include pinometostat (EPZ-5676). Examples of HDM inhibitors include pargyline and tranylcypromine. Examples of HAT inhibitors include 5-chloro-2-(4-nitrophenyl)-3(2H)-isothiazolone (CCT077791) and garcinol.

The terms “treating” or “treatment” refer to any indicia of clinical success in the therapy or amelioration of a disease (e.g., cancer), including any objective or subjective parameter such as abatement; remission; diminishing of symptoms or making the condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination. “Treating” does not include preventing.

A “effective amount” is an amount sufficient for a compound to accomplish a stated purpose relative to the absence of the compound (e.g. achieve the effect for which it is administered, treat a disease, or reduce one or more symptoms of a disease or condition). An example of an “effective amount” is an amount sufficient to contribute to the treatment or reduction of a symptom or symptoms of a disease, which could also be referred to as a “therapeutically effective amount.” A “reduction” of a symptom or symptoms means decreasing of the severity or frequency of the symptom(s), or elimination of the symptom(s). The exact amounts will depend on the purpose of the treatment, and will be ascertainable by one skilled in the art using known techniques.

The term “administering” is used in accordance with its plain and ordinary meaning and includes oral, topical, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, or transdermal). Parenteral administration includes, e.g., intravenous, intramuscular, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any therapeutic agent other than the recited therapeutic agent.

“Surgery” refers to a medical specialty that uses operative manual and instrumental techniques on a person to investigate or treat a pathological condition such as a disease or injury. The act of performing surgery may be called a surgical procedure, operation, or simply “surgery.” The adjective surgical means pertaining to surgery; e.g. surgical instruments or surgical nurse. The term “ablation” refer to the removal of a part of biological tissue, usually by surgery. The term “resection” refers to surgical procedure to partially remove an organ or other bodily structure.

“Anti-cancer agent” and “anticancer agent” are used in accordance with their plain ordinary meaning and refers to a composition (e.g. compound, drug, antagonist, inhibitor, modulator) having antineoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-1, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin I1 (including recombinant interleukin II, or rlL2), interferon alfa-2a; interferon alfa-2b; interferon alfa-n1; interferon alfa-n3; interferon beta-1a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-310705), 21-hydroxyepothilone D (i.e. Desoxyepothilone F and dEpoF), 26-fluoroepothilone, Auristatin PE (i.e. NSC-654663), Soblidotin (i.e. TZT-1027), LS-4559-P (Pharmacia, i.e. LS-4577), LS-4578 (Pharmacia, i.e. LS-477-P), LS-4477 (Pharmacia), LS-4559 (Pharmacia), RPR-112378 (Aventis), Vincristine sulfate, DZ-3358 (Daiichi), FR-182877 (Fujisawa, i.e. WS-9885B), GS-164 (Takeda), GS-198 (Takeda), KAR-2 (Hungarian Academy of Sciences), BSF-223651 (BASF, i.e. ILX-651 and LU-223651), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (i.e. LY-355703), AC-7739 (Ajinomoto, i.e. AVE-8063A and CS-39·HCl), AC-7700 (Ajinomoto, i.e. AVE-8062, AVE-8062A, CS-39-L-Ser·HCl, and RPR-258062A), Vitilevuamide, Tubulysin A, Canadensol, Centaureidin (i.e. NSC-106969), T-138067 (Tularik, i.e. T-67, TL-138067 and TI-138067), COBRA-1 (Parker Hughes Institute, i.e. DDE-261 and WHI-261), H10 (Kansas State University), H16 (Kansas State University), Oncocidin A1 (i.e. BTO-956 and DIME), DDE-313 (Parker Hughes Institute), Fijianolide B, Laulimalide, SPA-2 (Parker Hughes Institute), SPA-1 (Parker Hughes Institute, i.e. SPIKET-P), 3-IAABU (Cytoskeleton/Mt. Sinai School of Medicine, i.e. MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, i.e. MF-191), TMPN (Arizona State University), Vanadocene acetylacetonate, T-138026 (Tularik), Monsatrol, lnanocine (i.e. NSC-698666), 3-IAABE (Cytoskeleton/Mt. Sinai School of Medicine), A-204197 (Abbott), T-607 (Tuiarik, i.e. T-900607), RPR-115781 (Aventis), Eleutherobins (such as Desmethyleleutherobin, Desaetyleleutherobin, lsoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (i.e. NSCL-96F037), D-68838 (Asta Medica), D-68836 (Asta Medica), Myoseverin B, D-43411 (Zentaris, i.e. D-81862), A-289099 (Abbott), A-318315 (Abbott), HTI-286 (i.e. SPA-110, trifluoroacetate salt) (Wyeth), D-82317 (Zentaris), D-82318 (Zentaris), SC-12983 (NCI), Resverastatin phosphate sodium, BPR-OY-007 (National Health Research Institutes), and SSR-250411 (Sanofi)), steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), immunotherapy (e.g., cellular immunotherapy, antibody therapy, cytokine therapy, combination immunotherapy, etc.), radioimmunotherapy (e.g., anti-CD20 monoclonal antibody conjugated to ¹¹In, ⁹⁰Y, or ¹³¹I, etc.), immune checkpoint inhibitors (e.g., CTLA4 blockade, PD-1 inhibitors, PD-L1 inhibitors, etc.), triptolide, homoharringtonine, dactinomycin, doxorubicin, epirubicin, topotecan, itraconazole, vindesine, cerivastatin, vincristine, deoxyadenosine, sertraline, pitavastatin, innotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

### Confirmatory Diagnostics

In embodiments, the methods described herein comprise performing a confirmatory diagnostic procedure on the subject.

“Confirmatory diagnostic procedure” as used herein refer to medical tests or procedures used to confirm a medical diagnosis. A confirmatory diagnostic procedure can be, e.g., a angiography, an alfa-fetoprotein (AFP) protein blood test, a tumor marker test, a microsatellite instability (MSI) test, an esophagusgastroduodenoscopy (EGD), an abdominal ultrasound, an endoscopic ultrasound, a bronchoscopy, a tissue biopsy, a fine needle aspiration, an esophagogastroduodenoscopy (EGD), a tissue biopsy, a CA19-9 antigen test, a fine needle aspiration, an endoscopy, biopsy collection, a blood test, a fecal test, a fecal occult blood test, a magnetic resonance imaging scan (MRI scan) (e.g. a cholangiopancreatography), a computed tomography scan (CT scan), a positron emission tomography scan (PET scan), or a carcinoembryonic antigen (CEA) test.

“Biopsy” refers to a medical test which involves extraction of sample cells or tissues for examination to determine the presence or extent of a disease in a subject. The extracted tissue is generally examined under a microscope by a pathologist, and it may also be analyzed chemically. When an entire lump or suspicious area is removed, the procedure is called an excisional biopsy. An incisional biopsy or core biopsy samples a portion of the abnormal tissue without attempting to remove the entire lesion or tumor. When a sample of tissue or fluid is removed with a needle in such a way that cells are removed without preserving the histological architecture of the tissue cells, the procedure is called a needle aspiration biopsy. The terms “biopsy material” refer to the sample extracted from the subject. The terms “tissue biopsy” refer to the extraction of tissue from a subject.

“Needle aspiration” refer to diagnostic procedure used to investigate lumps or masses. In this procedure a thin, hollow needle and a syringe are used to extract cells, fluid or tissue from a suspicious lump or other abnormal area of the body. The material is then examined under a microscope or tested in the laboratory to determine the cause of the abnormality. The sampling and biopsy considered together are called needle aspiration biopsy or needle aspiration cytology (the latter to emphasize that any aspiration biopsy involves cytopathology, not histopathology).

The terms “fecal test” or “stool test” refer to the collection and analysis of fecal matter to diagnose the presence or absence of a medical condition. The terms “fecal occult blood test” refer to a test checking for blood that is not visibly apparent (occult), in the feces of a subject. The terms “fecal DNA test” refer to a DNA test realized on fecal material obtained from a subject.

The terms “DNA test” or “genetic test” refer to test of DNA material obtaining from a subject or sample, which is used to identify changes in DNA sequence or chromosome structure. Genetic testing can also include measuring the results of genetic changes, such as DNA methylation analysis, or RNA or protein analysis as an output of gene expression. In a medical setting, genetic testing can be used to diagnose or rule out suspected cancers or genetic disorders, predict risks for specific cancer, or gain information that can be used to customize medical treatments based on an individual's cancer.

The terms “blood test” refer to a laboratory analysis performed on a blood sample. A blood test can be used to detect DNA methylation as described herein. Blood tests are often used in health care to determine physiological and biochemical states, such as disease, mineral content, pharmaceutical drug effectiveness, and organ function. Blood tests can involve different tests on the blood sample, such as biochemal analyses, molecular profiling, and cellular evaluation.

The terms “ultrasound” refers to an ultrasound-based diagnostic medical imaging technique used to visualize muscles, tendons, and many internal organs to capture their size, structure and any pathological lesions with real time tomographic images. “Abdominal ultrasound” is a form of medical ultrasonography (medical application of ultrasound technology) to visualise abdominal anatomical structures. “Endoscopic ultrasound” refers to a medical procedure in which endoscopy (insertion of a probe into a hollow organ) is combined with ultrasound to obtain images of the internal organs in the chest, abdomen and colon. It can be used to visualize the walls of these organs, or to look at adjacent structures. Combined with Doppler imaging, nearby blood vessels can also be evaluated.

The term “embolization” refer to the passage and lodging of an embolus within the bloodstream. It may be of natural origin (pathological), in which sense it is also called embolism, for example a pulmonary embolism; or it may be artificially induced (therapeutic), as a hemostatic treatment for bleeding or as a treatment for some types of cancer by deliberately blocking blood vessels to starve the tumor cells. The term “embolus” refers to an unattached mass that travels through the bloodstream and is capable of creating blockages. When an embolus occludes a blood vessel, it is called an embolism or embolic event.

The terms “endoscopic therapy” refer to treatments performed using an endoscope. An endoscope is a small, tube-like instrument that is inserted into the body through a tiny incision or a body opening, such as the mouth. The term “endoscopic mucosal resection” refer to a procedure to remove precancerous, early-stage cancer or other abnormal tissues (e.g. lesions or precancerous growths) from the digestive tract, using an endoscope.

The term “gastrectomy” refers to the partial or total surgical removal of the stomach. A gastrectomy may be done to a patient to treat cancer of the stomach. There are three main types of gastrectomy: a partial gastrectomy is the removal of a part of the stomach, a full gastrectomy or total gastrectomy is the removal of the entire stomach, and a sleeve gastrectomy is the removal of the left side of the stomach. The terms “partial gastrectomy,” “partial (distal) gastrectomy,” “distal gastrectomy,” and “antrectomy” are used interchangeably to refer to a procedure that involves surgical removal of the lower 30% of the stomach (antrum). Distal gastrectomy is a type of partial gastrectomy that involves the surgical removal of only a portion of the stomach.

The terms “computed tomography scan” or “CT scan” refer to a medical imaging technique that uses computer-processed combinations of multiple X-ray measurements taken from different angles to produce tomographic (cross-sectional) images (virtual “slices”) of a body, allowing the user to see inside the body without cutting.

The terms “X-ray” or “X-radiation” refer to a penetrating form of high-energy electromagnetic radiation. Most X-rays have a wavelength ranging from 10 picometers to 10 nanometers, corresponding to frequencies in the range 30 petahertz to 30 exahertz (30×1015 Hz to 30×1018 Hz) and energies in the range 124 eV to 124 keV. X-ray wavelengths are shorter than those of UV rays and typically longer than those of gamma rays.

The terms “PET”, “PET scan”, “positron emission tomography”, or “positron emission tomography scan” refer to a functional imaging technique that uses radioactive substances known as radiotracers to visualize and measure changes in metabolic processes, and in other physiological activities including blood flow, regional chemical composition, and absorption. Different tracers are used for various imaging purposes, depending on the target process within the body. PET scan is a common imaging technique, a medical scintillography technique used in nuclear medicine. A radiopharmaceutical—a radioisotope attached to a drug is injected into the body as a tracer. Gamma rays are emitted and detected by gamma cameras to form a three-dimensional image, in a similar way that an X-ray image is captured.

The terms “MRI” or “magnetic resonance imaging” refer to a medical imaging technique used in radiology to form pictures of the anatomy and the physiological processes of the body. MRI scanners use strong magnetic fields, magnetic field gradients, and radio waves to generate images of the organs in the body. MRI does not involve X-rays or the use of ionizing radiation, which distinguishes it from CT and PET scans. MRI is a medical application of nuclear magnetic resonance (NMR) which can also be used for imaging in other NMR applications, such as NMR spectroscopy.

The term “cholangiopancreatography” refers to the visualization and examination of the bile ducts and pancreas. For example, an endoscopic retrograde cholangiopancreatography (ERCP) is a technique that combines the use of endoscopy and fluoroscopy to diagnose and treat certain problems of the biliary or pancreatic ductal systems. Another example of cholangiopancreatography is the magnetic resonance cholangiopancreatography (MRCP), which is a medical imaging technique that uses magnetic resonance imaging to visualize the biliary and pancreatic ducts in a non-invasive manner.

The term “angiography” or “arteriography” refer to a medical imaging technique used to visualize the inside, or lumen, of blood vessels and organs of the body, with particular interest in the arteries, veins, and the heart chambers. This is traditionally done by injecting a radio-opaque contrast agent into the blood vessel and imaging using X-ray based techniques such as fluoroscopy.

The terms “esophagus-gastric-duodenoscopy,” “esophagogastroduodenoscopy” and “EGD” refer to a diagnostic endoscopic procedure that visualizes the upper part of the gastrointestinal tract down to the duodenum.

The term “bronchoscopy” refers to an endoscopic technique of visualizing the inside of the airways for diagnostic and therapeutic purposes. An instrument (bronchoscope) is inserted into the airways, usually through the nose or mouth, or occasionally through a tracheostomy. This allows the practitioner to examine the patient's airways for abnormalities such as foreign bodies, bleeding, tumors, or inflammation. Samples may be taken from inside the lungs.

The terms “CAT9-9” or “carbohydrate antigen 19-9” refer to a tetrasaccharide which is usually attached to 0-glycans on the surface of cells, and it is known to play a vital role in cell-to-cell recognition processes. CA19-9, also known as “sialyl-LewisA” tumor marker used primarily in the management of pancreatic cancer. A “CAT9-9 antigen test” refer to a blood test aimed at the detection and measurement of CA19-9 in a blood sample from a subject.

The terms “alfa-fetoprotein” or “AFP” refer to a protein that in humans is encoded by the AFP gene. The AFP gene is located on the q arm of chromosome 4 (4q25). Maternal AFP serum level is used to screen for Down syndrome, neural tube defects, and other chromosomal abnormalities. AFP is a major plasma protein produced by the yolk sac and the fetal liver during fetal development. It is thought to be the fetal analog of serum albumin. AFP binds to copper, nickel, fatty acids and bilirubin and is found in monomeric, dimeric and trimeric forms. An “alfa-fetoprotein (AFP) protein blood test” or ““alfa-fetoprotein (AFP) protein blood test” refer to a blood test aimed at the detection and measurement of AFP in a blood sample from a subject.

The terms “carcinoembryonic antigen” or “CEA” as used here refers to a set of highly related glycoproteins involved in cell adhesion. CEA is normally produced in gastrointestinal tissue during fetal development, but the production stops before birth. Consequently, CEA is usually present at very low levels in the blood of healthy adults. However, the serum levels are raised in some types of cancer, which means that it can be used as a tumor marker in clinical tests. Serum levels can also be elevated in heavy smokers. The terms “carcinoembryonic antigen (CEA) test”, “carcinoembryonic antigen test” or “CEA test” refer to a test aimed at the detection and measurement of CEA amounts in a blood sample from a subject.

The term “microsatellite” refers to a repeated sequences of DNA. Microsatellite sequences can be made of repeating units of one to six base pairs in length. Although the length of these microsatellites is highly variable from person to person and contributes to the individual DNA “fingerprint”, each individual has microsatellites of a set length. The most common microsatellite in humans is a dinucleotide repeat of the nucleotides C and A, which occurs tens of thousands of times across the genome. Microsatellites are also known as simple sequence repeats (SSRs). The terms “microsatellite instability” or “MSI” refer to a condition of genetic hypermutability (predisposition to mutation) that results from impaired DNA mismatch repair (MMR). The presence of MSI represents phenotypic evidence that MMR is not functioning normally. MMR corrects errors that spontaneously occur during DNA replication, such as single base mismatches or short insertions and deletions. The proteins involved in MMR correct polymerase errors by forming a complex that binds to the mismatched section of DNA, excises the error, and inserts the correct sequence in its place. Cells with abnormally functioning MMR are unable to correct errors that occur during DNA replication and consequently accumulate errors. This causes the creation of novel microsatellite fragments. Polymerase chain reaction-based assays can reveal these novel microsatellites and provide evidence for the presence of MSI. The terms “microsatellite instability test”, “MSI test”, “microsatellite instability screen” or “MSI screen” refer to a test aimed at the measurement of genes implicated in the hereditary nonpolyposis colorectal cancer (“HNPCC”, also known as “Lynch syndrome”). HNPCC is an autosomal dominant genetic condition that is associated with a high risk of colon cancer as well as other cancers including endometrial cancer (second most common), ovary, stomach, small intestine, hepatobiliary tract, upper urinary tract, brain, and skin. The hallmark of HNPCC is defective DNA mismatch repair, which leads to microsatellite instability (MSI).

The terms “tumor marker” refer to a biomarker (a measurable indicator of the severity or presence of some disease state) found in blood, urine, or body tissues that can be elevated by the presence of one or more types of cancer. There are many different tumor markers, each indicative of a particular disease process, and they are used in oncology to help detect the presence of cancer. An elevated level of a tumor marker can indicate cancer; however, there can also be other causes of the elevation (false positive values). Tumor markers can be produced directly by the tumor or by non-tumor cells as a response to the presence of a tumor.

### Computer Systems

In embodiments, the disclosure provides a computer program product comprising a machine-readable medium storing instructions that, when executed by at least one programmable processor, cause the at least one programmable processor to perform operations comprising the methods described herein, including all embodiments thereof.

In embodiments, the disclosure provides a system comprising computer hardware configured to perform operations comprising the methods described herein, including all embodiments thereof.

In embodiments, the disclosure provides a computer-implemented method comprising the methods described herein, including all embodiments thereof.

In embodiments, the disclosure provides computer control systems that are programmed to implement the methods of the disclosure, including all embodiments thereof. A computer system can be programmed or otherwise configured to implements methods of the disclosure, including all embodiments thereof. The computer system can be integral to implementing methods provided herein, which may be otherwise difficult to perform in the absence of the computer system. The computer system can be an electronic device of a user or a computer system that is remotely located with respect to the electronic device. The electronic device can be a mobile electronic device. As an alternative, the computer system can be a computer server.

The computer system includes a central processing unit (CPU, also “processor” and “computer processor”), which can be a single core or multi-core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. The computer system can be operatively coupled to a computer network (“network”) with the aid of the communication interface. The network can be the internet, an internet and/or extranet, or an intranet and/or extranet that is in communication with the internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the internet.

The computer system can communicate with one or more remote computer systems through the network. For instance, the computer system can communicate with a remote computer system of a user (e.g., patient, healthcare provider, or service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The memory can be part of a database. The machine executable or machine readable code can be provided in the form of software. During use, the code can be executed by the processor. In embodiments, the code can be retrieved from the storage unit and stored on the memory for ready access by the processor. In embodiments, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk.

“Storage” media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, genetic information, such as an identification of disease-causing alleles in single individuals or groups of individuals. Examples of UI's include, without limitation, a graphical user interface (GUI) and web-based user interface (or web interface).

Methods and systems of the present disclosure can be implemented by way of one or more algorithms. An algorithm can be implemented by way of software upon execution by the central processing unit. The algorithm can, for example, rank the relatedness of a DMR pattern with a subject's cancer status.

Disclosed herein, in embodiments, are reports, such as CpG methylation reports. The reports are generated using the methods and systems described herein, to provide the user with results from the analyses of the degree of methylation of CpG sites within a plurality of DMRs from a subject. In some aspects, the reports comprise an indication of a higher risk of developing a gastrointestinal cancer relative to a standard control. In some cases, the reports comprise a treatment recommendation based on the identified gastrointestinal cancer.

In embodiments, the report comprises a result from the analysis that is represented in a range (e.g., normal to high) of risk for developing or having a gastrointestinal cancer, which is relative to a control population. In aspects, the control population made up of individuals of the same ancestry as the subject. In aspects, the reference population is not ancestry-specific to the subject. In general, a normal result indicates that the subject is not predisposed to developing or having the gastrointestinal cancer. In contrast, a high result indicates that the subject has a higher risk of developing or having a gastrointestinal cancer, as compared to standard control. A low risk indicates that the subject is predisposed not to have or develop a gastrointestinal cancer. A slightly high or slightly low result indicates a score between a normal score and a high or a low score, respectively.

The reports described herein, in some cases, provide the user with diagnosis or treatment recommendations based on the gastrointestinal cancer for which a subject found to be at a higher risk. In a non-limiting example, a confirmatory diagnostic procedure, such as a fine needle aspiration, may be recommended for a subject found at a higher risk of developing gastrointestinal cancer. In a non-limiting example, a treatment, such as surgery, may be recommended for a subject found at a higher risk of developing gastrointestinal cancer.

The reports are formatted for delivery to the user using any suitable method, including electronically or by mail. In embodiments, the reports are electronic reports. Electronic reports, in some cases, are formatted to transmit via a computer network to a personal electronic device of the individual (e.g., tablet, laptop, smartphone, fitness tracking device). In embodiments, the report is integrated into a mobile application on the personal electronic device. In embodiments, the App is interactive, and permits the individual to click on hyperlinks embedded within the report that automatically redirect the user to an online resource. In embodiments, the reports are encrypted or otherwise secured to protect the privacy of the individual. In embodiments, the reports are printed and mailed to the user.

In embodiments, the software programs described herein include a web application. In light of the disclosure provided herein, those of skill in the art will recognize that a web application may utilize one or more software frameworks and one or more database systems. A web application, for example, is created upon a software framework such as Microsoft®.NET or Ruby on Rails (RoR). A web application, in embodiments, utilizes one or more database systems including, by way of non-limiting examples, relational, non-relational, feature oriented, associative, and XML database systems. Suitable relational database systems include, by way of non-limiting examples, Microsoft® SQL Server, mySQL™, and Oracle®. Those of skill in the art will also recognize that a web application may be written in one or more versions of one or more languages. In embodiments, a web application is written in one or more markup languages, presentation definition languages, client-side scripting languages, server-side coding languages, database query languages, or combinations thereof. In embodiments, a web application is written to some extent in a markup language such as Hypertext Markup Language (HTML), Extensible Hypertext Markup Language (XHTML), or extensible Markup Language (XML). In embodiments, a web application is written to some extent in a presentation definition language such as Cascading Style Sheets (CSS). In embodiments, a web application is written to some extent in a client-side scripting language such as Asynchronous Javascript and XML (AJAX), Flash® Actionscript, Javascript, or Silverlight®. In embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In embodiments, a web application is written to some extent in a database query language such as Structured Query Fanguage (SQF). A web application may integrate enterprise server products such as IBM® Fotus Domino®. A web application may include a media player element. A media player element may utilize one or more of many suitable multimedia technologies including, by way of non limiting examples, Adobe® Flash®, HTMF 5, Apple® QuickTime®, Microsoft® Silverlight®, Java™, and Unity®.

In embodiments, software programs described herein include a mobile application provided to a mobile digital processing device. The mobile application may be provided to a mobile digital processing device at the time it is manufactured. The mobile application may be provided to a mobile digital processing device via the computer network described herein.

A mobile application is created by techniques known to those of skill in the art using hardware, languages, and development environments known to the art. Those of skill in the art will recognize that mobile applications may be written in several languages. Suitable programming languages include, by way of non limiting examples, C, C++, C #, Featureive-C, Java™, Javascript, Pascal, Feature Pascal, Python™, Ruby, VB.NET, WMF, and XHTMF/HTMF with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Fite, .NET Compact Framework, Rhomobile, and WorkFight Mobile Platform. Other development environments may be available without cost including, by way of non-limiting examples, Fazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In embodiments, the software programs described herein include a standalone application, which is a program that may be run as an independent computer process, not an add-on to an existing process, e.g., not a plug-in. Those of skill in the art will recognize that standalone applications are sometimes compiled. In embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Perl, R, Python™, Visual Basic, and VB .NET, or combinations thereof. Compilation may be often performed, at least in part, to create an executable program. In embodiments, a computer program includes one or more executable complied applications.

Disclosed herein are software programs that, in embodiments, include a web browser plug-in. In computing, a plug-in, in embodiments, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands. Those skilled in the art will recognize that several plug-in frameworks are available that enable development of plug-ins in various programming languages, including, by way of non-limiting examples, C++, Delphi, Java™, PHP, Python™, and VB .NET, or combinations thereof.

In embodiments, web browsers (also called internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. The web browser, in embodiments, is a mobile web browser. Mobile web browsers (also called mircrobrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft®. Internet Explorer® Mobile, Amazon® Kindle® Basic Web, Nokia® Browser, Opera Software® Opera® Mobile, and Sony® PSP™ browser.

The medium, method, and system disclosed herein comprise one or more softwares, servers, and database modules, or use of the same. In view of the disclosure provided herein, software modules may be created by techniques known to those of skill in the art using machines, software, and languages known to the art. The software modules disclosed herein may be implemented in a multitude of ways. In embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprises a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

The medium, method, and system disclosed herein comprise one or more databases, such as the phenotypic and/or genotypic-associated database described herein, or use of the same. In embodiments, the database are used for rare genetic variants, and optionally common genetic variants. Those of skill in the art will recognize that many databases are suitable for storage and retrieval of information. Suitable databases include, by way of non-limiting examples, relational databases, non-relational databases, feature oriented databases, feature databases, entity-relationship model databases, associative databases, and XML databases. In embodiments, a database is internet-based. In embodiments, a database is web-based. In embodiments, a database is cloud computing-based. A database may be based on one or more local computer storage devices.

The methods, systems, and media described herein, are configured to be performed in one or more facilities at one or more locations. Facility locations are not limited by country and include any country or territory. In embodiments, one or more steps of a method herein are performed in a different country than another step of the method. In embodiments, one or more steps for obtaining a sample are performed in a different country than one or more steps for analyzing a genotype of a sample. In embodiments, one or more method steps involving a computer system are performed in a different country than another step of the methods provided herein. In embodiments, data processing and analyses are performed in a different country or location than one or more steps of the methods described herein. In embodiments, one or more articles, products, or data are transferred from one or more of the facilities to one or more different facilities for analysis or further analysis. An article includes, but is not limited to, one or more components obtained from a sample of a subject and any article or product disclosed herein as an article or product. Data includes, but is not limited to, information regarding genotype and any data produced by the methods disclosed herein. In embodiments of the methods and systems described herein, the analysis is performed and a subsequent data transmission step will convey or transmit the results of the analysis.

In embodiments, any step of any method described herein is performed by a software program or module on a computer. In embodiments, data from any step of any method described herein is transferred to and from facilities located within the same or different countries, including analysis performed in one facility in a particular location and the data shipped to another location or directly to an individual in the same or a different country. In embodiments, data from any step of any method described herein is transferred to and/or received from a facility located within the same or different countries, including analysis of a data input, such as cellular material, performed in one facility in a particular location and corresponding data transmitted to another location, or directly to an individual, such as data related to the diagnosis, prognosis, responsiveness to therapy, or the like, in the same or different location or country.

Embodiments disclosed herein provide one or more non-transitory computer readable storage media encoded with a software program including instructions executable by the operating system. In embodiments, software encoded includes one or more software programs described herein. In embodiments, a computer readable storage medium is a tangible component of a computing device. In embodiments, a computer readable storage medium is optionally removable from a computing device. In embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In embodiments, the program and instructions are permanently, substantially permanently, semi-permanently, or non-transitorily encoded on the media.

### Embodiments 1-87

Embodiment 1. A method of detecting a level of DNA methylation in a subject at risk of developing a gastrointestinal cancer, said method comprising: determining the degree of methylation of CpG sites within a plurality of gene regions in a DNA sample from said subject, wherein the plurality of gene regions comprises at least 50 different gene regions in Table PGI.

Embodiment 2. The method of Embodiment 1, wherein the plurality of gene regions comprises at least 100 gene regions in Table PGI.

Embodiment 3. The method of Embodiment 1, wherein the plurality of gene regions comprises at least 150 gene regions in Table PGI.

Embodiment 4. The method of Embodiment 1, wherein the plurality of gene regions comprises the first 150 gene regions in Table PGI.

Embodiment 5. The method of any of the above Embodiments, further comprising performing a confirmatory diagnostic procedure on said subject.

Embodiment 6. The method of Embodiment 5, wherein said confirmatory diagnostic procedure is a fine needle aspiration, an endoscopy, or biopsy collection.

Embodiment 7. The method of Embodiment 5, wherein said confirmatory diagnostic procedure is an X-Ray, a CT scan, an MRI, a PET Scan, a blood test or a fecal test.

Embodiment 8. The method of any of the above Embodiments, further comprising treating said subject for a gastrointestinal cancer.

Embodiment 9. The method of Embodiment 8, wherein said treating comprises surgery, systemic chemotherapy, radiotherapy or targeted therapy.

Embodiment 10. The method of any of Embodiments 1 to 8, wherein an increased level of methylation of CpG sites relative to a standard control indicates a higher risk of gastrointestinal cancer.

Embodiment 11. A method of detecting a level of DNA methylation in a subject at risk of developing a colorectal cancer, said method comprising: determining the degree of methylation of CpG sites within a plurality of gene regions in a DNA sample from said subject, wherein the plurality of gene regions comprises at least 5 different gene regions in Table CRC.

Embodiment 12. The method of Embodiment 11, wherein the plurality of gene regions comprises at least 10 DMRs in Table CRC.

Embodiment 13. The method of Embodiment 12, wherein the plurality of gene regions comprises the first 10 DMRs in Table CRC.

Embodiment 14. The method of any of Embodiments 11 to 13, further comprising performing a confirmatory diagnostic procedure on said subject.

Embodiment 15. The method of Embodiment 14, wherein said confirmatory diagnostic procedure is a fine needle aspiration, an endoscopy, or a tissue biopsy.

Embodiment 16. The method of Embodiment 14, wherein said confirmatory diagnostic procedure is a fecal DNA test or a Carcinoembryonic Antigen (CEA) test.

Embodiment 17. The method of any of Embodiments 11 to 16, further comprising treating said subject for colorectal cancer.

Embodiment 18. The method of Embodiment 17, wherein said treating comprises surgery, ablation, embolization, or radiotherapy.

Embodiment 19. The method of Embodiment 17, wherein said treating comprises chemotherapy, targeted therapy, or immunotherapy.

Embodiment 20. The method of any of Embodiments 11 to 17, wherein an increased level of methylated CpG sites relative to a standard control indicates a higher risk of colorectal cancer.

Embodiment 21. A method of detecting a level of DNA methylation in a subject at risk of developing a hepatocellular carcinoma, said method comprising: determining the degree of methylation of CpG sites within a plurality of gene regions in a DNA sample from said subject, wherein the plurality of gene regions comprises at least 5 different gene regions in Table HCC.

Embodiment 22. The method of Embodiment 21, wherein the plurality of gene regions comprises at least 10 DMRs in Table HCC.

Embodiment 23. The method of Embodiment 21, wherein the plurality of gene regions comprises the first 10 DMRs in Table HCC.

Embodiment 24. The method of any of Embodiments 21 to 23, further comprising performing a confirmatory diagnostic procedure on said subject.

Embodiment 25. The method of Embodiment 24, wherein said confirmatory diagnostic procedure is a tissue biopsy.

Embodiment 26. The method of Embodiment 24, wherein said confirmatory diagnostic procedure is an ultrasound, a CT scan, an MRI, angiography, or alfa-fetoprotein (AFP) protein blood test.

Embodiment 27. The method of any of Embodiments 21 to 26, further comprising treating said subject for a hepatocellular carcinoma.

Embodiment 28. The method of Embodiment 27, wherein said treating comprises surgery, radiotherapy, chemotherapy, targeted therapy, immunotherapy.

Embodiment 29. The method of any of Embodiments 21 to 28, wherein an increased level of methylated CpG sites relative to a standard control indicates a higher risk of colorectal cancer.

Embodiment 30. A method of detecting a level of DNA methylation in a subject at risk of developing a esophageal squamous cell carcinoma, said method comprising: determining the degree of methylation of CpG sites within a plurality of gene regions in a DNA sample from said subject, wherein the plurality of gene regions comprises at least 5 different gene regions in Table ESCC.

Embodiment 31. The method of Embodiment 30, wherein the plurality of gene regions comprises at least 10 DMRs in Table ESCC.

Embodiment 32. The method of Embodiment 30, wherein the plurality of gene regions comprises the first 10 DMRs in Table ESCC.

Embodiment 33. The method of any of Embodiments 30 to 32, further comprising performing a confirmatory diagnostic procedure on said subject.

Embodiment 34. The method of Embodiment 33, wherein said confirmatory diagnostic procedure is an esophagus-gastric-duodenoscopy (EGD), an endoscopic ultrasound, a bronchoscopy, or a tissue biopsy.

Embodiment 35. The method of Embodiment 33, wherein said confirmatory diagnostic procedure is a tumor marker test, a microsatellite instability (MSI) test, a CT scan, a MRI, a PET scan.

Embodiment 36. The method of any of Embodiments 30 to 35, further comprising treating said subject for esophageal squamous cell carcinoma.

Embodiment 37. The method of Embodiment 36, wherein said treating comprises surgery, endoscopic therapy, or radiation therapy.

Embodiment 38. The method of Embodiment 36, wherein said treating comprises chemotherapy, targeted therapy, or immunotherapy.

Embodiment 39. The method of any of Embodiments 30 to 38, wherein an increased level of methylated CpG sites relative to a standard control indicates a higher risk of esophageal squamous cell carcinoma.

Embodiment 40. A method of detecting a level of DNA methylation in a subject at risk of developing a gastric cancer, said method comprising: determining the degree of methylation of CpG sites within a plurality of gene regions in a DNA sample from said subject, wherein the plurality of gene regions comprises at least 5 different gene regions in Table GC.

Embodiment 41. The method of Embodiment 40, wherein the plurality of gene regions comprises at least 10 DMRs in Table GC.

Embodiment 42. The method of Embodiment 40, wherein the plurality of gene regions comprises the first 10 DMRs in Table GC.

Embodiment 43. The method of any of Embodiments 40 to 42, further comprising performing a confirmatory diagnostic procedure on said subject.

Embodiment 44. The method of Embodiment 43, wherein said confirmatory diagnostic procedure is a fine needle aspiration, an esophagogastroduodenoscopy (EGD), or tissue biopsy.

Embodiment 45. The method of Embodiment 43, wherein said confirmatory diagnostic procedure is a CT, a PET, a MRI, or fecal occult blood test.

Embodiment 46. The method of any of Embodiments 40 to 45, further comprising treating said subject for gastric cancer.

Embodiment 47. The method of Embodiment 46, wherein said treating comprises endoscopic mucosal resection, partial (Distal) Gastrectomy, or total Gastrectomy.

Embodiment 48. The method of Embodiment 46, wherein said treating comprises radiotherapy, chemotherapy, targeted therapy, or immunotherapy.

Embodiment 49. The method of any of Embodiments 40 to 48, wherein an increased level of methylated CpG sites relative to a standard control indicates a higher risk of gastric cancer.

Embodiment 50. A method of detecting a level of DNA methylation in a subject at risk of developing esophageal adenocarcinoma, said method comprising: determining the degree of methylation of CpG sites within a plurality of gene regions in a DNA sample from said subject, wherein the plurality of gene regions comprises at least 5 different gene regions in Table EAC.

Embodiment 51. The method of Embodiment 50, wherein the plurality of gene regions comprises at least 10 DMRs in Table EAC.

Embodiment 52. The method of Embodiment 50, wherein the plurality of gene regions comprises the first 10 DMRs in Table EAC.

Embodiment 53. The method of any of Embodiments 50 to 52, further comprising performing a confirmatory diagnostic procedure on said subject.

Embodiment 54. The method of Embodiment 53, wherein said confirmatory diagnostic procedure is an esophagus-gastric-duodenoscopy (EGD), an endoscopic ultrasound, a bronchoscopy, or a tissue biopsy.

Embodiment 55. The method of Embodiment 53, wherein said confirmatory diagnostic procedure is a tumor marker test, a microsatellite instability (MSI) test, a CT scan, a MRI, a PET scan.

Embodiment 56. The method of any of Embodiments 50 to 55, further comprising treating said subject for esophageal adenocarcinoma.

Embodiment 57. The method of Embodiment 56, wherein said treating comprises surgery, endoscopic therapy, or radiation therapy.

Embodiment 58. The method of Embodiment 56, wherein said treating comprises chemotherapy, targeted therapy, or immunotherapy.

Embodiment 59. The method of any of Embodiments 50 to 58, wherein an increased level of methylated CpG sites relative to a standard control indicates a higher risk of esophageal adenocarcinoma.

Embodiment 60. A method of detecting a level of DNA methylation in a subject at risk of developing pancreatic ductal adenocarcinoma, said method comprising: determining the degree of methylation of CpG sites within a plurality of gene regions in a DNA sample from said subject, wherein the plurality of gene regions comprises at least 5 different gene regions in Table PDAC.

Embodiment 61. The method of Embodiment 60, wherein the plurality of gene regions comprises at least 10 DMRs in Table PDAC.

Embodiment 62. The method of Embodiment 60, wherein the plurality of gene regions comprises the first 10 DMRs in Table PDAC.

Embodiment 63. The method of any of Embodiments 60 to 62, further comprising performing a confirmatory diagnostic procedure on said subject.

Embodiment 64. The method of Embodiment 63, wherein said confirmatory diagnostic procedure is an abdominal ultrasound, an endoscopic ultrasound, a fine needle aspiration, a tissue biopsy.

Embodiment 65. The method of Embodiment 63, wherein said confirmatory diagnostic procedure is a MRI (Cholangiopancreatography), a CT scan, a PET scan, a Carcinoembryonic Antigen (CEA) test, or a CA19-9 antigen test.

Embodiment 66. The method of any of Embodiments 60 to 65, further comprising treating said subject for pancreatic ductal adenocarcinoma.

Embodiment 67. The method of Embodiment 66, wherein said treating comprises surgery.

Embodiment 68. The method of Embodiment 66, wherein said treating comprises radiotherapy, chemotherapy, targeted therapy, or immunotherapy.

Embodiment 69. The method of any of Embodiments 60 to 68, wherein an increased level of methylated CpG sites relative to a standard control indicates a higher risk of pancreatic ductal adenocarcinoma.

Embodiment 70. A method of detecting a level of DNA methylation in a subject at risk of developing a gastrointestinal cancer and determining its likely tissue of origin, said method comprising: determining the level of methylation of CpG sites within a plurality of gene regions in a DNA sample from said subject, wherein the plurality of gene regions comprises at least 50 different gene regions set forth in Table MCC; and wherein the level of methylation of CpG sites identifies the tissue as colorectal, hepatic, esophageal, or pancreatic.

Embodiment 71. The method of Embodiment 70, wherein the plurality of gene regions comprises at least 100 gene regions in Table MCC.

Embodiment 72. The method of Embodiment 70, wherein the plurality of gene regions comprises at least 150 gene regions in Table MCC.

Embodiment 73. The method of Embodiment 70, wherein the plurality of gene regions comprises first 150 gene regions in Table MCC.

Embodiment 74. The method of any of Embodiments 70 to 73, further comprising performing a confirmatory diagnostic procedure on said subject.

Embodiment 75. The method of Embodiment 74, wherein said confirmatory diagnostic procedure is a fine needle aspiration, an endoscopy, or biopsy collection.

Embodiment 76. The method of Embodiment 74, wherein said confirmatory diagnostic procedure is an X-Ray, a CT scan, an MRI, a PET Scan, a blood test or a fecal test.

Embodiment 77. The method of any of Embodiments 70 to 76, further comprising treating said subject for a gastrointestinal cancer.

Embodiment 78. The method of Embodiment 77, wherein said treating comprises surgery, systemic chemotherapy, radiotherapy or targeted therapy.

Embodiment 79. The method of any of Embodiments 70 to 78, wherein an increased number of methylated CpG sites relative to a standard control indicates a higher risk of gastrointestinal cancer.

Embodiment 80. The method of any of the above Embodiments, wherein the DNA sample is substantially cell-free DNA.

Embodiment 81. The method of any of the above Embodiments, wherein the DNA sample is from a biological fluid.

Embodiment 82. The method of Embodiment 81, wherein the biological fluid is plasma.

Embodiment 83. The method of any of the above Embodiments, wherein the level of methylation of CpG sites is higher than a DNA sample from a standard control.

Embodiment 84. A computer program product comprising a machine-readable medium storing instructions that, when executed by at least one programmable processor, cause the at least one programmable processor to perform operations comprising the method of any of the above Embodiments.

Embodiment 85. A system comprising computer hardware configured to perform operations comprising the method of any of Embodiments 1 to 83.

Embodiment 86. A computer-implemented method comprising the method of any of Embodiments 1 to 83.

Embodiment 87. A method for preparing a DNA fraction from a subject at risk of developing a gastrointestinal cancer, said method comprising: (a) extracting DNA from a substantially cell-free sample of biological fluid of the subject to obtain extracellular DNA; and (b) determining a level of DNA methylation in a subject at risk according to any of Embodiments 1 to 79.

### Embodiments A1-A46

Embodiment A1. A method of diagnosing cancer in a patient, the method comprising: (a) detecting a level of methylated CpG sites within a plurality of gene regions in a DNA sample obtained from the patient, and (b) diagnosing the patient with cancer when the DNA sample has an increased level of methylated CpG sites, relative to a standard control, within the plurality of gene regions; wherein: (i) the cancer is a gastrointestinal cancer and the plurality of gene regions comprises at least 50 different gene regions in Table PGI; (ii) the cancer is colorectal cancer and the plurality of gene regions comprises at least 5 different gene regions in Table CRC; (iii) the cancer is hepatocellular carcinoma and the plurality of gene regions comprises at least 5 different gene regions in Table HCC; (iv) the cancer is esophageal squamous cell carcinoma and the plurality of gene regions comprises at least 5 different gene regions in Table ESCC; (v) the cancer is gastric cancer and the plurality of gene regions comprises at least 5 different gene regions in Table GC; (vi) the cancer is esophageal adenocarcinoma and the plurality of gene regions comprises at least 5 different gene regions in Table EAC; (vii) the cancer is pancreatic ductal adenocarcinoma and the plurality of gene regions comprises at least 5 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

Embodiment A2. A method of treating cancer in a patient in need thereof, the method comprising: (a) detecting an increased level of methylated CpG sites, relative to a standard control, within a plurality of gene regions in a DNA sample obtained from the patient; and (b) treating the patient for cancer; wherein: (i) the cancer is a gastrointestinal cancer, and the plurality of gene regions comprises at least 50 different gene regions in Table PGI; (ii) the cancer is colorectal cancer, and the plurality of gene regions comprises at least 5 different gene regions in Table CRC; (iii) the cancer is hepatocellular carcinoma, and the plurality of gene regions comprises at least 5 different gene regions in Table HCC; (iv) the cancer is esophageal squamous cell carcinoma, and the plurality of gene regions comprises at least 5 different gene regions in Table ESCC; (v) the cancer is gastric cancer, and the plurality of gene regions comprises at least 5 different gene regions in Table GC; (vi) the cancer is esophageal adenocarcinoma, and the plurality of gene regions comprises at least 5 different gene regions in Table EAC; (vii) the cancer is pancreatic ductal adenocarcinoma, and the plurality of gene regions comprises at least 5 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

Embodiment A3. A method of monitoring risk for developing cancer in a patient in need thereof or monitoring treatment in a patient having cancer, the method comprising: (a) detecting a level of methylated CpG sites within a plurality of gene regions in a DNA sample from the patient at a first time point; (b) detecting a level of methylated CpG sites within the plurality of gene regions in a DNA sample from the patient at a second time point, wherein the second time point is later than the first time point; and (c) comparing the level of methylated CpG sites at the second time point to the level of methylated CpG sites at the first time point, thereby monitoring risk or monitoring treatment; wherein: (i) the cancer is a gastrointestinal cancer and the plurality of gene regions comprises at least 50 different gene regions in Table PGI; (ii) the cancer is colorectal cancer and the plurality of gene regions comprises at least 5 different gene regions in Table CRC; (iii) the cancer is hepatocellular carcinoma and the plurality of gene regions comprises at least 5 different gene regions in Table HCC; (iv) the cancer is esophageal squamous cell carcinoma and the plurality of gene regions comprises at least 5 different gene regions in Table ESCC; (v) the cancer is gastric cancer and the plurality of gene regions comprises at least 5 different gene regions in Table GC; (vi) the cancer is esophageal adenocarcinoma and the plurality of gene regions comprises at least 5 different gene regions in Table EAC; (vii) the cancer is pancreatic ductal adenocarcinoma and the plurality of gene regions comprises at least 5 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

Embodiment A4. A method of detecting a level of DNA methylation in a patient at risk of developing a cancer, the method comprising determining the degree of methylation of CpG sites within a plurality of gene regions in a DNA sample from the patient; wherein: (i) the cancer is a gastrointestinal cancer and the plurality of gene regions comprises at least 50 different gene regions in Table PGI; (ii) the cancer is colorectal cancer and the plurality of gene regions comprises at least 5 different gene regions in Table CRC; (iii) the cancer is hepatocellular carcinoma and the plurality of gene regions comprises at least 5 different gene regions in Table HCC; (iv) the cancer is esophageal squamous cell carcinoma and the plurality of gene regions comprises at least 5 different gene regions in Table ESCC; (v) the cancer is gastric cancer and the plurality of gene regions comprises at least 5 different gene regions in Table GC; (vi) the cancer is esophageal adenocarcinoma and the plurality of gene regions comprises at least 5 different gene regions in Table EAC; (vii) the cancer is pancreatic ductal adenocarcinoma and the plurality of gene regions comprises at least 5 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

Embodiment A5. The method of Embodiment A1, wherein an increased level of methylation of CpG sites relative to a standard control indicates a higher risk of cancer.

Embodiment A6. The method of any one of Embodiments A1 to A5, wherein: (i) the cancer is a gastrointestinal cancer, and the plurality of gene regions comprises at least 100 different gene regions in Table PGI; (ii) the cancer is colorectal cancer, and the plurality of gene regions comprises at least 10 different gene regions in Table CRC; (iii) the cancer is hepatocellular carcinoma, and the plurality of gene regions comprises at least 10 different gene regions in Table HCC; (iv) the cancer is esophageal squamous cell carcinoma, and the plurality of gene regions comprises at least 10 different gene regions in Table ESCC; (v) the cancer is gastric cancer, and the plurality of gene regions comprises at least 5 different gene regions in Table GC; (vi) the cancer is esophageal adenocarcinoma, and the plurality of gene regions comprises at least 10 different gene regions in Table EAC; (vii) the cancer is pancreatic ductal adenocarcinoma, and the plurality of gene regions comprises at least 10 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 100 different gene regions in Table MCC.

Embodiment A7. The method of Embodiment A6, wherein: (i) the cancer is a gastrointestinal cancer, and the plurality of gene regions comprises at least 150 different gene regions in Table PGI; (ii) the cancer is colorectal cancer, and the plurality of gene regions comprises at least 50 different gene regions in Table CRC; (iii) the cancer is hepatocellular carcinoma, and the plurality of gene regions comprises at least 50 different gene regions in Table HCC; (iv) the cancer is esophageal squamous cell carcinoma, and the plurality of gene regions comprises at least 50 different gene regions in Table ESCC; (v) the cancer is gastric cancer, and the plurality of gene regions comprises at least 5 different gene regions in Table GC; (vi) the cancer is esophageal adenocarcinoma, and the plurality of gene regions comprises at least 50 different gene regions in Table EAC; (vii) the cancer is pancreatic ductal adenocarcinoma, and the plurality of gene regions comprises at least 50 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 150 different gene regions in Table MCC.

Embodiment A8. The method of Embodiment A7, wherein: (i) the cancer is a gastrointestinal cancer, and the plurality of gene regions comprises at least 250 different gene regions in Table PGI; (ii) the cancer is colorectal cancer, and the plurality of gene regions comprises at least 100 different gene regions in Table CRC; (iii) the cancer is hepatocellular carcinoma, and the plurality of gene regions comprises at least 100 different gene regions in Table HCC; (iv) the cancer is esophageal squamous cell carcinoma, and the plurality of gene regions comprises at least 100 different gene regions in Table ESCC; (v) the cancer is gastric cancer, and the plurality of gene regions comprises at least 5 different gene regions in Table GC; (vi) the cancer is esophageal adenocarcinoma, and the plurality of gene regions comprises at least 100 different gene regions in Table EAC; (vii) the cancer is pancreatic ductal adenocarcinoma, and the plurality of gene regions comprises at least 100 different gene regions in Table PDAC; or (viii) the cancer is a gastrointestinal cancer selected from the group consisting of colorectal cancer, hepatic cancer, esophageal cancer, and pancreatic cancer, and the plurality of gene regions comprises at least 250 different gene regions in Table MCC.

Embodiment A9. The method of any one of Embodiments A1 to A8, wherein: (i) the cancer is gastrointestinal cancer.

Embodiment A10. The method of Embodiment A9, wherein the plurality of gene regions comprise the first 50 gene regions in Table PGI.

Embodiment A11. The method of Embodiment A9, wherein the plurality of gene regions comprise the first 150 gene regions in Table PGI.

Embodiment A12. The method of any one of Embodiments A1 to A8, wherein: (ii) the cancer is colorectal cancer.

Embodiment A13. The method of Embodiment A12, wherein the plurality of gene regions comprise the first 10 gene regions in Table CRC.

Embodiment A14. The method of Embodiment A12, wherein the plurality of gene regions comprise the first 50 gene regions in Table CRC.

Embodiment A15. The method of any one of Embodiments A1 to A8, wherein: (iii) the cancer is hepatocellular carcinoma.

Embodiment A16. The method of Embodiment A15, wherein the plurality of gene regions comprise the first 10 gene regions in Table HCC.

Embodiment A17. The method of Embodiment A15, wherein the plurality of gene regions comprise the first 50 gene regions in Table HCC.

Embodiment A18. The method of any one of Embodiments A1 to A8, wherein: (iv) the cancer is esophageal squamous cell carcinoma.

Embodiment A19. The method of Embodiment A18, wherein the plurality of gene regions comprise the first 10 gene regions in Table ESCC.

Embodiment A20. The method of Embodiment A18, wherein the plurality of gene regions comprise the first 50 gene regions in Table ESCC.

Embodiment A21. The method of any one of Embodiments A1 to A8, wherein: (v) the cancer is gastric cancer.

Embodiment A22. The method of Embodiment A21, wherein the plurality of gene regions comprise the first 10 gene regions in Table GC.

Embodiment A23. The method of Embodiment A21, wherein the plurality of gene regions comprise the first 50 gene regions in Table GC.

Embodiment A24. The method of any one of Embodiments A1 to A8, wherein: (vi) the cancer is esophageal adenocarcinoma.

Embodiment A25. The method of Embodiment A24, wherein the plurality of gene regions comprise the first 10 gene regions in Table EAC.

Embodiment A26. The method of Embodiment A24, wherein the plurality of gene regions comprise the first 50 gene regions in Table EAC.

Embodiment A27. The method of any one of Embodiments A1 to A8, wherein: (vii) the cancer is pancreatic ductal adenocarcinoma.

Embodiment A28. The method of Embodiment A25, wherein the plurality of gene regions comprise the first 10 gene regions in Table PDAC.

Embodiment A29. The method of Embodiment A25, wherein the plurality of gene regions comprise the first 50 gene regions in Table PDAC.

Embodiment A30. The method of any one of Embodiments A1 to A8, where: (viii) the cancer is a gastrointestinal cancer selected from the group consisting of colorectal cancer, hepatic cancer, esophageal cancer, and pancreatic cancer.

Embodiment A31. The method of Embodiment A30, further comprising identifying the tissue of origin based on the plurality of gene regions having the increased levels of methylated CpG sites, thereby identifying the cancer as colorectal cancer, hepatic cancer, esophageal cancer, and pancreatic cancer.

Embodiment A32. The method of Embodiment A30 or A31, wherein the plurality of gene regions comprise the first 50 gene regions in Table MCC.

Embodiment A33. The method of Embodiment A30 or A31, wherein the plurality of gene regions comprise the first 150 gene regions in Table MCC.

Embodiment A34. The method of any one of Embodiments A1 to A33, wherein the DNA sample is cell-free-DNA.

Embodiment A35. The method of any one of Embodiments A1 to A33, wherein the DNA sample is cell-free-DNA in plasma.

Embodiment A36. The method of any one of Embodiments A1 to A35, wherein the cancer is Stage I.

Embodiment A37. The method of any one of Embodiments A1 to A35, wherein the cancer is Stage II.

Embodiment A38. The method of any one of Embodiments A1 to A35, wherein the cancer is Stage III.

Embodiment A39. The method of any one of Embodiments A1 to A38, wherein the standard control is a patient or population of patients that do not have cancer.

Embodiment A40. The method of any of Embodiments A1 to A39, further comprising performing a confirmatory diagnostic procedure on the patient.

Embodiment A41. The method of any one of Embodiments A1 and A3-A40, further comprising treating the patient for cancer.

Embodiment A42. The method of Embodiment A2 or A41, wherein treating the patient for cancer comprises surgically removing the cancer from the patient, administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Embodiment A43. The method of Embodiment A2 or A41, wherein treating the patient for cancer comprises administering to the patient an effective amount of radiotherapy, administering to the patient an effective amount of chemotherapy, administering to the patient an effective amount of targeted therapy, administering to the patient an effective amount of immunotherapy, or a combination of two or more thereof.

Embodiment A44. The method of Embodiment A2 or A41, wherein treating the patient for cancer comprises administering to the patient an effective amount of chemotherapy, administering to the patient an effective amount of targeted therapy, administering to the patient an effective amount of immunotherapy, or a combination of two or more thereof.

Embodiment A45. The method of Embodiment A2 or A41, wherein treating the patient for cancer comprises administering to the patient an effective amount of chemotherapy.

Embodiment A46. The method of any one of Embodiments A1 or A43, wherein detecting methylated CpG sites in the DNA sample obtained from the patient is performed in vitro.

### Examples

A genome-wide DNA methylation analysis for multiple gastrointestinal (GI) cancers was undertaken to develop a pan-gastrointestinal (panGI) diagnostic assay. By analyzing the DNA methylation data from 1940 tumor and adjacent normal tissues from TCGA and GSE72872 datasets, DMRs were first identified between individual GI cancers and adjacent normal tissues, as well as across all GI cancers. A list of 67,832 tissue DMRs was next prioritized encompassing a 25.6 Mb genomic region by incorporating all significant DMRs across various GI cancers to design a custom SeqCap Epi, targeted bisulfite sequencing platform. Subsequent investigation of these tissue-specific DMRs in 300 cf-DNA specimens and applying machine learning algorithms led to the development of three distinct categories of DMR panels: (1) Cancer-specific biomarker panels with an AUC values of 0.98 (Colorectal cancer, CRC), 0.94 (Esophageal squamous cell carcinoma, ESCC), 0.90 (Esophageal adenocarcinoma, EAC), 0.90 (Gastric cancer, GC), 0.98 (Hepatocellular carcinoma, HCC), and 0.85 (Pancreatic ductal adenocarcinoma, PDAC); (2) A pan-GI panel that detected all GI cancers with an AUC of 0.90; and (3) A multi-cancer prediction panel, EpiPanGI Dx, with a prediction accuracy around 0.85-0.95 for most GI cancers. Utilizing a novel biomarker discovery approach, the first evidence for a cell-free DNA methylation biomarker assay that offer a robust diagnostic accuracy for all gastrointestinal cancers is provided herein.

A genome-wide DNA methylation analysis was undertaken for multiple GI cancers, followed by development of a novel cf-DNA methylation biomarker panels for the early detection of individual GI cancers, a panGI diagnostic panel as well as multi GI cancer prediction panel (EpiPanGI Dx). Most studies so far investigated genome-wide methylation patterns at tissue level in individual cancers and subsequently selected most significant tissue markers and tested those in the cfDNA of corresponding cancer type. Hence, these single cancer study approaches fail to analyze DNA methylation patterns in an unbiased and comprehensive manner, and thereby lack the ability to discover cancer specific markers. To address this challenge and to identify methylation markers across GI cancers, the inventors analyzed Illumina 450k microarray methylation data of 1940 tumor and adjacent normal tissues and identified the DMRs between individual GI cancers and adjacent normal tissues, as well as across all GI cancers. The inventors next prioritized a list of DMRs encompassing a 25.6 Mb genomic region by incorporating all identified DMRs across various GI cancers to design a custom SeqCap Epi, targeted bisulfite sequencing platform, optimized for analysis of low-abundance cf-DNA derived from plasma specimens. Using this approach, the inventors sequenced 300 plasma specimens from all GI cancers, as well as age-matched healthy controls and identified unique DMR panels for the detection of various GI cancers.

Analysis of Genome-Wide Tissue Methylation Data Across GI Cancers and Development of a GI Targeted Bisulfite Sequencing Panel (gitBS)

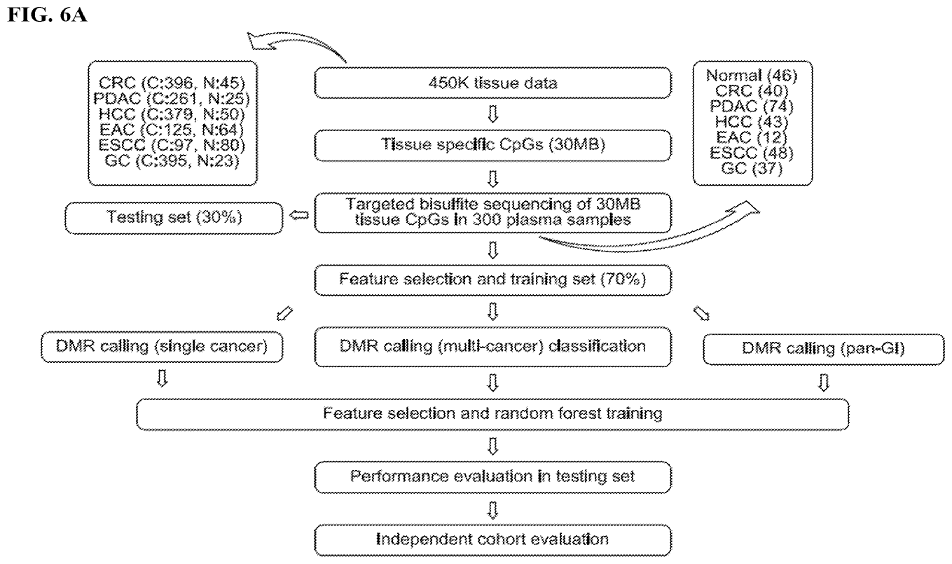

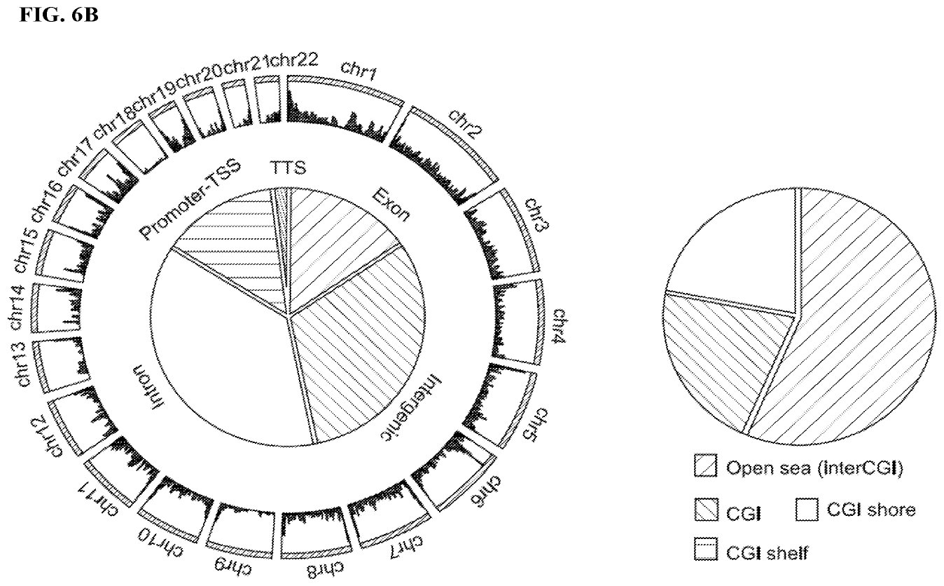

The study design describing the tissue discovery, followed by plasma cell-free DNA validation process is illustrated in FIG. 6A. The inventors first analyzed 450K methylation array data of tumor and adjacent normal tissues from six different GI cancers: colorectal cancer (CRC), pancreatic ductal adenocarcinoma (PDAC), hepatocellular carcinoma (HCC), esophageal adenocarcinoma (EAC), esophageal squamous cell carcinoma (ESCC) and gastric cancer (GC) consisting of a total of 1940 tumor and adjacent normal tissues. In normal and tumor comparison for each GI cancer as well as across GI cancers, the inventors totally identified 67,832 regions of interest (ROI) based on the significant differentially methylated probes with a p-value of less than 0.001 and an absolute delta beta of 0.20 across all the comparisons. The covered regions are highly enriched for promoter as well as gene body region, which is more susceptible to aberrant methylation alternation during oncogenesis. Circos plot showing the regions across the chromosomes are presented in FIG. 6B. The inventors then finalized the list of ROIs by merging the overlapped ROIs from different GI cancers at tissue level and used it to design a targeted bisulfite sequencing platform, which the inventors termed as “GI targeted bisulfite sequencing (gitBS)” panel. Unlike the earlier studies, the inventors have taken every significant probe on 450K tissue analysis across the comparisons to build gitBS, with the aim of profiling these regions in larger number of plasma samples with a greater coverage. Compared to a previously reported strategy (14), gitBS included much broader genome region, covering around 1% of human genome that's selected from meticulous analysis of all GI cancers at tissue level.

Evaluation of gitBS in Plasma cfDNA and Development of Various Cell-Free DNA Methylation Panels for GI Cancers Detection

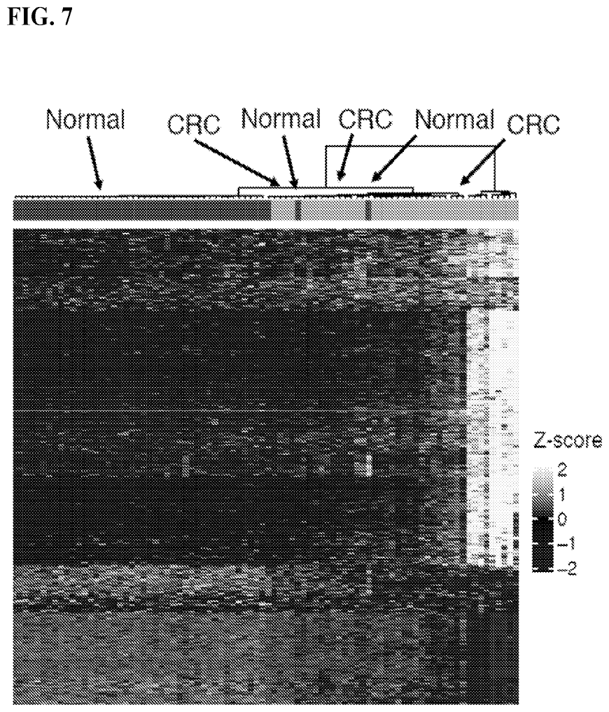

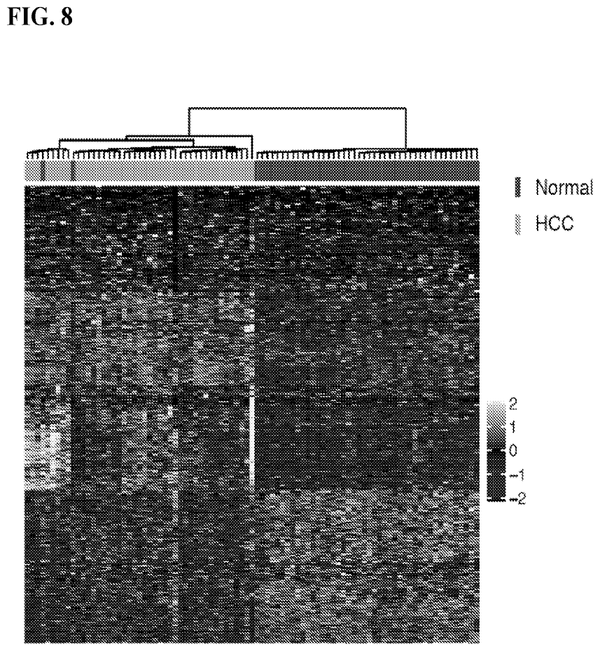

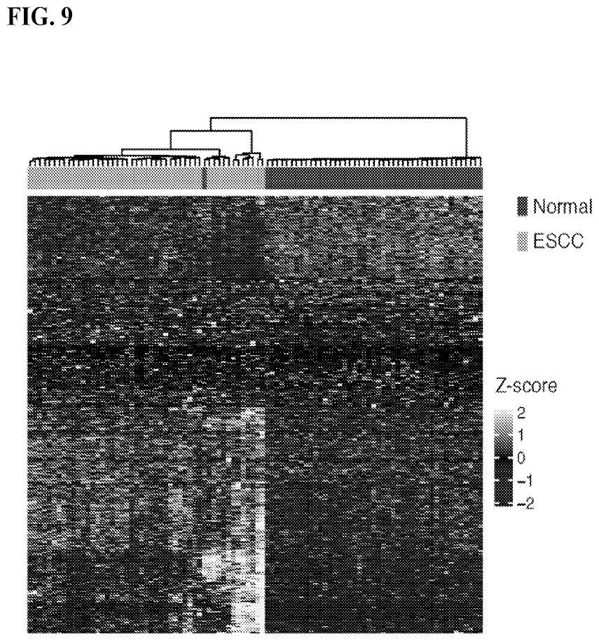

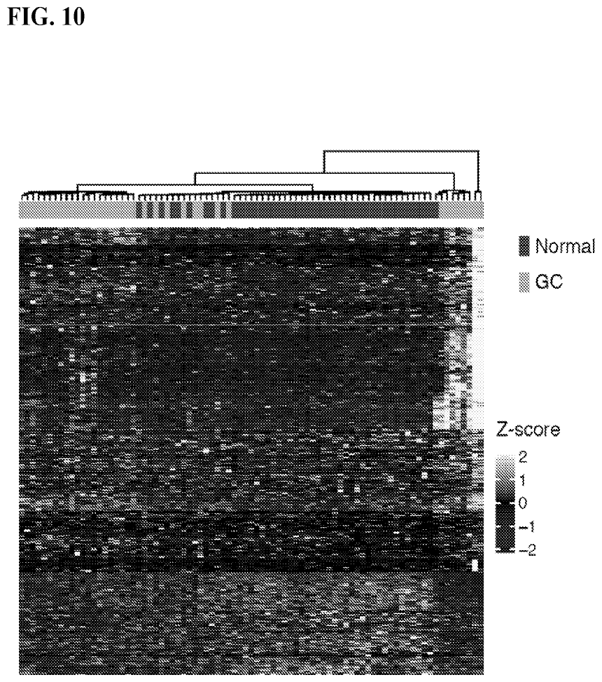

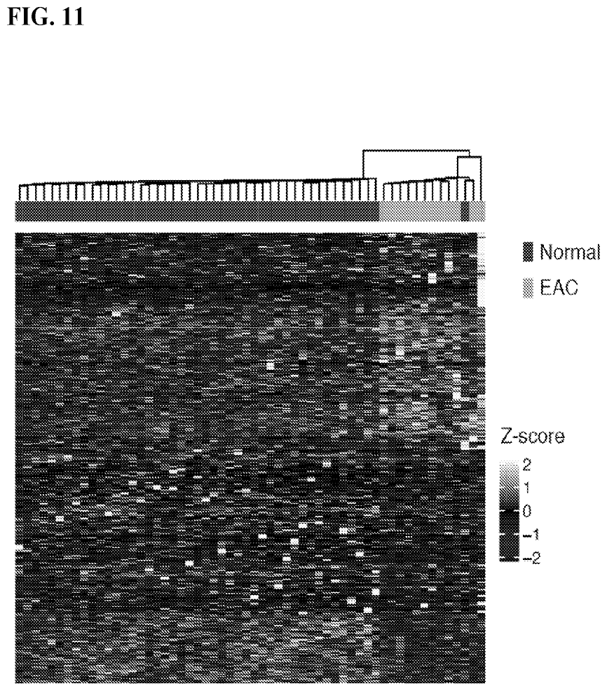

In a novel approach, here the inventors evaluated the comprehensive list of tissue derived markers (30 MB) identified across GI cancers in plasma cell-free DNA. Briefly, the inventors performed gitBS on 300 plasma samples in total collected from patients with six different GI cancers—CRC, PDAC, HCC, EAC, ESCC and GC, and healthy age-matched controls. The inventors achieved average 40× coverage for gitBS on all plasma samples at only $70 per sample, indicating that this strategy is feasible for large-scale studies. In the comparison of individual GI cancers with healthy controls, the inventors identified a total of 216,887 differentially methylated CpGs (DMC) consisting of 10,677 differentially methylated regions (DMR). The number of DMRs identified in CRC is 5689, EAC is 1177, ESCC is 1063, GC is 949, HCC is 1072, and PDAC is 1528. To investigate the diagnostic power of the identified DMR panels across each GI cancer, the inventors performed hierarchical clustering for each GI cancer based on the identified DMRs for that cancer type. For most GI cancers, the inventors observed clear separation of two clusters representing cancer and normal samples. As for PDAC, although the boundary between the cancer and normal clusters is less clear, most PDAC blood samples were clustered together (FIGS. 7-12), indicating that these DMRs could be used as biomarkers for GI cancer detection.

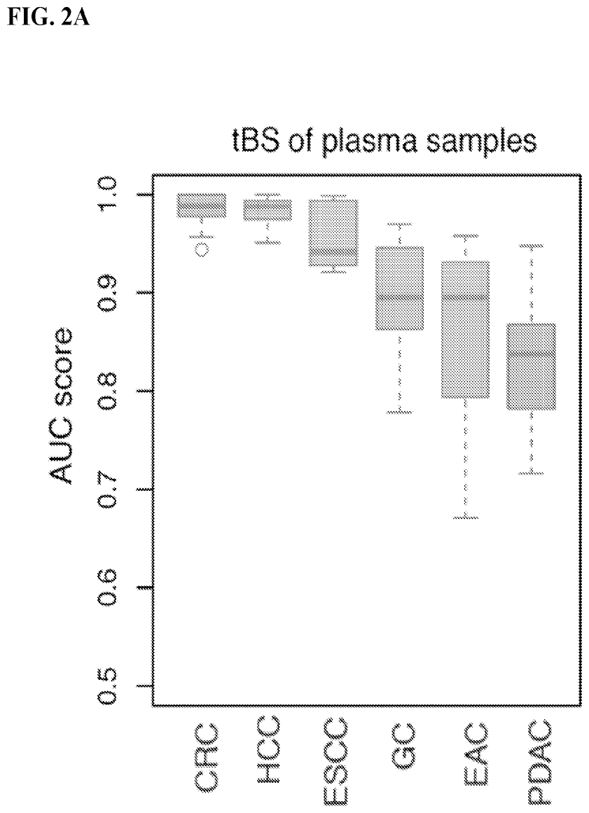

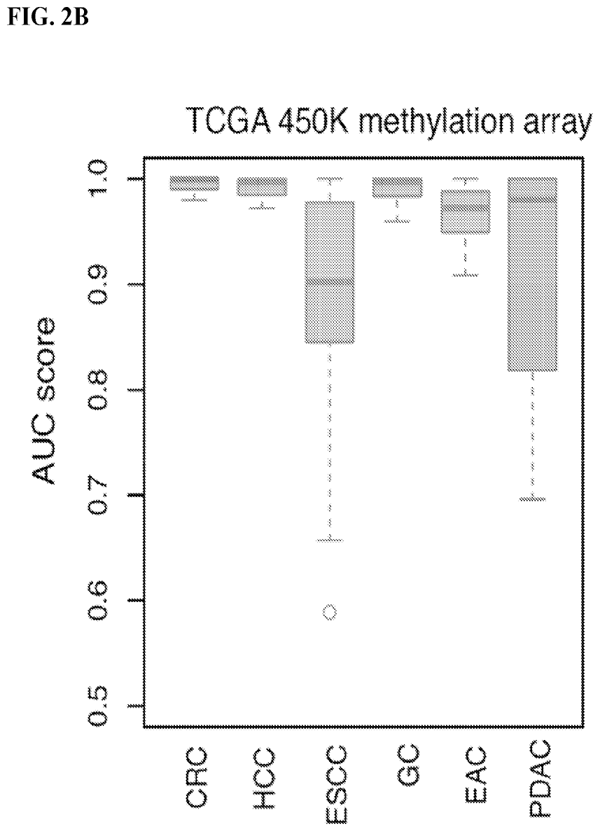

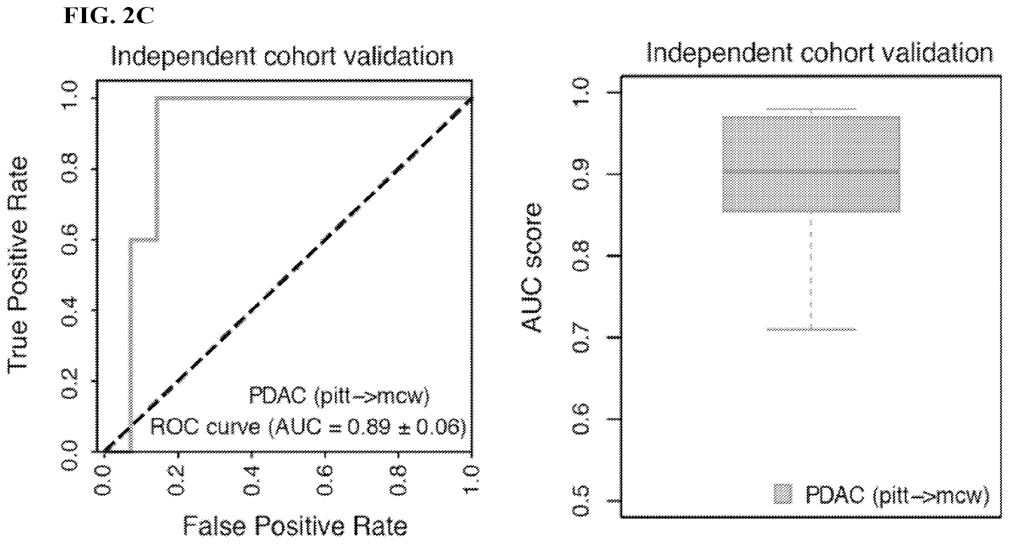

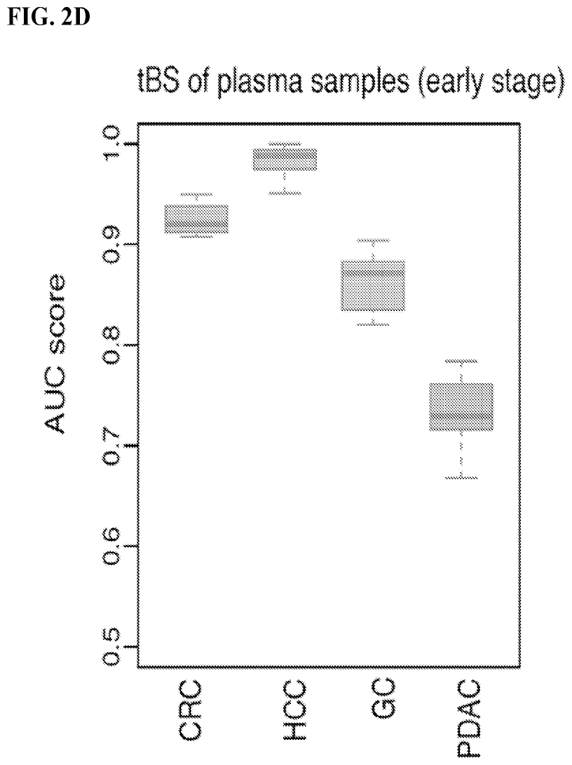

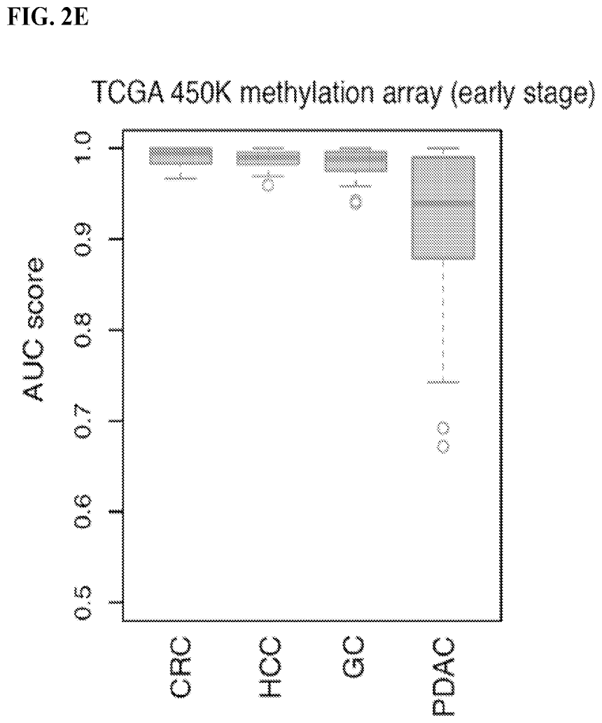

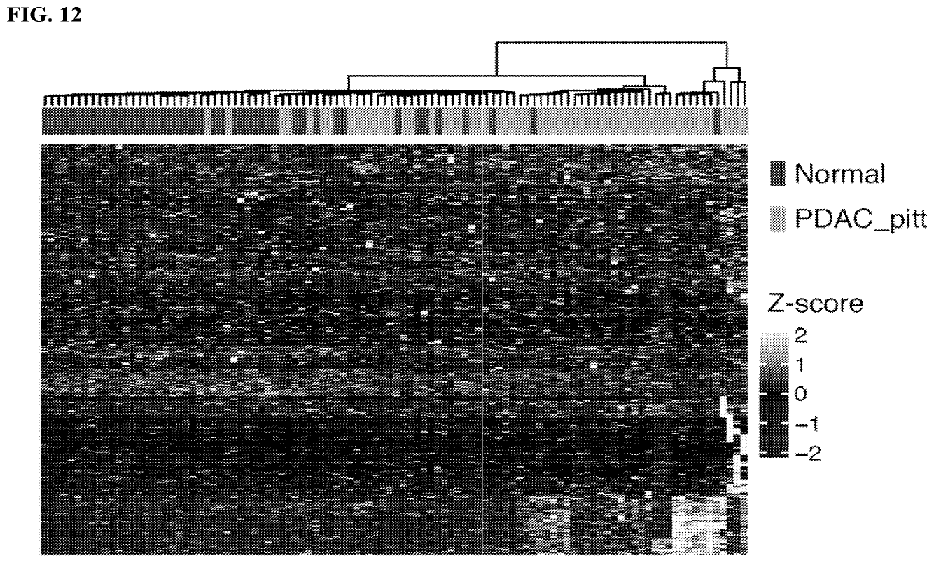

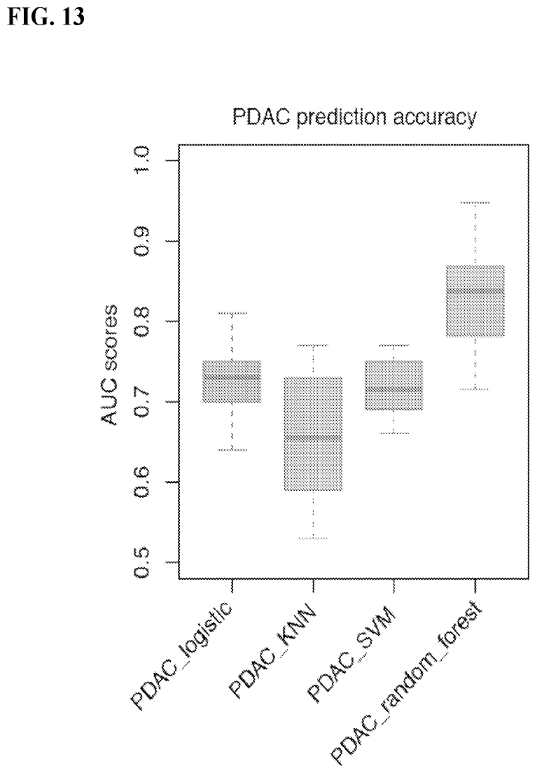

The inventors further exploited machine learning techniques to evaluate the DMRs in cancer detection for each GI cancer. The plasma samples of GI cancer patients and healthy controls were first split into training and test sets in a manner of 70%-30%. In order to avoid leaking information from test set to model training, the inventors called de novo DMRs between GI cancer and healthy control only with samples from training set rather than using the aforementioned DMRs identified with all samples. Next, the inventors performed feature selection based on the Boruta algorithm, which is shown to be powerful for biological features (15). The chosen DMRs were then used to train a random forest model, which outperforms several other machine learning techniques for GI cancer detection (FIG. 13). Finally, the inventors evaluated the model performance by the Area Under the ROC Curve (AUC) score with the test set samples. The entire process was repeated for 10 times to prevent biases due to data set splitting. For CRC and HCC, the inventors' cancer prediction models achieved the best performance with the median AUC scores of 0.99, while the prediction models for the other GI cancers were around 0.90, which is higher or comparable to what has been reported earlier (16, 17) (FIG. 2A).

The inventors next asked the question about the performance of these plasma derived DMR panels established using machine learning approaches in distinguishing GI cancer tissues from adjacent normal. As expected, the median AUC scores of models for most GI cancers were close to 1.0. In line with the plasma data, the model for predicting PDAC tissue has relatively low performance (FIG. 2B). Besides the prediction accuracy, the inventors also examined the reliability of the GI cancer prediction models by validating the DMR panels in an independent cohort of PDAC plasma samples as a proof of principle. The aforementioned machine learning model, trained and tested with PDAC plasma samples, achieved even higher prediction accuracy in the second independent PDAC cohort with an AUC of 0.89 (FIG. 2C).

Since the ultimate goal of cancer screening is to find cancer at early stage, the inventors then evaluated whether the plasma DMRs could detect early stage GI cancers in CRC, HCC, GC and PDAC. The inventors' models achieved median AUC scores of 0.92, 0.99, 0.87 and 0.73 for CRC, HCC, GC and PDAC respectively for early stage cancers. Once again, these DMR panels achieved excellent AUC values close to 1 when applied to the early stage tumor tissues in these cancers (FIGS. 2D-2E). Altogether, these results indicate that DNA methylation aberrations can be used for detecting single GI cancers.

### Pan-GI Cancer Detection and Multi GI-Cancer Classification

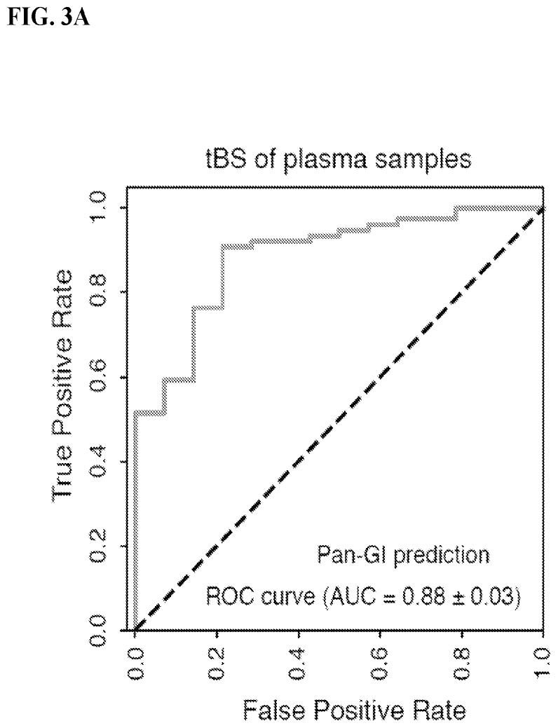

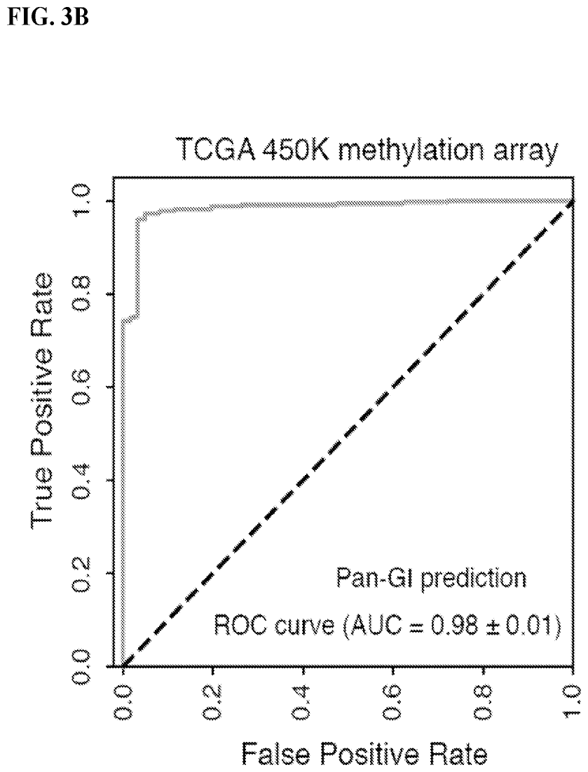

In clinical practice, it may be cumbersome to use different prediction models for each GI cancer. Having done this study at pan-GI level, the inventors then asked if such a classifier can be identified using the inventors' data. Therefore, the inventors pooled the training sets and test sets used for each single GI cancer prediction together as the pan-GI training set and test set, respectively. The DMRs identified from each GI cancers were also pooled for pan-GI cancer feature selection and model training. The inventors achieved a median AUC of 0.88 for pan-GI cancer prediction model in the test set plasma cohort (FIG. 3A). Similarly, the plasma DMRs achieved an excellent AUC of 0.98 in distinguishing pan-GI cancer tissues from normal tissues (FIG. 3B).

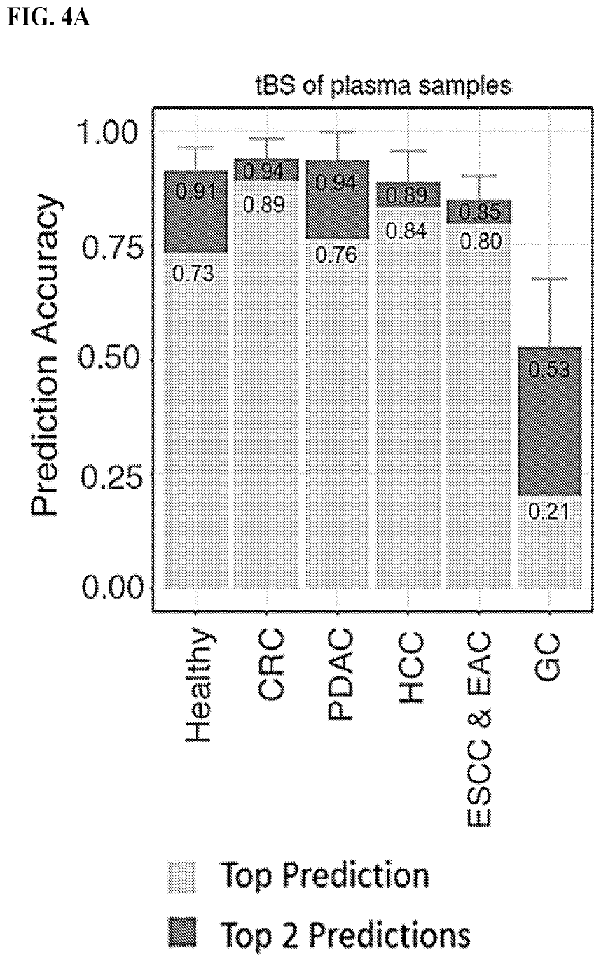

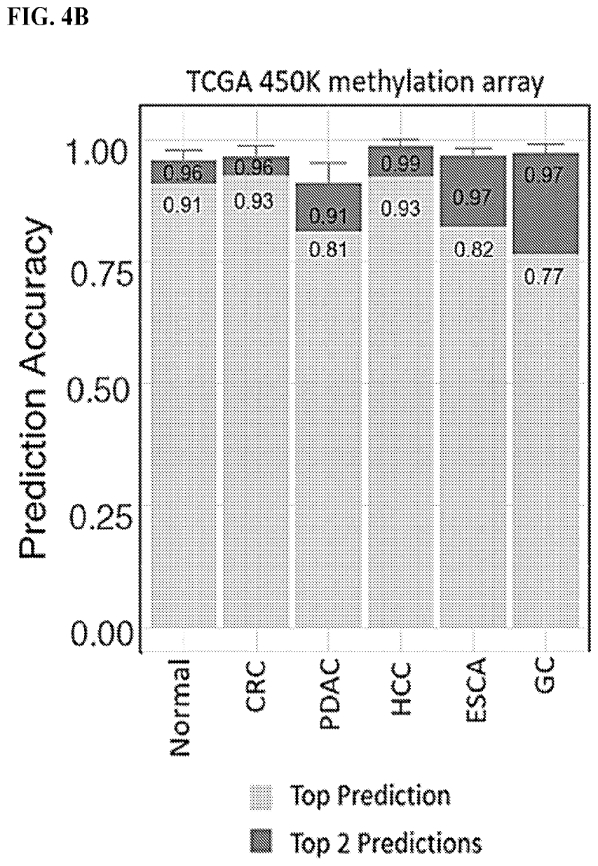

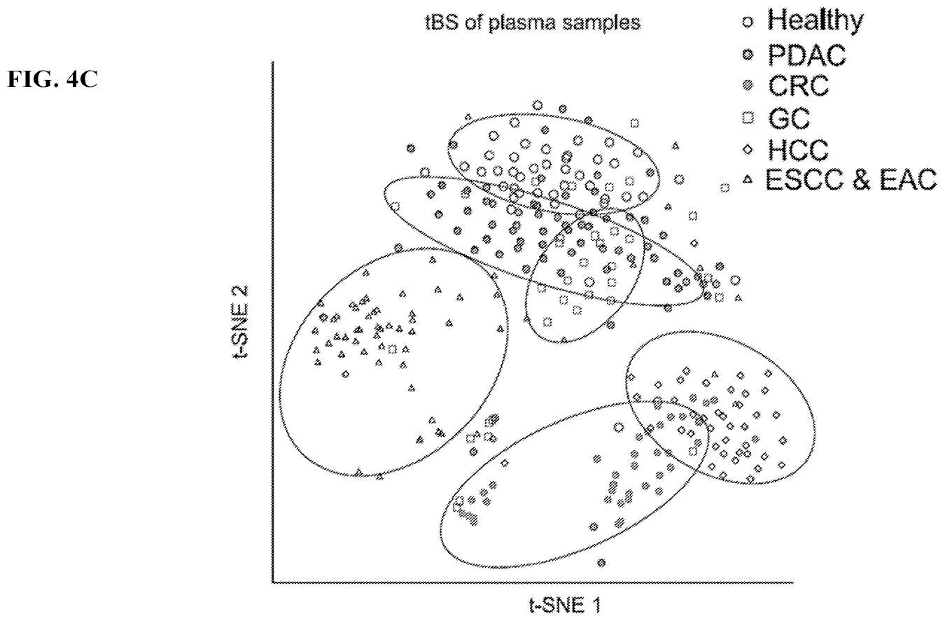

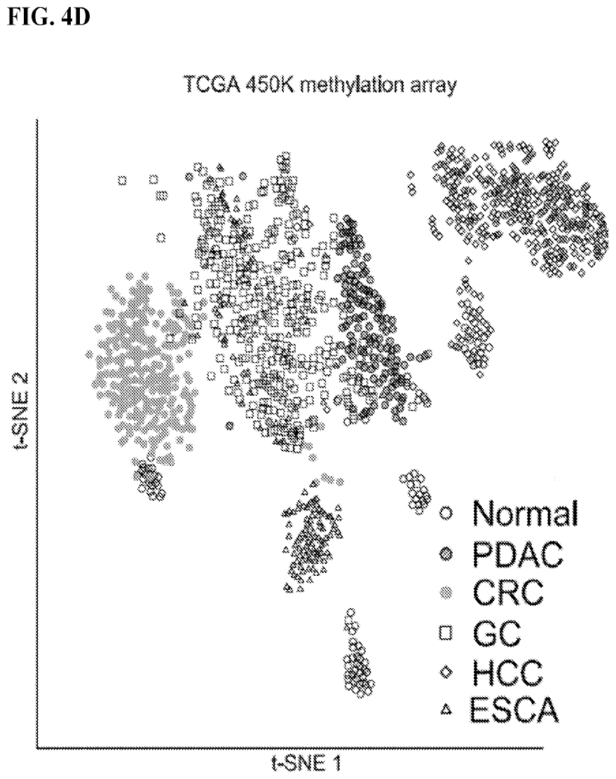

For a subject with positive pan-GI cancer screening result, a physician may also want to know which GI cancer this subject is likely bearing before prescribing further examinations. Therefore, the inventors further trained a random forest model for GI cancer classification. Given that ESCC and EAC are both developed from esophagus, the inventors treated them as the same class in the inventors' model. For each class versus the rest, the inventors identified class-specific plasma DMRs, which were then pooled for feature selection and model training. In the test set, the inventors' models classified samples into normal plasma, CRC, PDAC, HCC and ESCC/EAC with higher accuracy than previous studies (16) (FIG. 4A). The t-SNE plot also showed clear separation of most GI cancers (FIG. 4C). In addition, the class-specific plasma DMRs also classified GI cancer tissues with high accuracy (FIGS. 4B-4D). Collectively, these results prove the feasibility of utilizing cfDNA methylation markers for not only GI cancer screening but also locating the tissue of origin of GI cancers.

Identification of the Minimum Number of DMRs that Needed to Achieve the Best Accuracy Across the GI Cancers

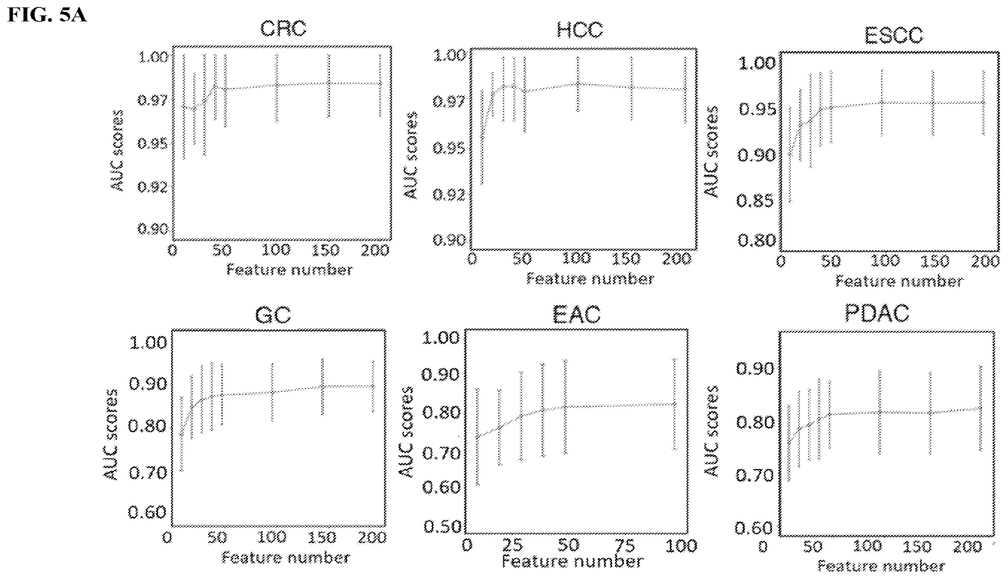

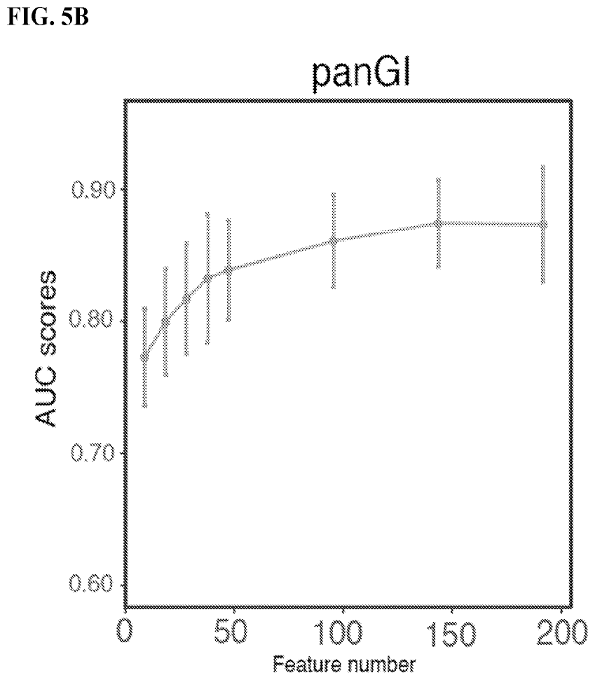

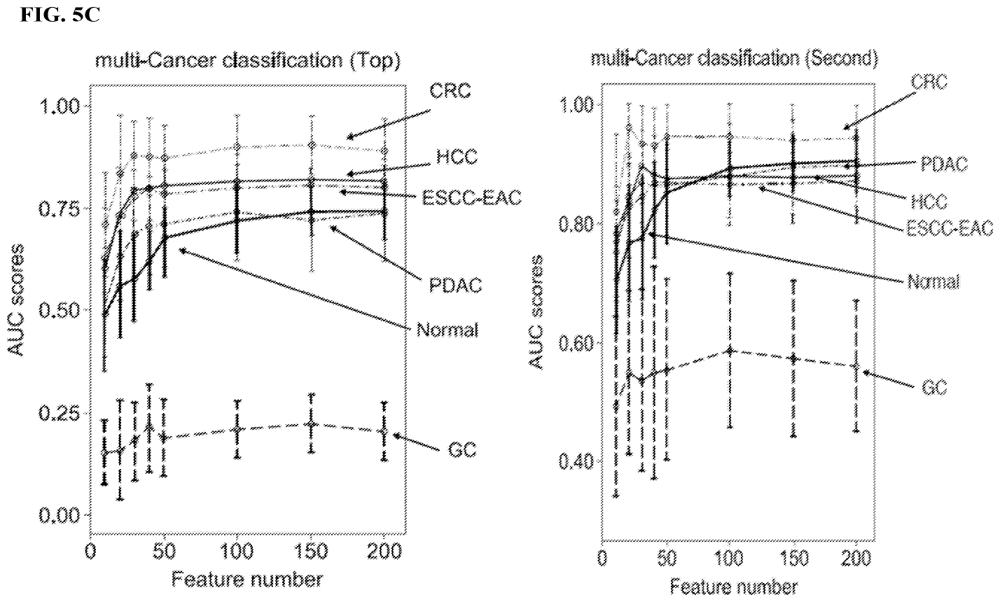

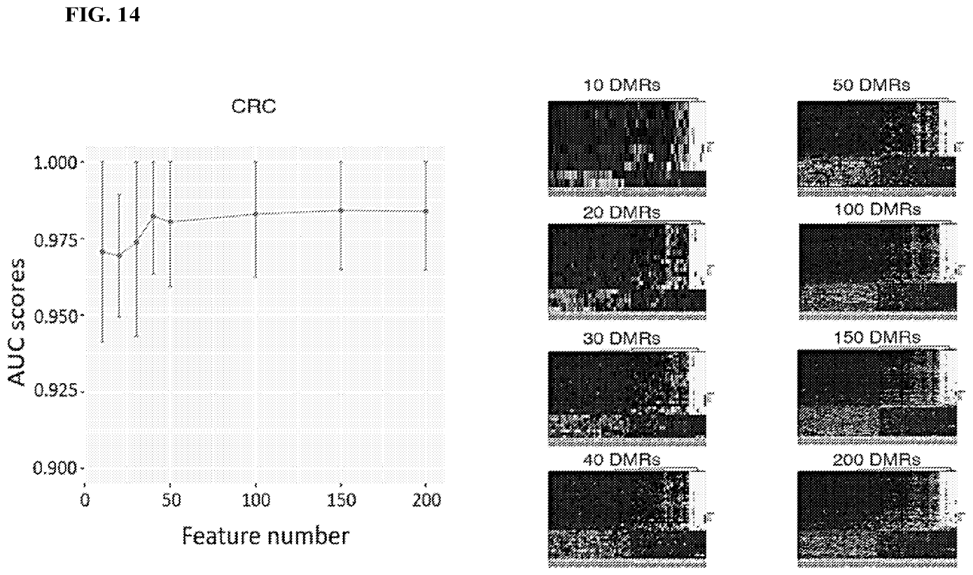

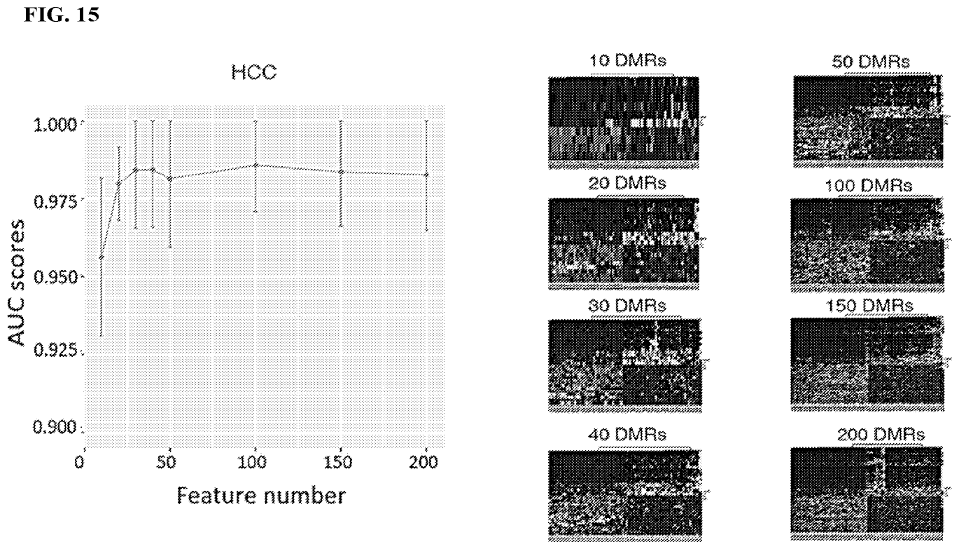

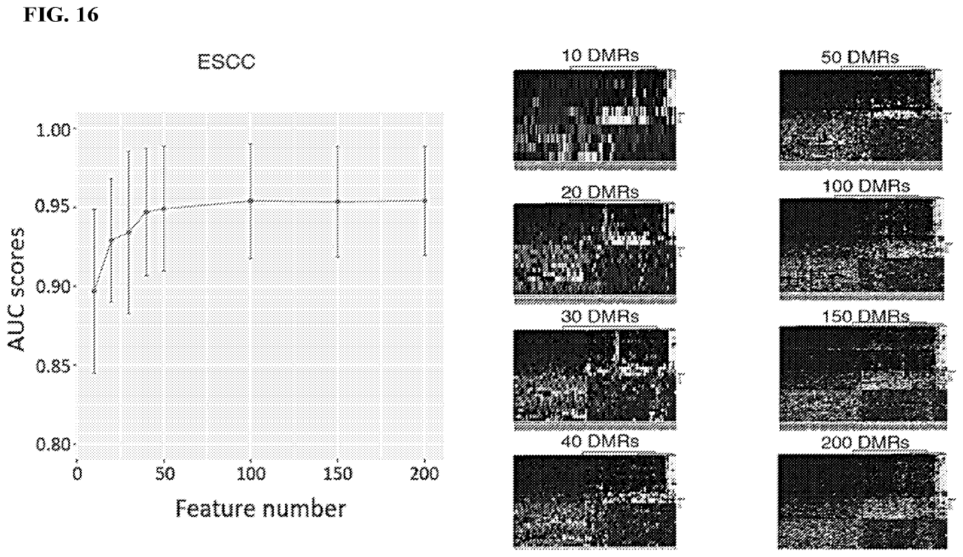

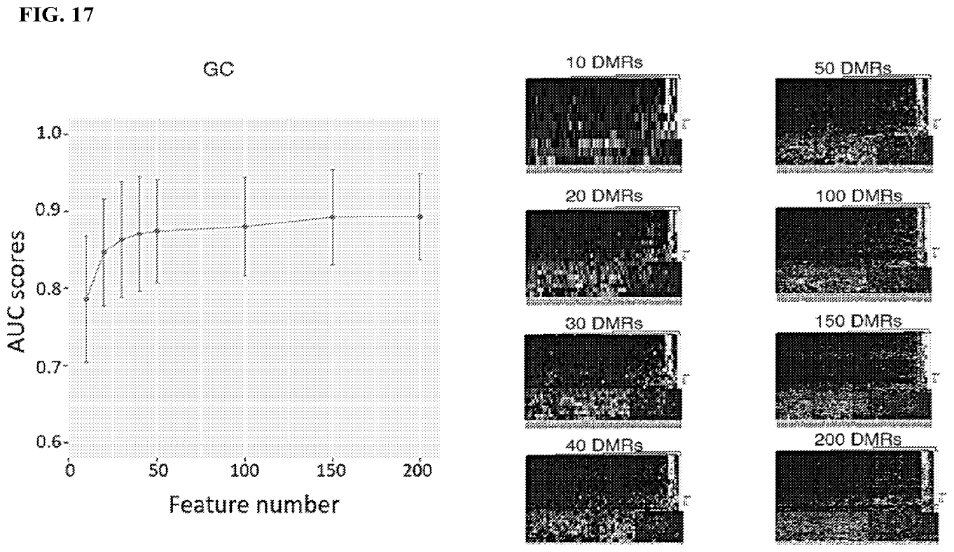

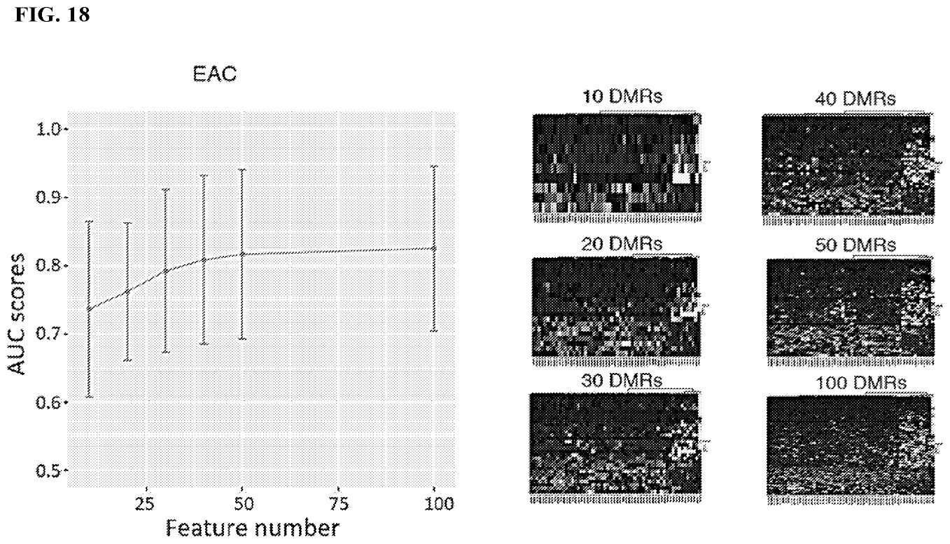

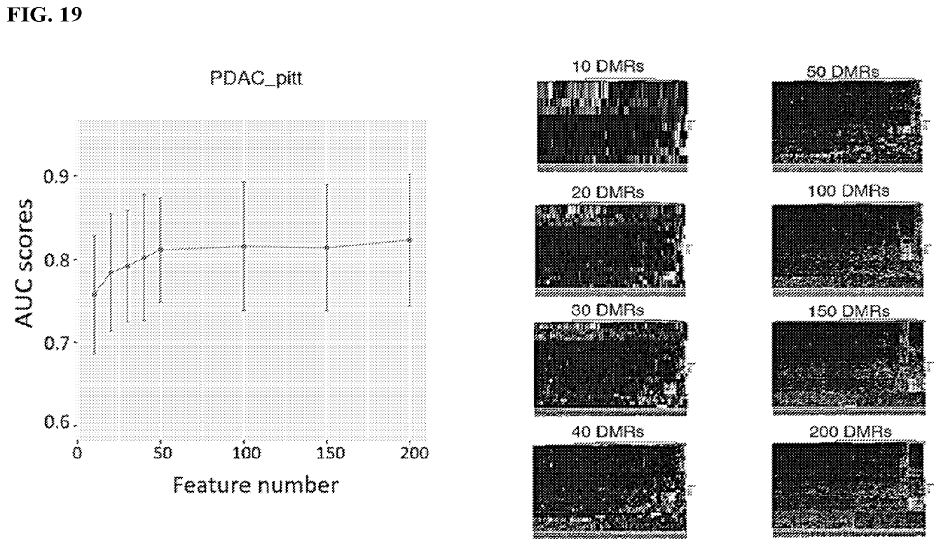

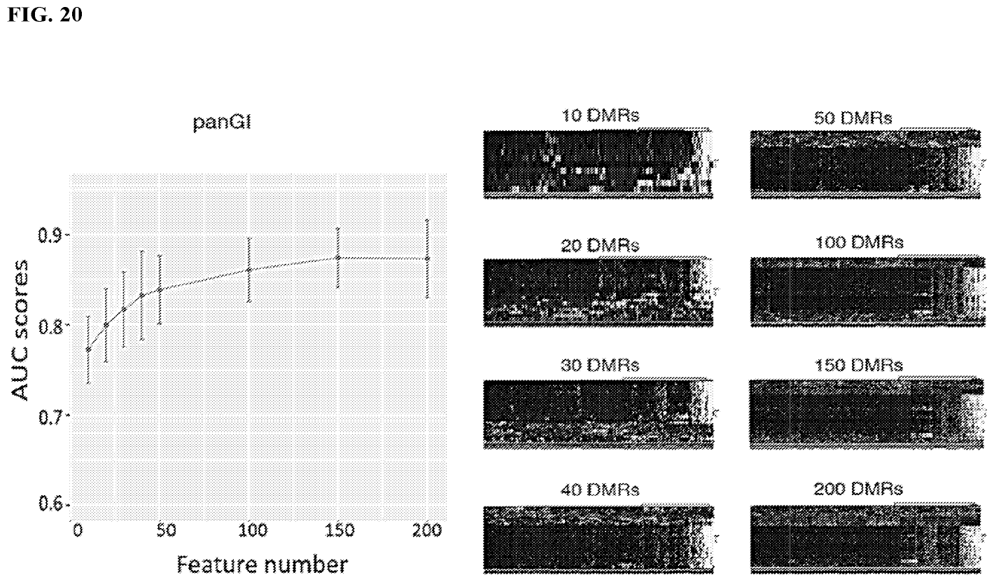

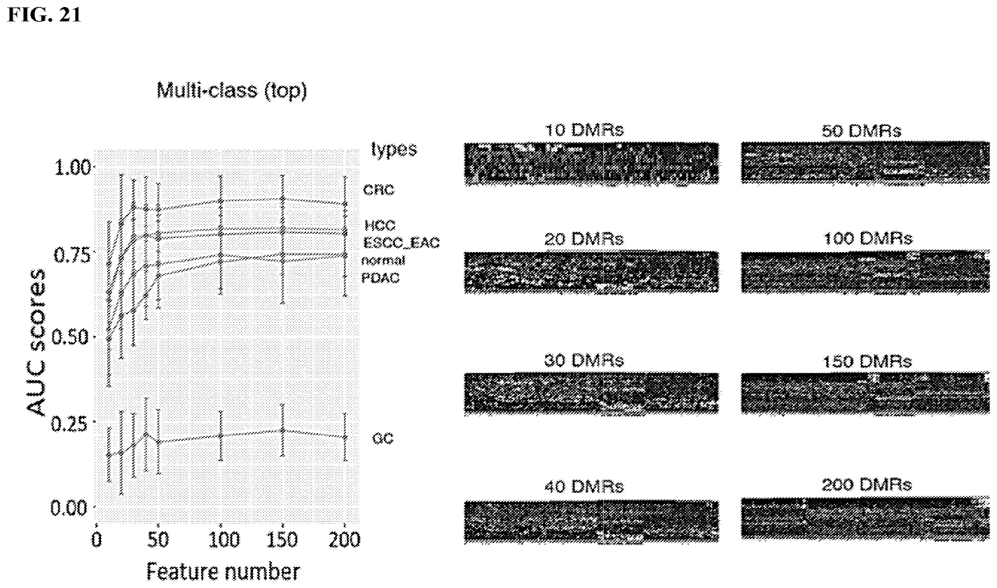

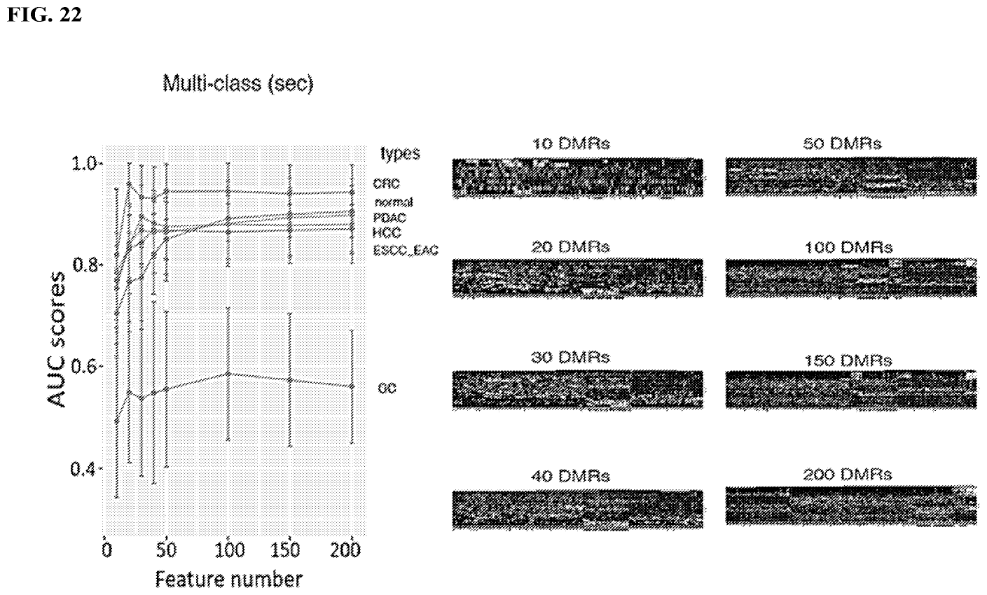

Finally, to assist the development of powerful and cost-effective cfDNA methylation biomarker panels for GI cancer detection, the inventors also evaluated the performance of the inventors' models when different number of informative DMRs were selected for model training. For single-GI cancer prediction models, the top 50 informatic DMRs were sufficient. Even with as few as 10 DMRs, models for HCC or CRC prediction still showed excellent performance with AUC scores more than 0.95 (FIGS. 5A-5C, and 14-19 and Tables PGI, CRC, HCC, ESCC, GC, EAC, PDAC, and MCC). As for panGI or multi GI classification models, optimal performance was achieved with at least the top 150 informative DMRs in this Example (FIGS. 5A-5C and 20-22 and Tables PGI, CRC, HCC, ESCC, GC, EAC, PDAC, and MCC).

Despite the increase in incidence of unscreened cancers, only a handful of cancers like breast, cervical, colon, lung and prostate cancer are screened in the general population. The lack of population-based screening for all cancers is attributed to the low prevalence of many cancers in the general population (3, 18). However, by developing sensitive multi-cancer or multi-organ diagnostic tests; population screening can be implemented even in the low prevalent cancers. In this regard, gastrointestinal cancers provide with a unique opportunity for developing a panGI diagnostic assay. Alquist et al., showed that by developing a panGI diagnostic assay, only 83 patients need to be screened to diagnose one positive patient with GI cancer (3). Here, the inventors performed a comprehensive study by first profiling genome-wide DNA methylation aberrations in all the GI cancer tissues and adjacent normal, followed by development of 30 MB gitBS which included all the significant tissue DMRs identified across GI cancers for a large-scale plasma validation and panel building in 300 plasma samples collected from six different GI cancers. Based on the identified plasma DMRs between GI cancers, machine learning models were trained to identify DMR panels that can detect single GI cancers, pan-GI cancer and also to locate the tissue of origin with high sensitivity and robustness.

Most of the previous studies either studied individual GI cancers (9, 10, 19) or selected a panel of significant tissue markers and subsequently validated in cell-free DNA using PCR based methods (20, 21). Hence, cancer specificity is not well studied, and these studies could not build multi-organ diagnostic assays to implement cost-effective population screening tests. In contrast, the inventors first identified every tissue significant CpG across gastrointestinal cancers and followed by development of plasma specific diagnostic panels for the accurate detection of GI cancers tissue of origin using a single targeted methylation test EpiPanGI Dx. Compared to previous studies (17), the inventors selected fewer number of DMRs for prediction, which makes the inventors' model more feasible for large-scale validation studies and clinical practice (FIGS. 5A-5C and 14-22). In addition, the $70 cost per sample as well as the low 10 ng input cell-free DNA makes the inventors' targeted methylation assay very feasible for clinical use.

Liu et al., identified tissue of origin methylation markers across 50 different cancers. In another study, plasma cfDNA markers were identified using targeted methylation sequencing that can differentiate colorectal cancer, non-small-cell lung cancer, breast cancer and melanoma (22). Shen et al., used cfMeDIP-seq method to discover plasma DMRs that can differentiate multiple solid cancers including pancreatic, colorectal, breast, lung, renal, and bladder cancers (17). However, ours is the first study where organ specific methylation markers are investigated to develop a multi-GI cancer cfDNA assay. Excitingly, the detection accuracy of EpiPanGI Dx assay with as little as 50 DMRs is quite high across all GI cancers considering it is a multi-cancer diagnostic test. Furthermore, the EpiPanGI Dx assay developed from the plasma cell-free DNA showed excellent diagnostic accuracy with an AUC between 0.91-0.99 when applied back to the TCGA GI cancer tissue cohorts. Hence the markers the inventors trained and validated in plasma cell-free DNA are highly cancer specific.

The strength of the inventors' study lies in the identification of GI cancer tissue markers first and then the development of plasma specific DMRs using machine learning algorithms with training and validation sets as well as using 10× cross-validation to compute the accuracy of the EpiPanGI Dx assay across GI cancers. In addition, the assay is quite cost-effective and can be done using 1-2 ml of plasma. Even though the plasma samples were collected from several different parts of the world, the detection accuracy of EpiPanGI Dx in cfDNA as well as the performance of the test in TCGA tissue data shows the robustness of the inventors' markers.

### Materials and Methods

Patients and clinicopathological data: Whole genome 450k tissue DNA methylation data across GI cancers and adjacent normal were obtained from the TCGA and GSE72872 (23).

Complete clinical, epidemiological, molecular, and histopathological data are available at the TCGA website: https://tcga-data.nci.nih.gov/tcga/. The retrospective plasma cfDNA specimens consisting of 300 patients across GI cancers and healthy age matched controls were collected from various institutes. Written informed consent was obtained from all patients and the study were approved by the institutional review boards of all participating institutions.

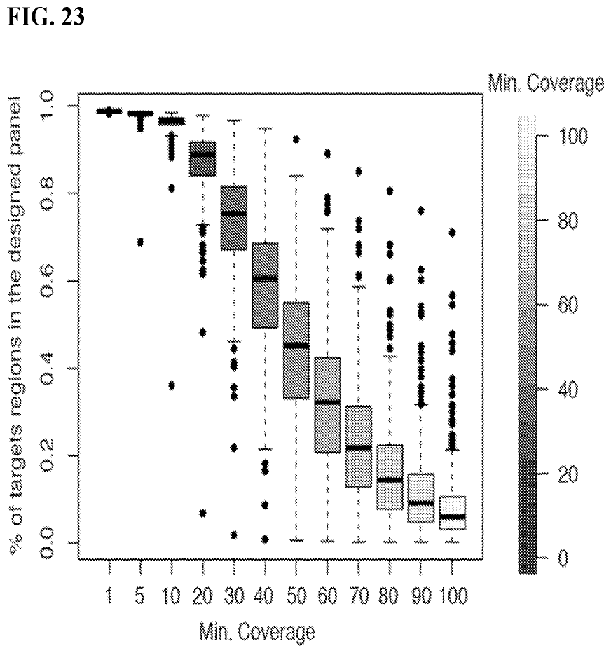

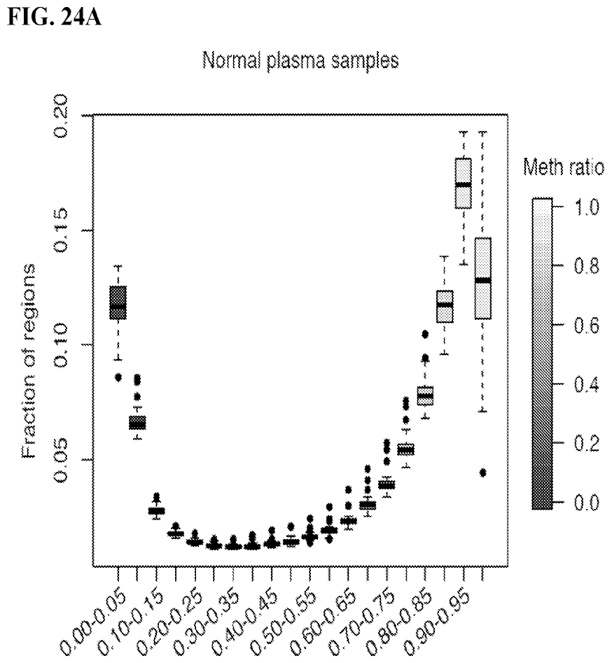

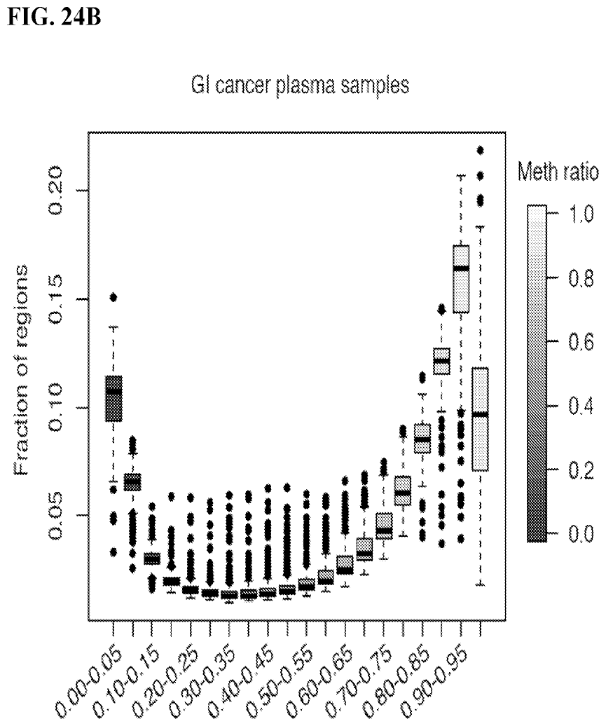

Specimen processing of patient plasma samples: The plasma was transferred to 2-mL microcentrifuge tube and centrifuged at 16,000 g for 10 minutes at 4° C. to remove any cellular debris. Circulating cell-free DNA (10-100 ng) was extracted from 1-2 ml plasma using the QIAamp Circulating Nucleic Acid kit (Qiagen) with slight modifications. At the last step of the protocol, the column filter containing cfDNA was incubated for 5 minutes (instead of 3 minutes) and was eluted with 50 ul of elution buffer (AVE, provided by the manufacturer) twice (instead of one). cfDNA was quantified using the Quant-iT high-sensitivity Picogreen double-stranded DNA Assay Kit (Invitrogen by Thermo Fisher Scientific) according to manufacturer instructions. For targeted methylation sequencing, 10 ng plasma cell-free DNA was first bisulfite treated using the ZYMO Gold Kit per the manufacturer's protocol. The inventors adapted Swift Bioscience Methyl-Seq library preparation kit to generate individual libraries incorporating 13 PCR cycles and overnight ligation. Custom targeted CpG methylation probes were designed using Roche Nimblegen target capture kit, Custom SeqCap Epi Choice 30 MB. Libraries were quantified using Quant-iT high-sensitivity Picogreen double-stranded DNA Assay Kit before equimolarly pooling 10 individual libraries per capture consisting of 2 ug total DNA. Hybridization and capture were performed using VK SeqCap Epi Reagent Kit Plus and SeqCap EZ hybridization/wash kit from Roche Nimblegen following manufacturer recommendations. For blocking, the inventors used universal blocker from IDT technologies. The inventors sequenced the pooled libraries on Illumina NovaSeq S4 using paired-end 100-base-pair reads incorporating 150 individual libraries per lane. Sequencing matrices including the coverage distribution and methylation ratio distribution of gitBS in all plasma samples are included in FIGS. 23 and 24A-24B.

Plasma targeted bisulfite data processing, DMR calling and visualization: For each plasma sample, after trimming adaptor and low-quality bases, the inventors used BSMAP (2.90) to align bisulfite sequencing reads to hg19 human genome assembly. The methylation ratio of CpG site is calculated by the methratio.py script (from BSMAP package). The CpG methylation ratios supported by less than 4 reads were discarded before the downstream analysis. Metilene (0.2-7) is used for calculating de novo DMRs between two conditions, e.g., normal vs. cancer. For each CpG site, at least 3 samples of each condition need to have non-missing value. Missing value is imputed by Metilene during DMR calling. Since methylation difference between normal and cancer tissue is usually diluted in the plasma, the inventors selected DMRs based on a relative loose cut-off (absolute methylation difference more than 0.1 and p-value less than 0.05) for the downstream analysis. Methylation level of a DMR is represented by the mean methylation ratio of its CpG sites. The z-score of DMR methylation level is used for heatmap visualization. The inventors used Ward clustering and Euclidean distance for heatmap plotting.

Machine learning methods used for developing various GI cancer detection panels: Feature selection for Single GI cancer detection and pan-GI cancer detection. For single GI cancer prediction, the normal and cancer plasma samples were randomly partitioned into training set and test set in a 70%-30% manner. DMR identification and feature selection (using ‘Boruta’ R package to select the top 200 informative DMRs) were performed with normal and cancer plasma samples for each GI cancer. Only samples from training set were used for the above steps. For pan-GI cancer detection, the samples from the aforementioned training sets or testing sets for each GI cancer were pooled into a single pan-GI training set or testing set, respectively. DMRs identified from each GI cancers were also pooled with total around 8000 DMRs for feature selection (using ‘Boruta’ R package to select the top 200 informative DMRs). Again, only samples from training set were used for the DMR identification and feature selection.

Feature selection for multi GI cancer classification. Plasma samples from six GI cancers and health people were used for classification analysis. The EAC and ESCC were combined as one class given their high similarity. Plasma samples from each class were randomly partitioned into training set and test set in a 70%-30% manner independently. Class specific DMRs were identified by one-versus-rest comparisons. Finally, around 4000 DMRs identified from all classes were pooled together and the top 200 informative DMRs were selected by using R package ‘Boruta’ with default parameters for the downstream GI cancer classification.

Feature selection with Boruta package. After splitting the data into training and test sets, Boruta package were used to select the most informative DMRs from the training set for cancer detection. Given the randomness introduced by the missing value imputation and random forest construction, the inventors repeated the feature selection step for 50 times and finally choose the top 200 DMRs that were most frequently selected by the Boruta algorithm for the following analysis.

Prediction model training and evaluation. The inventors used training sets to train random forest (R package ‘ranger’) models for single GI cancer prediction, pan-GI cancer prediction and multi GI cancer classification, respectively. The hyperparameters were tuned by 10-fold cross-validation. For model evaluation, the held-out test sets were used to plot the ROC curve and calculate the AUC scores for each random forest model. The training-test set split, DMR calling and feature selection were repeated for 10 times in order to avoid overestimating the model performance.

Independent cohort validation. PDAC patient samples were from two independent cohorts (Pittsburgh cohort and MCW cohort). The inventors used the PDAC Pittsburgh cohort, which has more patient samples, for DMR calling, feature selection (top 200 informative DMRs were selected) and model training. Finally, the AUC scores of this model in detecting cancer was calculated with the PDAC MCW cohort.

Early stage cancer prediction. Late stage (stage IV) cancer and 70% normal plasma samples were used for DMR calling, feature selection (top 200 informative DMRs were selected) and model training. The performance of the trained model was then evaluated with the early stage (stage I-III) cancer samples and the held-out normal samples.

Informative DMRs validation by cancer tissue data. Calculated beta values of 450K methylation array data for TCGA-COAD, TCGA-LIHC, TCGA-ESCA, TCGA-STAD and TCGA-PAAD were downloaded from the UCSC Xena database. Calculated beta values of 450K methylation array data for EAC was downloaded from GEO (GSE72872). The inventors mapped the 450K CpG sites to the informative DMRs selected for single GI cancer detection, pan-GI cancer detection and multi GI cancers classification. The methylation level of the informative DMRs for each cancer tissue sample was calculated by taking the mean of the mapped CpG sites beta values. The normal and cancer tissue samples were partitioned into training and test set in a 70%-30% manner. The inventors trained a random forest model with the training set and calculated the AUC scores of the model with the held-out test set.

While various embodiments and aspects are shown and described herein, it will be obvious to those skilled in the art that such embodiments and aspects are provided by way of example only. Numerous variations, changes, and substitutions will now occur to those skilled in the art without departing from the disclosure.

References: 1. Torre et al, Cancer Epidemiol Biomarkers Prev 25, 16-27 (2016); 2. Smith et al., CA Cancer J Clin 69, 184-210 (2019); 3. Ahlquist, NPJ Precis Oncol 2, 23 (2018); 4. van der Pol et al, Cancer Cell 36, 350-368 (2019); 5. Mroz et al, Cancer 123, 917-927 (2017); 6. Lam et al, Biochim Biophys Acta 1866, 106-120 (2016); 7. Kandimalla et al, Nat Rev Urol 10, 327-335 (2013); 8. Moss et al., Nat Commun 9, 5068 (2018); 9. Xu et al., Nat Mater 16, 1155-1161 (2017); 10. Luo et al., Sci Transl Med 12, (2020); 11. Shen et al., Nature 563, 579-583 (2018); 12. Guo et al., Nat Genet 49, 635-642 (2017); 13. Provenzale et al., J Natl Compr Canc Netw 16, 939-949 (2018); 14. Luo et al., Science Translational Medicine 12, (2020); 15. Degenhardt et al, Briefings in bioinformatics 20, 492-503 (2017); 16. Cohen et al., Science 359, 926-930 (2018); 17. Shen et al., Nature 563, 579 (2018); 18. Cole, et al, J Natl Cancer Inst 64, 1263-1272 (1980); 19. Qin et al., Clin Cancer Res 25, 7396-7404 (2019); 20. Eissa et al., Clin Epigenetics 11, 59 (2019); 21. Freitas et al., J Transl Med 16, 45 (2018); 22. Liu et al., Ann Oncol 29, 1445-1453 (2018); 23. Krause et al., Carcinogenesis 37, 356-365 (2016).

1-42. (canceled)

43. A method of treating cancer in a patient in need thereof, the method comprising:
(a) detecting an increased level of methylated CpG sites, relative to a standard control, within a plurality of gene regions in a DNA sample obtained from the patient; and
(b) administering to the patient an effective amount of radiotherapy, chemotherapy, targeted therapy, immunotherapy, or a combination of two or more thereof, thereby treating the patient for cancer;
wherein:
(i) the cancer is a gastrointestinal cancer, and the plurality of gene regions comprises at least 50 different gene regions in Table PGI;
(ii) the cancer is colorectal cancer, and the plurality of gene regions comprises at least 5 different gene regions in Table CRC;
(iii) the cancer is hepatocellular carcinoma, and the plurality of gene regions comprises at least 5 different gene regions in Table HCC;
(iv) the cancer is esophageal squamous cell carcinoma, and the plurality of gene regions comprises at least 5 different gene regions in Table ESCC;
(v) the cancer is gastric cancer, and the plurality of gene regions comprises at least 5 different gene regions in Table GC;
(vi) the cancer is esophageal adenocarcinoma, and the plurality of gene regions comprises at least 5 different gene regions in Table EAC;
(vii) the cancer is pancreatic ductal adenocarcinoma, and the plurality of gene regions comprises at least 5 different gene regions in Table PDAC; or
(viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

44. A method of detecting an increased level of DNA methylation in a patient at risk of developing a cancer, the method comprising detecting an increased level of methylation of CpG sites relative to a standard control within a plurality of gene regions in a DNA sample from the patient;
wherein:
(i) the cancer is a gastrointestinal cancer and the plurality of gene regions comprises at least 50 different gene regions in Table PGI;
(ii) the cancer is colorectal cancer and the plurality of gene regions comprises at least 5 different gene regions in Table CRC;
(iii) the cancer is hepatocellular carcinoma and the plurality of gene regions comprises at least 5 different gene regions in Table HCC;
(iv) the cancer is esophageal squamous cell carcinoma and the plurality of gene regions comprises at least 5 different gene regions in Table ESCC;
(v) the cancer is gastric cancer and the plurality of gene regions comprises at least 5 different gene regions in Table GC;
(vi) the cancer is esophageal adenocarcinoma and the plurality of gene regions comprises at least 5 different gene regions in Table EAC;
(vii) the cancer is pancreatic ductal adenocarcinoma and the plurality of gene regions comprises at least 5 different gene regions in Table PDAC; or
(viii) the cancer is a gastrointestinal cancer selected from the group consisting of colorectal cancer, hepatic cancer, esophageal cancer, and pancreatic cancer, and the plurality of gene regions comprises at least 50 different gene regions in Table MCC.

45. The method of claim 43, wherein:
(i) the cancer is a gastrointestinal cancer, and the plurality of gene regions comprises at least 100 different gene regions in Table PGI;
(ii) the cancer is colorectal cancer, and the plurality of gene regions comprises at least 10 different gene regions in Table CRC;
(iii) the cancer is hepatocellular carcinoma, and the plurality of gene regions comprises at least 10 different gene regions in Table HCC;
(iv) the cancer is esophageal squamous cell carcinoma, and the plurality of gene regions comprises at least 10 different gene regions in Table ESCC;
(v) the cancer is gastric cancer, and the plurality of gene regions comprises at least 5 different gene regions in Table GC;
(vi) the cancer is esophageal adenocarcinoma, and the plurality of gene regions comprises at least 10 different gene regions in Table EAC;
(vii) the cancer is pancreatic ductal adenocarcinoma, and the plurality of gene regions comprises at least 10 different gene regions in Table PDAC; or
(viii) the cancer is a gastrointestinal cancer selected from the group consisting of colorectal cancer, hepatic cancer, esophageal cancer, and pancreatic cancer, and the plurality of gene regions comprises at least 100 different gene regions in Table MCC.

46. The method of claim 43, wherein:
(i) the cancer is a gastrointestinal cancer, and the plurality of gene regions comprises at least 150 different gene regions in Table PGI;
(ii) the cancer is colorectal cancer, and the plurality of gene regions comprises at least 50 different gene regions in Table CRC;
(iii) the cancer is hepatocellular carcinoma, and the plurality of gene regions comprises at least 50 different gene regions in Table HCC;
(iv) the cancer is esophageal squamous cell carcinoma, and the plurality of gene regions comprises at least 50 different gene regions in Table ESCC;
(v) the cancer is gastric cancer, and the plurality of gene regions comprises at least 5 different gene regions in Table GC;
(vi) the cancer is esophageal adenocarcinoma, and the plurality of gene regions comprises at least 50 different gene regions in Table EAC;
(vii) the cancer is pancreatic ductal adenocarcinoma, and the plurality of gene regions comprises at least 50 different gene regions in Table PDAC; or
(viii) the cancer is a gastrointestinal cancer selected from the group consisting of colorectal cancer, hepatic cancer, esophageal cancer, and pancreatic cancer, and the plurality of gene regions comprises at least 150 different gene regions in Table MCC.

47. The method of claim 43, wherein: (i) the cancer is gastrointestinal cancer.

48. The method of claim 47, wherein the plurality of gene regions comprise the first 50 gene regions in Table PGI.

49. The method of claim 43, wherein: (ii) the cancer is colorectal cancer.

50. The method of claim 49, wherein the plurality of gene regions comprise the first 10 gene regions in Table CRC.

51. The method of claim 43, wherein: (iii) the cancer is hepatocellular carcinoma.

52. The method of claim 51, wherein the plurality of gene regions comprise the first 10 gene regions in Table HCC.

53. The method of claim 43, wherein: (iv) the cancer is esophageal squamous cell carcinoma.

54. The method of claim 53, wherein the plurality of gene regions comprise the first 10 gene regions in Table ESCC.

55. The method of claim 43, wherein: (v) the cancer is gastric cancer.

56. The method of claim 55, wherein the plurality of gene regions comprise the first 10 gene regions in Table GC.

57. The method of claim 43, wherein: (vi) the cancer is esophageal adenocarcinoma.

58. The method of claim 57, wherein the plurality of gene regions comprise the first 10 gene regions in Table EAC.

59. The method of claim 43, wherein: (vii) the cancer is pancreatic ductal adenocarcinoma.

60. The method of claim 59, wherein the plurality of gene regions comprise the first 10 gene regions in Table PDAC.

61. The method of claim 43, where: (viii) the cancer is a gastrointestinal cancer selected from the group consisting of colorectal cancer, hepatic cancer, esophageal cancer, and pancreatic cancer; and wherein the method further comprises identifying the tissue of origin based on the plurality of gene regions having the increased levels of methylated CpG sites, thereby identifying the cancer as colorectal cancer, hepatic cancer, esophageal cancer, and pancreatic cancer.

62. The method of claim 43, wherein the DNA sample is cell-free-DNA.
